SEQUENCE LISTING (7 sequences)

SEQ ID NO: 1 - DNA, 21 bp, Hepatitis B virus
ctgtaccaaaccttcggacgg

SEQ ID NO: 2 - DNA, 18 bp, Hepatitis B virus
aggagaaacgggctgagg

SEQ ID NO: 3 - DNA, 26 bp, Hepatitis B virus
ccatcatcctgggctttcggaaaatt

SEQ ID NO: 4 - DNA, 24 bp, Hepatitis B virus
tgcagaggtgaagcgaagtgcaca

SEQ ID NO: 5 - DNA, 24 bp, Hepatitis B virus
gacgtcctttgtttacgtcccgtc

SEQ ID NO: 6 - DNA, 25 bp, Hepatitis B virus
acggggcgcacctctctttacgcgg

SEQ ID NO: 7 - DNA, 26 bp, Hepatitis B virus
ctccccgtctgtgccttctcatctgc

(19) United States
(12) Patent Application Publication
(10) Pub. No.: US 2022/0363686 A1
(43) Pub. Date: Nov. 17, 2022

(54) NOVEL 6,7-DIHYDRO-4H-PYRAZOLO[1,5-A]PYRAZINE INDOLE-2-CARBOXAMIDES ACTIVE AGAINST THE HEPATITIS B VIRUS (HBV)

(71) Applicant: AiCuris GmbH & Co. KG, Wuppertal (DE)

(72) Inventors: Alastair DONALD, Wuppertal (DE); Andreas URBAN, Sprockhövel (DE); Susanne BONSMANN, Köln (DE); Jasper SPRINGER, Diepenveen (NL); Anita WEGERT, Aldenhofen (DE)

(73) Assignee: AiCuris GmbH & Co. KG, Wuppertal (DE)

(21) Appl. No.: 17/290,357

(22) PCT Filed: Nov. 1, 2019
(86) PCT No.: PCT/EP2019/079965

§ 371 Date: Apr. 30, 2021

(30) Foreign Application Priority Data
Nov. 2, 2018 (EP) 18000876.5

(51) Int. Cl. C07D 487/04; A61P 31/20

(52) U.S. Cl. CPC C07D 487/04; A61P 31/20

(57) ABSTRACT

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes and intermediates for making the compounds.

### TECHNICAL FIELD

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes for making the compounds.

### BACKGROUND OF THE INVENTION

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein to encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

HBV-CP related anti-HBV compounds have been reported. For example, phenylpropenamide derivatives, including compounds named AT-61 and AT-130 (Feld J. et al. Antiviral Res. 2007, 76, 168), and a class of thiazolidin-4-ones from Valeant (WO2006/033995), have been shown to inhibit pre-genomic RNA (pgRNA) packaging.

F. Hoffmann-LA Roche AG have disclosed a series of 3-substituted tetrahydro-pyrazolo[1,5-a]pyrazines for the therapy of HBV (WO2016/113273, WO2017/198744, WO2018/011162, WO2018/011160, WO2018/011163).

Heteroaryldihydropyrimidines (HAPs) were discovered in a tissue culture-based screening (Weber et al., Antiviral Res. 2002, 54, 69). These HAP analogs act as synthetic allosteric activators and are able to induce aberrant capsid formation that leads to degradation of HBV-CP (WO 99/54326, WO 00/58302, WO 01/45712, WO 01/6840). Further HAP analogs have also been described (J. Med. Chem. 2016, 59 (16), 7651-7666).

A subclass of HAPs from F. Hoffman-La Roche also shows activity against HBV (WO2014/184328, WO2015/132276, and WO2016/146598). A similar subclass from Sunshine Lake Pharma also shows activity against HBV (WO2015/144093). Further HAPs have also been shown to possess activity against HBV (WO2013/102655, Bioorg. Med. Chem. 2017, 25(3) pp. 1042-1056, and a similar subclass from Enanta Therapeutics shows similar activity (WO2017/011552). A further subclass from Medshine Discovery shows similar activity (WO2017/076286). A further subclass (Janssen Pharma) shows similar activity (WO2013/102655).

A subclass of pyridazones and triazinones (F. Hoffman-La Roche) also show activity against HBV (WO2016/023877), as do a subclass of tetrahydropyridopyridines (WO2016/177655). A subclass of tricyclic 4-pyridone-3-carboxylic acid derivatives from Roche also show similar anti-HBV activity (WO2017/013046).

A subclass of sulfamoyl-arylamides from Novira Therapeutics (now part of Johnson & Johnson Inc.) also shows activity against HBV (WO2013/006394, WO2013/096744, WO2014/165128, WO2014/184365, WO2015/109130, WO2016/089990, WO2016/109663, WO2016/109684, WO2016/109689, WO2017/059059). A similar subclass of thioether-arylamides (also from Novira Therapeutics) shows activity against HBV (WO2016/089990). Additionally, a subclass of aryl-azepanes (also from Novira Therapeutics) shows activity against HBV (WO2015/073774). A similar subclass of arylamides from Enanta Therapeutics show activity against HBV (WO2017/015451).

Sulfamoyl derivatives from Janssen Pharma have also been shown to possess activity against HBV (WO2014/033167, WO2014/033170, WO2017001655, J. Med. Chem, 2018, 61(14) 6247-6260) A subclass of glyoxamide substituted pyrrolamide derivatives also from Janssen Pharma have also been shown to possess activity against HBV (WO2015/011281). A similar class of glyoxamide substituted pyrrolamides (Gilead Sciences) has also been described (WO2018/039531).

A subclass of sulfamoyl- and oxalyl-heterobiaryls from Enanta Therapeutics also show activity against HBV (WO2016/161268, WO2016/183266, WO2017/015451, WO2017/136403 & US20170253609).

A subclass of aniline-pyrimidines from Assembly Biosciences also show activity against HBV (WO2015/057945, WO2015/172128). A subclass of fused tri-cycles from Assembly Biosciences (dibenzo-thiazepinones, dibenzo-diazepinones, dibenzo-oxazepinones) show activity against HBV (WO2015/138895, WO2017/048950).

A series of cyclic sulfamides has been described as modulators of HBV-CP function by Assembly Biosciences (WO2018/160878).

Arbutus Biopharma have disclosed a series of benzamides for the therapy of HBV (WO2018/052967, WO2018/172852).

It was also shown that the small molecule bis-ANS acts as a molecular ‘wedge’ and interferes with normal capsid-protein geometry and capsid formation (Zlotnick A et al. J. Virol. 2002, 4848).

Problems that HBV direct acting antivirals may encounter are toxicity, mutagenicity, lack of selectivity, poor efficacy, poor bioavailability, low solubility and difficulty of synthesis. There is a thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

### SUMMARY OF THE INVENTION

Provided herein are compounds useful for the treatment or prevention of HBV infection in a subject in need thereof, and intermediates useful in their preparation. The subject matter of the invention is a compound of Formula I:

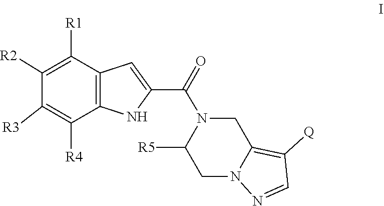

in which
R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C═C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C6-aryl, heteroaryl, C1-C6-alkyl, C1-C6-haloalkyl, C1-C6-alkyl-NH—C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N, wherein C3-C7-heterocycloalkyl is optionally substituted with 1 or 2 amino groups
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen, O-C1-C6-haloalkyl and C≡N.

In one embodiment of the invention subject matter of the invention is a compound of Formula I in which
R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C≡C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, C2-C6-alkyl-O—C1-C6-alkyl, optionally substituted with 1, 2, or 3 groups each independently selected from OH, halo, C3-C7-heterocycloalkyl, C6-aryl, heteroaryl, C1-C6-alkyl, C1-C6-haloalkyl, C1-C6-alkyl-NH—C1-C6-haloalkyl, C1-C6-hydroxyalkyl, C1-C6-alkyl-O-C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N, wherein C3-C7-heterocycloalkyl is optionally substituted with 1 or 2 amino groups
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen, O-C1-C6-haloalkyl and C≡N.

In one embodiment subject matter of the present invention is a compound according to Formula I in which R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C═C, C≡C, C≡N, C(CH₃)₂OH, SCH₃, OH, and OCH₃.

In one embodiment subject matter of the present invention is a compound according to Formula I in which R5 is selected from the group comprising H, and methyl.

In one embodiment subject matter of the present invention is a compound according to Formula I in which Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo.

In one embodiment subject matter of the present invention is a compound according to Formula I in which R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C6-aryl, heteroaryl, C1-C6-alkyl, C1-C6-haloalkyl, C1-C6-alkyl-NH—C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N, wherein C3-C7-heterocycloalkyl is optionally substituted with 1 or 2 amino groups

In one embodiment subject matter of the present invention is a compound according to Formula I in which R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen, O—C1-C6-haloalkyl and C≡N.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula I or a pharmaceutically acceptable salt thereof according to the invention, for use in the prevention or treatment of an HBV infection in subject in need thereof

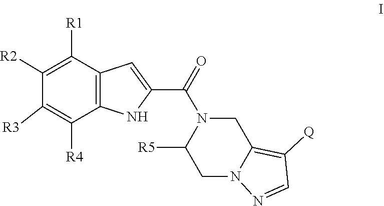

in which
R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C═C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen and C≡N.

In one embodiment of the invention subject matter of the invention is a compound of Formula I in which
R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C═C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen and C≡N.

In one embodiment subject matter of the present invention is a compound according to Formula I in which R1, R2, R3 and R4 are for each position independently selected from the group comprising H, CF₂H, CF₃, CF₂CH₃, F, Cl, Br, CH₃, Et, i-Pr, c-Pr, D, CH₂OH, CH(CH₃)OH, CH₂F, CH(F)CH₃, I, C═C, C≡C, C≡N, C(CH₃)₂OH, SCH₃, OH, and OCH₃.

In one embodiment subject matter of the present invention is a compound according to Formula I in which R5 is selected from the group comprising H, and methyl.

In one embodiment subject matter of the present invention is a compound according to Formula I in which Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo.

In one embodiment subject matter of the present invention is a compound according to Formula I in which R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N.

In one embodiment subject matter of the present invention is a compound according to Formula I in which R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen and C≡N.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula II or a pharmaceutically acceptable salt thereof according to the invention, for use in the prevention or treatment of an HBV infection in subject in need thereof

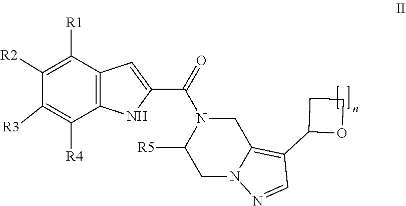

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H and methyl
n is 1, 2 or 3.

In one embodiment subject matter of the present invention is a compound according to Formula II in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃, CF₂CH₃, F, Cl, CH₃, and Et.

In one embodiment subject matter of the present invention is a compound according to Formula II in which R5 is selected from the group comprising H and methyl.

In one embodiment subject matter of the present invention is a compound according to Formula II in which n is 1, 2 or 3.

One embodiment of the invention is a compound of Formula II or a pharmaceutically acceptable salt thereof according to the invention, for use in the prevention or treatment of an HBV infection in subject.

One embodiment of the invention is a pharmaceutical composition comprising a compound of Formula II or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula II or a pharmaceutically acceptable salt thereof according to the present invention.

A further embodiment of the invention is a compound of Formula III or a pharmaceutically acceptable salt thereof according to the invention, for use in the prevention or treatment of an HBV infection in subject in need thereof.

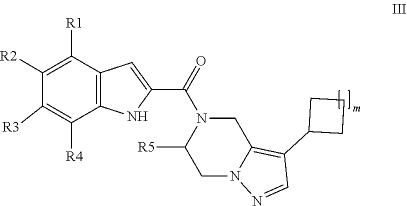

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H, methyl
m is 0, 1, 2 or 3.

In one embodiment subject matter of the present invention is a compound according to Formula III in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃, CF₂CH₃, F, Cl, CH₃, and Et.

In one embodiment subject matter of the present invention is a compound according to Formula III in which R5 is selected from the group comprising H and methyl.

In one embodiment subject matter of the present invention is a compound according to Formula III in which m is 0, 1, 2 or 3.

One embodiment of the invention is a compound of Formula III or a pharmaceutically acceptable salt thereof according to the invention, for use in the prevention or treatment of an HBV infection in subject.

One embodiment of the invention is a pharmaceutical composition comprising a compound of Formula III or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula III or a pharmaceutically acceptable salt thereof according to the present invention.

A further embodiment of the invention is a compound of Formula IV or a pharmaceutically acceptable salt thereof according to the invention, for use in the prevention or treatment of an HBV infection in subject in need thereof.

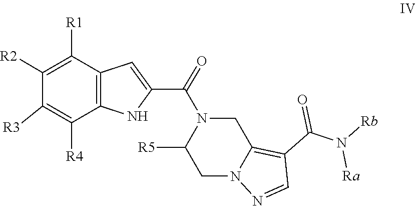

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H, and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N;
R^{a} and R^{b} are optionally connected to form a C3-C7-heterocycloalkyl ring, optionally substituted with 1, 2, or 3 groups selected from OH, halogen and C≡N.

In one embodiment subject matter of the present invention is a compound according to Formula IV in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃. CF₂CH₃, F, Cl, CH₃, and Et.

In one embodiment subject matter of the present invention is a compound according to Formula IV in which R5 is selected from the group comprising H and methyl.

In one embodiment subject matter of the present invention is a compound according to Formula IV in which R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, C1-C6-alkyl-C≡N.

In one embodiment subject matter of the present invention is a compound according to Formula IV in which R^{a} and R^{b} are optionally connected to form a C3-C7-heterocycloalkyl ring, optionally substituted with 1, 2, or 3 groups selected from OH, halogen and C≡N.

One embodiment of the invention is a compound of Formula IV or a pharmaceutically acceptable salt thereof according to the invention, for use in the prevention or treatment of an HBV infection in subject.

One embodiment of the invention is a pharmaceutical composition comprising a compound of Formula IV or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IV or a pharmaceutically acceptable salt thereof according to the present invention.

A further embodiment of the invention is a compound of Formula V or a pharmaceutically acceptable salt thereof according to the invention, for use in the prevention or treatment of an HBV infection in subject in need thereof.

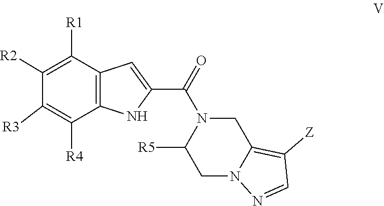

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H and methyl
Z is selected from C₆-C12-aryl and C1-C9-heteroaryl, optionally substituted with 1, 2, 3, or 4 groups each independently selected from —OH, halo, C1-C6-alkyl, C3-C7-cycloalkyl, C1-C6-haloalkyl, C1-C6-alkoxy, C1-C6-hydroxyalkyl, and C≡N.

In one embodiment subject matter of the present invention is a compound according to Formula V in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃, CF₂CH₃, F, Cl, CH₃, and Et.

In one embodiment subject matter of the present invention is a compound according to Formula V in which R5 is selected from the group comprising H and methyl.

In one embodiment subject matter of the present invention is a compound according to Formula V in which Z is selected from C6-C12-aryl and C1-C9-heteroaryl, wherein aryl and heteroaryl are optionally substituted with 1, 2, 3, or 4 groups each independently selected from —OH, halo, C1-C6-alkyl, C3-C7-cycloalkyl, C1-C6-haloalkyl, C1-C6-alkoxy, C1-C6-hydroxyalkyl, and C≡N.

One embodiment of the invention is a compound of Formula V or a pharmaceutically acceptable salt thereof according to the invention, for use in the prevention or treatment of an HBV infection in subject.

One embodiment of the invention is a pharmaceutical composition comprising a compound of Formula V or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula V or a pharmaceutically acceptable salt thereof according to the present invention.

A further embodiment of the invention is a compound of Formula VI or a pharmaceutically acceptable salt thereof according to the invention, for use in the prevention or treatment of an HBV infection in subject in need thereof.

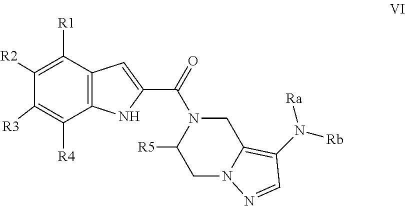

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring, optionally substituted with 1, 2, or 3 groups selected from OH, halogen and C≡N.

In one embodiment subject matter of the present invention is a compound according to Formula VI in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃, CF₂CH₃, F, Cl, CH₃, and Et.

In one embodiment subject matter of the present invention is a compound according to Formula VI in which R5 is selected from the group comprising H and methyl.

In one embodiment subject matter of the present invention is a compound according to Formula VI in which Ra and Rb are selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N.

In one embodiment subject matter of the present invention is a compound according to Formula VI in which R^{a} and R^{b} are optionally connected to form a C3-C7-heterocycloalkyl ring, optionally substituted with 1, 2, or 3 groups selected from OH, halogen and C≡N.

One embodiment of the invention is a compound of Formula VI or a pharmaceutically acceptable salt thereof according to the invention, for use in the prevention or treatment of an HBV infection in subject.

One embodiment of the invention is a pharmaceutical composition comprising a compound of Formula VI or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VI or a pharmaceutically acceptable salt thereof according to the present invention.

A further embodiment of the invention is a compound of Formula VII or a pharmaceutically acceptable salt thereof according to the invention, for use in the prevention or treatment of an HBV infection in subject in need thereof

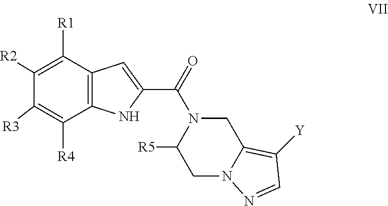

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H and methyl
Y is oxooxadiazabicyclo[3.3.1]nonanyl substituted by C1-C6-carboxyalkyl; or oxopyrrolidinyl, said oxopyrrolidinyl optionally being once substituted by N(C1-C6-carboxyalkyl)(C1-C6-alkyl), carboxyphenyl, carboxypyridinyl, carboxyphenylamino, halocarboxyphenyl or carboxypyrrolidinyl, or twice substituted by carboxypyrrolidinyl and C1-C6-alkyl.

In one embodiment subject matter of the present invention is a compound according to Formula VII in which R1, R2, R3 and R4 are for each position independently selected from the group comprising H, CF₂H, CF₃, CF₂CH₃, F, Cl, Br, CH₃, Et, i-Pr, c-Pr, D, and CH₂OH, preferably H, CF₂H, CF₃, CF₂CH₃, F, Cl, CH₃, and Et.

In one embodiment subject matter of the present invention is a compound according to Formula VII in which R5 is selected from the group comprising H and methyl.

In one embodiment subject matter of the present invention is a compound according to Formula VII in which Y is is oxooxadiazabicyclo[3.3.1]nonanyl substituted by C1-C6-carboxyalkyl; or oxopyrrolidinyl, said oxopyrrolidinyl optionally being once substituted by N(C1-C6-carboxyalkyl)(C1-C6-alkyl), carboxyphenyl, carboxypyridinyl, carboxyphenylamino, halocarboxyphenyl or carboxypyrrolidinyl, or twice substituted by carboxypyrrolidinyl and C1-C6-alkyl.

One embodiment of the invention is a compound of Formula VII or a pharmaceutically acceptable salt thereof according to the invention, for use in the prevention or treatment of an HBV infection in subject.

One embodiment of the invention is a pharmaceutical composition comprising a compound of Formula VII or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VII or a pharmaceutically acceptable salt thereof according to the present invention.

A further embodiment of the invention is a compound of Formula VIII or a pharmaceutically acceptable salt thereof according to the invention, for use in the prevention or treatment of an HBV infection in subject in need thereof

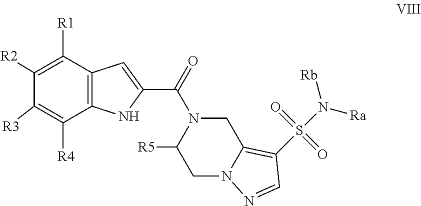

in which
R1, R2, R3 and R4 are for each position independently selected from the group comprising H, CF₂H, CF₃, CF₂CH₃, F, Cl, Br, CH₃, Et, i-Pr, c-Pr, D, and CH₂OH
R5 is selected from H and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring, optionally substituted with 1, 2, or 3 groups selected from OH, halogen and C≡N.

In one embodiment subject matter of the present invention is a compound according to Formula VIII in which R1, R2, R3 and R4 are for each position independently selected from the group comprising H, CF₂H, CF₃, CF₂CH₃, F, Cl, Br, CH₃, Et, and i-Pr, preferably H, CF₂H, CF₃, CF₂CH₃, F, Cl, CH₃, and Et.

In one embodiment subject matter of the present invention is a compound according to Formula VIII in which R5 is selected from the group comprising H and methyl.

In one embodiment subject matter of the present invention is a compound according to Formula VIII in which R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N.

In one embodiment subject matter of the present invention is a compound according to Formula VIII in which R^{a} and R^{b} are optionally connected to form a C3-C7-heterocycloalkyl ring, optionally substituted with 1, 2, or 3 groups selected from OH, halogen and C≡N.

One embodiment of the invention is a compound of Formula VIII or a pharmaceutically acceptable salt thereof according to the invention, for use in the prevention or treatment of an HBV infection in subject.

One embodiment of the invention is a pharmaceutical composition comprising a compound of Formula VIII or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIII or a pharmaceutically acceptable salt thereof according to the present invention.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof. All before mentioned doses refer to daily doses per patient.

In general it is contemplated that an antiviral effective daily amount would be from about 0.01 to about 50 mg/kg, or about 0.01 to about 30 mg/kg body weight. It maybe appropriate to administer the required dose as two, three, four or more sub-doses at appropriate intervals throughout the day. Said sub-doses may be formulated as unit dosage forms, for example containing about 1 to about 500 mg, or about 1 to about 300 mg or about 1 to about 100 mg, or about 2 to about 50 mg of active ingredient per unit dosage form.

The compounds of the invention may, depending on their structure, exist as salts, solvates or hydrates. The invention therefore also encompasses the salts, solvates or hydrates and respective mixtures thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

### Definitions

Listed below are definitions of various terms used to describe this invention. These definitions apply to the terms as they are used throughout this specification and claims unless otherwise limited in specific instances either individually or as part of a larger group.

Unless defined otherwise all technical and scientific terms used herein generally have the same meaning as commonly understood by one of ordinary skill in the art to which this invention belongs. Generally the nomenclature used herein and the laboratory procedures in cell culture, molecular genetics, organic chemistry and peptide chemistry are those well known and commonly employed in the art.

As used herein the articles “a” and “an” refer to one or to more than one (i.e. to at least one) of the grammatical object of the article. By way of example, “an element” means one element or more than one element. Furthermore, use of the term “including” as well as other forms such as “include”, “includes” and “included”, is not limiting.

As used herein the term “capsid assembly modulator” refers to a compound that disrupts or accelerates or inhibits or hinders or delays or reduces or modifies normal capsid assembly (e.g. during maturation) or normal capsid disassembly (e.g. during infectivity) or perturbs capsid stability, thereby inducing aberrant capsid morphology or aberrant capsid function. In one embodiment, a capsid assembly modulator accelerates capsid assembly or disassembly thereby inducing aberrant capsid morphology. In another embodiment a capsid assembly modulator interacts (e.g. binds at an active site, binds at an allosteric site or modifies and/or hinders folding and the like), with the major capsid assembly protein (HBV-CP), thereby disrupting capsid assembly or disassembly. In yet another embodiment a capsid assembly modulator causes a perturbation in the structure or function of HBV-CP (e.g. the ability of HBV-CP to assemble, disassemble, bind to a substrate, fold into a suitable conformation or the like which attenuates viral infectivity and/or is lethal to the virus).

As used herein the term “treatment” or “treating” is defined as the application or administration of a therapeutic agent i.e., a compound of the invention (alone or in combination with another pharmaceutical agent) to a patient, or application or administration of a therapeutic agent to an isolated tissue or cell line from a patient (e.g. for diagnosis or ex vivo applications) who has an HBV infection, a symptom of HBV infection, or the potential to develop an HBV infection with the purpose to cure, heal, alleviate, relieve, alter, remedy, ameliorate, improve or affect the HBV infection, the symptoms of HBV infection or the potential to develop an HBV infection. Such treatments may be specifically tailored or modified based on knowledge obtained from the field of pharmacogenomics.

As used herein the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein the term “patient”, “individual” or “subject” refers to a human or a non-human mammal. Non-human mammals include for example livestock and pets such as ovine, bovine, porcine, feline, and murine mammals. Preferably the patient, subject, or individual is human.

As used herein the terms “effective amount”, “pharmaceutically effective amount”, and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “pharmaceutically acceptable salt” refers to derivatives of the disclosed compounds wherein the parent compound is modified by converting an existing acid or base moiety to its salt form. Examples of pharmaceutically acceptable salts include but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent or in a mixture of the two; generally nonaqueous media like ether, ethyl acetate, ethanol, isopropanol, or acetonitrile are preferred. Lists of suitable salts are found in Remington's Pharmaceutical Sciences 17^{th} ed. Mack Publishing Company, Easton, Pa., 1985 p. 1418 and Journal of Pharmaceutical Science, 66, 2 (1977), each of which is incorporated herein by reference in its entirety.

As used herein the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent, solvent or encapsulating material involved in carrying or transporting a compound useful within the invention within or to the patient such that it may perform its intended function. Typically such constructs are carried or transported from one organ, or portion of the body, to another organ or portion of the body. Each carrier must be “acceptable” in the sense of being compatible with the other ingredients of the formulation including the compound use within the invention and not injurious to the patient. Some examples of materials that may serve as pharmaceutically acceptable carriers include: sugars, such as lactose, glucose and sucrose; starches such as corn starch and potato starch; cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar, buffering agents, such as magnesium hydroxide and aluminium hydroxide; surface active agents; alginic acid; pyrogen-free water, isotonic saline; Ringer's solution; ethyl alcohol; phosphate buffer solutions and other non-toxic compatible substances employed in pharmaceutical formulations.

As used herein “pharmaceutically acceptable carrier” also includes any and all coatings, antibacterial and antifungal agents and absorption delaying agents and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described for example in Remington's Pharmaceutical Sciences (Genaro, Ed., Mack Publishing Company, Easton, Pa., 1985) which is incorporated herein by reference.

As used herein, the term “substituted” means that an atom or group of atoms has replaced hydrogen as the substituent attached to another group.

As used herein, the term “comprising” also encompasses the option “consisting of”.

As used herein, the term “alkyl” by itself or as part of another substituent means, unless otherwise stated, a straight or branched chain hydrocarbon having the number of carbon atoms designated (i.e. C1-C6-alkyl means one to six carbon atoms) and includes straight and branched chains. Examples include methyl, ethyl, propyl, isopropyl, butyl, isobutyl, tert-butyl, pentyl, neopentyl, and hexyl. In addition, the term “alkyl” by itself or as part of another substituent can also mean a C1-C3 straight chain hydrocarbon substituted with a C3-C5-carbocylic ring. Examples include (cyclopropyl)methyl, (cyclobutyl)methyl and (cyclopentyl)methyl. For the avoidance of doubt, where two alkyl moieties are present in a group, the alkyl moieties may be the same or different.

As used herein the term “alkenyl” denotes a monovalent group derived from a hydrocarbon moiety containing at least two carbon atoms and at least one carbon-carbon double bond of either E or Z stereochemistry. The double bond may or may not be the point of attachment to another group. Alkenyl groups (e.g. C2-C8-alkenyl) include, but are not limited to for example ethenyl, propenyl, prop-1-en-2-yl, butenyl, methyl-2-buten-1-yl, heptenyl and octenyl. For the avoidance of doubt, where two alkenyl moieties are present in a group, the alkyl moieties may be the same or different.

As used herein, a C2-C6-alkynyl group or moiety is a linear or branched alkynyl group or moiety containing from 2 to 6 carbon atoms, for example a C2-C4 alkynyl group or moiety containing from 2 to 4 carbon atoms. Exemplary alkynyl groups include —C≡CH or —CH₂—C≡C, as well as 1- and 2-butynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, the term “halo” or “halogen” alone or as part of another substituent means unless otherwise stated a fluorine, chlorine, bromine, or iodine atom, preferably fluorine, chlorine, or bromine, more preferably fluorine or chlorine. For the avoidance of doubt, where two halo moieties are present in a group, they may be the same or different.

As used herein, a C1-C6-alkoxy group or C2-C6-alkenyloxy group is typically a said C1-C6-alkyl (e.g. a C1-C4 alkyl) group or a said C2-C6-alkenyl (e.g. a C2-4 alkenyl) group respectively which is attached to an oxygen atom.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. Preferred examples are phenyl (e.g. C6-aryl) and biphenyl (e.g. C12-aryl). In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” and “heteroaromatic” refer to a heterocycle having aromatic character containing one or more rings (typically one, two or three rings). Heteroaryl substituents may be defined by the number of carbon atoms e.g. C1-C9-heteroaryl indicates the number of carbon atoms contained in the heteroaryl group without including the number of heteroatoms. For example a C1-C9-heteroaryl will include an additional one to four heteroatoms. A polycyclic heteroaryl may include one or more rings that are partially saturated. Non-limiting examples of heteroaryls include:

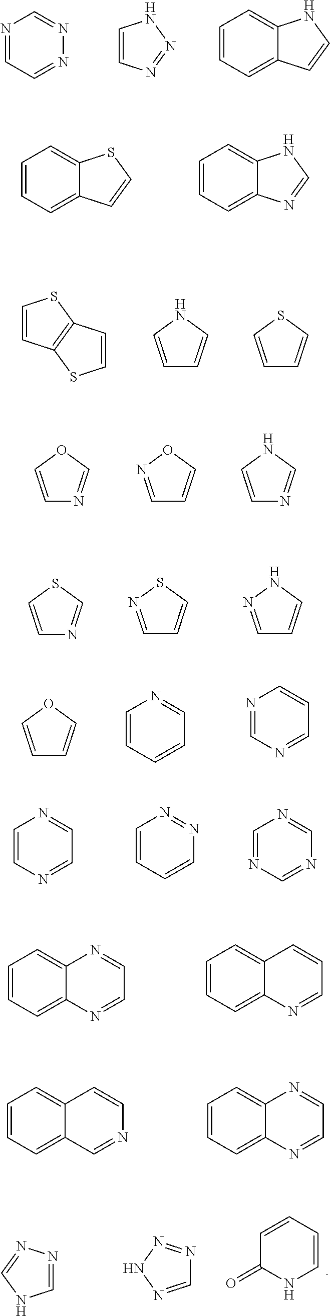

Additional non-limiting examples of heteroaryl groups include pyridyl, pyrazinyl, pyrimidinyl (including e.g. 2- and 4-pyrimidinyl), pyridazinyl, thienyl, furyl, pyrrolyl (including e.g., 2-pyrrolyl), imidazolyl, thiazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Non-limiting examples of polycyclic heterocycles and heteroaryls include indolyl (including 3-, 4-, 5-, 6- and 7-indolyl), indolinyl, quinolyl, tetrahydroquinolyl, isoquinolyl (including, e.g. 1- and 5-isoquinolyl), 1,2,3,4-tetrahydroisoquinolyl, cinnolinyl, quinoxalinyl (including, e.g 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g. 3-, 4-, 5-, 6-, and 7-benzofuryl), 2,3-dihydrobenzofuryl, 1,2-benzisoxazolyl, benzothienyl (including e.g. 3-, 4-, 5-, 6-, and 7-benzothienyl), benzoxazolyl, benzothiazolyl (including e.g. 2-benzothiazolyl and 5-benzothiazolyl), purinyl, benzimidazolyl (including e.g., 2-benzimidazolyl), benzotriazolyl, thioxanthinyl, carbazolyl, carbolinyl, acridinyl, pyrrolizidinyl and quinolizidinyl.

As used herein the term “haloalkyl” is typically a said alkyl, alkenyl, alkoxy or alkenoxy group respectively wherein any one or more of the carbon atoms is substituted with one or more said halo atoms as defined above. Haloalkyl embraces monohaloalkyl, dihaloalkyl, and polyhaloalkyl radicals. The term “haloalkyl” includes but is not limited to fluoromethyl, 1-fluoroethyl, difluoromethyl, 2,2-difluoroethyl, 2,2,2-trifluoroethyl, trifluoromethyl, chloromethyl, dichloromethyl, trichloromethyl, pentafluoroethyl, difluoromethoxy, and trifluoromethoxy.

As used herein, a C1-C6-hydroxyalkyl group is a said C1-C6 alkyl group substituted by one or more hydroxy groups. Typically, it is substituted by one, two or three hydroxyl groups. Preferably, it is substituted by a single hydroxy group.

As used herein, a C1-C6-aminoalkyl group is a said C1-C6 alkyl group substituted by one or more amino groups. Typically, it is substituted by one, two or three amino groups. Preferably, it is substituted by a single amino group.

As used herein, a C1-C6-carboxyalkyl group is a said C1-C4 alkyl group substituted by carboxyl group.

As used herein, a C1-C4-carboxamidoalkyl group is a said C1-C4 alkyl group substituted by a substituted or unsubstituted carboxamide group.

As used herein, a C1-C4-acylsulfonamido-alkyl group is a said C1-C4 alkyl group substituted by an acylsulfonamide group of general formula C(═O)NHSO₂CH₃ or C(═O)NHSO₂-c-Pr.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl) and groups having 3 to 6 ring atoms (C3-C6-cycloalkyl). Illustrative examples of cycloalkyl groups include, but are not limited to the following moieties:

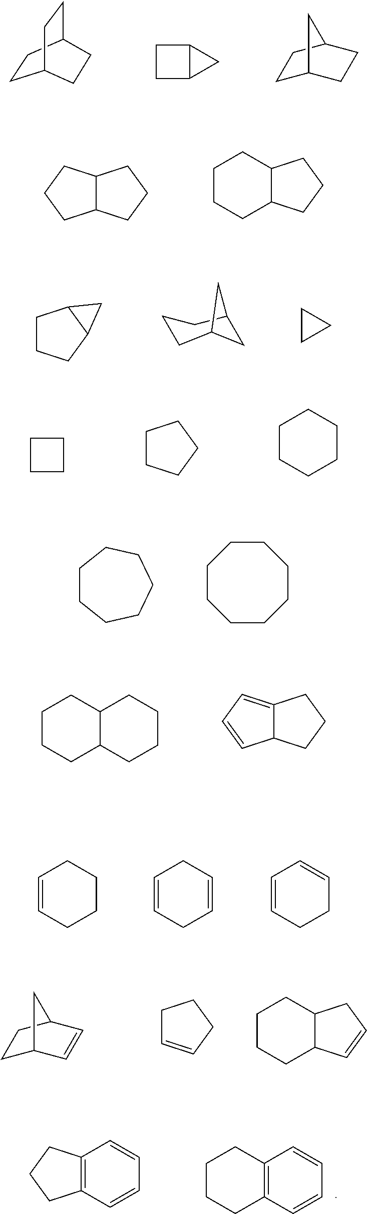

Monocyclic cycloalkyls include but are not limited to cyclopropyl, cyclobutyl, cyclopentyl, cyclohexyl, cycloheptyl, and cyclooctyl. Dicyclic cycloalkyls include but are not limited to tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups both of which refer to a nonaromatic carbocycle as defined herein which contains at least one carbon-carbon double bond or one carbon-carbon triple bond.

As used herein, the term “spirocyclic” refers to any compound containing two or more rings wherein two of the rings have one ring carbon in common.

As used herein the terms “heterocycloalkyl” and “heterocyclyl” refer to a heteroalicyclic group containing one or more rings (typically one, two or three rings), that contains one to four ring heteroatoms each selected from oxygen, sulfur and nitrogen. In one embodiment each heterocyclyl group has from 3 to 10 atoms in its ring system with the proviso that the ring of said group does not contain two adjacent oxygen or sulfur atoms. In one embodiment each heterocyclyl group has a fused bicyclic ring system with 3 to 10 atoms in the ring system, again with the proviso that the ring of said group does not contain two adjacent oxygen or sulfur atoms. In one embodiment each heterocyclyl group has a bridged bicyclic ring system with 3 to 10 atoms in the ring system, again with the proviso that the ring of said group does not contain two adjacent oxygen or sulfur atoms.

In one embodiment each heterocyclyl group has a spiro-bicyclic ring system with 3 to 10 atoms in the ring system, again with the proviso that the ring of said group does not contain two adjacent oxygen or sulfur atoms. Heterocyclyl substituents may be alternatively defined by the number of carbon atoms e.g. C2-C8-heterocyclyl indicates the number of carbon atoms contained in the heterocyclic group without including the number of heteroatoms. For example a C2-C8-heterocyclyl will include an additional one to four heteroatoms. In another embodiment the heterocycloalkyl group is fused with an aromatic ring. In another embodiment the heterocycloalkyl group is fused with a heteroaryl ring. In one embodiment the nitrogen and sulfur heteroatoms may be optionally oxidized and the nitrogen atom may be optionally quaternized. The heterocyclic system may be attached, unless otherwise stated, at any heteroatom or carbon atom that affords a stable structure. An example of a 3-membered heterocyclyl group includes and is not limited to aziridine. Examples of 4-membered heterocycloalkyl groups include, and are not limited to azetidine and a beta-lactam. Examples of 5-membered heterocyclyl groups include, and are not limited to pyrrolidine, oxazolidine and thiazolidinedione. Examples of 6-membered heterocycloalkyl groups include, and are not limited to, piperidine, morpholine, piperazine, N-acetylpiperazine and N-acetylmorpholine. Other non-limiting examples of heterocyclyl groups are

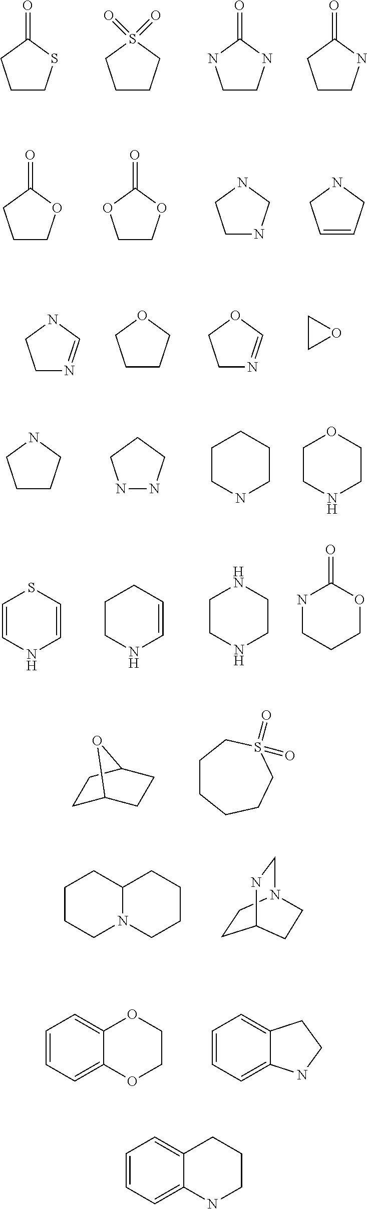

Examples of heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, 1,3-dioxolane, homopiperazine, homopiperidine, 1,3-dioxepane, 47-dihydro-1,3-dioxepin, and hexamethyleneoxide. The terms “C3-C7-heterocycloalkyl” includes but is not limited to tetrahydrofuran-2-yl, tetrahydrofuran-3-yl, 3-oxabicyclo[3.1.0]hexan-6-yl, 3-azabicyclo[3.1.0]hexan-6-yl, tetrahydropyran-4-yl, tetrahydropyran-3-yl, tetrahydropyran-2-yl, and azetidin-3-yl.

As used herein, the term “aromatic” refers to a carbocycle or heterocycle with one or more polyunsaturated rings and having aromatic character i.e. having (4n+2) delocalized π(pi) electrons where n is an integer.

As used herein, the term “acyl”, employed alone or in combination with other terms, means, unless otherwise stated, to mean to an alkyl, cycloalkyl, heterocycloalkyl, aryl or heteroaryl group linked via a carbonyl group.

As used herein, the terms “carbamoyl” and “substituted carbamoyl”, employed alone or in combination with other terms, means, unless otherwise stated, to mean a carbonyl group linked to an amino group optionally mono or di-substituted by hydrogen, alkyl, cycloalkyl, heterocycloalkyl, aryl or heteroaryl. In some embodiments, the nitrogen substituents will be connected to form a heterocyclyl ring as defined above.

As used herein, the term “carboxy” and by itself or as part of another substituent means, unless otherwise stated, a group of formula C(═O)OH.

As used herein, the term “carboxyl ester” by itself or as part of another substituent means, unless otherwise stated, a group of formula C(═O)OX, wherein X is selected from the group consisting of C1-C6-alkyl, C3-C7-cycloalkyl, and aryl.

As used herein the term “prodrug” represents a derivative of a compound of Formula I or Formula II or Formula III or Formula IV or Formula V or Formula VI or Formula VII or Formula VIII which is administered in a form which, once administered, is metabolised in vivo into an active metabolite also of Formula I or Formula II or Formula III or Formula IV or Formula V or Formula VI or Formula VII or Formula VIII.

Various forms of prodrug are known in the art. For examples of such prodrugs see: Design of Prodrugs, edited by H. Bundgaard, (Elsevier, 1985) and Methods in Enzymology, Vol. 42, p. 309-396, edited by K. Widder, et al. (Academic Press, 1985); A Textbook of Drug Design and Development, edited by Krogsgaard-Larsen and H. Bundgaard, Chapter 5 “Design and Application of Prodrugs” by H. Bundgaard p. 113-191 (1991); H. Bundgaard, Advanced Drug Delivery Reviews 8, 1-38 (1992); H. Bundgaard, et al., Journal of Pharmaceutical Sciences, 77, 285 (1988); and N. Kakeya, et al., Chem. Pharm. Bull., 32, 692 (1984).

Examples of prodrugs include cleavable esters of compounds of Formula I, II, III, IV, V, VI, VII and VIII.

An in vivo cleavable ester of a compound of the invention containing a carboxy group is, for example, a pharmaceutically acceptable ester which is cleaved in the human or animal body to produce the parent acid. Suitable pharmaceutically acceptable esters for carboxy include C1-C6 alkyl ester, for example methyl or ethyl esters; C1-C6 alkoxymethyl esters, for example methoxymethyl ester, C1-C6 acyloxymethyl esters; phthalidyl esters; C3-C8 cycloalkoxycarbonyloxyC1-C6 alkyl esters, for example 1-cyclohexylcarbonyloxyethyl; 1-3-dioxolan-2-ylmethylesters, for example 5-methyl-1,3-dioxolan-2-ylmethyl; C1-C6 alkoxycarbonyloxyethyl esters, for example 1-methoxycarbonyloxyethyl; aminocarbonylmethyl esters and mono-or di-N—(C1-C6 alkyl) versions thereof, for example N, N-dimethylaminocarbonylmethyl esters and N-ethylaminocarbonylmethyl esters; and may be formed at any carboxy group in the compounds of the invention.

An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include C1-C6-acyl esters, for example acetyl esters; and benzoyl esters wherein the phenyl group may be substituted with aminomethyl or N-substituted mono-or di-C1-C6 alkyl aminomethyl, for example 4-aminomethylbenzoyl esters and 4-N,N-dimethylaminomethylbenzoyl esters.

Preferred prodrugs of the invention include acetyloxy and carbonate derivatives. For example, a hydroxy group of compounds of Formula I, II, III, IV, V, VI, VII and VIII can be present in a prodrug as —O—CORⁱ or —O—C(O)ORⁱ where Rⁱ is unsubstituted or substituted C1-C4 alkyl. Substituents on the alkyl groups are as defined earlier. Preferably the alkyl groups in Rⁱ is unsubstituted, preferable methyl, ethyl, isopropyl or cyclopropyl.

Other preferred prodrugs of the invention include amino acid derivatives. Suitable amino acids include α-amino acids linked to compounds of Formula I, II, III, IV, V, VI, VII and VIII via their C(O)OH group. Such prodrugs cleave in vivo to produce compounds of Formula I bearing a hydroxy group. Accordingly, such amino acid groups are preferably employed positions of Formula I, II, III, IV, V, VI, VII and VIII where a hydroxy group is eventually required. Exemplary prodrugs of this embodiment of the invention are therefore compounds of Formula I, II, III, IV, V, VI, VII and VIII bearing a group of Formula —OC(O)—CH(NH₂)Rⁱⁱ where Rⁱⁱ is an amino acid side chain. Preferred amino acids include glycine, alanine, valine and serine. The amino acid can also be functionalised, for example the amino group can be alkylated. A suitable functionalised amino acid is N,N-dimethylglycine. Preferably the amino acid is valine.

Other preferred prodrugs of the invention include phosphoramidate derivatives. Various forms of phosphoramidate prodrugs are known in the art. For example of such prodrugs see Serpi et al., Curr. Protoc. Nucleic Acid Chem. 2013, Chapter 15, Unit 15.5 and Mehellou et al., ChemMedChem, 2009, 4 pp. 1779-1791. Suitable phosphoramidates include (phenoxy)-α-amino acids linked to compounds of Formula I, II, III, IV, V, VI, VII and VIII via their —OH group. Such prodrugs cleave in vivo to produce compounds of Formula I bearing a hydroxy group. Accordingly, such phosphoramidate groups are preferably employed positions of Formula I, II, III, IV, V, VI, VII and VIII where a hydroxy group is eventually required. Exemplary prodrugs of this embodiment of the invention are therefore compounds of Formula I bearing a group of Formula —OP(O)(ORⁱⁱⁱ)R^{iv} where Rⁱⁱⁱ is alkyl, cycloalkyl, aryl or heteroaryl, and R^{iv} is a group of Formula —NH—CH(R^{v})C(O)OR^{vi}. wherein R^{v} is an amino acid side chain and R^{vi} is alkyl, cycloalkyl, aryl or heterocyclyl. Preferred amino acids include glycine, alanine, valine and serine. Preferably the amino acid is alanine. R^{v} is preferably alkyl, most preferably isopropyl.

Subject matter of the present invention is also a method of preparing the compounds of the present invention. Subject matter of the invention is, thus, a method for the preparation of a compound of Formula I according to the present invention by reacting a compound of Formula IX

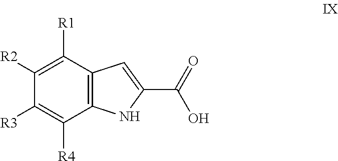

in which R1, R2, R3 and R4 are as above-defined, with a compound of Formula X

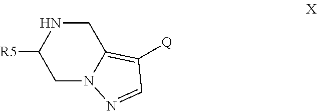

in which Q is as above-defined.

### EXAMPLES

The invention is now described with reference to the following Examples. These Examples are provided for the purpose of illustration only, and the invention is not limited to these Examples, but rather encompasses all variations that are evident as a result of the teachings provided herein.

The required substituted indole-2-carboxylic acids may be prepared in a number of ways; the main routes employed being outlined in Schemes 1-4. To the chemist skilled in the art it will be apparent that there are other methodologies that will also achieve the preparation of these intermediates.

Substituted indole-2-carboxylic acids can be prepared via the Hemetsberger-Knittel reaction (Organic Letters, 2011, 13(8) pp. 2012-2014, Journal of the American Chemical Society, 2007, pp. 7500-7501, and Monatshefte für Chemie, 103(1), pp. 194-204) (Scheme 1).

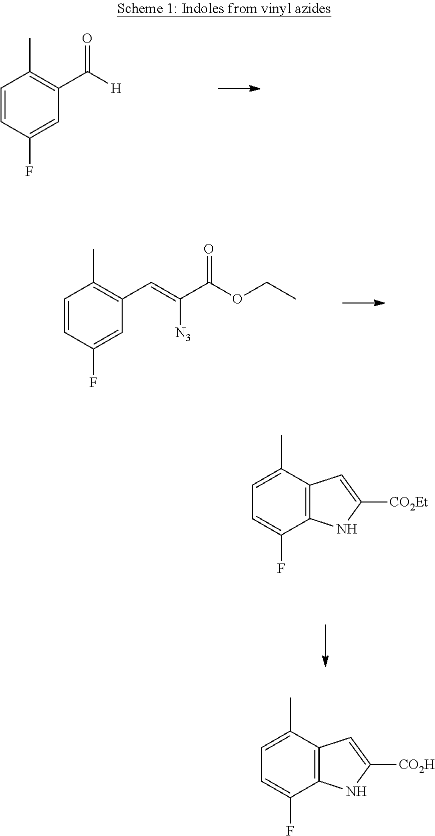

Substituted indoles may also be prepared using the Fischer method (Berichte der Deutschen Chemischen Gesellschaft. 17 (1): 559-568) (Scheme 2).

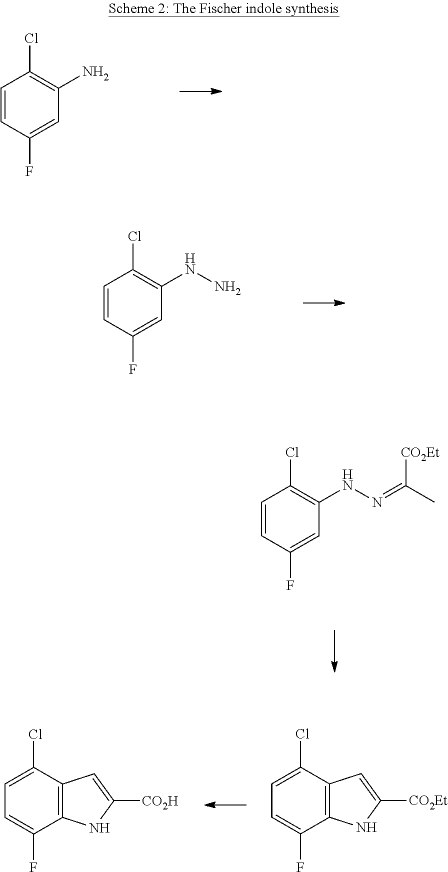 [SMILES: Nc1cc(F)ccc1Cl ; NNc1cc(F)ccc1Cl ; CCOC(=O)/C(C)=N\Nc1cc(F)ccc1Cl ; O=C(O)c1cc2c(Cl)ccc(F)c2[nH]1 ; CCOC(=O)c1cc2c(Cl)ccc(F)c2[nH]1]

A further method for the preparation of substituted indoles is the palladium catalysed alkyne annulation reaction (Journal of the American Chemical Society, 1991, pp. 6690-6692) (Scheme 3).

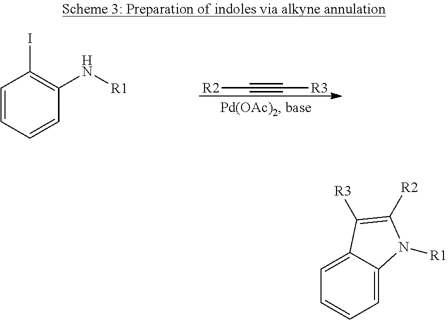

Additionally, indoles may be prepared from other suitably functionalized (halogenated) indoles (for example via palladium catalysed cross coupling or nucleophilic substitution reactions) as illustrated in Scheme 4.

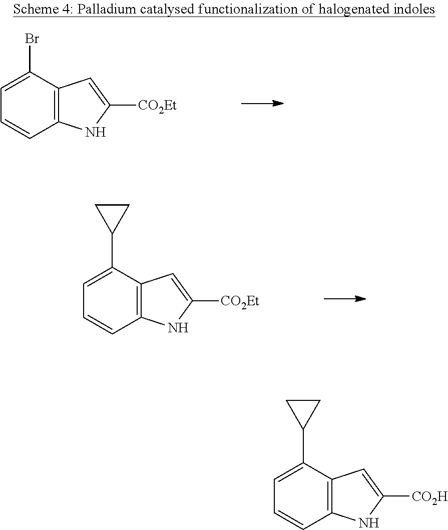 [SMILES: CCOC(=O)c1cc2c(Br)cccc2[nH]1 ; CCOC(=O)c1cc2c(C3CC3)cccc2[nH]1 ; O=C(O)c1cc2c(C3CC3)cccc2[nH]1]

Chemists skilled in the art will appreciate that other methods are available for the synthesis of suitably functionalized indole-2-carboxylic acids and activated esters thereof.

The HBV core protein modulators can be prepared in a number of ways. Schemes 5-12 illustrate the main routes employed for their preparation for the purpose of this application. To the chemist skilled in the art it will be apparent that there are other methodologies that will also achieve the preparation of these intermediates and Examples.

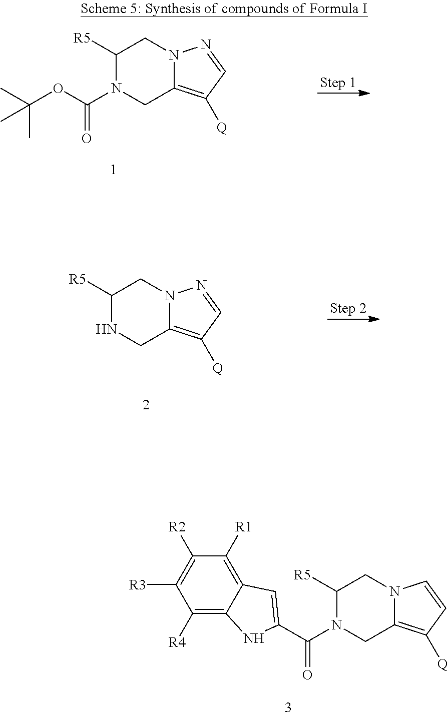

The nitrogen protective group of compound 1 in Scheme 5 is in step 1 deprotected (WO2018/011162, A. Isidro-Llobet et al., Chem. Rev., 2009, 109, 2455-2504), drawn as but not limited to Boc, e.g. with HCl to give an amine of general structure 2. An amide coupling in step 2 with methods known in literature (A. El-Faham, F. Albericio, Chem. Rev. 2011, 111, 6557-6602), e.g. with HATU results in compounds of Formula I.

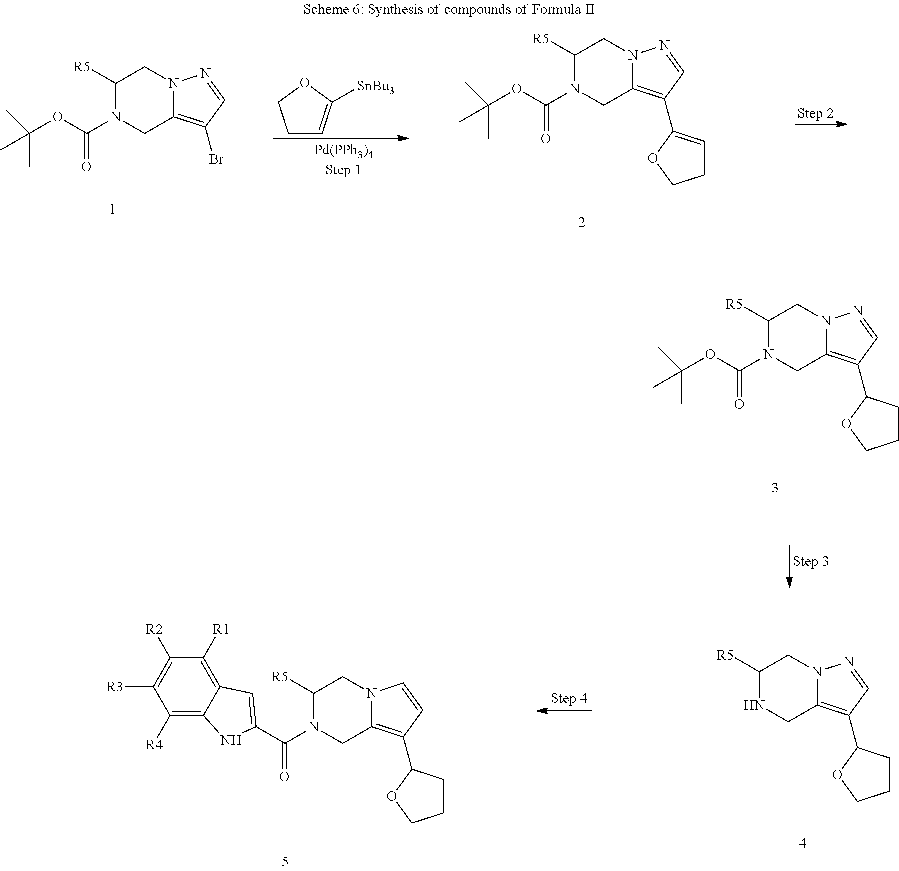

Compound 1 described in Scheme 6 (drawn as but not limited to a bromo substituted aromatic) is in step 1 coupled with a organo-metallate (drawn as, but not limited to a dihydrofuran-2-yl tributyl tin) under palladium catalysis e.g. with Pd(PPh₃)₄ to give compounds of general structure 2. Reduction of the double bond e.g. with H₂ and palladium on carbon gives compounds of general structure 3. The nitrogen protective group of 3 in Scheme 6 is in step 3 deprotected (WO2018/011162, A. Isidro-Llobet et al., Chem. Rev., 2009, 109, 2455-2504), drawn as but not limited to Boc, e.g. with HCl to give an amine of general structure 4. An amide coupling in step 4 with methods known in literature (A. El-Faham, F. Albericio, Chem. Rev. 2011, 111, 6557-6602), e.g. with HATU results in compounds of Formula II.

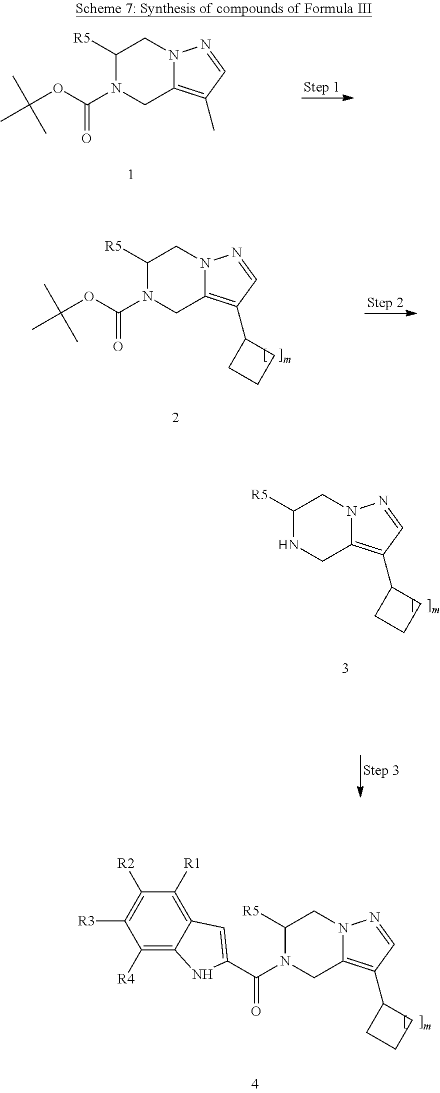

Compound 1 described in Scheme 7 (drawn as but not limited to an iodo substituted aromatic) is in step 1 coupled with e.g. a boronic acid pinacol ester under palladium catalysis e.g. with Pd(PPh₃)₄ to give compounds of general structure 2. The nitrogen protective group of compound of general structure 2 in Scheme 7 is in step 2 deprotected (WO2018/011162, A. Isidro-Llobet et al., Chem. Rev., 2009, 109, 2455-2504), drawn as but not limited to Boc, e.g. with HCl to give an amine of general structure 3. An amide coupling in step 3 with methods known in literature (A. El-Faham, F. Albericio, Chem. Rev. 2011, 111, 6557-6602), e.g. with HATU results in compounds of Formula III.

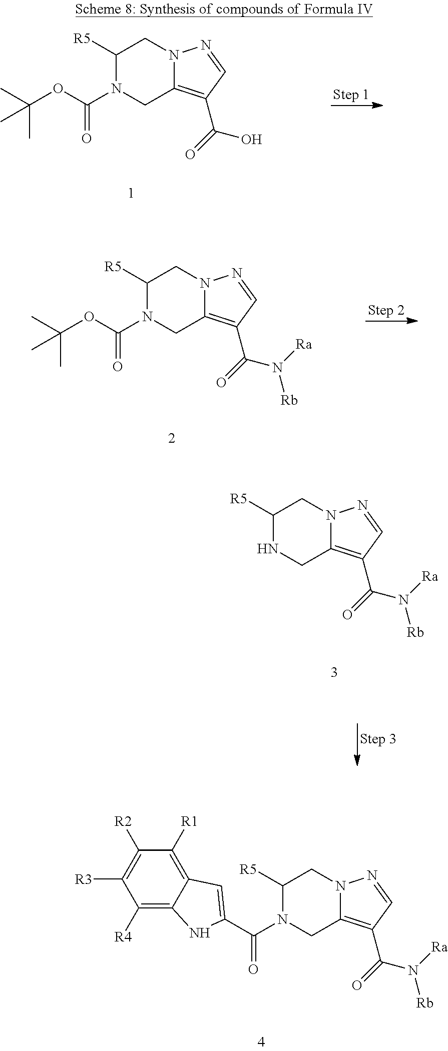

Compound 1 described in Scheme 8 is in step 1 coupled with an amine with methods known in literature (A. El-Faham, F. Albericio, Chem. Rev. 2011, 111, 6557-6602), e.g. with HATU to give a compound with the general structure 2. The nitrogen protective group of compound 2 in Scheme 8 is in step 2 deprotected (WO2018/011162, A. Isidro-Llobet et al., Chem. Rev., 2009, 109, 2455-2504), drawn as but not limited to Boc, e.g. with HCl to give an amine of general structure 3. An amide coupling in step 3 with methods known in literature (A. El-Faham, F. Albericio, Chem. Rev. 2011, 111, 6557-6602), e.g. with HATU results in compounds of Formula IV.

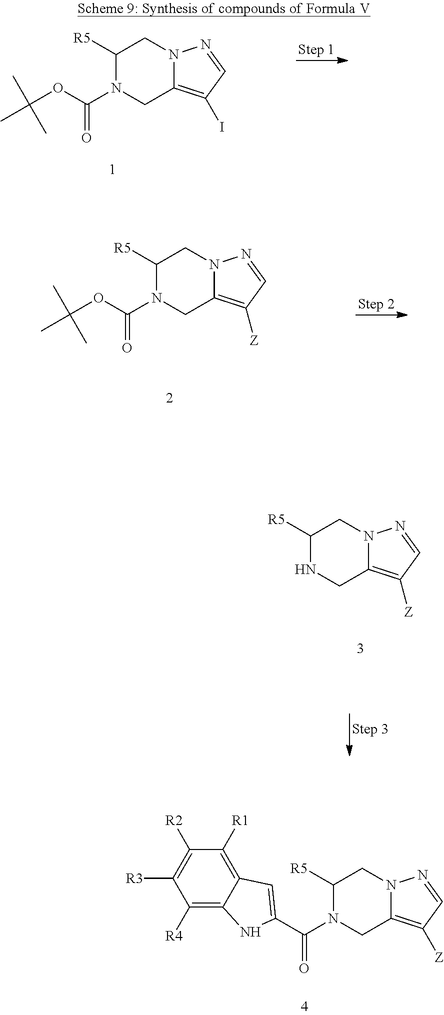

Compound 1 described in Scheme 9 (drawn as but not limited to an iodo substituted aromatic) is in step 1 coupled with e.g. a aryl boronic acid pinacol ester under palladium catalysis e.g. with Pd(PPh₃)₄ to give a compound of general structure 2. The nitrogen protective group of compound 2 in Scheme 9 is in step 2 deprotected (WO2018/011162, A. Isidro-Llobet et al., Chem. Rev., 2009, 109, 2455-2504), drawn as but not limited to Boc, e.g. with HCl to give an amine of general structure 3. An amide coupling in step 3 with methods known in literature (A. El-Faham, F. Albericio, Chem. Rev. 2011, 111, 6557-6602), e.g. with HATU results in compounds of Formula V.

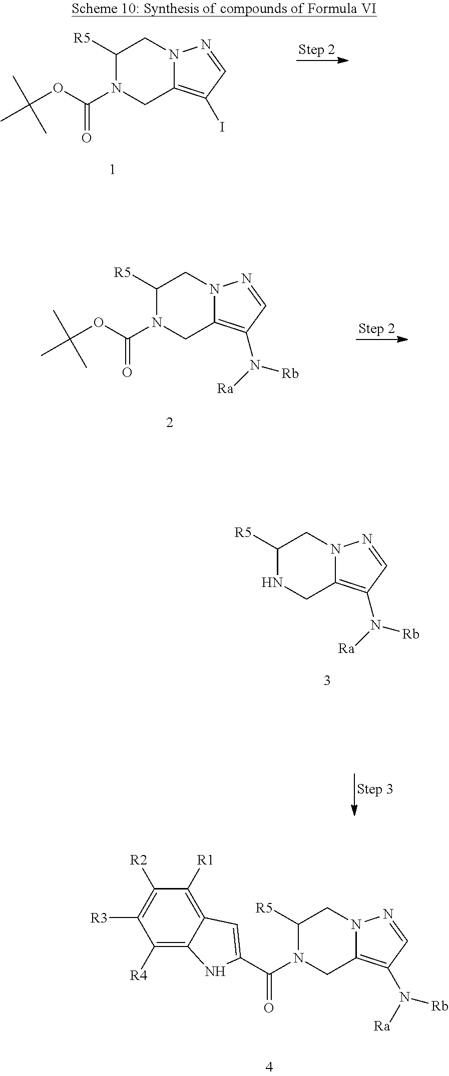

Compound 1 described in Scheme 10 (drawn as but not limited to an iodo substituted aromatic) is in step 1 coupled with e.g. an amine under copper catalysis e.g. with CuI to give compounds of general structure 2 (WO2016/113273). The nitrogen protective group of compound 2 in Scheme 10 is in step 2 deprotected (WO2018/011162, A. Isidro-Llobet et al., Chem. Rev., 2009, 109, 2455-2504), drawn as but not limited to Boc, e.g. with HCl to an amine of general structure 3. An amide coupling in step 3 with methods known in literature (A. El-Faham, F. Albericio, Chem. Rev. 2011, 111, 6557-6602), e.g. with HATU results in compounds of Formula VI.

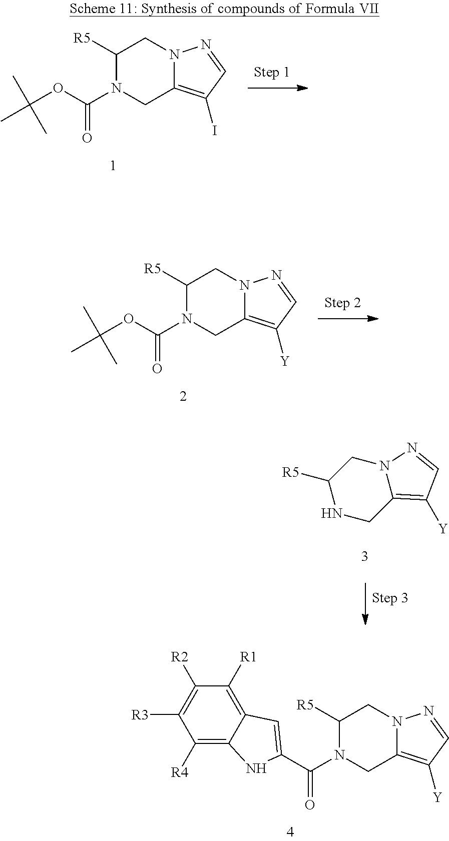

Compound 1 described in Scheme 11 (drawn as but not limited to an iodo substituted aromatic) is in step 1 coupled with e.g. an amide under copper catalysis e.g. with CuI to give compounds of general structure 2 (WO2018/011162). The nitrogen protective group of compound 2 in Scheme 11 is in step 2 deprotected (WO2018/011162, A. Isidro-Llobet et al., Chem. Rev., 2009, 109, 2455-2504), drawn as but not limited to Boc, e.g. with HCl to an amine of general structure 3. An amide coupling in step 3 with methods known in literature (A. El-Faham, F. Albericio, Chem. Rev. 2011, 111, 6557-6602), e.g. with HATU results in compounds of Formula VII.

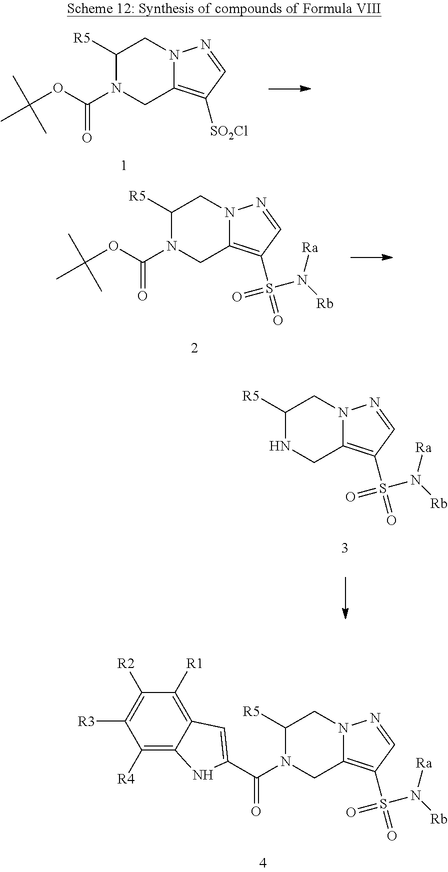

Compound 1 described in Scheme 12 is in step 1 coupled with an amine to give compounds of general structure 2 (WO2018/011162). The nitrogen protective group of compound 2 in Scheme 12 is in step 2 deprotected (WO2018/011162, A. Isidro-Llobet et al., Chem. Rev., 2009, 109, 2455-2504), drawn as but not limited to Boc, e.g. with HCl to give an amine of general structure 3. An amide coupling in step 3 with methods known in literature (A. El-Faham, F. Albericio, Chem. Rev. 2011, 111, 6557-6602), e.g. with HATU results in compounds of Formula VIII.

The following examples illustrate the preparation and properties of some specific compounds of the invention.

The following abbreviations are used:

A—DNA nucleobase adenine
ACN—acetonitrile
Ar—argon
BODIPY-FL—4,4-difluoro-5,7-dimethyl-4-bora-3a,4a-diaza-s-indacene-3-propionic acid (fluorescent dye)
Boc—tert-butoxycarbonyl
BnOH—benzyl alcohol
n-BuLi—n-butyl lithium
t-BuLi—t-butyl lithium
C—DNA nucleobase cytosine
CC₅₀—half-maximal cytotoxic concentration
CO₂—carbon dioxide
CuCN—copper (I) cyanide
DCE—dichloroethane
DCM—dichloromethane
Dess-Martin periodinane—1,1,1-triacetoxy-1,1-dihydro-1,2-benziodoxol-3(1H)-one
DIPEA—diisopropylethylamine
DIPE—di-isopropyl ether
DMAP—4-dimethylaminopyridine

### DMF—N,N-dimethylformamide

DMP—Dess-Martin periodinane
DMSO—dimethyl sulfoxide
DNA—deoxyribonucleic acid
DPPA—diphenylphosphoryl azide
DTIT—dithiothreitol
EC₅₀—half-maximal effective concentration
EDCI—N-(3-dimethylaminopropyl)-N′-ethylcarbodiimide hydrochloride
Et₂O—diethyl ether
EtOAc—ethyl acetate
EtOH—ethanol
FL—five prime end labled with fluorescein
NEt₃—triethylamine

### ELS—Evaporative Light Scattering

g—gram(s)
G—DNA nucleobase guanine
HBV—hepatitis B virus
HATU—2-(1H-7-azabenzotriazol-1-yl)-1,1,3,3-tetramethyl uronium hexafluorophosphate
HCl—hydrochloric acid
HEPES—4-(2-hydroxyethyl)-1-piperazineethanesulfonic acid
HOAt—1-hydroxy-7-azabenzotriazole
HOBt—1-hydroxybenzotriazole
HPLC—high performance liquid chromatography
IC₅₀—half-maximal inhibitory concentration
LC640—3 prime end modification with fluorescent dye LightCycler® Red 640
LC/MS—liquid chromatography/mass spectrometry
LiAlH₄—lithium aluminium hydride
LiOH—lithium hydroxide
MeOH—methanol
MeCN—acetonitrile
MgSO₄—magnesium sulfate
mg—milligram(s)
min—minutes
mol—moles
mmol—millimole(s)
mL—millilitre(s)
MTBE—methyl tert-butyl ether
N₂—nitrogen
Na₂CO₃—sodium carbonate
NaHCO₃—sodium hydrogen carbonate
Na₂SO₄—sodium sulfate
NdeI—restriction enzyme recognizes CA\hat{ }TATG sites
NEt₃—triethylamine
NaH—sodium hydride
NaOH—sodium hydroxide
NH₃—ammonia
NH₄Cl—ammonium chloride
NMR—nuclear magnetic resonance
PAGE—polyacrylamide gel electrophoresis
PCR—polymerase chain reaction
qPCR—quantitative PCR
Pd/C—palladium on carbon
—PH—3 prime end phosphate modification
pTSA—4-toluene-sulfonic acid
Rt—retention time
r.t.—room temperature
sat.—saturated aqueous solution
SDS—sodium dodecyl sulfate
SI—selectivity index (=CC₅₀/EC₅₀)
STAB—sodium triacetoxyborohydride
T—DNA nucleobase thymine
TBAF—tetrabutylammonium fluoride
TFA—trifluoroacetic acid
THF—tetrahydrofuran
TLC—thin layer chromatography
Tris—tris(hydroxymethyl)-aminomethane
XhoI—restriction enzyme recognizes C\hat{ }TCGAG sites

### Compound Identification—NMR

For a number of compounds, NMR spectra were recorded using a Bruker DPX400 spectrometer equipped with a 5 mm reverse triple-resonance probe head operating at 400 MHz for the proton and 100 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

### Compound Identification—HPLC/MS

For a number of compounds, LC-MS spectra were recorded using the following analytical methods.

### Method A

Column—Reverse phase Waters Xselect CSH C18 (50×2.1 mm, 3.5 micron)
Flow—0.8 mL/min, 25 degrees Celsius
Eluent A—95% acetonitrile+5% 10 mM ammonium carbonate in water (pH 9)
Eluent B—10 mM ammonium carbonate in water (pH 9)
Linear gradient t=0 min 5% A, t=3.5 min 98% A. t=6 min 98% A

### Method A2

Column—Reverse phase Waters Xselect CSH C18 (50×2.1 mm, 3.5 micron)
Flow—0.8 mL/min, 25 degrees Celsius
Eluent A—95% acetonitrile+5% 10 mM ammonium carbonate in water (pH 9)
Eluent B—10 mM ammonium carbonate in water (pH 9)
Linear gradient t=0 min 5% A, t=4.5 min 98% A. t=6 min 98% A

### Method B

Column—Reverse phase Waters Xselect CSH C18 (50×2.1 mm, 3.5 micron)
Flow—0.8 mL/min, 35 degrees Celsius
Eluent A—0.1% formic acid in acetonitrile
Eluent B—0.1% formic acid in water
Linear gradient t=0 min 5% A, t=3.5 min 98% A. t=6 min 98% A

### Method B2

Column—Reverse phase Waters Xselect CSH C18 (50×2.1 mm, 3.5 micron)
Flow—0.8 mL/min, 40 degrees Celsius
Eluent A—0.1% formic acid in acetonitrile
Eluent B—0.1% formic acid in water
Linear gradient t=0 min 5% A, t=4.5 min 98% A. t=6 min 98% A

### Method C

Column—Reverse phase Waters Xselect CSH C18 (50×2.1 mm, 3.5 micron)
Flow—1 mL/min, 35 degrees Celsius
Eluent A—0.1% formic acid in acetonitrile
Eluent B—0.1% formic acid in water
Linear gradient t=0 min 5% A, t=1.6 min 98% A. t=3 min 98% A

### Method D

Column—Phenomenex Gemini NX C18 (50×2.0 mm, 3.0 micron)
Flow—0.8 mL/min, 35 degrees Celsius
Eluent A—95% acetonitrile+5% 10 mM ammoniumbicarbonate in water
Eluent B—10 mM ammoniumbicarbonate in water pH=9.0
Linear gradient t=0 min 5% A, t=3.5 min 98% A. t=6 min 98% A

### Method E

Column—Phenomenex Gemini NX C18 (50×2.0 mm, 3.0 micron)
Flow—0.8 mL/min, 25 degrees Celsius
Eluent A—95% acetonitrile+5% 10 mM ammoniumbicarbonate in water
Eluent B—10 mM ammonium bicarbonate in water (pH 9)
Linear gradient t=0 min 5% A, t=3.5 min 30% A. t=7 min 98% A, t=10 min 98% A

### Method F

Column—Waters XSelect HSS C18 (150×4.6 mm, 3.5 micron)
Flow—1.0 mL/min, 25 degrees Celsius
Eluent A—0.1% TFA in acetonitrile
Eluent B—0.1% TFA in water
Linear gradient t=0 min 2% A, t=1 min 2% A, t=15 min 60% A, t=20 min 60% A

### Method G

Column—Zorbax SB-C18 1.8 μm 4.6×15 mm Rapid Resolution cartridge (PN 821975-932)
Flow—3 mL/min
Eluent A—0.1% formic acid in acetonitrile
Eluent B—0.1% formic acid in water
Linear gradient t=0 min 0% A, t=1.8 min 100% A

### Method H

Column—Waters Xselect CSH C18 (50×2.1 mm, 2.5 micron)
Flow—0.6 mL/min
Eluent A—0.1% formic acid in acetonitrile
Eluent B—0.1% formic acid in water
Linear gradient t=0 min 5% A, t=2.0 min 98% A, t=2.7 min 98% A

### Method J

Column—Reverse phase Waters Xselect CSH C18 (50×2.1 mm, 2.5 micron)
Flow—0.6 mL/min
Eluent A—100% acetonitrile
Eluent B—10 mM ammonium bicarbonate in water (pH 7.9)
Linear gradient t=0 min 5% A, t=2.0 min 98% A, t=2.7 min 98% A

### Preparation of 4-chloro-7-fluoro-1H-indole-2-carboxylic Acid

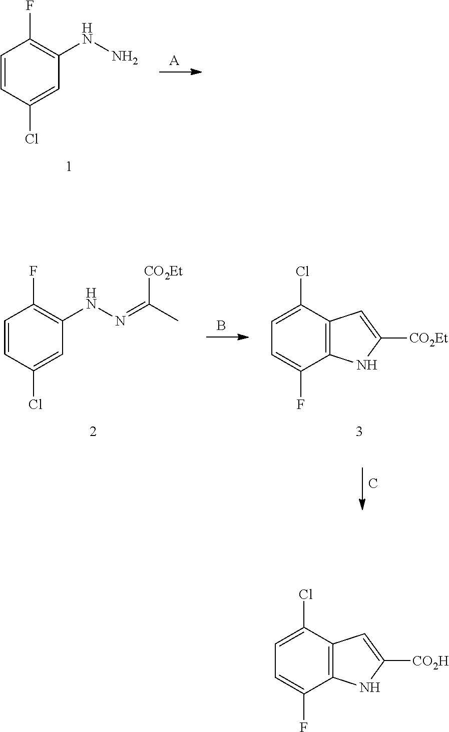

Step A: A mixture of compound 1.HCl (17.0 g, 86.2 mmol), sodium acetate (7.10 g, 86.6 mmol), and ethyl pyruvate (10.0 g, 86.1 mmol) in ethanol (100 mL) was refluxed for 1 h, cooled to r.t., and diluted with water (100 mL). The precipitated solid was collected by filtration and dried to obtain 20.0 g (77.3 mmol, 90%) of compound 2 as a mixture of cis- and trans-isomers.

Step B: A mixture of compound 2 (20.0 g, 77.3 mmol), obtained in the previous step, and BFr-Et₂O (50.0 g, 352 mmol) in acetic acid (125 mL) was refluxed for 18 h and evaporated under reduced pressure. The residue was mixed with water (100 mL) and extracted with MTBE (2×50 mL). The combined organic extracts were dried over Na₂SO₄ and evaporated under reduced pressure. The residue was purified by silica gel column chromatography to give 3.00 g (12.4 mmol, 16%) of compound 3.

Step C: A mixture of compound 3 (3.00 g, 12.4 mmol) and NaOH (0.500 g, 12.5 mmol) in ethanol (30 mL) was refluxed for 30 min and evaporated under reduced pressure. The residue was mixed with water (30 mL) and the insoluble material was filtered off. The filtrate was acidified with concentrated hydrochloric acid (5 mL). The precipitated solid was collected by filtration, washed with water (3 mL), and dried to obtain 2.41 g (11.3 mmol, 91%) of 4-chloro-7-fluoro-1H-indole-2-carboxylic acid.

Rt (Method G) 1.24 mins, W/z 212 [M−H]⁻

### Preparation of 7-fluoro-4-methyl-1H-indole-2-carboxylic Acid

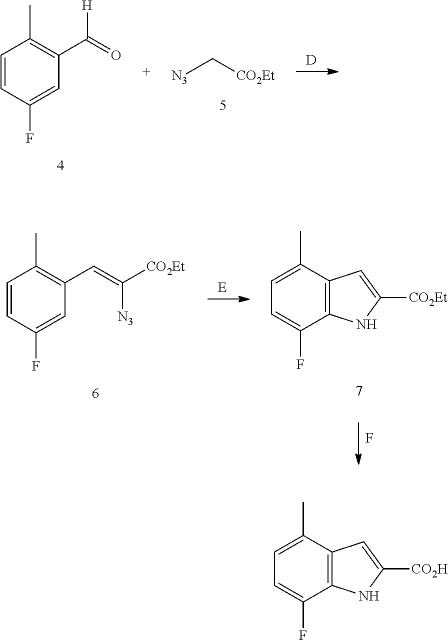

Step D: To a solution of sodium methoxide (21.6 g, 400 mmol) in methanol (300 mL) at at −10° C. was added dropwise a solution of compound 4 (26.4 g, 183 mmol) and compound 5 (59.0 g, 457 mmol) in methanol (100 mL). The reaction mass was stirred for 3 h maintaining temperature below 5° C. and then quenched with ice water. The resulting mixture was stirred for 10 min, filtered, and washed with water to afford 35.0 g (156 mmol, 72%) of compound 6 as a white solid.

Step E: A solution of compound 6, obtained in the previous step, (35.0 g, 156 mmol) in xylene (250 mL) was refluxed for 1 h under an argon atmosphere and then evaporated under reduced pressure. The residue was recrystallized form hexane-ethyl acetate mixture (60:40) to give 21.0 g (103 mmol, 60%) of compound 7.

Step F: To a solution of compound 7 (21.0 g, 101 mmol) in ethanol (200 mL) was added 2 N aqueous sodium hydroxide solution (47 mL). The mixture was stirred for 2 h at 60° C. The solvent was evaporated and the residue was acidified with aqueous hydrochloric acid to pH 5-6. The resulting precipitate was filtered, washed with water, and dried to obtain 18.0 g (93.2 mmol, 92%) of 7-fluoro-4-methyl-1H-indole-2-carboxylic acid.

Rt (Method G) 1.12 mins, m/z 192 [M−H]⁻

### Preparation of 6,7-difluoro-1H-Indole-2-carboxylic Acid

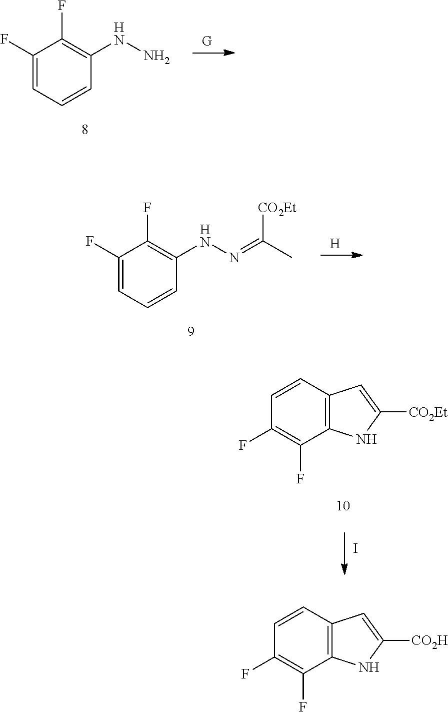

Step G: A mixture of compound 8 (5.00 g, 34.7 mmol), acetic acid (1 mL), and ethyl pyruvate (5.00 g, 43.1 mmol) in ethanol (20 mL) was refluxed for 1 h, cooled to r.t., and diluted with water (20 mL). The precipitated solid was collected by filtration and dried to obtain 5.50 g (22.7 mmol, 66%) of compound 9 as a mixture of cis- and trans-isomers.

Step H: A mixture of compound 9 (5.50 g, 22.7 mmol), obtained in the previous step, and BFr-Et₂O (10.0 g, 70.5 mmol) in acetic acid (25 mL) was refluxed for 18 h and evaporated under reduced pressure. The residue was mixed with water (30 mL) and extracted with MTBE (2×30 mL). The combined organic extracts were dried over Na₂SO₄ and evaporated under reduced pressure. The residue was purified by silica gel column chromatography to give 0.460 g (2.04 mmol, 9%) of compound 10.

Step I: A mixture of compound 10 (0.450 g, 2.00 mmol) and NaOH (0.100 g, 2.50 mmol) in ethanol (10 mL) was refluxed for 30 min and evaporated under reduced pressure. The residue was mixed with water (10 mL) and the insoluble material was filtered off. The filtrate was acidified with concentrated hydrochloric acid (1 mL). The precipitated solid was collected by filtration, washed with water (3 mL), and dried to obtain 0.38 g (1.93 mmol, 95%) of 6,7-difluoro-1H-indole-2-carboxylic acid.

Rt (Method G) 1.10 mins, m/z 196 [M−H]⁻

### Preparation of 4-cyano-1H-indole-2-carboxyl Acid

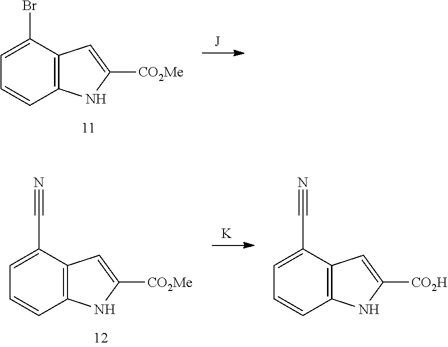

Step J: To a stirred solution of compound 11 (5.00 g, 19.7 mmol) in DMF (50 mL) was added CuCN (3.00 g, 33.5 mmol). The mixture was stirred for 4 h at 150° C. The mixture was then cooled to r.t., and water (100 mL) added. The resulting mixture was extracted with ethyl acetate (4×100 mL). The combined organic extracts were washed with water (50 mL) and brine (50 mL), dried over Na₂SO₄, and evaporated under reduced pressure to give 2.50 g (12.5 mmol, 63%) of compound 12, pure enough for the next step.

Step K: To a solution of compound 12 (2.50 g, 12.5 mmol) in ethanol (30 mL) was added LiOH H₂O (0.600 g, 13.0 mmol). The mixture was refluxed for 10 h. The solvent was evaporated under reduced pressure and the residue diluted with water (50 mL). The aqueous layer was acidified to pH 6 with 10% aq. hydrochloric acid and the precipitated solid was collected by filtration. The residue was washed with water and dried under vacuum to afford 1.20 g (6.45 mmol, 52%) of 4-cyano-1H-indole-2-carboxylic acid as a white solid.

Rt (Method G) 1.00 mins, m/z 197 [M+H]⁺

### Preparation of 4-cyano-7-fluoro-1H-indole-2-carboxylic Acid

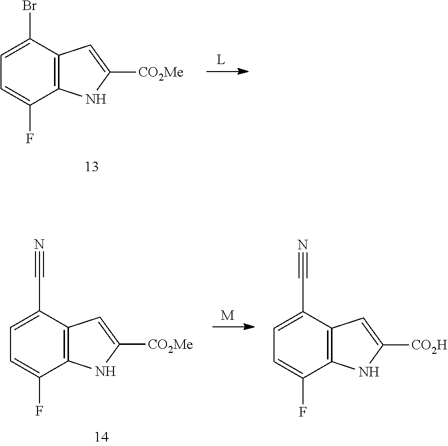

Step L: To a stirred solution of compound 13 (5.00 g, 18.4 mmol) in DMF (50 mL) was added CuCN (2.80 g, 31.2 mmol). The mixture was stirred for 4 h at 150° C. The mixture was then cooled to r.t., and water (100 mL) added. The resulting mixture was extracted with ethyl acetate (4×100 mL. The combined organic extracts were washed with water (50 mL) and brine (50 mL), dried over Na₂SO₄, and evaporated under reduced pressure to give 1.50 g (6.87 mmol, 37%) of compound 14, pure enough for the next step.

Step M: To a solution of compound 14 (1.50 g, 6.87 mmol) in ethanol (20 mL) was added LiOH H₂O (0.400 g, 9.53 mmol). The mixture was refluxed for 10 h. The solvent was evaporated under reduced pressure and the residue diluted with water (40 mL). The aqueous layer was acidified to pH 6.0 with 10% aq. hydrochloric acid and the precipitate was collected by filtration. The residue was washed with water and dried under vacuum to afford 0.400 g (1.95 mmol, 28%) of 4-cyano-7-fluoro-1H-indole-2-carboxylic acid as a white solid.

Rt (Method G) 1.02 mins, m/z 203 [M−H]⁻

### Preparation of 4-cyano-5-fluoro-1H-Indole-2-carboxylic Acid

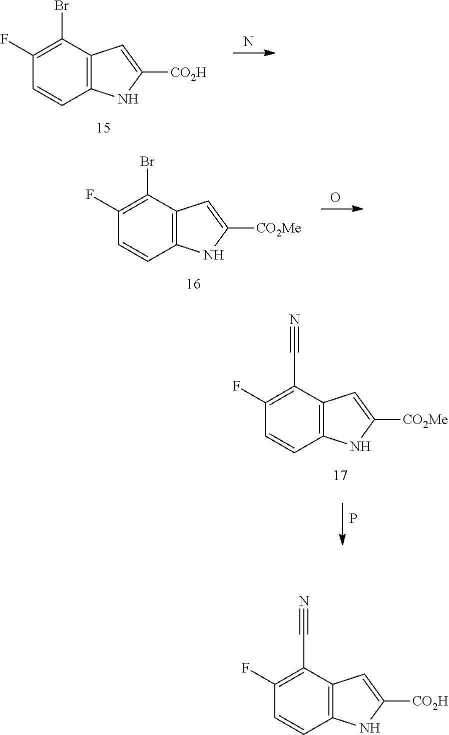

Step N: To a solution of compound 15 (5.00 g, 19.4 mmol) in DMF (50 mL) was added NaHCO₃ (1.59 g, 18.9 mmol) and iodomethane (3 mL). The resulting mixture was stirred overnight at r.t., then diluted with water (50 mL) and extracted with diethyl ether (3×50 mL). The combined organic extracts were dried over Na₂SO₄, and evaporated under reduced pressure to obtain 4.90 g (18.0 mmol, 90%) of compound 16 as white solid.

Step O: To a stirred solution of compound 16 (4.80 g, 17.6 mmol) in DMF (50 mL) was added CuCN (2.70 g, 30.1 mmol). The mixture was stirred for 4 h at 150° C. The mixture was then cooled to r.t., water (100 mL) added. The resulting mixture was extracted with ethyl acetate (4×100 mL). The combined organic extracts were washed with water (50 mL) and brine (50 mL), dried over Na₂SO₄, and evaporated under reduced pressure to give 1.40 g (6.42 mmol, 36%) of compound 17, pure enough for the next step.

Step P: To a solution of compound 17 (1.40 g, 6.42 mmol) in ethanol (20 mL) was added LiOH.H₂O (0.350 g, 8.34 mmol). The mixture was refluxed for 10 h. The solvent was evaporated under reduced pressure and the residue diluted with water (30 mL). The aqueous layer was acidified to pH 6.0 with 10% aq. hydrochloric acid and the precipitate collected by filtration. The residue was washed with water and dried under vacuum to afford 0.500 g (2.45 mmol, 38%) of 4-cyano-5-fluoro-1H-indole-2-carboxylic acid as a white solid.

Rt (Method G) 1.10 mins, m/z 203 [M−H]⁻

### Preparation of 4,5,6-trifluoro-1H-Indole-2-carboxylic Acid

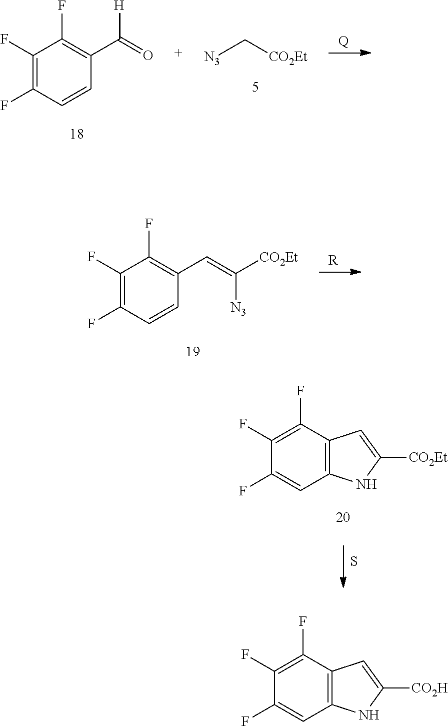

Step Q: To a solution of sodium methoxide (23.0 g, 426 mmol) in methanol (200 mL) at −10° C. was added dropwise a solution of compound 18 (15.0 g, 93.7 mmol) and compound 5 (26.0 g, 201 mmol) in methanol (100 mL). The reaction mixture was stirred for 3 h, maintaining the temperature below 5° C. and then quenched with ice water. The resulting mixture was stirred for 10 min, and the precipitate collected by filtration. The solid was washed with water and dried to afford 12.0 g (46.7 mmol, 72%) of compound 19 as a white solid.

Step R: A solution of compound 19, obtained in the previous step, (12.0 g, 46.7 mmol) in xylene (250 mL) was refluxed for 1 h under an argon atmosphere and then evaporated under reduced pressure. The residue was recrystallized form hexane-ethyl acetate mixture (60:40) to give 7.00 g (30.5 mmol, 65%) of compound 20.

Step S: To a solution of compound 20 (7.00 g, 30.5 mmol) in ethanol (50 mL) was added 2 N aqueous sodium hydroxide solution (18 mL). The mixture was stirred for 2 h at 60° C. The solvent was evaporated and the residue was acidified to pH 5-6 with aqueous hydrochloric acid. The resulting precipitate was collected by filtration, washed with water, and dried to obtain 5.00 g (23.2 mmol, 76%) 4,5,6-trifluoro-1H-indole-2-carboxylic acid.

1H NMR (400 MHz, d6-dmso) 7.17 (1H, s), 7.22 (1H, dd), 12.3 (1H, br s), 13.3 (1H, br s)

### Preparation of 4,6,7-trifluoro-1H-Indole-2-carboxylic Acid

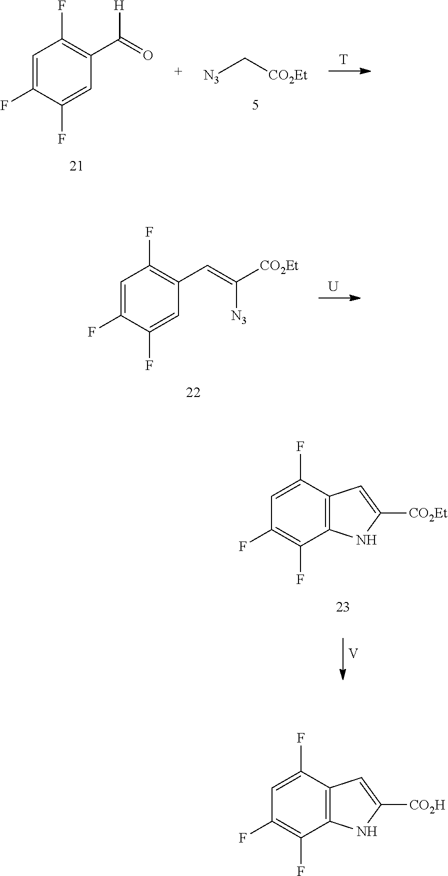

Step T: To a solution of sodium methoxide (23.0 g, 426 mmol) in methanol (200 mL) at −10° C. was added dropwise a solution of compound 21 (15.0 g, 90.3 mmol) and compound 5 (26.0 g, 201 mmol) in methanol (100 mL). The reaction mixture was stirred for 3 h maintaining the temperature below 5° C. and then quenched with ice water. The resulting mixture was stirred for 10 min. The precipitate was collected by filtration, washed with water and dried to afford 10.0 g (38.0 mmol, 42%) of compound 22 as a white solid.

Step U: A solution of compound 22, obtained in the previous step, (10.0 g, 38.0 mmol) in xylene (200 mL) was refluxed for 1 h under an argon atmosphere and then concentrated under reduced pressure. The residue was recrystallized form hexane-ethyl acetate mixture (60:40) to give 6.00 g (26.2 mmol, 69%) of compound 23.

Step V: To a solution of compound 23 (7.00 g, 30.5 mmol) in ethanol (40 mL) was added 2 N aqueous sodium hydroxide solution (16 mL). The mixture was stirred for 2 h at 60° C. The solvent was evaporated and the residue was acidified to pH 5-6 with aqueous hydrochloric acid. The resulting precipitate was collected by filtration, washed with water, and dried to obtain 4.10 g (19.1 mmol, 62%) of 4,6,7-trifluoro-1H-indole-2-carboxylic acid.

Rt (Method G) 1.16 mins, m/z 214 [M−H]⁻

### Preparation of 4-cyano-6-fluoro-1H-indole-2-carboxylic Acid

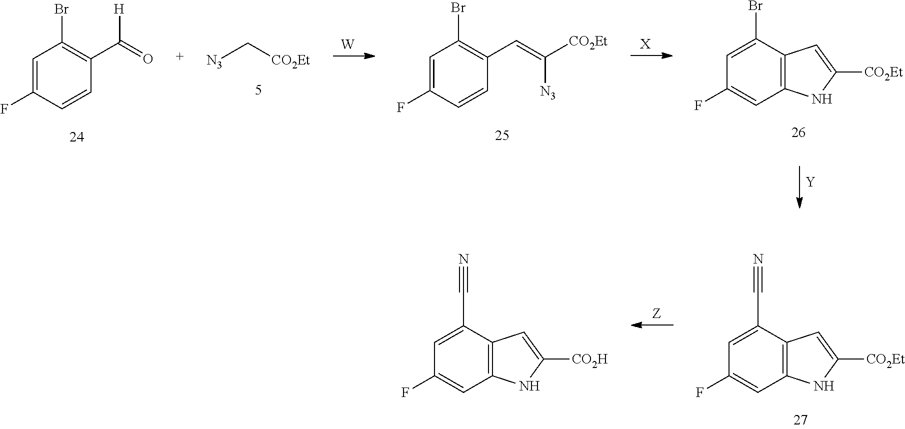

Step W: To a solution of sodium methoxide (65.0 g, 1203 mmol) in methanol (500 mL) at −10° C. was added dropwise a solution of compound 24 (60.0 g, 296 mmol) and compound 5 (85.0 g, 658 mmol) in methanol (200 mL). The reaction mixture was stirred for 3 h maintaining the temperature below 5° C. and then quenched with ice water. The resulting mixture was stirred for 10 min. The precipitate was collected by filtration, washed with water and dried to afford 45.0 g (143 mmol, 48%) of compound 25.

Step X: A solution of compound 25, obtained in the previous step, (35.0 g, 111 mmol) in xylene (250 mL) was refluxed for 1 h under an argon atmosphere and then evaporated under reduced pressure. The residue was recrystallized form hexane-ethyl acetate mixture (60:40) to give 11.0 g (38.4 mmol, 35%) of compound 26.

Step Y: To a stirred solution of compound 26 (11.0 g, 38.4 mmol) in DMF (20 mL) was added CuCN (6.60 g, 73.7 mmol). The mixture was stirred for 4 h at 150° C. The mixture was then cooled to r.t., and water (70 mL) added. The mixture was extracted with ethyl acetate (4×50 mL). The combined organic extracts were washed with water (50 mL) and brine (50 mL), dried over Na₂SO₄, and evaporated under reduced pressure to give 2.40 g (10.3 mmol, 27%) of compound 27, pure enough for the next step.

Step Z: To a solution of compound 27 (2.40 g, 6.42 mmol) in ethanol (30 mL) was added LiOH.H₂O (0.600 g, 14.3 mmol). The mixture was refluxed for 10 h. The mixture was concentrated under reduced pressure and the residue diluted with water (50 mL). The aqueous layer was acidified to pH 6 with 10% aq. hydrochloric acid and the precipitate was collected by filtration. The solid was washed with water and dried under vacuum to afford 1.20 g (5.88 mmol, 57%) of 4-cyano-6-fluoro-1H-indole-2-carboxylic acid as a white solid.

Rt (Method G) 1.06 mins, m/z 203 [M−H]⁻

### Preparation of 4-ethyl-1H-indole-2-carboxyli Acid

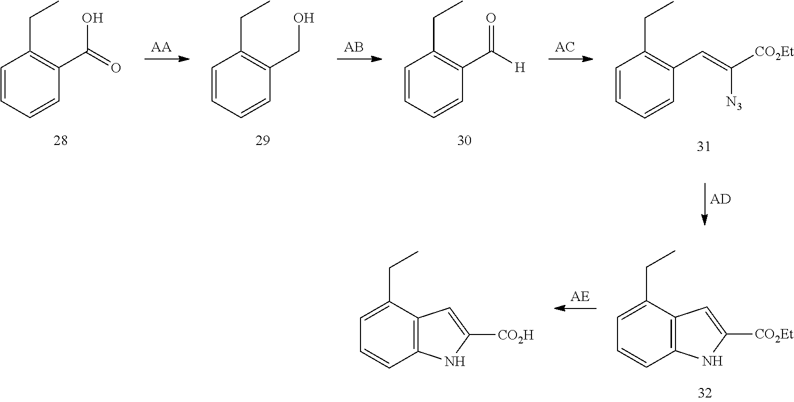

Step AA: A solution of compound 28 (70.0 g, 466 mmol) in dry THF (500 mL) was treated with 10 M solution of BH₃ in THF (53 mL, 53.0 mmol of BH₃) at 0° C. The reaction mass was stirred at r.t. for 24 h before methanol (150 mL) was slowly added thereto. The resulting mixture was stirred for 45 min, and evaporated under reduced pressure to yield 55.0 g (404 mmol, 87%) of compound 29, pure enough for the next step.

Step AB: To a cooled (0° C.) solution of compound 29 (55.0 g, 404 mmol) in CH₂Cl₂ (400 mL) was added Dess-Martin periodinane (177 g, 417 mmol) portionwise. After stirring for 1 h at r.t., the reaction mixture was quenched with saturated aqueous Na₂S₂O₃ (300 mL) and saturated aqueous NaHCO₃ (500 mL). The mixture was extracted with CH₂Cl₂ (3×300 mL). The combined organic extracts were washed with water and brine, dried over Na₂SO₄ and concentrated to yield 51.0 g of crude compound 30 as a yellow solid.

Step AC: To a solution of sodium methoxide (107 g, 1981 mmol) in methanol (600 mL) at −10° C. was added dropwise a solution of compound 30, obtained in the previous step, (51.0 g) and compound 5 (126 g, 976 mmol) in methanol (300 mL). The reaction mixture was stirred for 4 h maintaining temperature below 5° C., then quenched with ice water. The resulting mixture was stirred for 10 min, and the precipitate collected by filtration. The solid was washed with water and dried to afford 35.0 g (151 mmol, 37% over 2 steps) of compound 31.

Step AD: A solution of compound 31, obtained in the previous step, (35.0 g, 151 mmol) in xylene (500 mL) was refluxed for 1 h under an argon atmosphere and then concentrated under reduced pressure. The residue was recrystallized form hexane-ethyl acetate mixture (60:40) to give 21.0 g (103 mmol, 68%) of compound 32.

Step AE: To a solution of compound 32 (21.0 g, 103 mmol) in ethanol (200 mL) was added 2 N aqueous sodium hydroxide solution (47 mL). The mixture was stirred for 2 h at 60° C. The mixture was concentrated under reduced pressure, and the residue acidified to pH 5-6 with aqueous hydrochloric acid. The precipitate was collected by filtration, washed with water, and dried to obtain 19 g (100 mmol, 97%) of 4-ethyl-1H-indole-2-carboxylic acid.

Rt (Method G) 1.20 mins, m/z 188 [M−H]⁻

1H NMR (400 MHz, d6-dmso) δ 1.25 (t, 3H), 2.88 (q, 2H), 6.86 (1H, d), 7.08-7.20 (2H, m), 7.26 (1H, d), 11.7 (1H, br s), 12.9 (1H, br s)

### Preparation of 4-cyclopropyl-1H-indole-2-carboxylic Acid

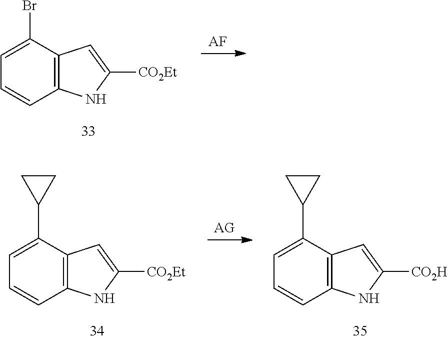 [SMILES: CCOC(=O)c1cc2c(Br)cccc2[nH]1 ; "AF" ; CCOC(=O)c1cc2c(C3CC3)cccc2[nH]1 ; "AG" ; O=C(O)c1cc2c(C3CC3)cccc2[nH]1]

Step AF: To a degassed suspension of compound 33 (2.00 g, 7.80 mmol), cyclopropylboronic acid (0.754 g, 8.78 mmol), K₃PO₄ (5.02 g, 23.6 mmol), tricyclohexyl phosphine (0.189 g, 0.675 mmol), and water (2.0 mL) in toluene (60.0 mL) was added palladium (II) acetate (0.076 g, 0.340 mmol). The reaction mixture was stirred at 100° C. for 4 h. The reaction progress was monitored by diluting an aliquot of the reaction mixture with water and extracting with ethyl acetate. The organic layer was spotted over an analytical silica gel TLC plate and visualized using 254 nm UV light. The reaction progressed to completion with the formation of a polar spot. The R_{f} values of the starting material and product were 0.3 and 0.2, respectively. The reaction mixture was allowed to cool to r.t. and filtered through a pad of celite. The filtrate was concentrated under reduced pressure and the crude product was purified by flash column using 230-400 mesh silica gel and eluted with 10% ethyl acetate in petroleum ether to afford 1.10 g (5.11 mmol, 63%) of compound 34 as a brown liquid. TLC system: 5% ethyl acetate in petroleum ether.

Step AG: A mixture of compound 34 (1.10 g, 5.11 mmol) in ethanol (40 mL) and 2 N aqueous sodium hydroxide (15 mL) was stirred for 2 h at 60° C. The mixture was concentrated under reduced pressure, and the residue acidified to pH 5-6 with aqueous hydrochloric acid. The precipitate was collected by filtration, washed with water, and dried to yield 1.01 g (5.02 mmol, 92%) of 4-cyclopropyl-1H-indole-2-carboxylic acid.

Rt (Method G) 1.17 mins, m/z 200 [M−H]⁻

### Preparation of 4-chloro-5-fluoro-1H-Indole-2-carboxylic Acid

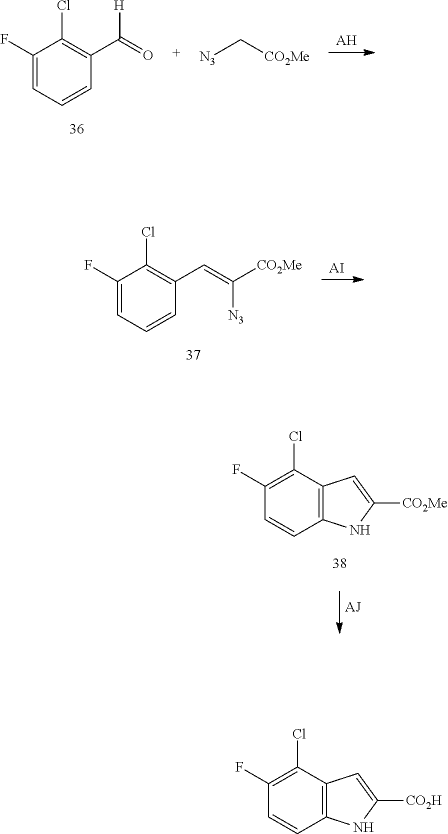

Step AH: To a solution of sodium methoxide (39.9 g, 738 mmol) in methanol (300 mL) at −10° C. was added dropwise a solution of compound 36 (28.8 g, 182 mmol) and methyl azidoacetate (52.1 g, 404 mmol) in methanol (150 mL). The reaction mixture was stirred for 3 h maintaining temperature below 5° C., then quenched with ice water. The resulting mixture was stirred for 10 min. The precipitate was collected by filtration, washed with water and dried to afford 20.0 g (78.2 mmol, 43%) of compound 37.

Step AI: A solution of compound 37 (19.4 g, 76.0 mmol) in xylene (250 mL) was refluxed for 1 h under an argon atmosphere and then concentrated under reduced pressure. The residue was recrystallized from hexane-ethyl acetate (50:50) to give 9.00 g (39.5 mmol, 52%) of compound 38.

Step AJ: To a solution of compound 38 (8.98 g, 39.4 mmol) in ethanol (100 mL) was added 2 N aqueous sodium hydroxide solution (18 mL). The mixture was stirred for 2 h at 60° C. The mixture was concentrated under reduced pressure, and the residue acidified to pH 5-6 with aqueous hydrochloric acid. The resulting precipitate was collected by filtration, washed with water, and dried to obtain 7.75 g (36.3 mmol, 92%) of 4-chloro-5-fluoro-1H-indole-2-carboxylic acid.

Rt (Method G) 1.15 mins, m/z 212 [M−H]⁻

1H NMR (400 MHz, d6-dmso) 7.08 (1H, s), 7.28 (1H, dd) 7.42 (1H, dd), 12.2 (1H, br s), 13.2 (1H, br s)

### Preparation of 5-fluoro-4-(1-hydroxyethyl)-1H-Indole-2-carboxylic Acid

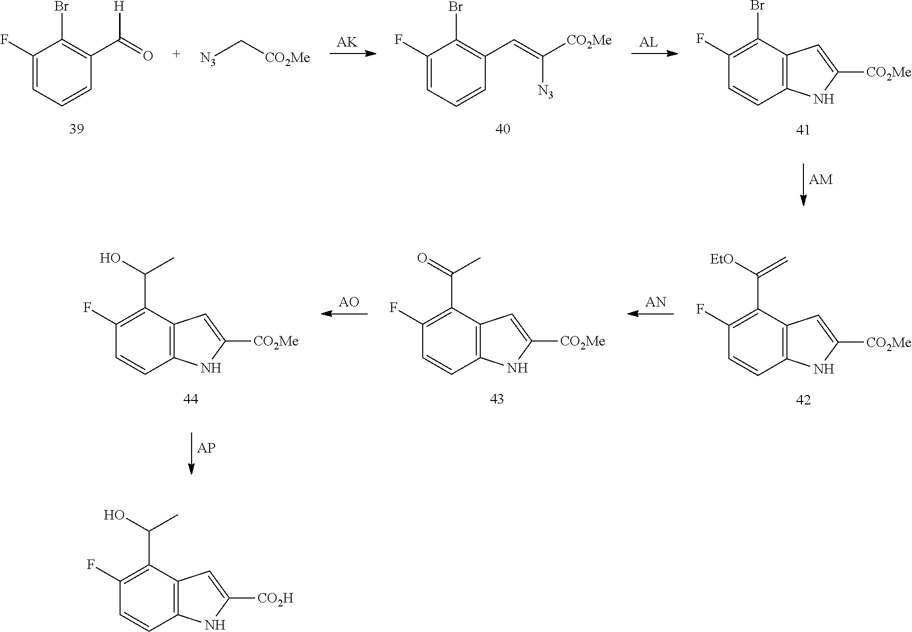

Step AK: To a solution of sodium methoxide (50.0 g, 926 mmol) in methanol (300 mL) at −10° C. was added dropwise a solution of compound 39 (45.0 g, 222 mmol) and methyl azidoacetate (59.0 g, 457 mmol) in methanol (100 mL). The reaction mixture was stirred for 3 h maintaining the temperature below 5° C., then quenched with ice water. The resulting mixture was stirred for 10 min. The precipitate was collected by filtration, washed with water and dried to afford 35.0 g (133 mmol, 60%) of compound 40 as a white solid.

Step AL: A solution of compound 40, obtained in the previous step, (35.0 g, 133 mmol) in xylene (250 mL) was refluxed for 1 h under an argon atmosphere and then evaporated under reduced pressure. The residue was recrystallized from hexane-ethyl acetate (60:40) to give 21.0 g (77.2 mmol. 58%) of compound 41.

Step AM: To a degassed solution of compound 41 (4.00 g, 14.7 mmol) and tributyl(1-ethoxyvinyl)stannane (5.50 g, 15.2 mmol) in toluene (50 mL) under nitrogen was added bis(triphenylphosphine) palladium(II) dichloride (1.16 g, 1.65 mmol). The reaction mixture was stirred at 60° C. for 20 h. The reaction mixture was cooled to room temperature and filtered. The filtrate was concentrated under under reduced pressure and the residue purified by silica gel chromatography to afford 2.50 g (9.50 mmol, 65%) of compound 42 as a pale yellow solid.

Step AN: To a solution of compound 42 (2.40 g, 9.12 mmol) in 1,4-dioxane (30 mL) was added 2M hydrochloric acid (15 mL). The resulting mixture was stirred at room temperature for 30 min. The mixture was concentrated under vacuum and the residue partitioned between ethyl acetate and water. The organic extract was washed with water and brine, dried over sodium sulfate, filtered, and evaporated. The residue was triturated with 5% ether in isohexane and dried to afford 1.80 g (7.65 mmol, 84%) of compound 43 as a white solid.

Step AO: A suspension of compound 43 (1.70 g, 7.23 mmol) and NaBH₄ (2.50 g, 66.1 mmol) in ethanol (13 mL) was refluxed for 2 h, then cooled to room temperature, and filtered. The filtrate was concentrated under reduced pressure and the residue dissolved in ethyl acetate. The solution was washed with 1N hydrochloric acid and brine, dried over Na₂SO₄, and evaporated under reduced pressure to give 1.60 g (6.74 mmol, 93%) of compound 44 as a colourless oil.

Step AP: To a solution of compound 44 (1.50 g, 6.32 mmol) in methanol (40 mL) was added 2N aqueous NaOH (10 mL). The mixture was stirred for 2 h at 60° C. The mixture was concentrated under reduced pressure and the residue acidified to pH 5-6 with 10% hydrochloric acid. The precipitate was collected by filtration, washed with water (3×15 mL), and dried to obtain 1.30 g (5.82 mmol, 92%) of 5-fluoro-4-(1-hydroxyethyl)-1H-indole-2-carboxylic acid.

Rt (Method G) 1.00 mins, m/z 222 [M−H]⁻

### Preparation of 4-ethyl-5-fluoro-1H-indole-2-carboxylic Acid

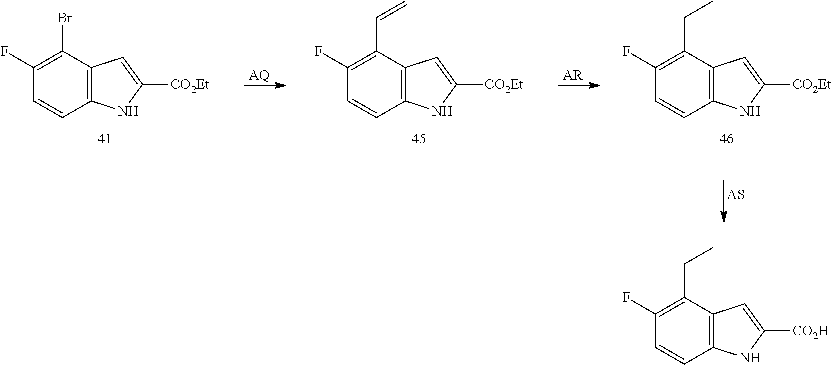

Step AQ: To a heated (90° C.) solution of compound 41 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was then cooled to room temperature and purified by silica gel column chromatography (60-80% ethyl acetate in hexane) to give 2.20 g (10.0 mmol, 68%) of compound 45 as yellow solid.

Step AR: A mixture of compound 45 (1.50 g, 6.84 mmol) and Pd/C (0.300 g, 10% wt.) in methanol (20 mL) was stirred under an atmosphere of hydrogen at room temperature for 16 h. The mixture was filtered, then concentrated under reduced pressure to give 1.45 g (6.55 mmol, 96%) of compound 46.

Step AS: To a solution of compound 46 (1.40 g, 6.33 mmol) in methanol (40 mL) was added 2N aqueous NaOH (10 mL). The mixture was stirred for 2 h at 60° C. The mixture was concentrated under vacuum, then the residue was acidified to pH 5-6 with 10% hydrochloric acid. The precipitate was collected by filtration, washed with water (3×15 mL), and dried to obtain 1.20 g (5.79 mmol, 91%) of target compound 4-ethyl-5-fluoro-1H-indole-2-carboxylic acid.

Rt (Method G) 1.33 mins, m/z 206 [M−H]⁻

### Preparation of 4-ethyl-6-fluoro-1H-indole-2-carboxylic Acid

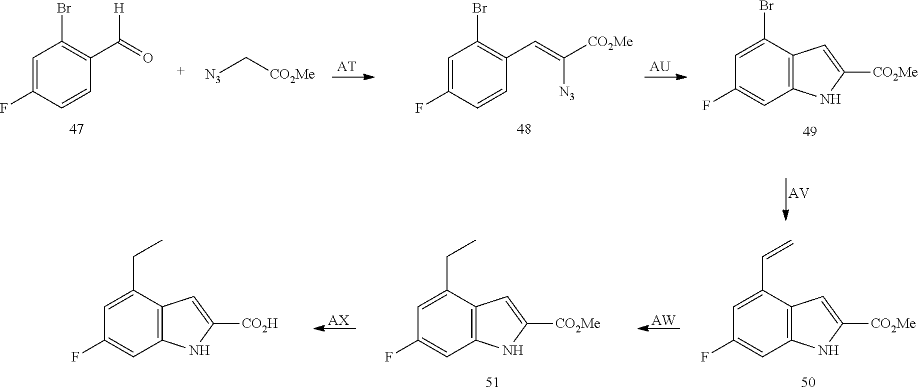

Step AT: To a solution of sodium methoxide (50.0 g, 926 mmol) in methanol (300 mL) at −10° C. was added dropwise a solution of compound 47 (45.0 g, 202 mmol) and methyl azidoacetate (59.0 g, 457 mmol) in methanol (100 mL). The reaction mixture was stirred for 3 h maintaining temperature below 5° C., then quenched with ice water. The resulting mixture was stirred for 10 min. The precipitate was collected by filtration, washed with water and dried to afford 38.5 g (128 mmol, 63%) of compound 48 as a white solid.

Step AU: A solution of compound 48, obtained in the previous step, (38.5 g, 128 mmol) in xylene (250 mL) was refluxed for 1 h under an argon atmosphere and then concentrated under reduced pressure. The residue was recrystallized hexane-ethyl acetate (60:40) to give 18.0 g (67.3 mmol, 53%) of compound 49.

Step AV: To a heated (90° C.) solution of compound 49 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was then cooled to room temperature and purified by silica gel column chromatography (60-80% ethyl acetate in hexane) to give 2.00 g (9.12 mmol, 62%) of compound 50 as yellow solid.

Step AW: A mixture of compound 50 (1.50 g, 6.84 mmol) and Pd/C (0.300 g, 10% wt.) in methanol (20 mL) was stirred under an atmosphere of hydrogen at room temperature for 16 h. The mixture was filtered and concentrated to give 1.40 g (6.33 mmol, 93%) of compound 51.

Step AX: To a solution of compound 51 (1.10 g, 4.97 mmol) in methanol (40 mL) was added 2N aqueous NaOH (10 mL). The mixture was stirred for 2 h at 60° C. The mixture was concentrated under reduced pressure, then acidified to pH 5-6 with 10% hydrochloric acid. The precipitate was collected by filtration, washed with water (3×15 mL), and dried to obtain 0.900 g (4.34 mmol, 87%) of target compound 4-ethyl-6-fluoro-1H-indole-2-carboxylic acid.

Rt (Method G) 1.29 mins, m/z 206 [M−H]⁻

### Preparation of 6-fluoro-4-(1-hydroxyethyl)-1H-Indole-2-carboxylic Acid

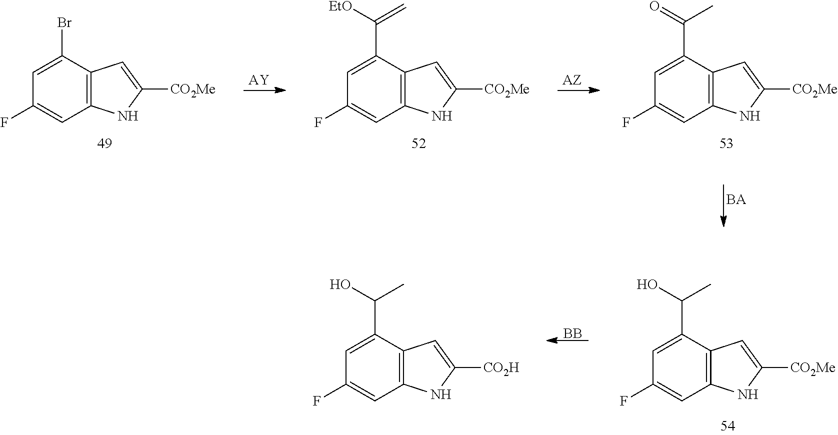

Step AY: To a degassed solution of compound 49 (4.00 g, 14.7 mmol) and tributyl(1-ethoxyvinyl)stannane (5.50 g, 15.2 mmol) in toluene (50 mL) under nitrogen were added bis(triphenylphosphine) palladium(II) dichloride (1.16 g, 1.65 mmol). The reaction mixture was stirred at 60° C. for 20 h. The reaction mixture was cooled to room temperature and filtered. The filtrate was concentrated under reduced pressure and the residue purified by silica gel chromatography to give 2.10 g (7.98 mmol, 54%) of compound 52 as a pale yellow solid.

Step AZ: To a solution of compound 52 (2.10 g, 7.98 mmol) in 1,4-dioxane (30 mL) was added 2M hydrochloric acid (15 mL). The resulting mixture was stirred at room temperature for 30 min. The mixture was concentrated under reduced pressure, and residue partitioned between ethyl acetate and water. The organic extract was washed with water and brine, dried over sodium sulfate, filtered, and concentrated. The residue was triturated with 5% ether in isohexane and dried to afford 1.70 g (7.23 mmol, 91%) of compound 53 as a white solid.

Step BA: A suspension of compound 53 (1.70 g, 7.23 mmol) and NaBH₄ (2.50 g, 66.1 mmol) in ethanol (13 mL) was refluxed for 2 h, cooled to room temperature, and filtered. The filtrate was concentrated under reduced pressure and the residue was dissolved in ethyl acetate. The solution was washed with 1N hydrochloric acid and brine, dried over Na₂SO₄, and concentrated under reduced pressure to give 1.60 g (6.74 mmol, 93%) of compound 54 as a colourless oil.

Step BB: To a solution of compound 54 (1.40 g, 5.90 mmol) in methanol (40 mL) was added 2N aqueous NaOH (10 mL). The mixture was stirred for 2 h at 60° C. The mixture was concentrated and the residue acidified to pH 5-6 with 10% hydrochloric acid. The precipitate was collected by filtration, washed with water (3×15 mL), and dried to obtain 1.10 g (4.93 mmol, 48%) of target compound 6-fluoro-4-(1-hydroxyethyl)-1H-indole-2-carboxylic acid.

Rt (Method G) 1.00 mins, m/z 222 [M−H]⁻

### Preparation of 4-ethyl-7-fluoro-1H-indole-2-carboxylic Acid

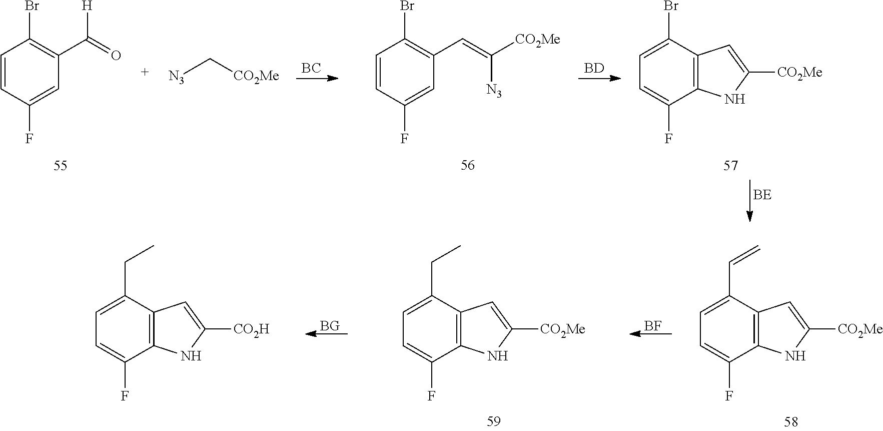

Step BC: To a solution of sodium methoxide (50.0 g, 926 mmol) in methanol (300 mL) −10° C. was added dropwise a solution of compound 55 (45.0 g, 222 mmol) and methyl azidoacetate (59.0 g, 457 mmol) in methanol (100 mL). The reaction mixture was stirred for 3 h maintaining temperature below 5° C., then quenched with ice water. The resulting mixture was stirred for 10 min. The precipitate was collected by filtration, washed with water and dried to afford 33.0 g (110 mmol, 50%) of compound 56 as a white solid.

Step BD: A solution of compound 56, obtained in the previous step, (33.0 g, 110 mmol) in xylene (250 mL) was refluxed for 1 h under an argon atmosphere and then concentrated under reduced pressure. The residue was recrystallized from hexane-ethyl acetate (60:40) to give 21.5 g (79.0 mmol, 72%) of compound 57.

Step BE: To a heated (90° C.) solution of compound 57 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was cooled to room temperature and purified by silica gel column chromatography (60-80% EtOAc in hexane). The combined product fractions of the product were concentrated, washed with water (3×100 mL), dried over Na₂SO₄, and concentrated to give 1.80 g (8.21 mmol, 56%) of compound 58 as yellow solid.

Step BF: A mixture of compound 58 (1.50 g, 6.84 mmol) and Pd/C (0.300 g, 10% wt.) in methanol (20 mL) was stirred under atmosphere of hydrogen at room temperature for 16 h. The mixture was filtered and concentrated to give 1.25 g (5.65 mmol, 83%) of compound 59.

Step BG: To a solution of compound 59 (1.40 g, 6.33 mmol) in methanol (40 mL) was added 2N aqueous NaOH (10 mL). The mixture was stirred for 2 h at 60° C. The mixture was concentrated under reduced pressure, and the residue acidified to pH 5-6 with 10% hydrochloric acid. The precipitate was collected by filtration, washed with water (3×15 mL), and dried to obtain 1.25 g (6.03 mmol, 95%) of target compound 4-ethyl-7-fluoro-1H-indole-2-carboxylic acid.

Rt (Method G) 1.27 mins, m/z 206 [M−H]⁻

### Preparation of 7-fluoro-4-(1-hydroxyethyl)-1H-Indole-2-carboxylic Acid

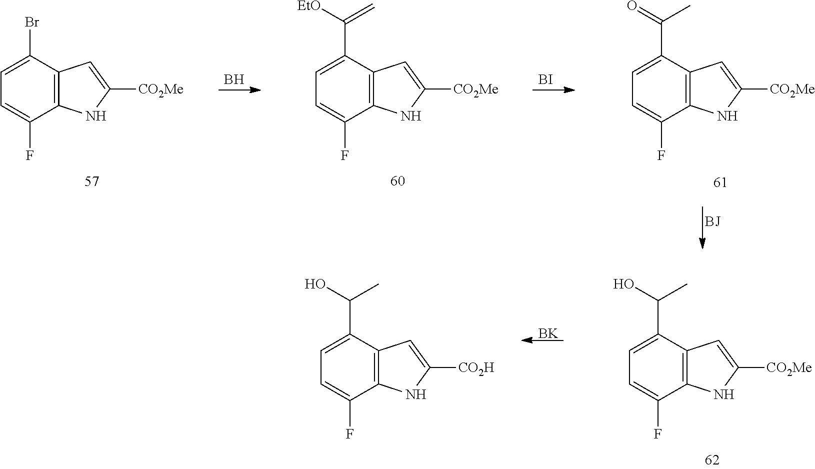

Step BH: To a degassed solution of compound 57 (4.00 g, 14.7 mmol) and tributyl(1-ethoxyvinyl)stannane (5.50 g, 15.2 mmol) in toluene (50 mL) under nitrogen was added bis(triphenylphosphine) palladium(II) dichloride (1.16 g, 1.65 mmol). The reaction mixture was stirred at 60° C. for 20 h. The mixture was cooled to room temperature and filtered. The filtrate was concentrated under reduced pressure and the residue purified by silica gel chromatography to afford 2.70 g (10.3 mmol, 70%) of compound 60 as a pale yellow solid.

Step BI: To a solution of compound 60 (2.40 g, 9.12 mmol) in 1,4-dioxane (30 mL) was added 2M hydrochloric acid (15 mL). The mixture was stirred at room temperature for 30 min. The majority of the solvent was evaporated and the residue was partitioned between ethyl acetate and water. The combined organic extracts were washed with water and brine, dried over sodium sulfate, filtered, and evaporated. The residue was triturated with 5% ether in isohexane and dried to afford 1.90 g (8.08 mmol, 86%) of compound 61 as a white solid.

Step BJ: A suspension of compound 61 (1.70 g, 7.23 mmol) and NaBH₄ (2.50 g, 66.1 mmol) in ethanol (13 mL) was refluxed for 2 h, cooled to room temperature, and filtered. The filtrate was evaporated under reduced pressure and the residue was dissolved in ethyl acetate. The solution was washed with 1N hydrochloric acid and brine, dried over Na₂SO₄, and evaporated under reduced pressure to give 1.50 g (6.32 mmol, 87%) of compound 62 as a colourless oil.

Step BK: To a solution of compound 62 (1.50 g, 6.32 mmol) in methanol (40 mL) was added 2N aqueous NaOH (10 mL). The mixture was stirred for 2 h at 60° C. The mixture was concentrated under reduced pressure and the residue acidified to pH 5-6 with 10% hydrochloric acid. The precipitate was collected by filtration, washed with water (3×15 mL), and dried to obtain 1.35 g (6.05 mmol, 96%) of target compound 7-fluoro-4-(1-hydroxyethyl)-1H-indole-2-carboxylic acid.

Rt (Method G) 0.90 mins, m/z 222 [M−H]⁻

### Preparation of 4-(hydroxymethyl)-1H-indole-2-carboxylic Acid

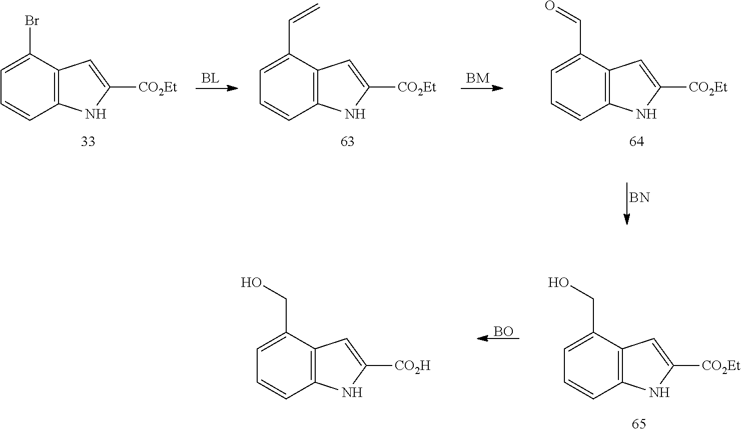

Step BL: To a solution of compound 33 (10.0 g, 39.4 mmol) in a mixture of dioxane (200 mL) and water (50 mL) were added potassium vinyltrifluoroborate (11.0 g, 82.1 mmol), triethylamine (30 mL, 248 mmol) and Pd(dppf)Cl₂ (1.0 g, 1.37 mmol). The mixture was stirred at 80° C. for 48 h. The mixture was concentrated under vacuum, and the residue was dissolved in ethyl acetate. The solution was washed with water and concentrated under reduced pressure. The obtained material was purified by silica gel column chromatography to give 2.50 g (12.4 mmol, 38%) of compound 63.

Step BM: To a mixture of compound 63 (2.50 g, 12.4 mmol), acetone (200 mL), and water (40 mL) were added OsO₄ (0.100 g, 0.393 mmol) and NaIO₄ (13.4 g, 62.6 mmol). The reaction was stirred for 10 h at room temperature. The acetone was distilled off and the remaining aqueous solution extracted with dichloromethane. The organic layer was washed with saturated NaHCO₃ solution (2×50 mL) and brine (2×50 mL), dried over Na₂SO₄, and concentrated under reduced pressure to obtain 1.50 g (7.40 mmol, 60%) of compound 64.

Step BN: To a cooled (0° C.) solution of compound 64 (1.50 g, 7.38 mmol) in THF/methanol mixture (100 mL) was added NaBH₄ (0.491 g, 13.0 mmol). The reaction mixture was stirred for 12 h at room temperature. Then the mixture was cooled to 0° C., treated with 2N hydrochloric acid (40 mL), and concentrated. The residue was extracted with ethyl acetate. The organic extract was washed with water, dried over Na₂SO₄, and concentrated under reduced pressure to obtain 1.00 g (4.87 mmol, 65%) of compound 65, pure enough for the next step.

Step BO: To a solution of compound 65, obtained in the previous step, (1.00 g, 4.87 mmol) in THF (50 mL), was added 1N aqueous LiOH (9 mL). The resulting mixture was stirred for 48 h at room temperature, then concentrated and diluted with 1N aqueous NaHSO₄ (9 mL). The mixture was extracted with ethyl acetate. The organic extract was dried over Na₂SO₄, and concentrated under reduced pressure. The residue was recrystallized from MTBE to obtain 0.250 g (1.30 mmol, 27%) of target compound 4-(hydroxymethyl)-1H-indole-2-carboxylic acid.

Rt (Method G) 0.98 mins, m/z 190 [M−H]⁻

### Preparation of 4-(2-hydroxypropan-2-yl)-1H-indole-2-carboxylic Acid

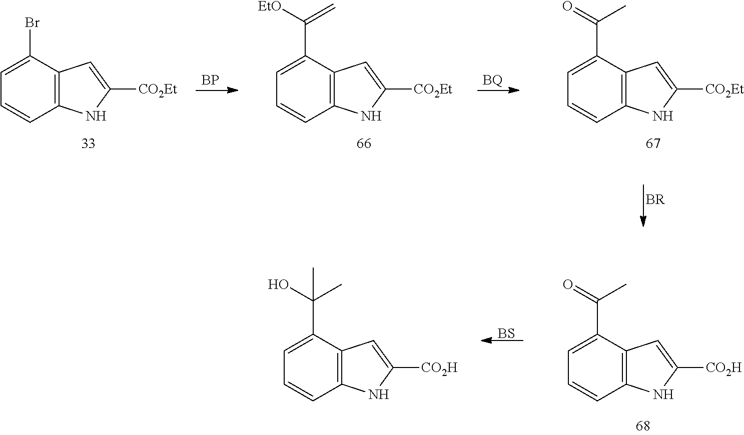

Steps BP and BQ: To a degassed solution of compound 33 (1.00 g, 3.94 mmol) and tributyl-(1-ethoxyvinyl)stannane (1.58 g, 4.37 mmol) in DMF (25 mL) under argon was added bis(triphenylphosphine)palladium(II) dichloride (0.100 g, 0.142 mmol). The reaction mixture was stirred at room temperature until TLC revealed completion of the reaction (approx. 7 days). The mixture was concentrated under reduced pressure and the residue partitioned between ethyl acetate and water. The organic layer was filtered through a plug of silica gel, dried over MgSO₄, and concentrated under reduced pressure. The resulting black oil was dissolved in methanol (100 mL), treated with 5N hydrochloric acid (100 mL), and stirred at room temperature overnight. The mixture was concentrated and the residue dissolved in ethyl acetate. The solution was washed with water, dried over Na₂SO₄, and concentrated under reduced pressure. The crude product was purified by silica gel column chromatography to give 0.500 g (2.30 mmol, 58%) of compound 67.

Step BR: To a solution of compound 67 (1.00 g, 4.60 mmol) in THF (50 mL), was added 1N aqueous LiOH (7 mL). The resulting mixture was stirred for 48 h at room temperature, then concentrated under reduced pressure and diluted with 1N aqueous NaHSO₄ (7 mL). The mixture was extracted with ethyl acetate. The organic extract was dried over MgSO₄, and concentrated under reduced pressure. The residue was recrystallized from MTBE to obtain 0.900 g (4.43 mmol, 96%) of compound 68.

Step BS: To a cooled (0° C.) solution of compound 68 (0.900 g, 4.43 mmol) in THF (50 mL) under argon was added a 1N solution of MeMgCl (16 mL) in hexane. The resulting mixture was stirred for 48 h at room temperature. The mixture was carefully quenched with 1N NaHSO₄ and extracted with ethyl acetate. The organic extract was dried over Na₂SO₄, and concentrated under reduced pressure. The residue was recrystallized from MTBE to obtain 0.250 g (1.14 mmol, 26%) of target compound 4-(2-hydroxypropan-2-yl)-1H-indole-2-carboxylic acid.

Rt (Method G) 0.99 mins, m/z 202 [M−H]⁻

### Preparation of 4-(1-hydroxyethyl)-1H-Indole-2-carboxylic Acid

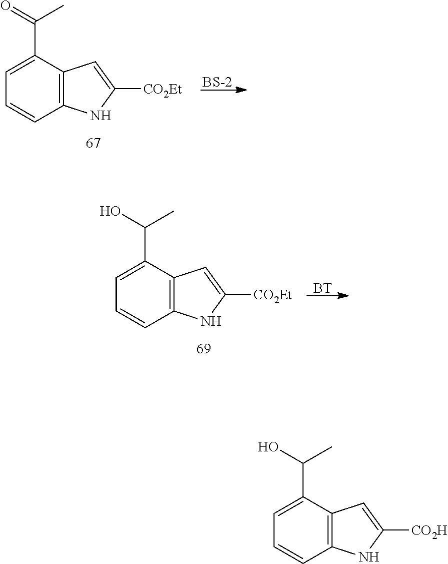

Step BS-2: To a cooled (0° C.) solution of compound 67 (1.00 g, 4.60 mmol) in THF/methanol mixture (50 mL) was added NaBH₄ (0.385 g, 10.2 mmol). The reaction mixture was stirred for 12 h at room temperature. The mixture was cooled to 0° C., treated with 2N hydrochloric acid (20 mL), and concentrated. The residue was extracted with ethyl acetate. The organic extract was washed with water, dried over Na₂SO₄, and evaporated under reduced pressure to obtain 0.800 g (3.65 mmol, 79%) of compound 69, pure enough for the next step.

Step BT: To a solution of compound 69, obtained in the previous step, (0.800 g, 3.65 mmol) in THF (50 mL), was added 1N aqueous LiOH (6 mL). The resulting mixture was stirred for 48 h at room temperature, then concentrated and diluted with 1N aqueous NaHSO₄ (6 mL). The mixture was extracted with ethyl acetate. The organic extract was dried over MgSO₄, and concentrated under reduced pressure. The residue was recrystallized from MTBE to obtain 0.300 g (1.46 mmol, 40%) of target compound 4-(1-hydroxyethyl)-1H-indole-2-carboxylic acid.

Rt (Method G) 0.82 mins, m/z 204 [M−H]⁻

### Preparation of 4-(propan-2-yl)-1H-indole-2-carboxylic Acid

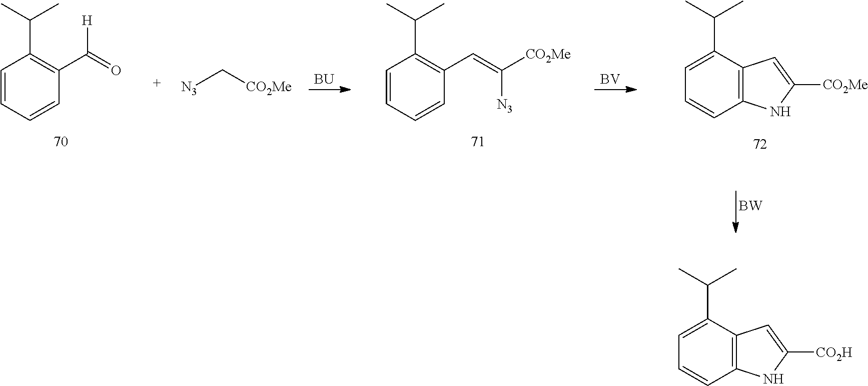

Step BU: To a solution of sodium methoxide (10.0 g, 185 mmol) in methanol (150 mL) at −10° C. was added dropwise a solution of compound 70 (15.0 g, 101 mmol) and methyl azidoacetate (12.0 g, 104 mmol) in methanol (100 mL). The reaction mixture was stirred for 3 h maintaining the temperature below 5° C., then quenched with ice water. The resulting mixture was stirred for 10 min. The precipitate was then collected by filtration, washed with water and dried to afford 7.00 g (23.3 mmol, 23%) of compound 71 as a white solid.

Step BV: A solution of compound 71, obtained in the previous step, (7.00 g, 23.3 mmol) in xylene (200 mL) was refluxed for 1 h under an argon atmosphere and then concentrated under reduced pressure. The residue was recrystallized from hexane-ethyl acetate (60:40) to give 3.50 g (16.1 mmol, 69%) of compound 72.

Step BW: To a solution of compound 72 (3.50 g, 16.1 mmol) in methanol (100 mL) was added 2N aqueous NaOH (40 mL). The mixture was stirred for 2 h at 60° C. The mixture was concentrated under reduced pressure, and then residue acidified to pH 5-6 with 10% hydrochloric acid. The precipitate was collected by filtration, washed with water (3×50 mL), and dried to obtain 2.70 g (13.3 mmol, 83%) of target compound 4-(propan-2-yl)-1H-indole-2-carboxylic acid.

Rt (Method G) 1.32 mins, W/z 202 [M−H]⁻

### Preparation of 4-ethenyl-1H-indole-2-carboxylic Acid

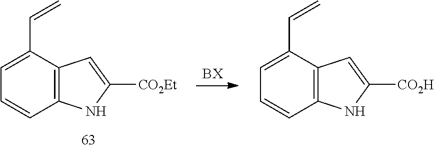 [SMILES: C=Cc1cccc2[nH]c(C(=O)OCC)cc12 ; C=Cc1cccc2[nH]c(C(=O)O)cc12]

Step BX: To a solution of compound 63 (0.900 g, 4.47 mmol) in THF (50 mL), was added 1N aqueous LiOH (8 mL). The resulting mixture was stirred for 48 h at room temperature, then concentrated under reduced pressure and diluted with 1N aqueous NaHSO₄ (8 mL). The mixture was extracted with ethyl acetate. The organic extract was dried over MgSO₄ and concentrated under reduced pressure. The residue was recrystallized from MTBE to obtain 0.500 g (2.67 mmol, 59%) of target compound 4-ethenyl-1H-indole-2-carboxylic acid.

Rt (Method G) 1.14 mins, m/z 186 [M−H]⁻

### Preparation of 4-ethynyl-1H-indole-2-carboxylic Acid

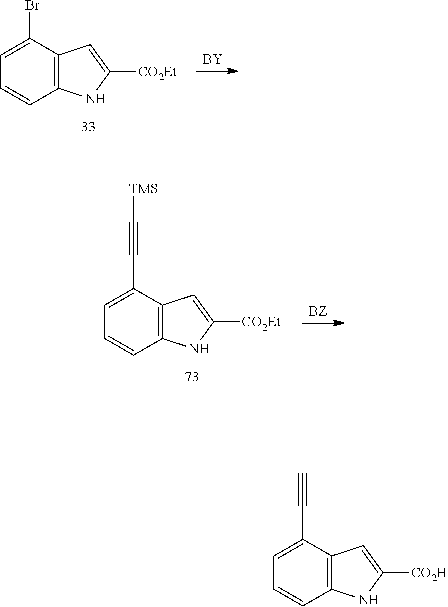 [SMILES: CCOC(=O)c1cc2c(Br)cccc2[nH]1 ; "BY" ; CCOC(=O)c1cc2c(C#C[Si](C)(C)C)cccc2[nH]1 ; "BZ" ; C#Cc1cccc2[nH]c(C(=O)O)cc12]

Step BY: To a solution of compound 33 (1.00 g, 3.94 mmol) in THF (50 mL) under argon were added TMS-acetylene (0.68 mL, 4.80 mmol), CuI (0.076 g, 0.399 mmol), triethylamine (2.80 mL, 20.0 mmol), and Pd(dppf)Cl₂ (0.100 g, 0.137 mmol). The mixture was stirred at 60° C. until TLC revealed completion of the reaction (approx. 5 days). The mixture was concentrated under reduced pressure, and the residue dissolved in ethyl acetate. The solution was washed with water, dried over Na₂SO₄, and concentrated under reduced pressure. The residue was purified by silica gel column chromatography to give 0.600 g (2.14 mmol, 56%) of compound 73.

Step BZ: To a solution of compound 73 (0.840 g, 3.10 mmol) in THF (50 mL), was added 1N aqueous LiOH (7 mL). The resulting mixture was stirred for 48 h at room temperature, then concentrated under reduced pressure and diluted with 1N aqueous NaHSO₄ (7 mL). The mixture was extracted with ethyl acetate. The organic extract was dried over MgSO₄ and concentrated under reduced pressure. The residue was recrystallized from MTBE to obtain 0.400 g (2.17 mmol, 70%) of target compound 4-ethynyl-1H-indole-2-carboxylic acid.

Rt (Method G) 1.12 mins, m/z 184 [M−H]⁻

### Preparation of 4-(1,1-difluoroethyl)-1H-indole-2-carboxylic Acid

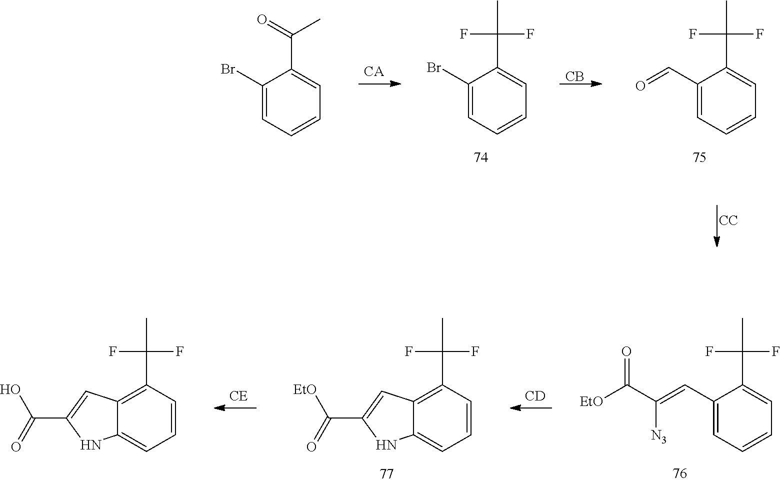

Step CA: To a mixture of 2-bromoacetophenone (63.0 g, 317 mmol), water (0.5 mL), and dichloromethane (100 mL) was added Morph-DAST (121 mL, 992 mmol). The resulting mixture was stirred for 28 days at room temperature. The reaction mixture was then poured into saturated aqueous NaHCO₃ (1000 mL) and extracted with ethyl acetate (2×500 mL). The organic layer was dried over Na₂SO₄ and concentrated under reduced pressure. The residue was purified by silica gel column chromatography to give 16.8 g (76.0 mmol, 12%) of compound 74.

Step CB: To a cooled (−85° C.) solution of compound 74 (16.8 g, 76.0 mmol) in THF (300 mL) under Ar was added 2.5M solution of n-BuLi in hexanes (36.5 mL, 91.5 mmol) over 30 min. The resulting mixture was stirred for 1 h at −85° C. DMF (8.80 mL, 114 mmol) was then added (maintaining temperature below −80° C.) and the reaction stirred for a further 45 min. The reaction was quenched with saturated aqueous NH₄Cl (100 mL) and diluted with water (600 mL). The obtained mixture was extracted with ethyl acetate (2×500 mL). The combined organic extracts were dried over Na₂SO₄, and concentrated under reduced pressure to obtain 12.5 g (73.6 mmol, 97%) of compound 75 (sufficiently pure for the next step).

Step CC: To a cooled (−30° C.) mixture of compound 75 (12.5 g, 73.5 mmol), ethanol (500 mL), and ethyl azidoacetate (28.5 g, 221 mmol) was added a freshly prepared solution of sodium methoxide (prepared by mixing Na (5.00 g, 217 mmol) and methanol (100 mL)) portionwise under Ar (maintaining the temperature below −25° C.). The reaction mixture was warmed to 15° C. and stirred for 12 h. The obtained mixture was poured into saturated aqueous NH₄Cl (2500 mL) and stirred for 20 min. The precipitate was collected by filtration, washed with water, and dried to obtain 10.0 g (35.6 mmol, 51%) of compound 76.

Step CD: A solution of compound 76 (10.0 g, 35.6 mmol) in xylene (500 mL) was refluxed until gas evolution ceased (approx. 2 h) and then concentrated under reduced pressure. The orange oil obtained was triturated with hexane/ethyl acetate (5:1), collected by filtration, and dried to obtain 1.53 g (6.04 mmol, 17%) of compound 77.

Step CE: To a solution of compound 77 (1.53 g, 6.04 mmol) in THF/water 9:1 mixture (100 mL) was added LiOH H₂O (0.590 g, 14.1 mmol). The resulting mixture was stirred overnight at r.t. The volatiles were evaporated and the residue mixed with water (50 mL) and 1N hydrochloric acid (10 mL). The mixture was extracted with ethyl acetate (2×100 mL). The combined organic extracts were dried over Na₂SO₄, and concentrated under reduced pressure. The crude product was purified by silica gel column chromatography to give 0.340 g (1.33 mmol, 24%) of 4-(1,1-difluoroethyl)-1H-indole-2-carboxylic acid.

Rt (Method G) 1.16 mins, m/z 224 [M−H]⁻

### Preparation of 4-(trimethylsilyl)-1H-indole-2-carboxylic Acid

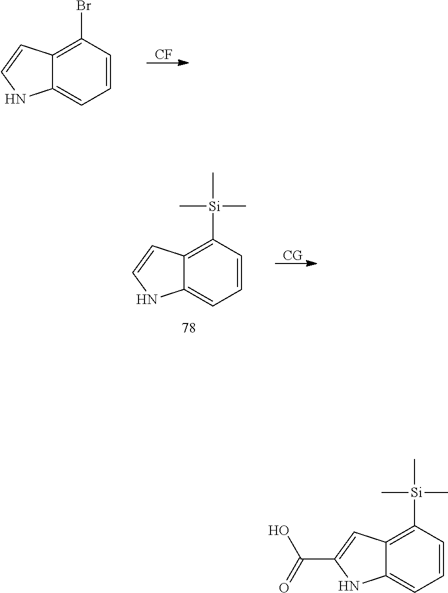

Step CF: To a cooled (−78° C.) solution of 4-bromo-1H-indole (5.00 g, 25.5 mmol) in THF (100 mL) under Ar was added a 2.5M solution of n-BuLi in hexanes (23 mL, 57.5 mmol). The resulting mixture was stirred for 30 min. TMSCl (16 mL, 126 mmol) was added and the reaction mixture warmed to room temperature. After 1 h the mixture was diluted with MTBE (250 mL), washed with water (2×200 mL) and brine (200 mL), then dried over Na₂SO₄, and concentrated under reduced pressure. The residue was refluxed in methanol (100 mL) for 1 h. The solvent was then distilled off to obtain 3.60 g (19.0 mmol, 74%) of compound 78.

Step CG: To a cooled (−78° C.) solution of compound 78 (1.50 g, 7.92 mmol) in THF (50 mL) under Ar was added a 2.5M solution of n-BuLi in hexanes (3.8 mL, 9.5 mmol). The resulting mixture was stirred for 20 min. CO₂ (2 L) was then bubbled through the mixture for 10 min, and the reaction mixture warmed to room temperature. The volatiles were evaporated and the residue dissolved in THF (50 mL). The solution was cooled to −78° C., and a 1.7M solution of t-BuLi (5.6 mL, 9.50 mmol) was added. The mixture was warmed to −30° C., then again cooled to −78° C. CO₂ (2 L) was bubbled through the solution for 10 min. The obtained solution was allowed to slowly warm to r.t. then concentrated under reduced pressure. The residue was dissolved in water (50 mL), washed with MTBE (2×50 mL), then acidified to pH 4, and extracted with ethyl acetate (2×50 mL). The organic extract was washed with water (2×50 mL), and brine (50 mL), dried over Na₂SO₄, and evaporated under reduced pressure. The crude product was washed with hexane and dried to obtain 1.24 g (5.31 mmol, 67%) of target compound 4-(trimethylsilyl)-1H-indole-2-carboxylic acid.

Rt (Method G) 1.47 mins, m/z 232 [M−H]⁻

### Preparation of 6-chloro-5-fluoro-1H-indole-2-carboxylic Acid

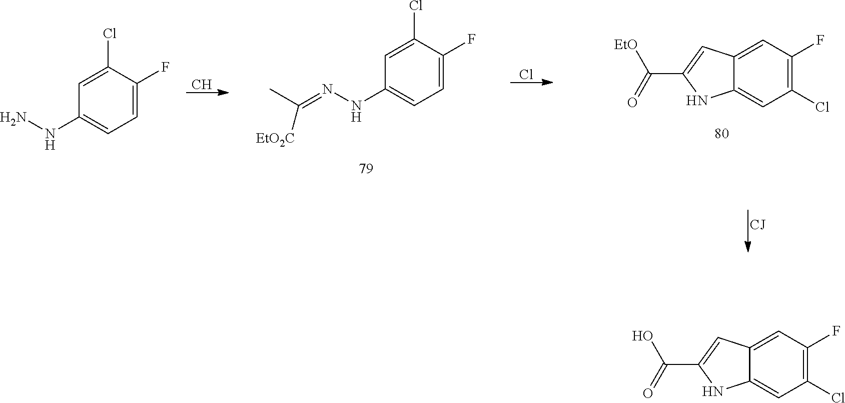

Step CH: To a solution of (3-chloro-4-fluorophenyl)hydrazine (80.0 g, 498 mmol) in ethanol (200 mL) was added ethyl pyruvate (58.0 g, 499 mmol). The mixture was refluxed for 1 h, then concentrated under reduced pressure, and diluted with water (300 mL). The solid was collected by filtration then dried to obtain 122 g (472 mmol, 95%) of compound 79.

Step CI: A suspension of compound 79 (122 g, 472 mmol) and pTSA (81.5 g, 473 mmol) in toluene (500 mL) was refluxed for 48 h, then cooled to room temperature. The precipitate was collected by filtration and purified by fractional crystallization from toluene to obtain 4.00 g (16.6 mmol, 4%) of compound 80.

Step CJ: To a refluxing solution of compound 80 (4.00 g, 16.6 mmol) in ethanol (30 mL) was added NaOH (0.660 g, 16.5 mmol). The mixture was refluxed for 1 h, then concentrated under reduced pressure. The residue was triturated with warm water (80° C., 50 mL) and the solution acidified (pH 2) with concentrated hydrochloric acid. The precipitate was collected by filtration, washed with water (2×10 mL), and dried to obtain 3.18 g (14.9 mmol, 90%) of target compound 6-chloro-5-fluoro-1H-indole-2-carboxylic acid.

Rt (Method G) 1.23 mins, m/z 212 [M−H]⁻

### Preparation of 4-(difluoromethyl)-6-fluoro-1H-Indole-2-carboxylic Acid

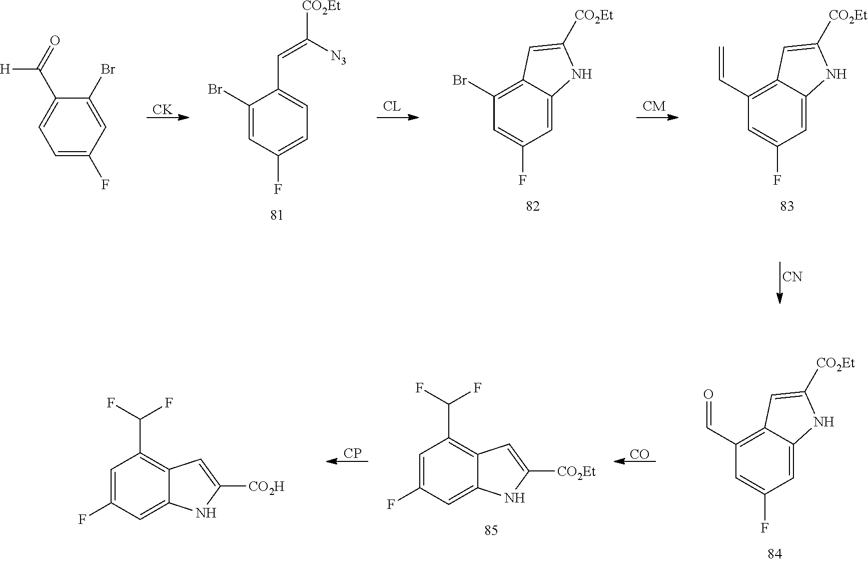

Step CK: To a solution of sodium methoxide (50.0 g, 926 mmol) in methanol (300 mL) at −10° C. was added dropwise a solution of 2-bromo-4-fluorobenzaldehyde (222 mmol) and methyl azidoacetate (59.0 g, 457 mmol) in methanol (100 mL). The reaction mixture was stirred for 3 h, maintaining the temperature below 5° C., then quenched with ice water. The resulting mixture was stirred for 10 min and the solid collected by filtration. The solid was washed with water to afford compound 81 as a white solid (62% yield).

Step CL: A solution of compound 81 (133 mmol) in xylene (250 mL) was refluxed for 1 h under an argon atmosphere and then concentrated under reduced pressure. The residue was recrystallized form hexane-ethyl acetate mixture (60:40) to give compound 82 (58% yield).

Step CM: To a heated (90° C.) solution of compound 82 (14.7 mmol) in anhydrous DMF (10 mL) tri-n-butyl(vinyl)tin (3.60 g, 11.4 mmol) and Pd(PPh₃)₂Cl₂ (0.301 g, 0.757 mmol) were added under nitrogen and the resulting mixture was stirred at 90° C. for 1 h. The mixture was cooled to room temperature and purified by silica gel column chromatography (60-80% ethyl acetate in hexane). The combined product fractions were concentrated, washed with water (3×100 mL), dried over Na₂SO₄, and concentrated under reduced pressure to afford compound 83 as a yellow solid (60% yield).

Step CN: To a mixture of compound 83 (12.4 mmol), acetone (200 mL), and water (40 mL) OsO₄ (0.100 g, 0.393 mmol) and NaIO₄ (13.4 g, 62.6 mmol) were added and the reaction was stirred for 10 h at room temperature. Acetone was distilled off and the aqueous solution was extracted with dichloromethane. The combined organic layer was washed with saturated NaHCO₃ solution (2×50 mL) and brine (2×50 mL), dried over Na₂SO₄, and concentrated under reduced pressure to afford compound 84 (33% yield).

Step CO: To a solution of compound 84 (11.0 mmol) in dichloromethane (50 mL) was added Morph-DAST (4.10 mL, 33.6 mmol). The resulting mixture was stirred until NMR of an aliquot revealed completion of the reaction (2-5 days). The reaction mixture was added dropwise to a cold saturated NaHCO₃ solution (1000 mL). The mixture obtained was extracted with ethyl acetate. The organic layer was dried over MgSO₄ and concentrated. The residue was purified by column chromatography to give compound 85 as yellow solid (48% yield).

Step CP: To a solution of compound 85 (4.50 mmol) in THF (50 mL), was added 1N aqueous LiOH (8 mL). The resulting mixture was stirred for 48 h at room temperature then concentrated under reduced pressure and diluted with 1N aqueous NaHSO₄ (8 mL). The obtained mixture was extracted with ethyl acetate. The organic extract was dried over MgSO₄ and concentrated under reduced pressure. The residue was recrystallized from MTBE to obtain 4-(difluoromethyl)-6-fluoro-1H-indole-2-carboxylic acid (87%).

Rt (Method G) 1.22 mins, m/z 228 [M−H]⁻

### Preparation of 4-(difluoromethyl)-7-fluoro-1H-indole-2-carboxylic Acid

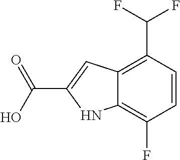 [SMILES: O=C(O)c1cc2c(C(F)F)ccc(F)c2[nH]1]

Prepared as described for 4-(difluoromethyl)-6-fluoro-1H-indole-2-carboxylic acid, starting from 2-bromo-5-fluorobenzaldehyde (2.5% overall yield).

Rt (Method G) 1.13 mins, m/z 228 [M−H]⁻

### Preparation of 4-(1,1-difluoroethyl)-6-fluoro-1H-indole-2-carboxylic Acid

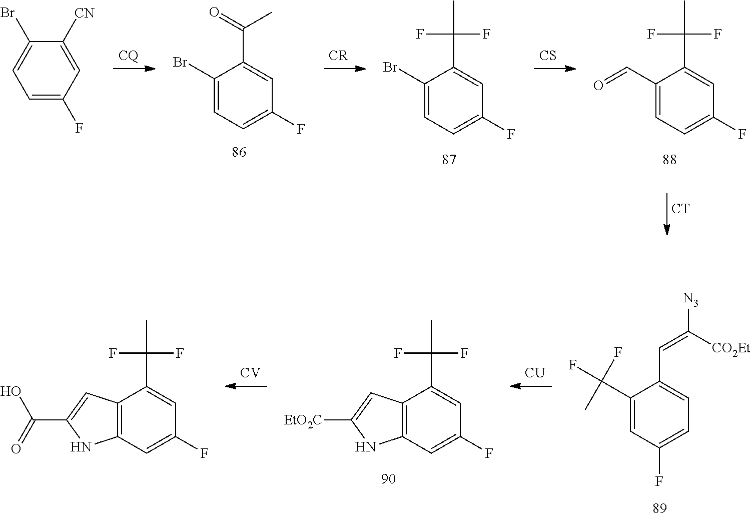

Step CQ: To a solution of 2-bromo-5-fluorobenzonitrile (10.0 g, 48.5 mmol) in anhydrous tetrahydrofuran (100 mL) under nitrogen was added methylmagnesium bromide (3.2M in ether, 19 mL, 60.0 mmol). The resulting mixture was heated to reflux for 4 h. The reaction mixture was then cooled, poured into 2N hydrochloric acid (100 mL), and diluted with methanol (100 mL). The organic solvents were removed and the crude product precipitated out. The reaction mixture was extracted with ethyl acetate, dried over MgSO₄, and concentrated. The residue was purified by column chromatography (heptane/dichloromethane) to give 4.88 g (21.9 mmol, 45%) of compound 86 as a pink oil.

Step CR: To a solution of compound 86 (110 mmol) in dichloromethane (50 mL) at room temperature was added Morph-DAST (41 mL, 336 mmol) and a few drops of water. The resulting mixture was stirred for 48 days at room temperature; every 7 days an additional portion of Morph-DAST (41 mL, 336 mmol) was added. After the reaction was complete, the mixture was carefully added dropwise to cold saturated aqueous NaHCO₃. The product was extracted with ethyl acetate and the organic extract dried over MgSO₄ and concentrated. The residue was purified by column chromatography to give 87 as a colorless liquid (37% yield).

Step CS: To a cooled (−80° C.) solution of compound 87 (21.0 mmol) in THF (150 mL) was added slowly a 2.5M solution of n-BuLi in hexanes (10.0 mL, 25.0 mmol of n-BuLi). The mixture was stirred for 1 h, then DMF (2.62 mL, 33.8 mmol) was added and the mixture stirred for a further 1 h. The reaction was quenched with saturated aqueous NH₄Cl (250 mL) and extracted with Et₂O (3×150 mL). The organic layer was dried over Na₂SO₄ and concentrated under reduced pressure. The residue was purified by silica gel chromatography (ethyl acetate/hexane 1:9) to give compound 88 (52% yield).

Step CT: To a solution of sodium methoxide (50.0 g, 926 mmol) in methanol (300 mL) at −10° C. was added dropwise a solution of compound 88 (222 mmol) and methyl azidoacetate (59.0 g, 457 mmol) in methanol (100 mL). The reaction mixture was stirred for 3 h, maintaining the temperature below 5° C., then quenched with ice water. The resulting mixture was stirred for 10 min. The solid obtained was collected by filtration, and washed with water to afford compound 89 as a white solid (66% yield).

Step CU: A solution of compound 89 (120 mmol) in xylene (250 mL) was refluxed for 1 h under an argon atmosphere and then concentrated under reduced pressure. The residue was recrystallized from hexane-ethyl acetate to give compound 90 (70% yield).

Step CV: To a solution of compound 90 (4.40 mmol) in THF (50 mL) was added 1N aqueous LiOH (8 mL). The resulting mixture was stirred for 48 h at room temperature, then concentrated under reduced pressure and diluted with 1N aqueous NaHSO₄ (8 mL). The residue obtained was extracted with ethyl acetate. The organic extract was dried over MgSO₄ and concentrated under reduced pressure. The residue was recrystallized from MTBE to obtain target compound 4-(1,1-difluoroethyl)-6-fluoro-1H-indole-2-carboxylic acid (95% yield).

Rt (Method G) 1.26 mins, m/z 242 [M−H]⁻

### Preparation of 6,6-difluoro-4-azaspiro[2.4]heptane

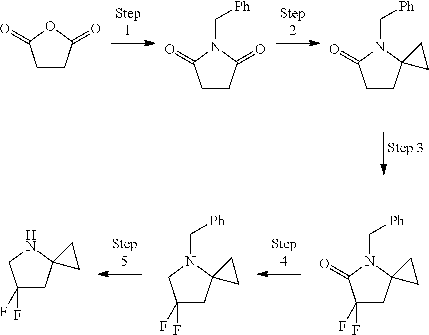 [SMILES: O=C1CCC(=O)O1 ; O=C1CCC(=O)N1Cc1ccccc1 ; O=C1CCC2(CC2)N1Cc1ccccc1 ; FC1(F)CNC2(CC2)C1 ; FC1(F)CN(Cc2ccccc2)C2(CC2)C1 ; O=C1N(Cc2ccccc2)C2(CC2)CC1(F)F]

Step 1: To a solution of succinic anhydride (100 g, 1000 mmol) in toluene (3000 mL) was added benzylamine (107 g, 1000 mmol). The solution was stirred at room temperature for 24 h, then heated at reflux with a Dean-Stark apparatus for 16 hours. The mixture was then concentrated under reduced pressure to give 1-benzylpyrrolidine-2,5-dione (170 g, 900 mmol, 90% yield).

Step 2: To a cooled (0° C.) mixture of 1-benzylpyrrolidine-2,5-dione (114 g, 600 mmol) and Ti(Oi-Pr)₄ (170.5 g, 600 mmol) in dry THF (2000 mL) under argon atmosphere was added dropwise a 3.4M solution of ethylmagnesium bromide in THF (1200 mmol). The mixture was warmed to room temperature and stirred for 4 h. BF₃.Et₂O (170 g, 1200 mmol) was then added dropwise and the solution stirred for 6 h. The mixture was cooled (0° C.) and 3N hydrochloric acid (500 mL) was added. The mixture was extracted twice with Et₂O, and the combined organic extracts washed with brine, dried and concentrated under reduced pressure to give 4-benzyl-4-azaspiro[2.4]heptan-5-one (30.2 g, 150 mmol, 25% yield).

Step 3: To a cooled (−78° C.) solution of 4-benzyl-4-azaspiro[2.4]heptan-5-one (34.2 g, 170 mmol) in dry THF (1000 mL) under argon was added LiHMDS in THF (1.1M solution, 240 mmol). The mixture was stirred for 1 h, then a solution of N-fluorobenzenesulfonimide (75.7 g, 240 mmol) in THF (200 mL) was added dropwise. The mixture was warmed to room temperature and stirred for 6 h. The mixture was then re-cooled (−78° C.) and LiHMDS added (1.1M solution in THF, 240 mmol).

The solution was stirred for 1 h, then N-fluorobenzenesulfonimide (75.7 g, 240 mmol) in THF (200 mL) was added dropwise. The mixture was warmed to room temperature and stirred for 6 h. The mixture was poured into a saturated solution of NH₄Cl (300 mL) and extracted twice with Et₂O. The combined organic extracts were washed with brine and concentrated under reduced pressure. Product was purified by column chromatography to provide 4-benzyl-6,6-difluoro-4-azaspiro[2.4]heptan-5-one (18 g, 75.9 mmol, 45% yield).

Step 4: To a warmed (40° C.) solution of BH₃.Me₂S (3.42 g, 45 mmol) in THF (200 mL) was added dropwise 4-benzyl-6,6-difluoro-4-azaspiro[2.4]heptan-5-one (11.9 g, 50 mmol). The mixture was stirred for 24 h at 400° C., then cooled to room temperature. Water (50 mL) was added dropwise, and the mixture extracted with Et₂O (2×200 mL). The combined organic extracts were washed brine, diluted with 10% solution of HCl in dioxane (50 mL) and evaporated under reduced pressure to give 4-benzyl-6,6-difluoro-4-azaspiro[2.4]heptane (3 g, 13.4 mmol, 27% yield).

Step 5: 4-benzyl-6,6-difluoro-4-azaspiro[2.4]heptane (2.68 g, 12 mmol) and palladium hydroxide (0.5 g) in methanol (500 mL) were stirred at room temperature under an atmosphere of H₂ for 24 h. The mixture was filtered and then filtrate concentrated under reduced pressure to obtain 6,6-difluoro-4-azaspiro[2.4]heptane (0.8 g, 6.01 mmol, 50% yield).

### Preparation of 7,7-difluoro-4-azaspiro[2.4]heptane

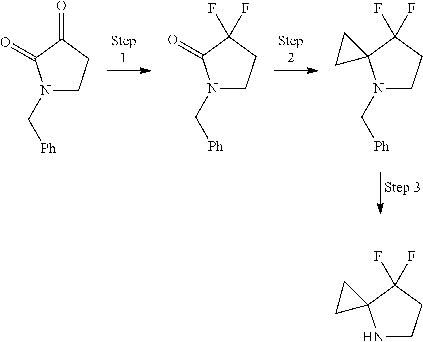 [SMILES: O=C1CCN(Cc2ccccc2)C1=O ; O=C1N(Cc2ccccc2)CCC1(F)F ; FC1(F)CCN(Cc2ccccc2)C12CC2 ; FC1(F)CCNC12CC2]

Step 1: To a cooled (00° C.) solution of 1-benzylpyrrolidine-2,3-dione (8 g, 42.3 mmol) in DCM (100 mL) was added dropwise over 30 minutes DAST (20.4 g, 127 mmol). The mixture was stirred at room temperature overnight, then quenched by dropwise addition of saturated NaHCO₃. The organic layer was separated, and the aqueous fraction extracted twice with DCM (2×50 mL). The combined organic layers were dried over Na₂SO₄ and concentrated under reduced pressure to afford 1-benzyl-3,3-difluoropyrrolidin-2-one (26.0 mmol, 61% yield), which used in the next step without further purification.

Step 2: To a solution of crude 1-benzyl-3,3-difluoropyrrolidin-2-one (5.5 g, 26 mmol) and Ti(Oi-Pr)₄ (23.4 mL, 78 mmol) in THF (300 mL) was added dropwise under argon atmosphere 3.4 M solution of EtMgBr in 2-MeTHF (45.8 mL, 156 mmol). After stirring for 12 h, water (10 mL) was added to obtain a white precipitate. The precipitate was washed with MTBE (3×50 mL). The combined organic fractions were dried over Na₂SO₄ concentrated and purified by flash chromatography (hexanes-EtOAc 9:1) to obtain 4-benzyl-7,7-difluoro-4-azaspiro[2.4]heptane (1.3 g, 5.82 mmol, 22% yield) as a pale yellow oil.

Step 3: 4-benzyl-7,7-difluoro-4-azaspiro[2.4]heptane (0.55 g, 2.46 mmol) was dissolved in solution of CHCl₃ (1 mL) and MeOH (20 mL) and Pd/C (0.2 g, 10%) was added. This mixture was stirred under and an H₂ atmosphere for 5 h, then filtered. The filtrate was concentrated to give 7,7-difluoro-4-azaspiro[2.4]heptane (0.164 g, 1.23 mmol, 50% yield)

### Synthesis of 1-[(difluoromethoxy)methyl]-N-methylcyclopropan-1-amine

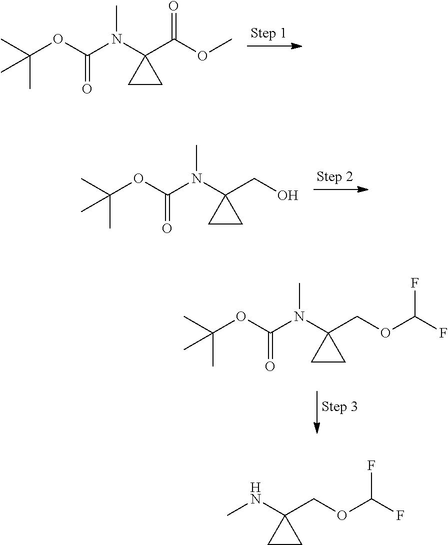

Step 1: To a solution of methyl 1-((tertbutoxycarbonyl)(methyl)amino)cyclopropane-1-carboxylate (1.05 g, 4.58 mmol) in dry THF (5 ml) under N2 was added lithium borohydride (1.259 ml, 4 M in THF, 5.04 mmol). The mixture was stirred at rt for 4 days. Sodium sulfate and water were added, the mixture was filtered over a pad of sodium sulfate which was rinsed with dichloromethane. The filtrate was concentrated, to give tert-butyl (1-(hydroxymethyl)cyclopropyl)(methyl)carbamate as a white solid (0.904 g, 95% yield).

Step 2: To a solution of tert-butyl (1-(hydroxymethyl)cyclopropyl)(methyl)carbamate (0.100 g, 0.497 mmol) and (bromodifluoromethyl)trimethylsilane (0.155 ml, 0.994 mmol) in dichloromethane (0.5 ml) was added one drop of a solution of potassium acetate (0.195 g, 1.987 mmol) in water (0.5 ml). The mixture was stirred for 40 h. The mixture was diluted with dichloromethane and water, the organic layer was separated and concentrated. Purification by flash chromatography (20% ethyl acetate in heptane) gave a tert-butyl N-{1[(difluoromethoxy)methyl]cyclopropyl}-N-methylcarbamate as colorless oil (0.058 g, 46% yield)

Step 3: To tert-butyl (1-((difluoromethoxy)methyl)cyclopropyl)(methyl)carbamate (0.058 g, 0.231 mmol) was added HCl in dioxane (4M solution, 2 ml, 8.00 mmol). The mixture was stirred for 30 min at rt, then concentrated to yield the desired product which was used without further purification

LC-MS: m/z 152.2 (M+H)+

### Synthesis of tert-butyl 3-{bicyclo[3.1.0]hexane-2-carbonyl}-4H,5H,6H,7H-pyrazolo[1,5-a]pyrazine-5-carboxylate

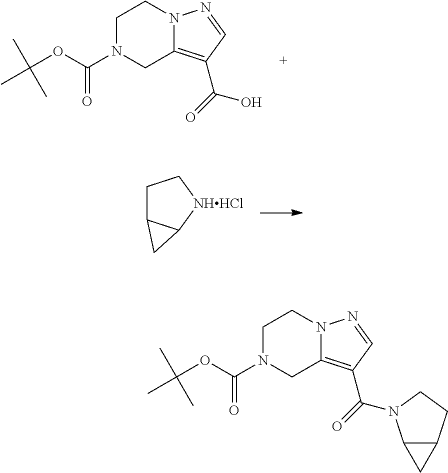

To a stirred solution of 5-[(tert-butoxy)carbonyl]-4H,5H,6H,7H-pyrazolo[1,5-a]pyrazine-3-carboxylic acid (535.0 mg, 2.0 mmol) and triethylamine (445.37 mg, 4.4 mmol, 610.0 μl) in dry DMF (20 mL) was added HATU (836.76 mg, 2.2 mmol) in one portion. The resulting mixture was stirred for 10 min, then 2-azabicyclo[3.1.0]hexane hydrochloride (239.26 mg, 2.0 mmol) was added and the stirring was continued overnight. The reaction mixture was partitioned between EtOAc (70 mL) and water (150 mL). The organic phase was washed with water (2×50 mL), and brine, then dried over sodium sulfate and concentrated under reduced pressure to give a residue which was purified by HPLC to give tert-butyl 3-2-azabicyclo[3.1.0]hexane-2-carbonyl-4H,5H,6H,7H-pyrazolo[1,5-a]pyrazine-5-carboxylate (286.4 mg, 861.62 μmol, 43.1% yield).

¹H NMR (400 MHz, d6-DMSO) δ 0.63 (m, 1H), 0.98 (m, 1H), 1.43 (s, 9H), 1.75 (m, 0H), 1.87 (m, 1H), 2.07 (m, OH), 3.32 (m, 1H), 3.69 (m, 4H), 4.12 (s, 3H), 4.75 (m, 3H), 7.89 (m, 1H).

### Synthesis of tert-butyl 3-{6,6-difluorobicyclo[3.1.0]hexane-2-carbonyl}-4H,5H,6H,7H-pyrazolo[1,5-a]pyrazine-5-carboxylate

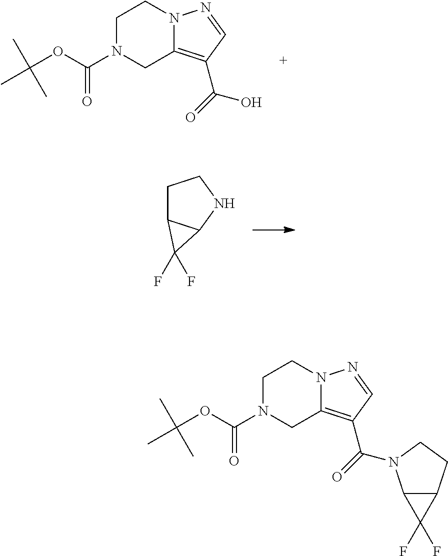 [SMILES: CC(C)(C)OC(=O)N1CCn2ncc(C(=O)O)c2C1 ; FC1(F)C2CCNC21 ; CC(C)(C)OC(=O)N1CCn2ncc(C(=O)N3CCC4C3C4(F)F)c2C1]

To a cooled (−5° C.) solution of 5-[(tert-butoxy)carbonyl]-4H,5H,6H,7H-pyrazolo[1,5-a]pyrazine-3-carboxylic acid (45.86 mg, 171.56 μmol) and 2-chloro-4,6-dimethoxy-1,3,5-triazine (30.12 mg, 171.56 μmol) in dry DCM (5 mL) was added 4-methylmorpholine (17.7 mg, 174.99 μmol, 20.0 μl). The mixture was stirred at 0° C. for 2 h. 4-methylmorpholine (17.7 mg, 174.99 μmol, 20.0 μL) and 6,6-difluoro-2-azabicyclo[3.1.0]hexane 4-methylbenzene-1-sulfonate (50.0 mg, 171.64 μmol) were added to the reaction mixture. Stirring was continued for 1 h, then the mixture was left at r.t. for 10 h. The reaction mixture was partitioned between EtOAc (70 mL) and water (150 mL). The organic phase was washed with water (2×50 mL, and brine, then dried over sodium sulfate and concentrated under reduced pressure to give a residue which was purified by HPLC to give tert-butyl 3-{6,6-difluorobicyclo[3.1.0]hexane-2-carbonyl}-4H,5H,6H,7H-pyrazolo[1,5-a]pyrazine-5-carboxylate.

¹H NMR (d6-DMSO), δ 3.02 (d, 3H), 7.27 (t, 1H), 7.37 (d, 1H), 7.83 (d, 1H), 8.00 (s, 1H), 8.06 (d, 1H), 8.41 (s, 1H), 8.57 (d, 1H), 8.72 (d, 1H), 12.50 (s, 1H), 12.86 (s, 1H).

LCMS (m/z): 268.2

### Synthesis of tert-butyl 3-{bicyclo[3.1.0]hexane-3-carbonyl}-4H,5H,6H,7H-pyrazolo[1,5-a]pyrazine-5-carboxylate

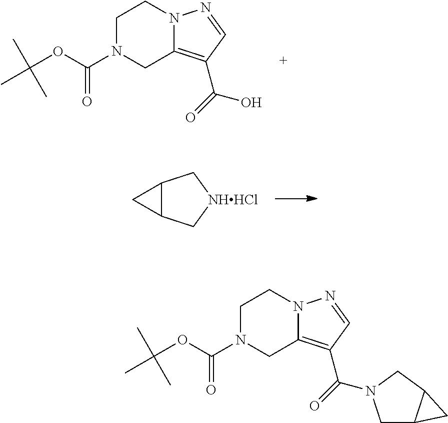

5-[(Tert-butoxy)carbonyl]-4H,5H,6H,7H-pyrazolo[1,5-a]pyrazine-3-carboxylic acid (250.0 mg, 935.34 μmol), HATU (391.22 mg, 1.03 mmol) and triethylamine (236.62 mg, 2.34 mmol, 330.0 μl) were mixed in dry DMF (5 mL) at r.t. and the resulting mixture was stirred for 10 minutes. 3-azabicyclo[3.1.0]hexane hydrochloride (123.05 mg, 1.03 mmol) was added thereto and the resulting mixture was stirred at r.t. overnight. The resulting mixture was partitioned between water (50 mL) and EtOAc (50 mL). The organic phase was separated, dried over Na₂SO₄ and evaporated. The residue was purified by HPLC to give tert-butyl 3-3-azabicyclo[3.1.0]hexane-3-carbonyl-4H,5H,6H,7H-pyrazolo[1,5-a]pyrazine-5-carboxylate (152.0 mg, 457.28 μmol, 48.9% yield) as white solid.

¹H NMR (400 MHz, d6-DMSO) δ 0.03 (m, 1H), 0.69 (m, 1H), 1.42 (s, 9H), 1.55 (m, 1H), 1.63 (m, 1H), 3.78 (m, 1H), 3.80 (m, 4H), 4.10 (m, 2H), 4.68 (m, 1H), 4.74 (m, 2H), 7.81 (s, 1H).

LCMS (m/z): 333.2

### tert-butyl 3-{6,6-difluoro-3-azabicyclo[3.1.0]hexane-3-carbonyl}-4H,5H,6H,7H-pyrazolo[1,5-a]pyrazine-5-carboxylate

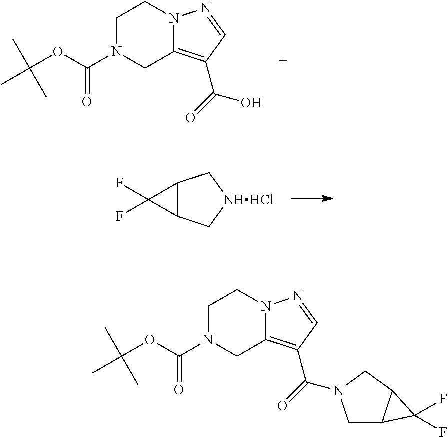

5-[(Tert-butoxy)carbonyl]-4H,5H,6H,7H-pyrazolo[1,5-a]pyrazine-3-carboxylic acid (250.0 mg, 935.34 μmol), HATU (391.26 mg, 1.03 mmol) and triethylamine (236.65 mg, 2.34 mmol, 330.0 μl) were mixed in dry DMF (5 mL) at r.t. and the resulting mixture was stirred for 10 minutes. 6,6-Difluoro-3-azabicyclo[3.1.0]hexane hydrochloride (160.08 mg, 1.03 mmol) was added thereto and the resulting mixture was stirred at r.t. overnight. The resulting mixture was partitioned between water (50 mL) and EtOAc (50 mL). The organic phase was separated, dried over Na₂SO₄ and evaporated. The residue was purified by HPLC to give tert-butyl 3-6,6-difluoro-3-azabicyclo[3.1.0]hexane-3-carbonyl-4H,5H,6H,7H-pyrazolo[1,5-a]pyrazine-5-carboxylate (173.0 mg, 469.63 μmol, 50.2% yield) as white solid.

¹H NMR (400 MHz, d6-DMSO) δ 1.43 (s, 9H), 2.67 (m, 2H), 3.70 (m, 1H), 3.80 (m, 2H), 3.98 (m, 2H), 4.11 (m, 3H), 4.69 (m, 1H), 4.75 (m, 1H), 7.87 (s, 1H).

LCMS: m/z 369.2

### Synthesis of tert-butyl 3-{methyl[1-(pyridin-3-yl)cyclopropyl]carbamoyl}-4H,5H,6H,7H-pyrazolo[1,5-a]pyrazine-5-carboxylate

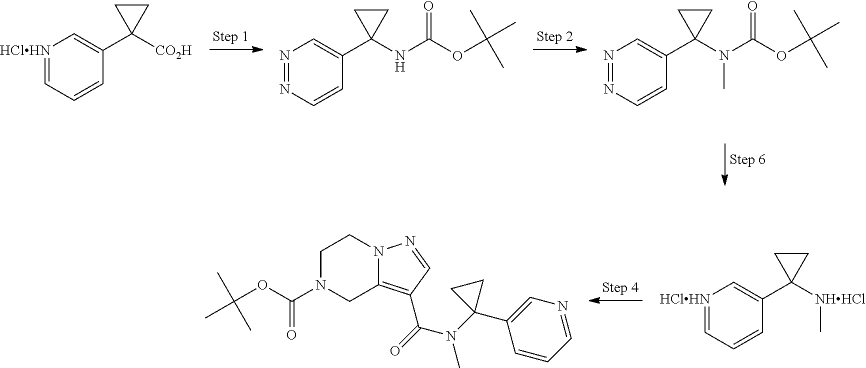

Step 1: To a solution of 1-(pyridin-3-yl)cyclopropane-1-carboxylic acid hydrochloride (498.46 mg, 2.5 mmol) in a mixture of toluene (30 mL) and t-BuOH (10 mL) were added diphenylphosphoryl azide (687.14 mg, 2.5 mmol) and triethylamine (631.62 mg, 6.24 mmol, 870.0 μL). The reaction mixture was heated at reflux overnight. The reaction mixture was cooled and filtered. The filtrate was washed with water (3×10 mL), dried over Na₂SO₄ and concentrated in vacuo to give tert-butyl N-[1-(pyridin-3-yl)cyclopropyl]carbamate (250.0 mg, 95.0% purity, 1.01 mmol, 40.6% yield) as light brown oil.

Step 2: Sodium hydride (154.24 mg, 6.43 mmol) was suspended in dry DMF (5 mL) and then cooled to 0° C. A solution of tert-butyl N-[1-(pyridin-3-yl)cyclopropyl]carbamate (1.51 g, 6.43 mmol) in dry DMF (5 mL) was added dropwise. The resulting mixture was stirred until gas evolution ceased. Iodomethane (1.0 g, 7.07 mmol, 440.0 μl) was added dropwise at that same temperature; the resulting mixture was warmed to r.t. and then stirred overnight. After consumption of the starting material (¹H NMR control) the reaction mixture was poured into water. The resulting mixture was extracted twice with MTBE (2×50 mL). The organic phases were combined, washed with water, dried over sodium sulfate and concentrated to give tert-butyl N-methyl-N-[1-(pyridin-3-yl)cyclopropyl]carbamate (1.1 g, 4.43 mmol, 68.9% yield). The product was used in the next step without further purification.

Step 3: To a solution of tert-butyl N-methyl-N-[1-(pyridin-3-yl)cyclopropyl]carbamate (1.1 g, 4.43 mmol) in methanol (10 mL) was added 4M HCl solution in dioxane (2 mL). The resulting solution was stirred for 12 h at 25° C. Upon completion of the reaction (monitored by ¹H NMR or LCMS), the reaction mixture was concentrated under reduced pressure. The product was triturated with MTBE and collected by filtration, then dried in vacuo at 40° C., to give N-methyl-1-(pyridin-3-yl)cyclopropan-1-amine dihydrochloride (900.0 mg, 95.0% purity, 3.87 mmol, 87.2% yield).

Step 4: To a stirred solution of N-methyl-1-(pyridin-3-yl)cyclopropan-1-amine dihydrochloride (398.89 mg, 1.8 mmol) and 5-[(tert-butoxy)carbonyl]-4H,5H,6H,7H-pyrazolo[1,5-a]pyrazine-3-carboxylic acid (482.15 mg, 1.8 mmol) in DMF (2 mL) were added HATU (891.67 mg, 2.35 mmol) and triethylamine (638.88 mg, 6.31 mmol, 880.0 μl). The mixture was stirred overnight at r.t. and then poured onto water and extracted with MTBE (2×15 mL). The combined organic fractions were washed three times with water, dried over anhydrous sodium sulfate, and the solvent was removed in vacuum. The crude product was purified by HPLC to give tert-butyl 3-methyl[1-(pyridin-3-yl)cyclopropyl]carbamoyl-4H,5H,6H,7H-pyrazolo[1,5-a]pyrazine-5-carboxylate (230.0 mg, 82.0% purity, 474.5 μmol, 26.3% yield).

¹H NMR (400 MHz, d6-DMSO) δ 1.41 (m, 2H), 1.43 (s, 9H), 1.56 (m, 2H), 3.07 (m, 3H), 3.82 (m, 2H), 4.07 (m, 2H), 4.75 (m, 2H), 6.99 (m, 1H), 7.37 (m, 1H), 7.48 (d, 1H), 8.31 (s, 1H), 8.44 (s, 1H).

LCMS: m/z 398.2

### Synthesis of tert-butyl 3-{methyl[1-(pyridin-4-yl)cyclopropyl]carbamoyl}-4H,5H,6H,7H-pyrazolo[1,5-a]pyrazine-5-carboxylate

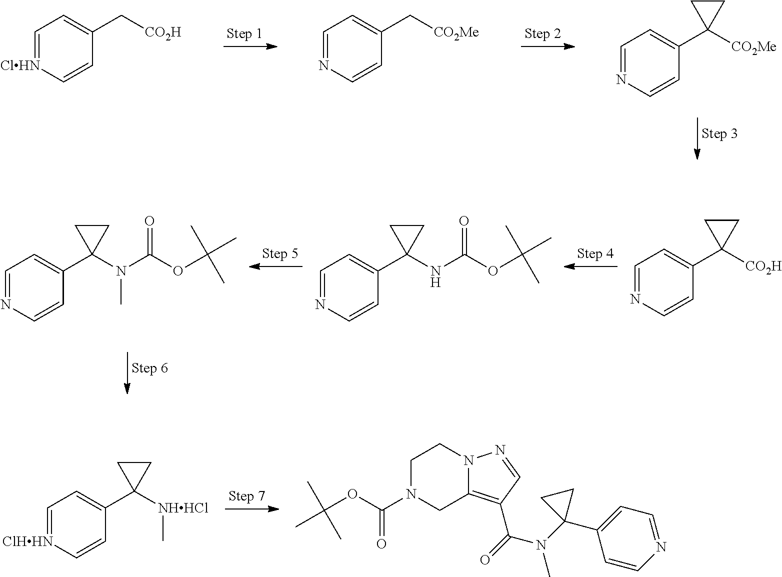

Step 1: 2-(Pyridin-4-yl)acetic acid hydrochloride (5.0 g, 28.8 mmol) was dissolved in MeOH (20 mL), then H₂SO₄ (0.5 mL) was added. The reaction mixture was heated at 85° C. overnight. The MeOH was removed to give a residue which was carefully neutralized with saturated aqueous NaHCO₃ solution and then extracted with EtOAc (3×100 mL). The organic extracts were combined, dried and concentrated to give methyl 2-(pyridin-4-yl)acetate (4.0 g, 95.0% purity, 25.14 mmol, 87.3% yield) as a yellow oil, which was used in the next step without further purification.

Step 2: Methyl 2-(pyridin-4-yl)acetate (4.0 g, 26.46 mmol) was dissolved in DMF (5 mL) and added dropwise to a cooled (0° C.) suspension of sodium hydride (825.52 mg, 34.4 mmol) in DMF (5 mL). The resulting mixture was stirred at 0° C. for 30 min and then treated with 1,2-dibromoethane (6.46 g, 34.4 mmol) at the same temperature. The reaction mixture was stirred at r.t. for 12 h. The reaction mixture was then diluted with ethyl acetate and washed with water and brine. The organic phase was separated, dried over Na₂SO₄ and filtered; the filtrate was concentrated. The resulting oil was triturated with hexane to give methyl 1-(pyridin-4-yl)cyclopropane-1-carboxylate (2.3 g, 12.98 mmol, 49.1% yield) as a solid.

Step 3: Methyl 1-(pyridin-4-yl)cyclopropane-1-carboxylate (2.3 g, 12.98 mmol) was dissolved in MeOH (20 mL), to which was added a solution of sodium hydroxide (778.67 mg, 19.47 mmol) in water (20 mL). The mixture was stirred at 20° C. for 20 h. MeOH was removed by evaporation and the aqueous residue was neutralized under ice cooling with hydrochloric acid (to pH 7). The mixture was concentrated to dryness, the residue was triturated three times with CHCl₃, and the combined filtrates concentrated to dryness to give 1-(pyridin-4-yl)cyclopropane-1-carboxylic Acid hydrochloride (2.0 g, 10.02 mmol, 77.2% yield).

Step 4: To solution of 1-(pyridin-4-yl)cyclopropane-1-carboxylic acid (599.43 mg, 3.67 mmol) in mixture of toluene (30 mL) and t-BuOH (10 mL) were added diphenylphosphoryl azide (1.01 g, 3.67 mmol) and triethylamine (929.28 mg, 9.18 mmol, 1.28 mL). The reaction mixture was refluxed overnight, then cooled and filtered. The filtrate was washed with water (3×10 mL), dried over Na₂SO₄ and concentrated to give tert-butyl N-[1-(pyridin-4-yl)cyclopropyl]carbamate (300.0 mg, 1.28 mmol, 34.9% yield) as light brown oil. The product was used in the next step without further purification.

Step 5: Sodium hydride (94.22 mg, 3.93 mmol) was suspended in DMF (5 mL) and then cooled to 0° C. A solution of tert-butyl N-[1-(pyridin-4-yl)cyclopropyl]carbamate (919.93 mg, 3.93 mmol) in DMF (5 mL) was then added dropwise. The resulting mixture was stirred until gas evolution ceased. Iodomethane (613.04 mg, 4.32 mmol) was added dropwise at that same temperature; the resulting mixture was warmed to r.t. and then stirred overnight. After consumption of the starting material (¹H NMR control) the reaction mixture was poured into water.

The mixture was extracted twice with MTBE (50 mL). The organic phases were combined, washed with water, dried over sodium sulfate and concentrated to give tert-butyl N-methyl-N-[1-(pyridin-4-yl)cyclopropyl]carbamate (900.0 mg, 98.0% purity, 3.55 mmol, 90.5% yield). The product was used in the next step without further purification.

Step 6: To a solution of tert-butyl N-methyl-N-[1-(pyridin-4-yl)cyclopropyl]carbamate (900.0 mg, 3.62 mmol) in methanol (10 mL) was added 4M HCl in dioxane (2 mL) and the resulting solution was stirred for 12 h at 25° C. Upon completion of the reaction (monitored by ¹H NMR), the reaction mixture was concentrated under reduced pressure. The product was treated with MTBE and collected by filtration, then dried in vacuo at 40° C., to give N-methyl-1-(pyridin-4-yl)cyclopropan-1-amine dihydrochloride (600.0 mg, 2.71 mmol, 74.9% yield).

Step 7: To a stirred solution of N-methyl-1-(pyridin-4-yl)cyclopropan-1-amine dihydrochloride (600.0 mg, 2.71 mmol) and 5-[(tert-butoxy)carbonyl]-4H,5H,6H,7H-pyrazolo[1,5-a]pyrazine-3-carboxylic acid (724.91 mg, 2.71 mmol) in DMF (5 mL) were added HATU (1.34 g, 3.53 mmol) and triethylamine (960.55 mg, 9.49 mmol, 1.32 ml). The mixture was stirred overnight at r.t. and then poured into water and extracted with MTBE (3×15 mL). The combined organic fractions were washed three times with water, dried over anhydrous sodium sulfate, and concentrated. The crude product was purified by HPLC to give tert-butyl 3-methyl[1-(pyridin-4-yl)cyclopropyl]carbamoyl-4H,5H,6H,7H-pyrazolo[1,5-a]pyrazine-5-carboxylate (169.0 mg, 425.19 μmol, 15.7% yield).

¹H NMR (400 MHz, d6-DMSO) δ 1.38 (m, 1H), 1.44 (s, 9H), 1.60 (m, 3H), 3.03 (m, 3H), 3.71 (m, 1H), 3.84 (m, 1H), 4.06 (m, 2H), 4.75 (m, 2H), 6.92 (m, 1H), 7.07 (m, 2H), 8.52 (m, 2H).

LCMS: m/z 398.4

### Synthesis of tert-butyl 3-{methyl[1-(pyrimidin-2-yl)cyclopropyl]carbamoyl}-4H,5H,6H,7H-pyrazolo[1,5-a]pyrazine-5-carboxylate

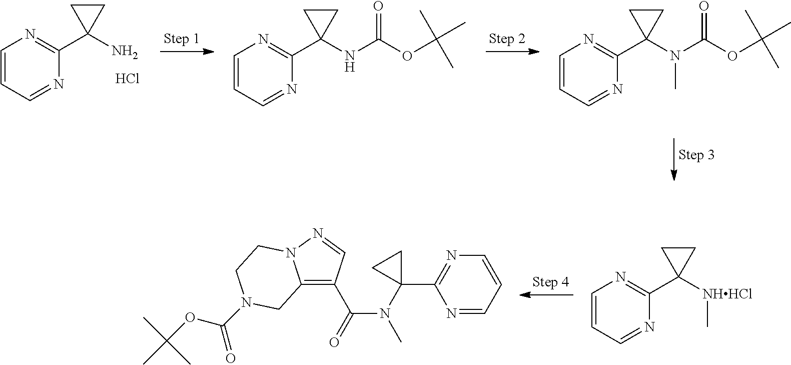

Step 1: To a cooled (0° C.) suspension of 1-(pyrimidin-2-yl)cyclopropan-1-amine hydrochloride (996.43 mg, 5.81 mmol) in dry DCM (30 mL) was added di-tert-butyl dicarbonate (1.27 g, 5.81 mmol). Triethylamine (646.14 mg, 6.39 mmol, 890.0 L) was then added dropwise. The reaction mixture was stirred overnight at r.t and diluted with water (5 mL). The organic phase was separated, washed with water, dried over sodium sulfate, filtered and concentrated to afford tert-butyl N-[1-(pyrimidin-2-yl)cyclopropyl]carbamate (1.17 g, 4.97 mmol, 85.7% yield) as a light yellow solid.

Step 2: To a stirred solution of tert-butyl n-[1-(pyrimidin-2-yl)cyclopropyl]carbamate (499.99 mg, 2.13 mmol) in dry DMF (4 mL) was added sodium hydride (127.49 mg, 5.31 mmol). The reaction mixture was stirred at r.t. for 1 h, then cooled to 0° C. Iodomethane (603.26 mg, 4.25 mmol) was added. The mixture was stirred at r.t. overnight. The mixture was poured into brine; then iextracted with EtOAc (2×10 mL). The combined organic phases were washed with brine, dried over Na₂SO₄, filtered and concentrated to afford tert-butyl N-methyl-N-[1-(pyrimidin-2-yl)cyclopropyl]carbamate (400.0 mg, 1.6 mmol, 75.5% yield) as yellow solid.

Step 3: To a stirred solution of tert-butyl N-methyl-N-[1-(pyrimidin-2-yl)cyclopropyl]carbamate (400.0 mg, 1.6 mmol) in dry DCM (5 mL) was added 4M HCl in dioxane (2 mL, 8 mmol). The reaction mixture was stirred at r.t. for 5 h. The mixture was concentrated, the residue was triturated with hexane and filtered off to afford N-methyl-1-(pyrimidin-2-yl)cyclopropan-1-amine hydrochloride (280.0 mg, 1.51 mmol, 94% yield) as grey solid.

Step 4: To a cooled (0° C.) solution of HATU (573.46 mg, 1.51 mmol) and 5-[(tert-butoxy)carbonyl]-4H,5H,6H,7H-pyrazolo[1,5-a]pyrazine-3-carboxylic acid (403.11 mg, 1.51 mmol) in DMF (3 mL) were added successively N-methyl-1-(pyrimidin-2-yl)cyclopropan-1-amine hydrochloride (280.0 mg, 1.51 mmol) and N,N-diisopropylethylamine (779.69 mg, 6.03 mmol) dropwise. The reaction mixture was stirred at r.t. overnight and diluted with brine. The mixture was extracted with EtOAc (2×10 mL), the combined organic phases were washed with brine, dried over Na₂SO₄ and concentrated. The residue was purified by HPLC to give tert-butyl 3-methyl[1-(pyrimidin-2-yl)cyclopropyl]carbamoyl-4H,5H,6H,7H-pyrazolo[1,5-a]pyrazine-5-carboxylate (332.9 mg, 835.47 μmol, 55.4% yield) as yellow solid.

¹H NMR (400 MHz, d6-DMSO) δ 1.43 (s, 9H), 1.57 (m, 2H), 1.89 (m, 1H), 3.31 (m, 2H), 3.71 (m, 1H), 3.83 (m, 2H), 4.03 (m, 2H), 4.12 (m, 1H), 4.69 (m, 1H), 4.78 (m, 1H), 6.78 (s, 1H), 7.36 (t, 1H), 8.78 (d, 2H).

LCMS: m/z 399.2

### Synthesis of tert-butyl 3-{methyl[1-(pyrimidin-4-yl)cyclopropyl]carbamoyl}-4H,5H,6H,7H-pyrazolo[1,5-a]pyrazine-5-carboxylate

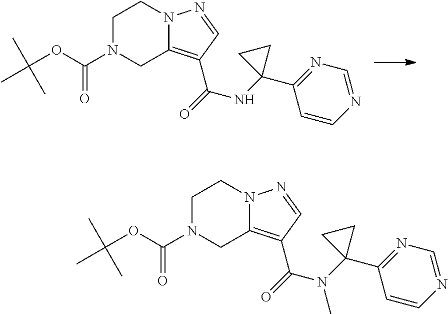 [SMILES: CC(C)(C)OC(=O)N1CCn2ncc(C(=O)NC3(c4ccncn4)CC3)c2C1 ; CN(C(=O)c1cnn2c1CN(C(=O)OC(C)(C)C)CC2)C1(c2ccncn2)CC1]

To a solution of tert-butyl 3-[1-(pyrimidin-4-yl)cyclopropyl]carbamoyl-4H,5H,6H,7H-pyrazolo[1,5-a]pyrazine-5-carboxylate (58.0 mg, 150.87 μmol) in DMF (5 mL) was added sodium hydride (12.07 mg, 502.94 μmol) in one portion. After gas evolution ceased iodomethane (22.49 mg, 158.43 μmol, 10.0 μL) was added and the resulting mixture was left to stir overnight at r.t..

The reaction mixture was poured into water (50 mL) and extracted with EtOAc (2×30 mL). The organic phases were washed with water (30 mL) and brine, dried over Na₂SO₄ and concentrated in vacuo to give crude product, which was purified by HPLC to give tert-butyl 3-methyl[1-(pyrimidin-4-yl)cyclopropyl]carbamoyl-4H,5H,6H,7H-pyrazolo[1,5-a]pyrazine-5-carboxylate (20.0 mg, 50.19 μmol, 33.3% yield).

¹H NMR (400 MHz, CDCl3) δ 3.96 (s, 2H), 7.52 (m, 1H), 7.69 (m, 2H), 7.78 (m, 1H).

LCMS: m/z 399.2

### Synthesis of 2-(1-{5-[(tert-butoxy)carbonyl]-4H,5H,6H,7H-pyrazolo[1,5-a]pyrazin-3-yl}-5-oxopyrrolidin-3-yl)benzoic Acid

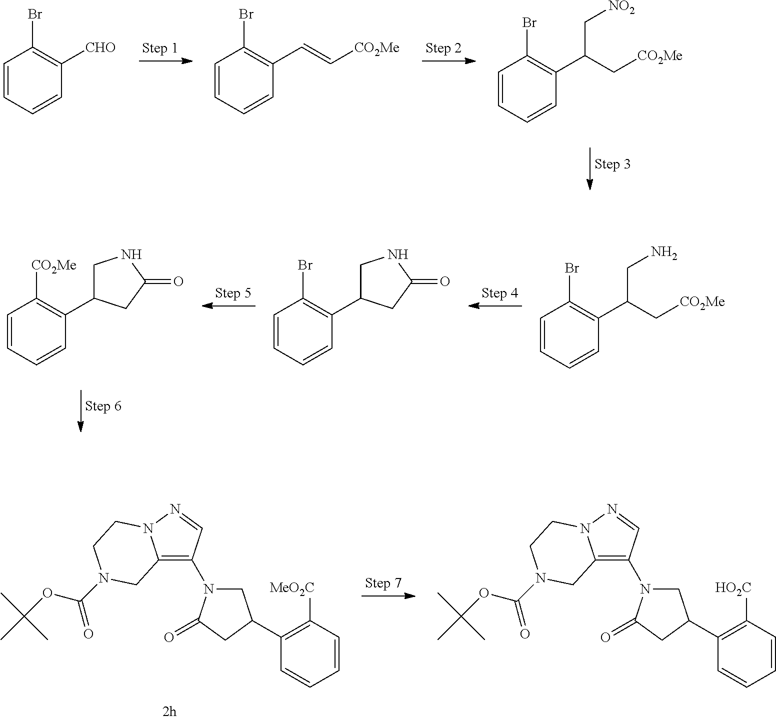

Step 1: 2-Bromobenzaldehyde (10.0 g, 54.05 mmol) and methyl 2-(triphenyl-lambda5-phosphanylidene)acetate (18.07 g, 54.05 mmol) were mixed in DCM (10 mL) and the resulting mixture was stirred at r.t. overnight. The resulting mixture was evaporated to dryness. The residue was triturated with hexane. All insoluble materials were filtered off and the filtrate was evaporated to dryness to obtain crude methyl (2E)-3-(2-bromophenyl)prop-2-enoate (12.5 g, 51.85 mmol, 95.9% yield) which was used in next step without purification.

Step 2: To a solution of methyl (2E)-3-(2-bromophenyl)prop-2-enoate (12.5 g, 51.85 mmol) in nitromethane (50 mL) was added 1,1,3,3-tetramethylguanidine (1.19 g, 10.37 mmol) and the resulting mixture was stirred at r.t. After consumption of the starting material (HNMR control) the resulting mixture was evaporated to dryness to obtain crude methyl 3-(2-bromophenyl)-4-nitrobutanoate (13.0 g, 43.03 mmol, 83% yield), which was used in next step without purification.

Step 3: Methyl 3-(2-bromophenyl)-4-nitrobutanoate (18.0 g, 59.58 mmol) was dissolved in acetic acid (150 mL). Zinc (19.48 g, 297.89 mmol) was added portionwise thereto with water bath cooling. The resulting mixture was stirred at r.t. overnight. All insoluble materials were filtered off. The filtrate was concentrated to dryness to give crude methyl 4-amino-3-(2-bromophenyl)butanoate (10.0 g, 30.1 mmol, 50.5% yield) which was used in next step without purification.

Step 4: The product of the previous step (10.0 g, 30.1 mmol) was mixed with sodium hydrogen carbonate (12.64 g, 150.52 mmol) in methanol (100 mL) and the resulting mixture was heated at reflux overnight. After consumption of the starting material the resulting mixture was cooled to r.t. and concentrated. The residue was partitioned between H₂O (100 mL) and EtOAc (100 mL). The organic layer was separated, dried over Na₂SO₄ and concentrated. The residue was purified by column chromatography to give 4-(2-bromophenyl)pyrrolidin-2-one (4.3 g, 17.91 mmol, 59.5% yield).

Step 5: 4-(2-Bromophenyl)pyrrolidin-2-one (4.3 g, 17.91 mmol) was carbonylated in MeOH (100 mL) at 130° C. and 50 atm. CO pressure with Pd(dppf)Cl₂ as catalyst. After consumption of the starting material (TLC control) the resulting mixture was evaporated and the residue was partitioned between water (100 mL) and EtOAc (100 mL). The organic layer was collected, dried over Na₂SO₄ and concentrated to give methyl 2-(5-oxopyrrolidin-3-yl)benzoate (2.5 g, 11.4 mmol, 63.7% yield).

Step 6: Methyl 2-(5-oxopyrrolidin-3-yl)benzoate (999.9 mg, 4.56 mmol), tert-butyl 3-iodo-4H,5H,6H,7H-pyrazolo[1,5-a]pyrazine-5-carboxylate (1.59 g, 4.56 mmol), tripotassium phosphate (2.42 g, 11.4 mmol), 1-N,2-N-dimethylcyclohexane-1,2-diamine (32.44 mg, 228.04 μmol) and copper(I) iodide (21.72 mg, 114.02 μmol) were placed in the tube with a magnetic stirrer. Dry dioxane (20 mL) was added thereto. Argon was bubbled through the mixture for 5 minutes. The tube was sealed and the resulting mixture was heated at 110° C. for 12 h. The resulting solution was concentrated to dryness and the residue was purified by column chromatography to give tert-butyl 3-4-[2-(methoxycarbonyl)phenyl]-2-oxopyrrolidin-1-yl-4H,5H,6H,7H-pyrazolo[1,5-a]pyrazine-5-carboxylate (570.0 mg, 1.29 mmol, 28.4% yield).

Step 7: Tert-butyl 3-4-[2-(methoxycarbonyl)phenyl]-2-oxopyrrolidin-1-yl-4H,5H,6H,7H-pyrazolo[1,5-a]pyrazine-5-carboxylate (570.16 mg, 1.29 mmol) was dissolved in dry MeOH (5 mL). Lithium hydroxide monohydrate (271.58 mg, 6.47 mmol) was added thereto and the resulting mixture was stirred at r.t. until completion (monitored by LCMS). The resulting mixture was concentrated to dryness. The residue was dissolved in H₂O (5 mL) and extracted with EtOAc (3×10 mL). The aqueous layer was collected and acidified with aqueous NaHSO₄ to pH5. The resulting mixture was extracted with EtOAc (2×15 mL). The combined organic extracts were washed with brine, dried over Na₂SO₄ and concentrated to give 2-(1-5-[(tert-butoxy)carbonyl]-4H,5H,6H,7H-pyrazolo[1,5-a]pyrazin-3-yl-5-oxopyrrolidin-3-yl)benzoic acid (156.4 mg, 366.73 μmol, 28.3% yield).

¹H NMR (500 MHz, d6-DMSO) δ 1.42 (m, 9H), 2.57 (m, 1H), 2.85 (m, 1H), 3.70 (m, 1H), 3.80 (m, 2H), 4.07 (m, 3H), 4.43 (m, 1H), 4.60 (m, 2H), 7.37 (m, 1H), 7.56 (m, 3H), 7.79 (m, 1H), 12.86 (br s, 1H).

LCMS: m/z 427.2

### Synthesis of 2-(1-{5-[(tert-butoxy)carbonyl]-4H,5H,6H,7H-pyrazolo[1,5-a]pyrazin-3-yl}-5-oxopyrrolidin-3-yl)-3-fluorobenzoic Acid

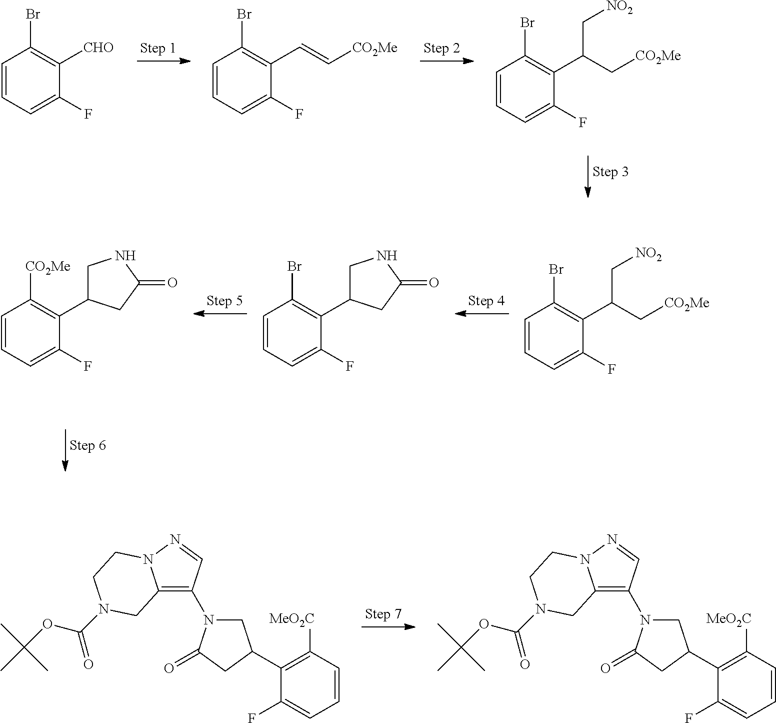

Step 1: 2-Bromo-6-fluorobenzaldehyde (10.0 g, 49.26 mmol) and methyl 2-(triphenyl-lambda5-phosphanylidene)acetate (17.29 g, 51.72 mmol) were mixed in DCM (200 mL) and the resulting mixture was stirred at r.t. overnight, then concentrated to dryness. The residue was triturated with hexane. All insoluble materials were filtered off and the filtrate was evaporated to dryness to obtain crude methyl (2E)-3-(2-bromo-6-fluorophenyl)prop-2-enoate (13.0 g, 50.18 mmol, 101.9% yield) which was used in the next step without purification.

Step 2: To a solution of methyl (2E)-3-(2-bromo-6-fluorophenyl)prop-2-enoate (13.0 g, 50.18 mmol) in nitromethane (50 mL) was added 1,1,3,3-tetramethylguanidine (577.95 mg, 5.02 mmol) and the resulting mixture was stirred at r.t. After consumption of the starting material (HNMR control) the resulting mixture was evaporated to dryness to obtain crude methyl 3-(2-bromo-6-fluorophenyl)-4-nitrobutanoate (17.0 g, 53.11 mmol, 105.8% yield) which was used in next step without purification.

Step 3: Methyl 3-(2-bromo-6-fluorophenyl)-4-nitrobutanoate (16.0 g, 49.98 mmol) was dissolved in acetic acid (150 mL). Zinc (16.35 g, 249.91 mmol) was added thereto portionwise with water bath cooling. The resulting mixture was stirred at r.t. overnight. All insoluble materials were filtered off. The filtrate was evaporated to dryness to obtain crude product (15.0 g, 42.83 mmol, 85.7% yield) which was used in next step without purification.

Step 4: The product of the previous step (15.0 g, 42.84 mmol) was mixed with sodium hydrogen carbonate in methanol (100 mL) and the resulting mixture was heated at reflux overnight. After consumption of the starting material the resulting mixture was cooled to r.t. and evaporated. The residue was partitioned between H₂O (100 mL) and EtOAc (100 mL). The organic layer was separated, dried over Na₂SO₄ and concentrated. The residue was purified by flash chromatography to give 4-(2-bromo-6-fluorophenyl)pyrrolidin-2-one (3.5 g, 13.56 mmol, 31.7% yield).

Step 5: 4-(2-Bromo-6-fluorophenyl)pyrrolidin-2-one (3.5 g, 13.56 mmol) was carbonylated in MeOH (100 mL) at 130° C. and 50 atm. CO pressure with Pd(dppf)Cl₂ as catalyst. After consumption of the starting material (TLC control) the resulting mixture was concentrated and the residue was partitioned between water (100 mL) and EtOAc (100 mL). The organic layer was collected, dried over Na₂SO₄ and concentrated to give a mixture of methyl 3-fluoro-2-(5-oxopyrrolidin-3-yl)benzoate (1.5 g, 6.32 mmol, 46.6% yield) and corresponding benzoic acid which was used without purification.

Step 6: Methyl 3-fluoro-2-(5-oxopyrrolidin-3-yl)benzoate (1.0 g, 4.22 mmol), tert-butyl 3-iodo-4H,5H,6H,7H-pyrazolo[1,5-a]pyrazine-5-carboxylate (1.47 g, 4.22 mmol), tripotassium phosphate (2.24 g, 10.54 mmol), 1-N,2-N-dimethylcyclohexane-1,2-diamine (29.99 mg, 210.8 μmol) and copper(I) iodide (20.07 mg, 105.4 μmol) were placed in a tube with a magnetic stirrer. Dry dioxane (20 mL) was added thereto. Argon was bubbled through the mixture for 5 minutes. The tube was sealed and the resulting mixture was heated at 110° C. for 12 h. The resulting solution was evaporated to dryness and the residue was purified by column chromatography to obtain tert-butyl 3-4-[2-fluoro-6-(methoxycarbonyl)phenyl]-2-oxopyrrolidin-1-yl-4H,5H,6H,7H-pyrazolo[1,5-a]pyrazine-5-carboxylate (650.0 mg, 1.42 mmol, 33.6% yield).

Step 7: Tert-butyl 3-4-[2-fluoro-6-(methoxycarbonyl)phenyl]-2-oxopyrrolidin-1-yl-4H,5H,6H,7H-pyrazolo[1,5-a]pyrazine-5-carboxylate (649.88 mg, 1.42 mmol) was dissolved in dry MeOH (5 mL). Lithium hydroxide monohydrate (297.41 mg, 7.09 mmol) was added thereto and the resulting mixture was stirred at r.t. After consumption of starting material, the mixture was evaporated to dryness. The residue was dissolved in H₂O (5 mL) and extracted with EtOAc (3×10 mL). The aqueous layer was collected and acidified with sat. aq. NaHSO₄ to pH 5. The resulting mixture was extracted with EtOAc (2×15 mL). The combined organic extracts were washed with brine, dried over Na₂SO₄ and concentrated to give 2-(1-5-[(tert-butoxy)carbonyl]-4H,5H,6H,7H-pyrazolo[1,5-a]pyrazin-3-yl-5-oxopyrrolidin-3-yl)-3-fluorobenzoic acid (123.0 mg, 276.74 μmol, 19.5% yield).

¹H NMR (400 MHz, d6-DMSO) δ 1.44 (s, 9H), 2.61 (m, 1H), 2.86 (m, 1H), 3.72 (m, 1H), 3.81 (m, 2H), 4.08 (m, 3H), 4.56 (m, 1H), 4.59 (m, 2H), 7.43 (m, 2H), 7.56 (m, 2H), 13.46 (s, 1H).

LCMS: m/z 445.0

### Synthesis of tert-butyl 3-{6-oxo-5-azaspiro[2.4]heptan-5-yl}-4H,5H,6H,7H-pyrazolo[1,5-a]pyrazine-5-carboxylate

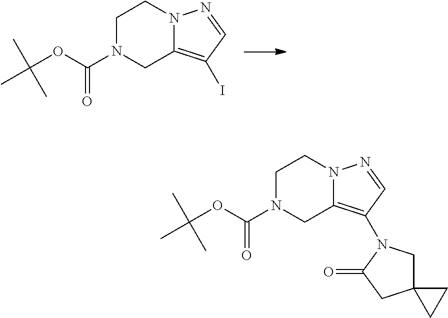 [SMILES: CC(C)(C)OC(=O)N1CCn2ncc(I)c2C1 ; CC(C)(C)OC(=O)N1CCn2ncc(N3CC4(CC4)CC3=O)c2C1]

A mixture of tert-butyl 3-iodo-4H,5H,6H,7H-pyrazolo[1,5-a]pyrazine-5-carboxylate (1.0 g, 2.86 mmol), 5-azaspiro[2.4]heptan-6-one (477.47 mg, 4.3 mmol), copper(I) iodide (38.18 mg, 200.48 μmol), tripotassium phosphate (1.22 g, 5.73 mmol) and methyl[2-(methylamino)ethyl]amine (35.35 mg, 400.97 μmol) in dioxane (10 mL) under argon was heated at 130° C. for 8 hours. The reaction mixture was diluted with EtOAc (20 mL) and washed with water and brine. The organic layer was concentrated. The crude product was purified by HPLC to give tert-butyl 3-6-oxo-5-azaspiro[2.4]heptan-5-yl-4H,5H,6H,7H-pyrazolo[1,5-a]pyrazine-5-carboxylate (800.0 mg, 12.0% purity, 288.81 μmol, 10.1% yield).

¹H NMR (400 MHz, d6-DMSO) δ 0.68 (s, 4H), 1.43 (s, 9H), 2.45 (s, 2H), 3.61 (s, 2H), 3.79 (t, 2H), 4.07 (t, 2H), 4.58 (s, 2H), 7.54 (s, 1H).

LCMS: m/z 333.4

### Synthesis of tert-butyl 3-{4-oxo-5-azaspiro[2.4]heptan-5-yl}-4H,5H,6H,7H-pyrazolo[1,5-a]pyrazine-5-carboxylate

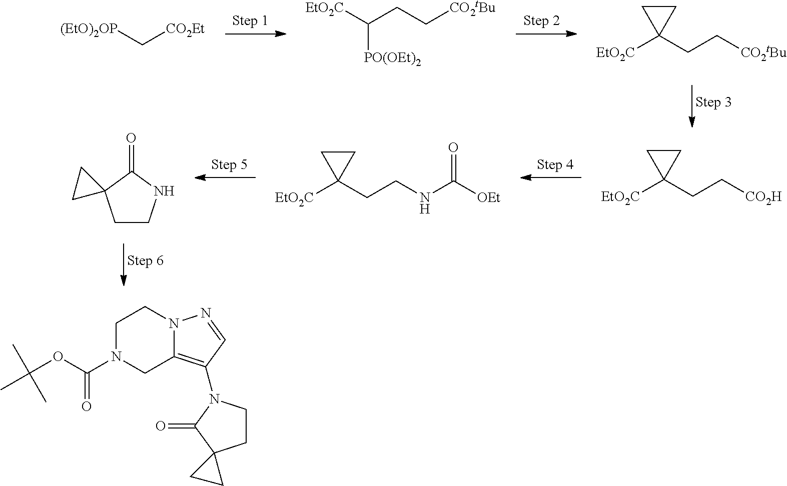

Step 1: Sodium hydride (7.01 g, 291.96 mmol) was suspended in THF (150 mL) under an atmosphere of argon. Ethyl 2-(diethyl phosphono)acetate (30.0 g, 133.81 mmol) in THF (50 mL) was added at r.t. After a further 90 min the solution became homogeneous and tert-butyl acrylate (17.15 g, 133.81 mmol) in THF (50 mL) was added slowly. After addition was complete the reaction mixture was refluxed for 5 h. The reaction was then cooled to r.t., carefully quenched with aqueous NH₄Cl (10 mL), and concentrated. The residue was partitioned between H₂O (25 mL) and MTBE (50 mL), and the aqueous layer was extracted with MTBE (3×50 mL). The combined organic layers were washed with brine (50 mL), dried and concentrated to give 5-tert-butyl 1-ethyl 2-(diethyl phosphono)pentanedioate (43.0 g, 80.0% purity, 97.63 mmol, 73% yield). The product was used in the next step without purification.

Step 2: Sodium hydride (7.99 g, 332.82 mmol) was suspended in dry toluene (150 mL) under an atmosphere of argon in the flask equipped with a Dewar-type condenser. 5-Tert-butyl 1-ethyl 2-(diethyl phosphono)pentanedioate (43.0 g, 122.03 mmol) in toluene (120 mL) was added via syringe over 20 mins with accompanying evolution of gas. After 2 h of stirring at 23° C. the reaction mixture became homogeneous and was cooled in an ice bath for 30 min prior to addition of oxirane. The Dewar-type condenser was charged with dry ice and acetone, and ethylene oxide (11.83 g, 268.47 mmol), previously condensed into a separate flask, was cannulated into the reaction mixture. The contents of the flask were brought to a gentle reflux (bath temperature 40° C.) for 3 h and then cooled to 23° C. and quenched by careful addition of aqueous NH₄Cl (70 mL, 1N) and H₂O (50 mL). The aqueous layers was extracted with MTBE (3×70 mL), the organic layers were combined, washed with brine, dried (Na₂SO₄), filtered, and concentrated in vacuo, and the crude product was distilled under reduce pressure (60-65° C. at 0.5 mmHg) to give ethyl 1-[3-(tert-butoxy)-3-oxopropyl]cyclopropane-1-carboxylate (5.0 g, 50.0% purity, 10.32 mmol, 8.5% yield).

Step 3: Ethyl 1-[3-(tert-butoxy)-3-oxopropyl]cyclopropane-1-carboxylate (3.0 g, 12.38 mmol) was dissolved in 2,2,2-trifluoroacetic acid (16.94 g, 148.55 mmol, 11.47 mL) and heated at reflux for 12 h. After the mixture was cooled to r.t. the CF₃COOH was removed in vacuo. After evaporation to dryness the residue was dissolved in sat. NaHCO₃ (15 mL), washed with CH₂Cl₂ (2×25 mL), acidified (pH 2) with citric acid, and extracted twice with CH₂Cl₂ (25 ml). The organic layer was washed with water (30 mL), dried (over Na₂SO₄) and evaporated under reduced pressure to yield 3-[1-(ethoxycarbonyl)cyclopropyl]propanoic acid (1.3 g, 80.0% purity, 5.59 mmol, 45.1% yield).

Step 4: 3-[1-(Ethoxycarbonyl)cyclopropyl]propanoic acid (1.3 g, 6.96 mmol) in dry toluene (30 mL) and triethylamine (704.22 mg, 6.96 mmol, 970.0 μl) were mixed at r.t. under an atmosphere of argon. Diphenylphosphoryl azide (1.92 g, 6.96 mmol) in toluene (5 mL) was added via syringe, and the contents of the flask were warmed to 75° C. (bath temperature) for 4 h. EtOH (10 mL) was added, and the reaction mixture was maintained at reflux for 12 h, the reaction mixture was cooled to r.t., and the remaining EtOH was removed in vacuo. Water (50 mL) was added to the organic residue, the layers were separated, the aqueous layer was extracted with MTBE (2×50 mL); the combined organic layers were washed with brine, dried (over Na₂SO₄), filtered, and concentrated in vacuo to give ethyl 1-2-[(ethoxycarbonyl)amino]ethylcyclopropane-1-carboxylate (1.4 g, 70.0% purity, 4.27 mmol, 61.4% yield). The product was used in the next step without purification.

Step 5: Ethyl 1-2-[(ethoxycarbonyl)amino]ethylcyclopropane-1-carboxylate (1.0 g, 4.36 mmol) was dissolved in CH₃OH (10 mL) and barium hydroxide octahydrate (1.42 g, 4.49 mmol) was added. The solution was heated at reflux for 14 h, cooled with ice, and acidified with concentrated H₂SO₄, and the resulting BaSO₄ precipitate was removed by filtration. The aqueous filtrate was extracted with EtOAc (3×30 mL), and the organic extract was dried and concentrated in vacuo to give 5-azaspiro[2.4]heptan-4-one (1.0 g, 55.0% purity, 4.95 mmol, 113.4% yield). The product was used in the next step without purification.

Step 6: A mixture of tert-butyl 3-iodo-4H,5H,6H,7H-pyrazolo[1,5-a]pyrazine-5-carboxylate (1.01 g, 2.89 mmol), 5-azaspiro[2.4]heptan-4-one (700.34 mg, 6.3 mmol), (1R,2R)—N1,N2-dimethylcyclohexane-1,2-diamine (41.08 mg, 288.81 μmol), copper(I) iodide (55.0 mg, 288.81 μmol) and potassium carbonate (1.2 g, 8.66 mmol) in DMSO (10 mL) under argon was heated at 130° C. for 16 hours. The reaction mixture was cooled and diluted with MTBE (20 mL), then washed with water and brine. The organic layer was concentrated. The crude product was purified by HPLC to give tert-butyl 3-4-oxo-5-azaspiro[2.4]heptan-5-yl-4H,5H,6H,7H-pyrazolo[1,5-a]pyrazine-5-carboxylate (200.0 mg, 601.69 μmol, 20.8% yield).

¹H NMR (400 MHz, d6-DMSO) δ 0.83 (m, 2H), 0.91 (m, 2H), 1.42 (s, 9H), 2.20 (t, 2H), 3.78 (m, 4H), 4.07 (t, 2H), 4.56 (s, 2H), 7.57 (s, 1H).

LCMS: m/z 332.4

### Synthesis of 5-(1H-Indole-2-carbonyl)-4H,5H,6H,7H-pyrazolo[1,5-a]pyrazine-3-carboxylic Acid

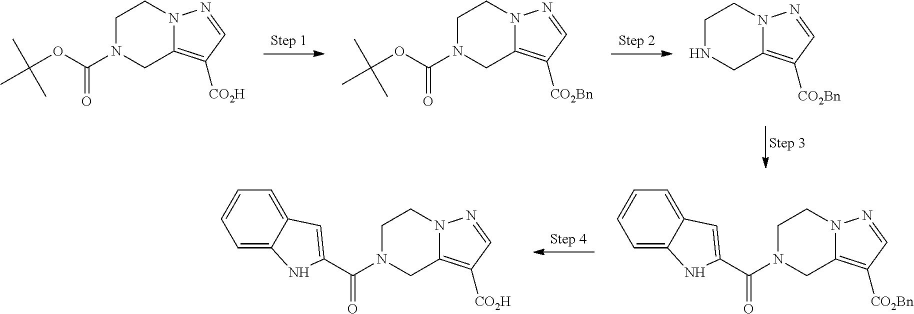 [SMILES: CC(C)(C)OC(=O)N1CCn2ncc(C(=O)O)c2C1 ; CC(C)(C)OC(=O)N1CCn2ncc(C(=O)OCc3ccccc3)c2C1 ; O=C(OCc1ccccc1)c1cnn2c1CNCC2 ; O=C(O)c1cnn2c1CN(C(=O)c1cc3ccccc3[nH]1)CC2 ; O=C(OCc1ccccc1)c1cnn2c1CN(C(=O)c1cc3ccccc3[nH]1)CC2]

Step 1: To a solution of 5-[(tert-butoxy)carbonyl]-4H,5H,6H,7H-pyrazolo[1,5-a]pyrazine-3-carboxylic acid (15.4 g, 57.62 mmol) in MeCN (500 mL) was added potassium carbonate (10.35 g, 74.9 mmol) in one portion at r.t., followed by portionwise addition of (bromomethyl)benzene (9.56 g, 55.89 mmol, 6.65 ml). The resulting viscous slurry was stirred overnight at r.t., and progress of the reaction was monitored by ¹H NMR. Once complete, the mixture was concentrated under reduced pressure. The residue was taken up in MTBE (200 mL), the resulting suspension was washed with water (3×200 mL), brine, dried over Na₂SO₄ and evaporated in vacuo to give 3-benzyl 5-tert-butyl 4H,5H,6H,7H-pyrazolo[1,5-a]pyrazine-3,5-dicarboxylate (17.0 g, 47.57 mmol, 82.6% yield) as colorless solid.

Step 2: 3-Benzyl 5-tert-butyl 4H,5H,6H,7H-pyrazolo[1,5-a]pyrazine-3,5-dicarboxylate (17.0 g, 47.57 mmol) was dissolved in 4M HCl/dioxane (500 mL) at r.t. and the resulting mixture was stirred overnight. Upon completion of the reaction (monitored by ¹H NMR), the resulting mixture was evaporated to dryness to obtain benzyl 4H,5H,6H,7H-pyrazolo[1,5-a]pyrazine-3-carboxylate (10.0 g, 38.87 mmol, 71.6% yield) as light yellow solid residue.

Step 3: To a solution of indole-2-carboxylic acid (6.1 g, 37.82 mmol) and triethylamine (9.57 g, 94.56 mmol, 13.18 ml) in dry DMF (200 mL) at r.t. was added HATU (15.1 g, 39.72 mmol) in one portion. The resulting mixture was stirred for 10 min before benzyl 4H,5H,6H,7H-pyrazolo[1,5-a]pyrazine-3-carboxylate hydrochloride (10.0 g, 34.04 mmol) was added and the stirring was continued overnight. The reaction mixture was poured into 1000 mL of stirring water and the resulting mixture was filtered. The filter cake was washed with MeOH/H₂O (1:2 v:v, 3×100 mL) dried under reduced pressure to give benzyl 5-(1H-indole-2-carbonyl)-4H,5H,6H,7H-pyrazolo[1,5-a]pyrazine-3-carboxylate (13.0 g, 32.47 mmol, 85.8% yield) as light yellow powder.

Step 4: Benzyl 5-(1H-indole-2-carbonyl)-4H,5H,6H,7H-pyrazolo[1,5-a]pyrazine-3-carboxylate (12.75 g, 31.84 mmol) was dissolved in DMF (2500 mL), then 10% Pd on carbon (2 g) was added. The whole system was flushed with hydrogen gas and a balloon with hydrogen was connected to the neck of the flask. The reaction mixture was stirred at 50° C. overnight. When the ¹H NMR indicated absence of starting material, the reaction mixture was filtered and the filtrate was concentrated under reduced pressure to total volume of about 100-150 mL. This residue was diluted with MeOH (500 mL) and filtered. The filter cake was washed with MeOH (2×200 mL) and dried under reduced pressure, to give 5-(1H-indole-2-carbonyl)-4H,5H,6H,7H-pyrazolo[1,5-a]pyrazine-3-carboxylic acid (9.57 g, 30.84 mmol, 96.9% yield) as light yellow powder.

¹H NMR (500 MHz, d6-DMSO) δ 4.25 (m, 2H), 4.33 (m, 2H), 5.17 (br.s, 2H), 6.96 (s, 1H), 7.07 (m, 1H), 7.22 (m, 1H), 7.45 (dd, J=8.2, 2.9 Hz, 1H), 7.64 (dd, J=8.1, 2.5 Hz, 1H), 7.84 (s, 1H), 11.66 (s, 1H), 12.42 (s, 1H).

### Synthesis of tert-butyl 3-({1-[(2-hydroxyethoxy)methyl]cyclopropyl}(methyl)carbamoyl)-4H,5H,6H,7H-pyrazolo[1,5-a]pyrazine-5-carboxylate

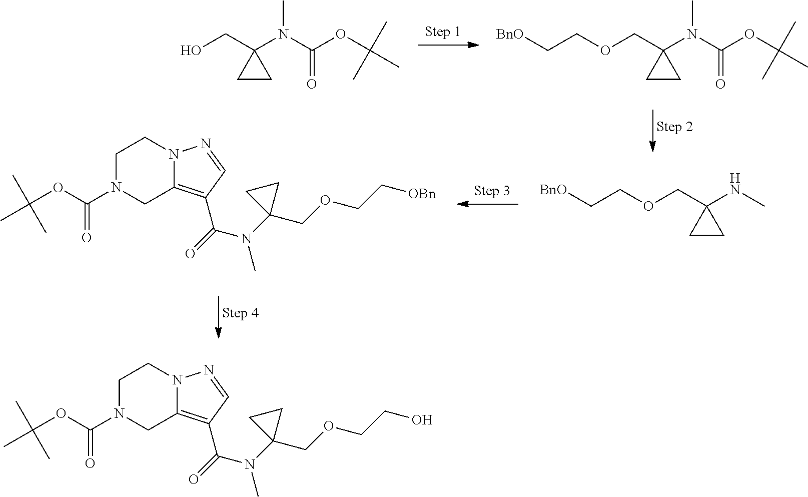 [SMILES: CN(C(=O)OC(C)(C)C)C1(CO)CC1 ; CN(C(=O)OC(C)(C)C)C1(COCCOCc2ccccc2)CC1 ; CN(C(=O)c1cnn2c1CN(C(=O)OC(C)(C)C)CC2)C1(COCCOCc2ccccc2)CC1 ; CNC1(COCCOCc2ccccc2)CC1 ; CN(C(=O)c1cnn2c1CN(C(=O)OC(C)(C)C)CC2)C1(COCCO)CC1]

Step 1: To a solution of tert-butyl N-[1-(hydroxymethyl)cyclopropyl]-N-methylcarbamate (2.0 g, 9.94 mmol) and [(2-bromoethoxy)methyl]benzene (2.35 g, 10.93 mmol, 1.73 ml) in dry DMF (40 mL) was added sodium hydride (476.9 mg, 19.87 mmol) in small portions, maintaining temperature below 15° C. The resulting mixture was left to stir overnight at r.t., then the reaction mixture was poured into water (400 mL) and extracted with EtOAc (100 mL). The organic phase was washed with water (2×50 mL), brine, dried over Na₂SO₄ and concentrated in vacuo. The residue was purified by column chromatography (80 g silica, petroleum ether/MTBE gradient from 0 to 70%) to give tert-butyl N-(1-[2-(benzyloxy)ethoxy]methylcyclopropyl)-N-methylcarbamate (1.05 g, 3.13 mmol, 31.5% yield).

Step 2: Tert-butyl N-(1-[2-(benzyloxy)ethoxy]methylcyclopropyl)-N-methylcarbamate (1.0 g, 2.98 mmol) was dissolved in 4M HCl in dioxane (30 mL) at r.t. and the resulting mixture was stirred overnight. Upon completion of the reaction (monitored by ¹H NMR), the mixture was evaporated to dryness to obtain 1-[2-(benzyloxy)ethoxy]methyl-N-methylcyclopropan-1-amine hydrochloride (800.0 mg, 2.94 mmol, 98.8% yield) as solid residue that was used in the next step without further purification.

Step 3: To a solution of 5-[(tert-butoxy)carbonyl]-4H,5H,6H,7H-pyrazolo[1,5-a]pyrazine-3-carboxylic acid (943.84 mg, 3.53 mmol) and triethylamine (744.43 mg, 7.36 mmol, 1.03 ml) in DMF (20 mL) at r.t. was added HATU (1.68 g, 4.41 mmol). The resulting mixture was stirred for 10 min, then 1-[2-(benzyloxy)ethoxy]methyl-N-methylcyclopropan-1-amine hydrochloride (800.0 mg, 2.94 mmol) was added and the stirring was continued overnight. The reaction mixture was partitioned between EtOAc (50 mL) and water (200 mL). The organic phase was washed with water (2×30 mL), brine, dried over sodium sulfate and concentrated under reduced pressure. The residue was purified by column chromatography (40 g silica, chloroform/acetonitrile with acetonitrile from 0-30%) to give tert-butyl 3-[(1-[2-(benzyloxy)ethoxy]methylcyclopropyl)(methyl)carbamoyl]-4H,5H,6H,7H-pyrazolo[1,5-a]pyrazine-5-carboxylate (800.0 mg, 1.65 mmol, 56.1% yield).

Step 4: Tert-butyl 3-[(1-[2-(benzyloxy)ethoxy]methylcyclopropyl)(methyl)carbamoyl]-4H,5H,6H,7H-pyrazolo[1,5-a]pyrazine-5-carboxylate (800.0 mg, 1.65 mmol) and palladium on carbon (5%, 100 mg) were mixed together in dry MeOH (20 mL). The flask was evacuated and backfilled with hydrogen gas from a connected balloon. The reaction mixture was stirred at r.t. overnight. The mixture was filtered and the filtrate was concentrated in vacuo. The residue was purified by HPLC to give tert-butyl 3-(1-[(2-hydroxyethoxy)methyl]cyclopropyl(methyl)carbamoyl)-4H,5H,6H,7H-pyrazolo[1,5-a]pyrazine-5-carboxylate (450.0 mg, 1.14 mmol, 69.1% yield).

¹H NMR (400 MHz, d6-DMSO) δ 7.67 (m, 1H), 8.50 (d, 1H), 8.69 (s, 1H), 8.79 (d, 2H), 9.21 (s, 1H), 9.33 (s, 1H).

LCMS: m/z 395.2

### Synthesis of tert-butyl 3-({1-[(3-hydroxypropoxy)methyl]cyclopropyl}(methyl)carbamoyl)-4H,5H,6H,7H-pyrazolo[1,5-a]pyrazine-5-carboxylate

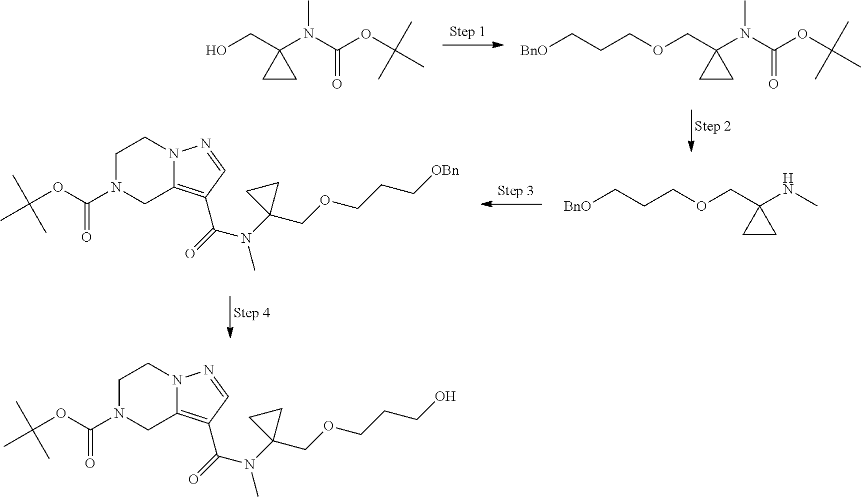 [SMILES: CN(C(=O)OC(C)(C)C)C1(CO)CC1 ; CN(C(=O)OC(C)(C)C)C1(COCCCOCc2ccccc2)CC1 ; CN(C(=O)c1cnn2c1CN(C(=O)OC(C)(C)C)CC2)C1(COCCCOCc2ccccc2)CC1 ; CNC1(COCCCOCc2ccccc2)CC1 ; CN(C(=O)c1cnn2c1CN(C(=O)OC(C)(C)C)CC2)C1(COCCCO)CC1]

Step 1: To a solution of tert-butyl N-[1-(hydroxymethyl)cyclopropyl]-N-methylcarbamate (1.57 g, 7.8 mmol) and [(3-bromopropoxy)methyl]benzene (1.97 g, 8.58 mmol, 1.51 ml) in DMF (30 mL) sodium hydride (374.39 mg, 15.6 mmol) was added in few portions, maintaining temperature below 15° C. and the resulting mixture was left to stir overnight at r.t.. The reaction mixture was poured into water (300 mL) and extracted with EtOAc (50 mL). Organic phase was washed with water (2×30 mL), brine, dried over Na₂SO₄ and concentrated in vacuo. The residue was purified by column chromatography (40 g silica, petroleum ether/MTBE 0-35%) to give tert-butyl N-(1-[3-(benzyloxy)propoxy]methylcyclopropyl)-N-methylcarbamate (320.0 mg, 915.69 μmol, 11.7% yield).

Step 2: Tert-butyl N-(1-[3-(benzyloxy)propoxy]methylcyclopropyl)-N-methylcarbamate (320.0 mg, 915.69 μmol) was dissolved in 4M HCl in dioxane (20 mL) at r.t. and the resulting mixture was stirred overnight. The resulting mixture was evaporated to dryness to obtain 1-[3-(benzyloxy)propoxy]methyl-N-methylcyclopropan-1-amine hydrochloride (350.0 mg, 60.0% purity, 734.75 μmol, 92.1% yield) as solid residue that was used in the next step without further purification.

Step 3: To a solution of 5-[(tert-butoxy)carbonyl]-4H,5H,6H,7H-pyrazolo[1,5-a]pyrazine-3-carboxylic acid (228.36 mg, 854.37 μmol) and triethylamine (216.13 mg, 2.14 mmol, 300.0 μl) in DMF (20 mL) was added (1H-1,2,3-benzotriazol-1-yloxy)tris(dimethylamino)phosphonium hexafluorophosphate (415.66 mg, 939.8 μmol). The resulting mixture was stirred for 10 mins, then 1-[3-(benzyloxy)propoxy]methyl-N-methylcyclopropan-1-amine hydrochloride (220.0 mg, 769.74 μmol) was added and the stirring was continued overnight. The reaction mixture was partitioned between EtOAc (50 mL) and water (200 mL). The organic phase was washed with water (2×30 mL), brine, dried over sodium sulfate and concentrated under reduced pressure. The residue was purified by column chromatography (40 g silica, chloroform/acetonitrile from 0-50%) to give tert-butyl 3-[(1-[3-(benzyloxy)propoxy]methylcyclopropyl)(methyl)carbamoyl]-4H,5H,6H,7H-pyrazolo[1,5-a]pyrazine-5-carboxylate (200.0 mg, 401.11 μmol, 46.9% yield).

Step 4: Tert-butyl 3-[(1-[3-(benzyloxy)propoxy]methylcyclopropyl)(methyl)carbamoyl]-4H,5H,6H,7H-pyrazolo[1,5-a]pyrazine-5-carboxylate (200.0 mg, 401.11 μmol) and palladium on carbon (5%, 50 mg) were mixed together in dry MeOH (20 mL). The flask was evacuated and backfilled with hydrogen gas from a connected balloon. The reaction mixture was stirred at r.t. overnight then filtered. The filtrate was concentrated in vacuo. The residue was purified by HPLC to give tert-butyl 3-(1-[(3-hydroxypropoxy)methyl]cyclopropyl(methyl)carbamoyl)-4H,5H,6H,7H-pyrazolo[1,5-a]pyrazine-5-carboxylate (120.0 mg, 293.76 μmol, 73.2% yield).

¹H NMR (400 MHz, CDCl₃) δ 0.93 (m, 4H), 1.47 (s, 9H), 1.80 (p, 2H), 1.93 (m, 1H), 3.16 (m, 3H), 3.62 (m, 4H), 3.71 (t, 2H), 3.87 (m, 2H), 4.14 (s, 2H), 4.86 (s, 2H), 7.90 (m, 1H).

LCMS: m/z 408

### Synthesis of tert-butyl 3-[(1-{[(2,2-difluoroethyl)amino]methyl}cyclopropyl)(methyl)carbamoyl]-4H,5H,6H,7H-pyrazolo[1,5-a]pyrazine-5-carboxylate

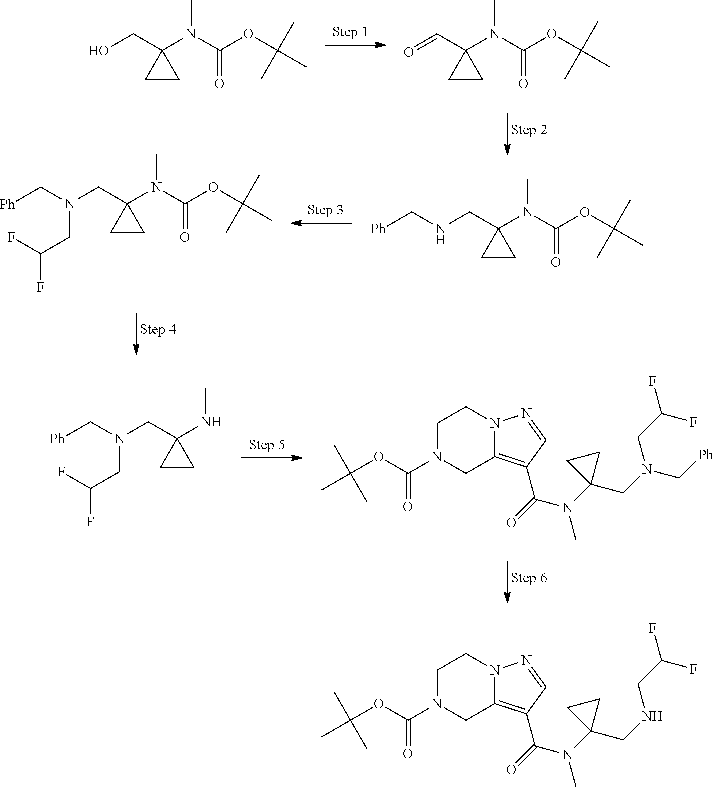

Step 1: To a stirred solution of tert-butyl N-[1-(hydroxymethyl)cyclopropyl]-N-methylcarbamate (2.25 g, 11.18 mmol) in dry DCM (30 mL) at r.t. was added 1,1,1-tris(acetoxy)-1,1-dihydro-1,2-benziodoxol-3(1H)-one (4.74 g, 11.18 mmol) portionwise. The reaction mixture was stirred at r.t. for 1 h and then cooled to 0° C. A solution of sodium hydroxide (2.01 g, 50.3 mmol) in water (5 mL) was then added dropwise and the mixture was stirred at r.t. for 15 min. The organic phase was separated, dried over Na₂SO₄, filtered and concentrated to afford tert-butyl N-(1-formylcyclopropyl)-N-methylcarbamate (2.2 g, 11.04 mmol, 98.8% yield) as yellow oil.

Step 2: To a stirred solution of tert-butyl N-(1-formylcyclopropyl)-N-methylcarbamate (2.2 g, 11.04 mmol) in dry DCM (50 mL) was added phenylmethanamine (1.18 g, 11.04 mmol). The mixture was stirred at r.t. for 5 h. To the cooled reaction mixture was added sodium bis(acetyloxy)boranuidyl acetate (7.02 g, 33.12 mmol) in one portion and stirring was continued for 5 h. The mixture was cooled to 0° C. and 15% aq. solution of NaOH (20 mL) was added. The mixture was stirred for 30 min and organic phase was separated, dried over Na₂SO₄, filtered and concentrated to afford tert-butyl N-1-[(benzylamino)methyl]cyclopropyl-N-methylcarbamate (2.75 g, 85% yield) as yellow oil.

Step 3: To a stirred, cooled (0° C.) solution of tert-butyl N-1-[(benzylamino)methyl]cyclopropyl-N-methylcarbamate (1.75 g, 6.02 mmol) in dry acetonitrile (10 mL) was added potassium carbonate (1.67 g, 12.05 mmol) followed by dropwise addition of 2,2-difluoroethyl trifluoromethanesulfonate (1.68 g, 7.83 mmol). The reaction mixture was warmed to r.t. and stirred overnight. The mixture was poured into water (30 mL) and extracted with DCM (3×10 mL). The combined organic phases was dried over Na₂SO₄, filtered and concentrated. The residue was purified by flash column chromatography on silica with hexane-MTBE (4:1) as eluent to afford tert-butyl N-(1-[benzyl(2,2-difluoroethyl)amino]methylcyclopropyl)-N-methylcarbamate (900.0 mg, 2.54 mmol, 42.2% yield) as colorless oil.

Step 4: To a solution of tert-butyl N-(1-[benzyl(2,2-difluoroethyl)amino]methylcyclopropyl)-N-methylcarbamate (199.9 mg, 564.0 μmol) in CH₂Cl₂ (3 mL) was added 4M HCl in dioxane (1 mL). The resulting solution was stirred for 12 h at r.t., then concentrated. The residue was triturated with hexane and collected by filtration, to give 1-[benzyl(2,2-difluoroethyl)amino]methyl-N-methylcyclopropan-1-amine dihydrochloride (156.0 mg, 95.1% yield) as white solid.

Step 5: To a solution of 1-[benzyl(2,2-difluoroethyl)amino]methyl-N-methylcyclopropan-1-amine dihydrochloride (155.96 mg, 476.58 μmol) and [(dimethylamino)(3H-[1,2,3]triazolo[4,5-b]pyridin-3-yloxy)methylidene]dimethylazanium; hexafluoro-lambda5-phosphanuide (181.21 mg, 476.58 μmol) in DMF (2 mL) was added triethylamine (241.13 mg, 2.38 mmol). The mixture was stirred at r.t. for 15 mins. 5-[(Tert-butoxy)carbonyl]-4H,5H,6H,7H-pyrazolo[1,5-a]pyrazine-3-carboxylic acid (127.38 mg, 476.58 μmol) was added, and the reaction stirred at r.t. for 24 h, then diluted with brine. The mixture was extracted with EtOAc (2×20 mL). The combined organic phases was washed with brine, dried over Na₂SO₄, filtered and concentrated to afford crude tert-butyl 3-[(1-[benzyl(2,2-difluoroethyl)amino]methylcyclopropyl)(methyl)carbamoyl]-4H,5H,6H,7H-pyrazolo[1,5-a]pyrazine-5-carboxylate (200.0 mg, 397.15 μmol, 83.3% yield) as brown oil that was used in the next step without further purification.

Step 6: To a stirred solution of tert-butyl 3-[(1-[benzyl(2,2-difluoroethyl)amino]methylcyclopropyl)(methyl)carbamoyl]-4H,5H,6H,7H-pyrazolo[1,5-a]pyrazine-5-carboxylate (200.0 mg, 397.15 μmol) in MeOH (5 mL) was added palladium on carbon (10%, 0.05 g). The mixture was stirred at r.t. under hydrogen (balloon) for 48 h. The mixture was purged with nitrogen, then filtered, and the filtrate concentrated. The residue was purified by HPLC to give tert-butyl 3-[(1-[(2,2 difluoroethyl)amino]methylcyclopropyl)(methyl)carbamoyl]-4H,5H,6H,7H-pyrazolo[1,5-a]pyrazine-5-carboxylate (70.0 mg, 42.7% yield) as colorless oil.

¹H NMR (400 MHz, d6-DMSO) δ 0.76 (m, 3H), 1.43 (s, 9H), 2.26 (m, 1H), 2.90 (m, 4H), 3.05 (s, 3H), 3.80 (s, 2H), 4.10 (d, 2H), 4.71 (s, 2H), 5.96 (tt, 1H), 7.84 (s, 1H).

LCMS: m/z 414.1

### Synthesis of tert-butyl 3-[methyl(1-{[(2,2,2-trifluoroethyl)amino]methyl}cyclopropyl)carbamoyl]-4H,5H,6H,7H-pyrazolo[1,5-a]pyrazine-5-carboxylate

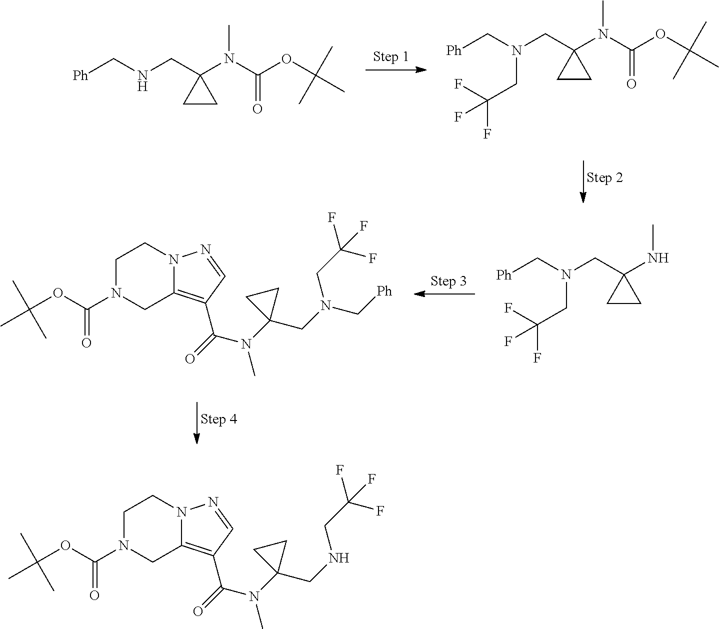 [SMILES: CN(C(=O)OC(C)(C)C)C1(CNCc2ccccc2)CC1 ; CN(C(=O)OC(C)(C)C)C1(CN(Cc2ccccc2)CC(F)(F)F)CC1 ; CN(C(=O)c1cnn2c1CN(C(=O)OC(C)(C)C)CC2)C1(CN(Cc2ccccc2)CC(F)(F)F)CC1 ; CNC1(CN(Cc2ccccc2)CC(F)(F)F)CC1 ; CN(C(=O)c1cnn2c1CN(C(=O)OC(C)(C)C)CC2)C1(CNCC(F)(F)F)CC1]

Step 1: To a stirred solution of tert-butyl N-1-[(benzylamino)methyl]cyclopropyl-N-methylcarbamate (537.25 mg, 1.85 mmol) in dry acetonitrile (10 mL) was added potassium carbonate (767.06 mg, 5.55 mmol) followed by 2,2,2-trifluoroethyl trifluoromethanesulfonate (644.56 mg, 2.78 mmol, 400.0 μL). The reaction mixture was stirred at 80° C. overnight. The mixture was then cooled, concentrated, and the residue obtained was dissolved in DCM (10 mL). The organic phase was washed with water (3 mL), dried over Na₂SO₄ and concentrated. The residue was purified by flash column chromatography on (hexane-MTBE 10:1) to afford tert-butyl N-(1-[benzyl(2,2,2-trifluoroethyl)amino]methylcyclopropyl)-N-methylcarbamate (410.0 mg, 1.1 mmol, 59.5% yield) as colorless oil.

Step 2: To a stirred solution of tert-butyl N-(1-[benzyl(2,2,2-trifluoroethyl)amino]methylcyclopropyl)-N-methylcarbamate (410.0 mg, 1.1 mmol) in DCM (5 mL) was added 4M HCl in dioxane (3 mL, 12 mmol). The resulting mixture was stirred overnight, then evaporated to dryness to give 1-[benzyl(2,2,2-trifluoroethyl)amino]methyl-N-methylcyclopropan-1-amine dihydrochloride (330.0 mg, 955.88 μmol, 86.8% yield) as yellow oil.

Step 3: To a solution of HATU (381.96 mg, 1.0 mmol) in DMF (3 mL) were added triethylamine (484.05 mg, 4.78 mmol) and 5-[(tert-butoxy)carbonyl]-4H,5H,6H,7H-pyrazolo[1,5-a]pyrazine-3-carboxylic acid (255.71 mg, 956.72 μmol). The reaction mixture was stirred at r.t. for 30 mins, then a solution of 1-[benzyl(2,2,2-trifluoroethyl)amino]methyl-N-methylcyclopropan-1-amine dihydrochloride (330.29 mg, 956.72 μmol) in DMF (1 mL) was added. The reaction mixture was stirred at r.t. overnight and poured into water (5 mL). The mixture was extracted with EtOAc (2×5 mL). The combined organic phases was washed with water, aq. NaHCO₃, dried over Na₂SO₄, filtered and concentrated to afford crude tert-butyl 3-[(1-[benzyl(2,2,2-trifluoroethyl)amino]methylcyclopropyl)(methyl)carbamoyl]-4H,5H,6H,7H-pyrazolo[1,5-a]pyrazine-5-carboxylate (600.0 mg, 77.0% purity, 885.78 μmol, 92.6% yield) as brown oil, that was used in the next step without further purification.

Step 4: To a stirred solution of tert-butyl 3-[(1-[benzyl(2,2,2-trifluoroethyl)amino]methylcyclopropyl)(methyl)carbamoyl]-4H,5H,6H,7H-pyrazolo[1,5-a]pyrazine-5-carboxylate (600.0 mg, 1.15 mmol) in MeOH (10 mL) was added palladium on carbon (10%, 70 mg). The mixture was stirred under H₂ (balloon) for 5 days. The mixture was filtered, concentrated, and purified by HPLC to give tert-butyl 3-[methyl(1-[(2,2,2-trifluoroethyl)amino]methylcyclopropyl)carbamoyl]-4H,5H,6H,7H-pyrazolo[1,5-a]pyrazine-5-carboxylate (218.5 mg, 506.43 μmol, 44.1% yield) as brown oil.

¹H NMR (400 MHz, d6-DMSO) δ 0.76 (s, 3H), 1.43 (s, 9H), 2.65 (m, 1H), 2.90 (m, 1H), 3.11 (m, 3H), 3.27 (m, 3H), 3.80 (m, 2H), 4.10 (m, 2H), 4.71 (m, 2H), 7.83 (m, 1H).

LCMS: m/z 432.2

### Synthesis of 4-{4H,5H,6H,7H-pyrazolo[1,5-a]pyrazine-3-carbonyl}-8-oxa-4-azaspiro[2.6]nonane

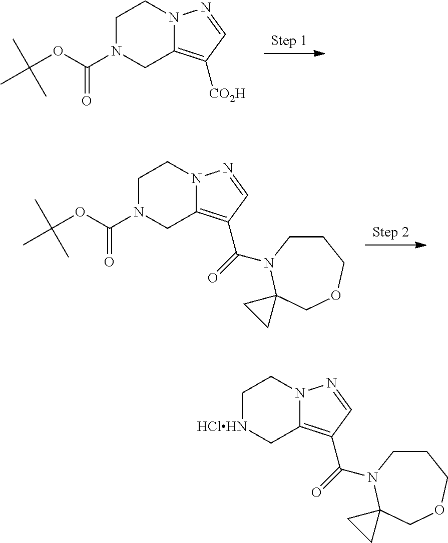

Step 1: To a stirred solution of 5-[(tert-butoxy)carbonyl]-4H,5H,6H,7H-pyrazolo[1,5-a]pyrazine-3-carboxylic acid (489.9 mg, 1.83 mmol) and 8-oxa-4-azaspiro[2.6]nonane hydrochloride (300.0 mg, 1.83 mmol) in DMF (5 mL) were added HATU (906.01 mg, 2.38 mmol) and triethylamine (649.15 mg, 6.42 mmol, 890.0 μL). Schem The mixture was stirred overnight at r.t. and then poured into water and extracted with MTBE (2×15 mL). The combined organic fractions were washed three times with water (20 mL), dried over Na₂SO₄, and concentrated to give tert-butyl 3-8-oxa-4-azaspiro[2.6]nonane-4-carbonyl-4H,5H,6H,7H-pyrazolo[1,5-a]pyrazine-5-carboxylate (500.0 mg, 91.0% purity, 1.21 mmol, 65.9% yield).

Step 2: To a solution of tert-butyl 3-8-oxa-4-azaspiro[2.6]nonane-4-carbonyl-4H,5H,6H,7H-pyrazolo[1,5-a]pyrazine-5-carboxylate (500.0 mg, 1.33 mmol) in MeOH (10 mL) was added 4M HCl in dioxane (2 mL, 8 mmol). The resulting solution was stirred for 12 h, and then concentrated under reduced pressure. The product was treated with MTBE (50 mL) and collected by filtration, then dried in vacuo at 40° C., to give 4-4H,5H,6H,7H-pyrazolo[1,5-a]pyrazine-3-carbonyl-8-oxa-4-azaspiro[2.6]nonane hydrochloride (220.0 mg, 90.0% purity, 633.0 μmol, 54% yield).

¹H NMR (500 MHz, d6-DMSO) δ 0.90 (m, 4H), 1.95 (m, 2H), 3.50 (m, 3H), 3.64 (m, 5H), 4.37 (m, 2H), 4.47 (m, 2H), 7.77 (s, 1H), 10.09 (m, 2H).

LCMS: m/z 277.2

### Synthesis of 4-{4H,5H,6H,7H-pyrazolo[1,5-a]pyrazine-3-carbonyl}-7-oxa-4-azaspiro[2.6]nonane

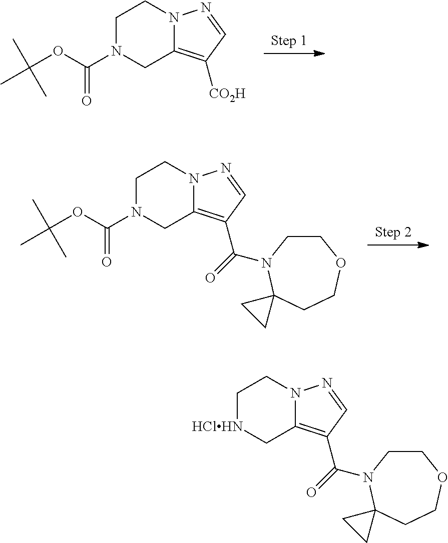

Step 1: To a stirred solution of 5-[(tert-butoxy)carbonyl]-4H,5H,6H,7H-pyrazolo[1,5-a]pyrazine-3-carboxylic acid (489.9 mg, 1.83 mmol) and 7-oxa-4-azaspiro[2.6]nonane hydrochloride (300.0 mg, 1.83 mmol) in DMF (5 mL) were added HATU (906.01 mg, 2.38 mmol) and triethylamine (649.15 mg, 6.42 mmol, 890.0 μL). The mixture was stirred overnight at r.t. and then poured into water and extracted with MTBE (2×15 mL). The combined organic fractions were washed three times with water, dried over anhydrous sodium sulfate, and concentrated to give tert-butyl 3-7-oxa-4-azaspiro[2.6]nonane-4-carbonyl-4H,5H,6H,7H-pyrazolo[1,5-a]pyrazine-5-carboxylate (350.0 mg, 95.0% purity, 883.25 μmol, 48.2% yield).

Step 2: To a solution of tert-butyl 3-7-oxa-4-azaspiro[2.6]nonane-4-carbonyl-4H,5H,6H,7H-pyrazolo[1,5-a]pyrazine-5-carboxylate (350.0 mg, 929.74 μmol) in methanol (10 ml) was added 4N HCl solution in dioxane (2 mL) and the resulting solution was stirred for 12 h at 25° C. Upon completion of the reaction (monitored by HNMR), the reaction mixture was concentrated under reduced pressure. The product was treated with MTBE and collected by filtration, then dried in vacuo at 40° C., to give 4-4H,5H,6H,7H-pyrazolo[1,5-a]pyrazine-3-carbonyl-7-oxa-4-azaspiro[2.6]nonane hydrochloride (110.0 mg, 91.0% purity, 320.02 μmol, 34.4% yield).

¹H NMR (400 MHz, D₂O) δ 0.87 (m, 4H), 1.73 (m, 1H), 3.71 (m, 5H), 3.93 (m, 2H), 4.39 (m, 2H), 4.55 (m, 3H), 7.82 (m, 1H).

LCMS: m/z 277.2

### Synthesis of 2,2-difluoro-4-{4H,5H,6H,7H-pyrazolo[1,5-a]pyrazine-3-carbonyl}morpholine

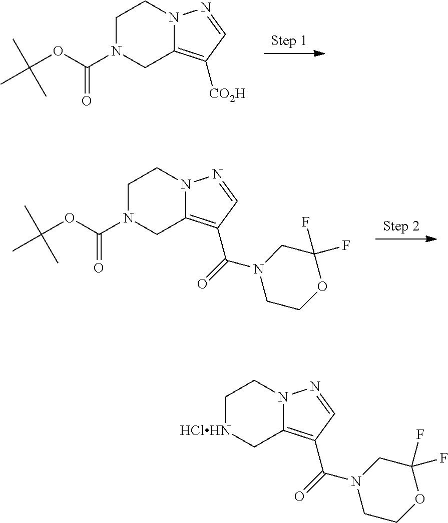

Step 1: To a stirred solution of 2,2-difluoromorpholine hydrochloride (500.0 mg, 3.13 mmol) and 5-[(tert-butoxy)carbonyl]-4H,5H,6H,7H-pyrazolo[1,5-a]pyrazine-3-carboxylic Acid (837.66 mg, 3.13 mmol) in DMF (5 mL) were added HATU (1.55 g, 4.07 mmol) and triethylamine (1.05 g, 10.34 mmol, 1.44 mL). The mixture was stirred at r.t. overnight, and then poured in water (50 mL). Product was extracted with MTBE (2×50 mL). The combined organic fractions were washed three times with water, dried over anhydrous sodium sulfate, and the solvent was removed under vacuum. The product was purified by HPLC to give tert-butyl 3-(2,2-difluoromorpholine-4-carbonyl)-4H,5H,6H,7H-pyrazolo[1,5-a]pyrazine-5-carboxylate (315.0 mg, 98.0% purity, 829.02 μmol, 26.5% yield) as yellow oil.

Step 2: To tert-butyl 3-(2,2-difluoromorpholine-4-carbonyl)-4H,5H,6H,7H-pyrazolo[1,5-a]pyrazine-5-carboxylate (315.0 mg, 845.94 μmol) was added 4M HCl in dioxane (4 mL, 16 mmol). The resulting mixture was stirred overnight, then concentrated to dryness to give 3-(2,2-difluoromorpholine-4-carbonyl)-4H,5H,6H,7H-pyrazolo[1,5-a]pyrazin-5-ium chloride (185.0 mg, 98.0% purity, 587.28 μmol, 69.5% yield) as a solid.

¹H NMR (500 MHz, d6-DMSO) δ 3.64 (m, 2H), 3.80 (m, 2H), 4.04 (m, 2H), 4.13 (m, 2H), 4.38 (m, 2H), 4.45 (m, 2H), 7.91 (s, 1H), 10.21 (s, 2H).

LCMS: m/z 273

### Synthesis of N-{1-[(difluoromethoxy)methyl]cyclopropyl}-4H,5H,6H,7H-pyrazolo[1,5-a]pyrazine-3-carboxamide

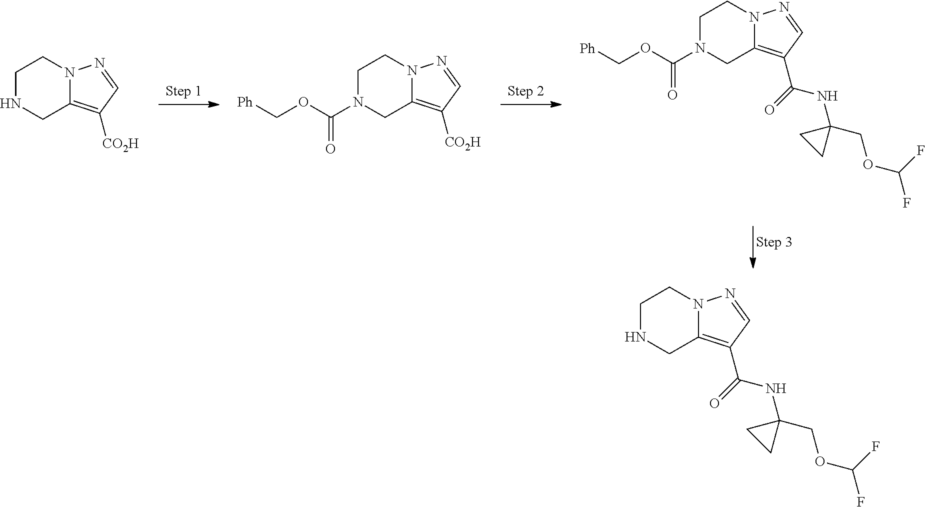 [SMILES: O=C(O)c1cnn2c1CNCC2 ; O=C(O)c1cnn2c1CN(C(=O)OCc1ccccc1)CC2 ; O=C(NC1(COC(F)F)CC1)c1cnn2c1CN(C(=O)OCc1ccccc1)CC2 ; O=C(NC1(COC(F)F)CC1)c1cnn2c1CNCC2]

Step 1: To a solution of 4H,5H,6H,7H-pyrazolo[1,5-a]pyrazine-3-carboxylic acid dihydrochloride (5.0 g, 20.83 mmol) in THF/H₂O (9/1) (100 mL) was added triethylamine (9.48 g, 93.72 mmol, 13.06 mL). The resulting mixture was stirred for 5 mins, then N-(benzyloxycarbonyloxy)succinimide (5.71 g, 22.91 mmol) was added and the resulting mixture stirred overnight. The mixture was then concentrated and the residue was partitioned between EtOAc (50 mL) and water (50 mL). The organic layer was washed with brine, dried over sodium sulfate and concentrated to give crude 5-[(benzyloxy)carbonyl]-4H,5H,6H,7H-pyrazolo[1,5-a]pyrazine-3-carboxylic acid (6.5 g) as a yellow solid.

Step 2: 1-[(Difluoromethoxy)methyl]cyclopropan-1-amine (796.69 mg, 5.81 mmol), HATU (971.99 mg, 2.56 mmol) and triethylamine (352.74 mg, 3.49 mmol, 490.0 μL) were mixed in dry DMF (10 mL) and the resulting mixture was stirred at r.t. for 10 minutes. 5-[(benzyloxy)carbonyl]-4H,5H,6H,7H-pyrazolo[1,5-a]pyrazine-3-carboxylic acid (700.0 mg, 2.32 mmol) was then added, and the resulting mixture was stirred at r.t. overnight. The mixture was then poured into water (60 mL). The resulting precipitate was collected by filtration, washed with H₂O (2×10 mL) and dried. The resulting material was purified by HPLC to give benzyl 3-(1-[(difluoromethoxy)methyl]cyclopropylcarbamoyl)-4H,5H,6H,7H-pyrazolo[1,5-a]pyrazine-5-carboxylate (630.0 mg, 1.5 mmol, 64.5% yield).

Step 3: To a solution of benzyl 3-(1-[(difluoromethoxy)methyl]cyclopropylcarbamoyl)-4H,5H,6H,7H-pyrazolo[1,5-a]pyrazine-5-carboxylate (630.0 mg, 1.5 mmol) in dry MeOH (5 mL) was added 10% palladium on carbon (20 mg). The resulting mixture was hydrogenated at 1 atm pressure. After consumption of the starting material (¹H NMR control) the mixture was filtered. The filtrate was evaporated to dryness to obtain N-1-[(difluoromethoxy)methyl]cyclopropyl-4H,5H,6H,7H-pyrazolo[1,5-a]pyrazine-3-carboxamide (330.0 mg, 1.15 mmol, 76.9% yield).

¹H NMR (500 MHz, d6-DMSO) δ 1.95 (s, 3H), 4.42 (s, 2H), 6.56 (s, 1H), 6.73 (s, 1H), 8.88 (s, 1H).

LCMS: m/z 287.2

### Synthesis of tert-butyl 3-{7-hydroxy-4-azaspiro[2.5]octane-4-carbonyl}-4H,5H,6H,7H-pyrazolo[1,5-a]pyrazine-5-carboxylate

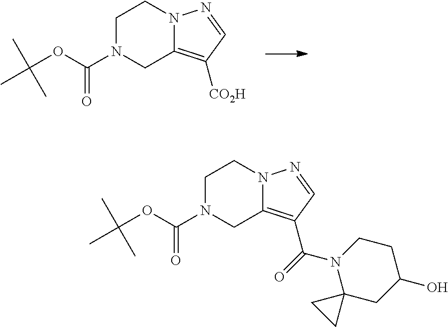 [SMILES: CC(C)(C)OC(=O)N1CCn2ncc(C(=O)O)c2C1 ; CC(C)(C)OC(=O)N1CCn2ncc(C(=O)N3CCC(O)CC34CC4)c2C1]

To a solution of 5-[(tert-butoxy)carbonyl]-4H,5H,6H,7H-pyrazolo[1,5-a]pyrazine-3-carboxylic acid (1.13 g, 4.22 mmol) and triethylamine (1.07 g, 10.55 mmol, 1.47 ml) in MeCN (20 mL) was added HATU (1.77 g, 4.64 mmol). The resulting mixture was stirred for 10 min then 4-azaspiro[2.5]octan-7-ol hydrochloride (760.0 mg, 4.64 mmol) was added and the stirring was continued overnight. The reaction mixture was partitioned between EtOAc (50 mL) and water (100 mL). The organic phase was washed with water (2×20 mL), brine, dried over sodium sulfate and concentrated under reduced pressure. The product was purified by HPLC to give tert-butyl 3-7-hydroxy-4-azaspiro[2.5]octane-4-carbonyl-4H,5H,6H,7H-pyrazolo[1,5-a]pyrazine-5-carboxylate (275.0 mg, 730.51 μmol, 17.3% yield).

¹H NMR (400 MHz, d6-DMSO) δ 0.56 (m, 2H), 0.82 (m, 1H), 0.92 (m, 1H), 1.20 (m, 1H), 1.43 (s, 9H), 1.81 (m, 2H), 3.75 (m, 1H), 3.83 (m, 3H), 4.11 (m, 4H), 4.62 (m, 1H), 4.71 (m, 1H), 4.76 (m, 1H), 7.70 (s, 1H).

LCMS: m/z 377.2

### Synthesis of tert-butyl 3-{[(2R)-1,1,1-trifluoropropan-2-yl]carbamoyl}-4H,5H,6H,7H-pyrazolo[1,5-a]pyrazine-5-carboxylate

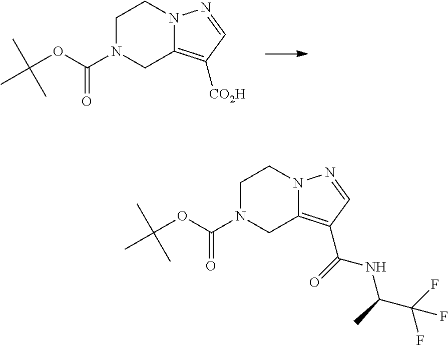 [SMILES: CC(C)(C)OC(=O)N1CCn2ncc(C(=O)O)c2C1 ; C[C@@H](NC(=O)c1cnn2c1CN(C(=O)OC(C)(C)C)CC2)C(F)(F)F]

To a solution of 5-[(tert-butoxy)carbonyl]-4H,5H,6H,7H-pyrazolo[1,5-a]pyrazine-3-carboxylic acid (804.39 mg, 3.01 mmol) and triethylamine (609.07 mg, 6.02 mmol, 840.0 μL) in dry DMF (30 mL) was added HATU (1.22 g, 3.21 mmol). The resulting mixture was stirred for 10 min then (2R)-1,1,1-trifluoropropan-2-amine hydrochloride (300.0 mg, 2.01 mmol) was added and the stirring was continued overnight. The reaction mixture was partitioned between EtOAc (50 mL) and H₂O (300 mL). The organic phase was washed with H₂O (2×50 mL), brine, dried over sodium sulfate and concentrated under reduced pressure to give a viscous brown residue, which was purified by HPLC to give tert-butyl 3-[(2R)-1,1,1-trifluoropropan-2-yl]carbamoyl-4H,5H,6H,7H-pyrazolo[1,5-a]pyrazine-5-carboxylate (353.2 mg, 974.76 μmol, 48.6% yield).

¹H NMR (500 MHz, CDCl₃) δ 1.40 (d, 3H), 1.50 (s, 9H), 3.86 (m, 1H), 3.94 (m, 1H), 4.19 (m, 2H), 4.92 (m, 3H), 5.85 (m, 1H), 7.70 (s, 1H).

LCMS: m/z 363.4

### Synthesis of benzyl 3-{[2-(difluoromethoxy)ethyl]carbamoyl}-4H,5H,6H,7H-pyrazolo[1,5-a]pyrazine-5-carboxylate

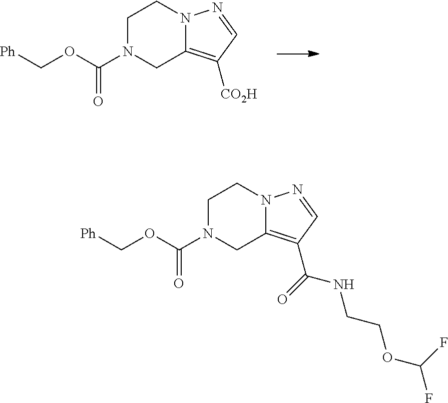 [SMILES: O=C(O)c1cnn2c1CN(C(=O)OCc1ccccc1)CC2 ; O=C(NCCOC(F)F)c1cnn2c1CN(C(=O)OCc1ccccc1)CC2]

2-(difluoromethoxy)ethan-1-amine (368.45 mg, 3.32 mmol), HATU (693.6 mg, 1.82 mmol) and triethylamine (184.59 mg, 1.82 mmol, 250.0 μl) were mixed in dry DMF (5 mL) at r.t. and the resulting mixture was stirred for 10 minutes. 5-[(Benzyloxy)carbonyl]-4H,5H,6H,7H-pyrazolo[1,5-a]pyrazine-3-carboxylic acid (500.0 mg, 1.66 mmol) was added thereto and the resulting mixture was stirred at r.t. overnight. The resulting mixture was partitioned between H₂O (50 mL) and EtOAc (50 mL). The organic phase was separated, dried over sodium sulfate and concentrated. The residue was purified by HPLC to give benzyl 3-[2-(difluoromethoxy)ethyl]carbamoyl-4H,5H,6H,7H-pyrazolo[1,5-a]pyrazine-5-carboxylate (437.6 mg, 1.11 mmol, 66.9% yield) as white solid.

¹H NMR (400 MHz, d6-DMSO) δ 3.42 (m, 2H), 3.89 (m, 4H), 4.13 (t, 2H), 4.86 (m, 2H), 5.15 (s, 2H), 6.67 (t, 1H), 7.34 (m, 1H), 7.39 (m, 4H), 7.98 (s, 1H), 8.28 (t, 1H).

LCMS: m/z 395.2

### Synthesis of tert-butyl 3-{[1-(trifluoromethyl)cyclopropyl]carbamoyl}-4H,5H,6H,7H-pyrazolo[1,5-a]pyrazine-5-carboxylate

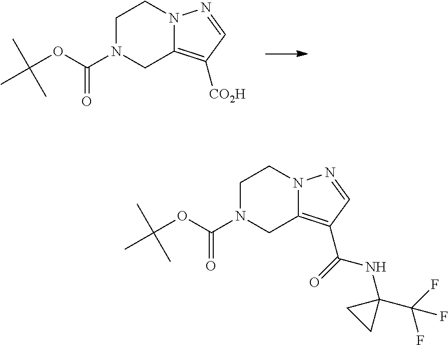 [SMILES: CC(C)(C)OC(=O)N1CCn2ncc(C(=O)O)c2C1 ; CC(C)(C)OC(=O)N1CCn2ncc(C(=O)NC3(C(F)(F)F)CC3)c2C1]

To a solution of 5-[(tert-butoxy)carbonyl]-4H,5H,6H,7H-pyrazolo[1,5-a]pyrazine-3-carboxylic acid (827.49 mg, 3.1 mmol) in dry DMF (3 mL) was added HATU (1.29 g, 3.41 mmol). The resulting mixture was stirred for 30 min then 1-(trifluoromethyl)cyclopropanamine hydrochloride (750.0 mg, 4.64 mmol) and triethylamine (1.25 g, 12.38 mmol, 1.73 ml) were added and the stirring was continued overnight. The reaction mixture was partitioned between EtOAc (50 mL) and water (30 mL). The organic phase was washed with water (2×20 mL), brine, dried over sodium sulfate and concentrated under reduced pressure. The residue was purified by HPLC to give tert-butyl 3-[1-(trifluoromethyl)cyclopropyl]carbamoyl-4H,5H,6H,7H-pyrazolo[1,5-a]pyrazine-5-carboxylate (439.1 mg, 1.17 mmol, 37.9% yield) as yellow solid.

¹H NMR (400 MHz, CDCl₃) δ 1.18 (m, 2H), 1.37 (m, 2H), 1.47 (s, 9H), 3.85 (t, 2H), 4.14 (t, 2H), 4.88 (s, 2H), 6.32 (s, 1H), 7.63 (s, 1H).

LCMS: m/z 375.2

### Synthesis of tert-butyl 3-{[1-(trifluoromethyl)cyclobutyl]carbamoyl}-4H,5H,6H,7H-pyrazolo[1,5-a]pyrazine-5-carboxylate

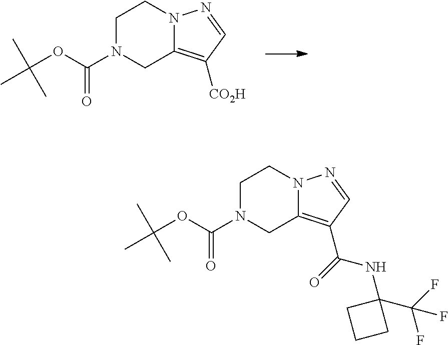 [SMILES: CC(C)(C)OC(=O)N1CCn2ncc(C(=O)O)c2C1 ; CC(C)(C)OC(=O)N1CCn2ncc(C(=O)NC3(C(F)(F)F)CCC3)c2C1]

To a solution of 5-[(tert-butoxy)carbonyl]-4H,5H,6H,7H-pyrazolo[1,5-a]pyrazine-3-carboxylic acid (761.13 mg, 2.85 mmol) in dry DMF (3 mL) was added HATU (1.19 g, 3.13 mmol). The resulting mixture was stirred for 30 min then 1-(trifluoromethyl)cyclobutan-1-amine hydrochloride (750.0 mg, 4.27 mmol) and triethylamine (1.15 g, 11.39 mmol) were added and the stirring was continued overnight. The reaction mixture was partitioned between EtOAc (50 mL) and water (30 mL). The organic phase was washed with water (2×20 mL), brine, dried over sodium sulfate and concentrated under reduced pressure. The residue was purified by HPLC to give tert-butyl 3-[1-(trifluoromethyl)cyclobutyl]carbamoyl-4H,5H,6H,7H-pyrazolo[1,5-a]pyrazine-5-carboxylate (448.2 mg, 1.15 mmol, 40.5% yield) as yellow solid.

¹H NMR (400 MHz, CDCl₃) δ 1.46 (s, 9H), 2.01 (m, 2H), 2.58 (m, 4H), 3.85 (t, 2H), 4.15 (t, 2H), 4.88 (s, 2H), 5.83 (s, 1H), 7.63 (s, 1H).

LCMS: m/z 389.2

### Example 1

### 5-(1H-indole-2-carbonyl)-N-[1-(methoxymethyl)cyclopropyl]-N,6-dimethyl-4H,5H,6H,7H-pyrazolo[1,5-a]pyrazine-3-carboxamide

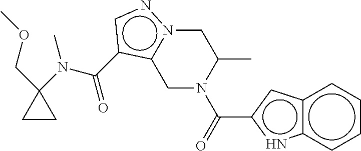

Rt (Method A) 3.03 mins, m/z 422 [M+H]+

¹H NMR (400 MHz, DMSO-d6) δ 11.68 (s, 1H), 8.13-7.79 (m, 1H), 7.66 (d, J=7.9 Hz, 1H), 7.45 (d, J=8.3 Hz, 1H), 7.25-7.18 (m, 1H), 7.11-7.04 (m, 1H), 6.94 (s, 1H), 5.82-5.36 (m, 1H), 5.32-5.23 (m, 1H), 4.98-4.54 (m, 1H), 4.46-4.25 (m, 1H), 4.18 (d, J=13.0 Hz, 1H), 3.62-3.48 (m, 2H), 3.28 (s, 3H), 3.20-2.88 (m, 3H), 1.32-0.67 (m, 7H).

### Example 2

### N-cyclopropyl-5-(1H-indole-2-carbonyl)-N,6-dimethyl-4H,5H,6H,7H-pyrazolo[1,5-a]pyrazine-3-carboxamide (racemate)

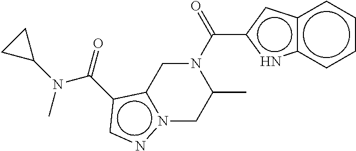

Rt (Method A) 2.96 mins, m/z 378 [M+H]+

¹H NMR (400 MHz, DMSO-d6) δ 11.68 (s, 1H), 8.03 (s, 1H), 7.66 (d, J=7.9 Hz, 1H), 7.45 (d, J=8.2 Hz, 1H), 7.25-7.18 (m, 1H), 7.11-7.04 (m, 1H), 6.96-6.91 (m, 1H), 5.57 (d, J=18.5 Hz, 1H), 5.34-5.24 (m, 1H), 4.90-4.60 (m, 1H), 4.42-4.31 (m, 1H), 4.19 (d, J=12.9 Hz, 1H), 3.13-3.04 (m, 1H), 2.94 (s, 3H), 1.24 (d, J=6.9 Hz, 3H), 0.85-0.77 (m, 2H), 0.64-0.56 (m, 2H).

### Example 3

### 2-{3-cyclobutyl-4H,5H,6H,7H-pyrazolo[1,5-a]pyrazine-5-carbonyl}-4,5-difluoro-1H-indole

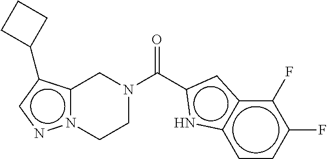

Rt (Method A) 3.44 mins, m/z 357 [M+H]+

¹H NMR (400 MHz, DMSO-d6) δ 12.09 (s, 1H), 7.43 (s, 1H), 7.31-7.20 (m, 2H), 7.09 (s, 1H), 5.11-4.70 (m, 2H), 4.30-4.12 (m, 4H), 3.40-3.34 (m, 1H), 2.29-2.17 (m, 2H), 2.09-1.86 (m, 3H), 1.85-1.76 (m, 1H).

### Example 4

### 2-{3-cyclobutyl-4H,5H,6H,7H-pyrazolo[1,5-a]pyrazine-5-carbonyl}-6-fluoro-4-methyl-1H-indole

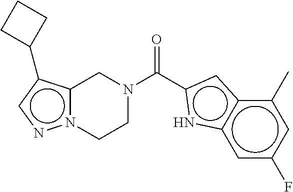

Rt (Method A) 3.5 mins, m/z 353 [M+H]+

¹H NMR (400 MHz, DMSO-d6) δ 11.72 (s, 1H), 7.42 (s, 1H), 7.02 (s, 1H), 7.00-6.94 (m, 1H), 6.80-6.73 (m, 1H), 5.01-4.81 (m, 2H), 4.27-4.16 (m, 4H), 3.41-3.34 (m, 1H), 2.52 (s, 3H), 2.30-2.19 (m, 2H), 2.09-1.87 (m, 3H), 1.86-1.76 (m, 1H).

### Example 5

### 2-{3-cyclobutyl-4H,5H,6H,7H-pyrazolo[1,5-a]pyrazine-5-carbonyl}-4,6-difluoro-1H-indole

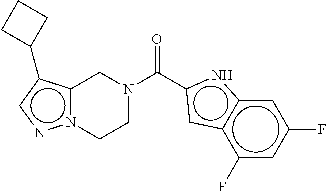

To 4,6-difluoro-1H-indole-2-carboxylic acid (22.24 mg, 0.113 mmol) was added a solution of HATU (47.2 mg, 0.124 mmol) in dry DMSO (400 μL) and the mixture was stirred for 10 min. Then a solution of 3-cyclobutyl-4,5,6,7-tetrahydropyrazolo[1,5-a]pyrazine (20 mg, 0.113 mmol) in dry DMSO (400 μL) was added followed by triethylamine (100 μL, 0.717 mmol). The mixture was stirred for 1H, then a few drops of water were added and the mixture was purified directly by reverse phase column chromatography, to give the product as a white solid (0.0141 g, 35% yield).

Rt (Method A) 3.47 mins, m/z 357 [M+H]+

¹H NMR (400 MHz, DMSO-d6) δ 12.10 (s, 1H), 7.43 (s, 1H), 7.09-7.02 (m, 2H), 6.92 (td, J=10.4, 1.9 Hz, 1H), 5.06-4.75 (m, 2H), 4.29-4.12 (m, 4H), 3.41-3.33 (m, 1H), 2.24 (d, J=8.0 Hz, 2H), 2.05-1.87 (m, 3H), 1.85-1.73 (m, 1H).

### Example 6

### 2-{3-cyclobutyl-4H,5H,6H,7H-pyrazolo[1,5-a]pyrazine-5-carbonyl}-1H-indole

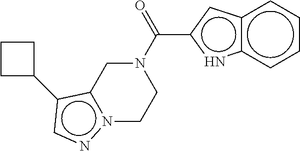

Rt (Method A) 3.32 mins, m/z 321 [M+H]+

¹H NMR (400 MHz, DMSO-d6) δ 11.67 (s, 1H), 7.65 (d, J=8.0 Hz, 1H), 7.47-7.41 (m, 2H), 7.21 (t, J=7.6 Hz, 1H), 7.07 (t, J=7.5 Hz, 1H), 6.97 (s, 1H), 5.06-4.77 (m, 2H), 4.33-4.11 (m, 4H), 3.41-3.34 (m, 1H), 2.29-2.18 (m, 2H), 2.08-1.86 (m, 3H), 1.85-1.77 (m, 1H).

### Example 7

### 5-(4-chloro-1H-indole-2-carbonyl)-N-[1-(methoxymethyl)cyclopropyl]-N-methyl-4H,5H,6H,7H-pyrazolo[1,5-a]pyrazine-3-carboxamide

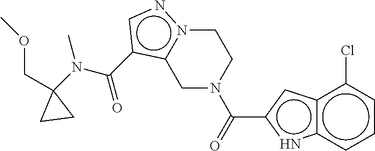

Rt (Method A) 3.27 mins. mz 442/444 [M+H]+

¹H NMR (400 MHz, DMSO-d6) δ 12.09 (s, 1H), 7.93 (s, 1H), 7.42 (d, J=8.1 Hz, 1H), 7.21 (t, J=7.8 Hz, 1H), 7.16 (d, J=7.4 Hz, 1H), 6.92 (s, 1H), 5.49-4.78 (m, 2H), 4.50-3.93 (m, 4H), 3.65-3.43 (m, 2H), 3.27 (s, 3H), 3.01 (s, 3H), 1.27-0.52 (m, 4H).

### Example 8

### 5-(4,6-difluoro-1H-indole-2-carbonyl)-N-[1-(methoxymethyl)cyclopropyl]-N-methyl-4H,5H,6H,7H-pyrazolo[1,5-a]pyrazine-3-carboxamide

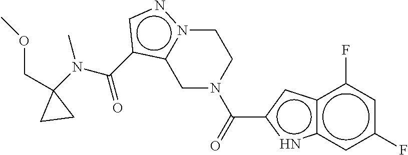

Rt (Method A) 3.23 mins, m/z 444 [M+H]+

¹H NMR (400 MHz, DMSO-d6) δ 12.14 (s, 1H), 7.94 (s, 1H), 7.13-6.99 (m, 2H), 6.93 (t, J=10.2 Hz, 1H), 5.13 (s, 2H), 4.53-3.97 (m, 4H), 3.65-3.43 (m, 2H), 3.27 (s, 3H), 3.01 (s, 3H), 1.18-0.47 (m, 4H).

### Example 9

### 5-(1H-indole-2-carbonyl)-N-[1-(methoxymethyl)cyclopropyl]-N-methyl-4H,5H,6H,7H-pyrazolo[1,5-a]pyrazine-3-carboxamide

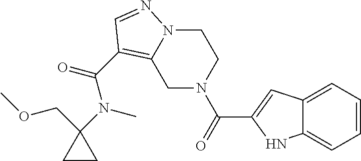 [SMILES: COCC1(N(C)C(=O)c2cnn3c2CN(C(=O)c2cc4ccccc4[nH]2)CC3)CC1]

Rt (Method A) 3.04 mins, m/z 408 [M+H]+

¹H NMR (400 MHz, DMSO-d6) δ 11.71 (s, 1H), 7.94 (s, 1H), 7.66 (d, J=8.0 Hz, 1H), 7.45 (d, J=8.2 Hz, 1H), 7.22 (t, J=7.6 Hz, 1H), 7.08 (t, J=7.5 Hz, 1H), 6.95 (s, 1H), 5.15 (s, 2H), 4.49-3.98 (m, 4H), 3.67-3.42 (m, 2H), 3.28 (s, 3H), 3.01 (s, 3H), 1.33-0.59 (m, 4H).

### Example 10

### 5-(1H-indole-2-carbonyl)-N-{[1-(methoxymethyl)cyclopropyl]methyl}-4H,5H,6H,7H-pyrazolo[1,5-a]pyrazine-3-carboxamide

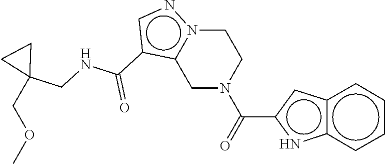

Rt (Method A) 3.03 mins, m/z 408 [M+H]+

¹H NMR (400 MHz, DMSO-d6) δ 11.71 (s, 1H), 8.04 (s, 1H), 8.02-7.98 (m, 1H), 7.65 (d, J=8.0 Hz, 1H), 7.44 (d, J=8.3 Hz, 1H), 7.22 (t, J=7.6 Hz, 1H), 7.07 (t, J=7.5 Hz, 1H), 6.95 (s, 1H), 5.20 (s, 2H), 4.26 (d, J=24.2 Hz, 4H), 3.27-3.15 (m, 7H), 0.49 (s, 2H), 0.35 (q, J=4.1 Hz, 2H).

### Example 11

### 5-(1H-indole-2-carbonyl)-N-methyl-N-{1-[(propan-2-yloxy)methyl]cyclopropyl}-4H,5H,6H,7H-pyrazolo[1,5-a]pyrazine-3-carboxamide

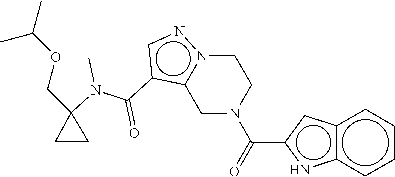

Rt (Method J) 1.42 mins, m/z 436 [M+H]+

¹H NMR (400 MHz, DMSO-d6) δ 11.69 (s, 1H), 8.02 (s, 1H), 7.65 (d, J=8.0 Hz, 1H), 7.44 (d, J=8.3 Hz, 1H), 7.21 (ddd, J=8.3, 6.9, 1.2 Hz, 1H), 7.07 (ddd, J=7.9, 6.8, 1.0 Hz, 1H), 6.94 (s, 1H), 5.14 (m, 2H), 4.29 (m, 4H), 3.54 (m, 3H), 3.00 (m, 3H), 1.16-0.92 (m, 7H), 0.81 (m, 3H).

### Example 12

### N-[1-(ethoxymethyl)cyclopropyl]-5-(1H-indole-2-carbonyl)-N-methyl-4H,5H,6H,7H-pyrazolo[1,5-a]pyrazine-3-carboxamide

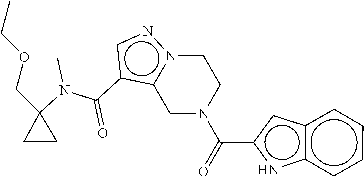

Rt (Method J) 1.33 mins, m/z 422 [M+H]+

¹H NMR (400 MHz, DMSO-d6) δ 11.69 (s, 1H), 7.99 (s, 1H), 7.65 (d, J=7.9 Hz, 1H), 7.44 (d, J=8.3 Hz, 1H), 7.21 (dd, J=8.1, 6.8 Hz, 1H), 7.07 (t, J=7.5 Hz, 1H), 6.94 (s, 1H), 5.40-4.89 (m, 2H), 4.30 (m, 4H), 3.57 (m, 2H), 3.49-3.41 (m, 2H), 3.02 (m, 3H), 1.09 (m, 4H), 0.82 (m, 3H).

### Example 13

### 5-(4-chloro-1H-indole-2-carbonyl)-N-[1-(hydroxymethyl)cyclopropyl]-N-methyl-4H,5H,6H,7H-pyrazolo[1,5-a]pyrazine-3-carboxamide

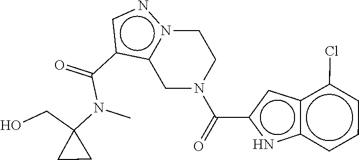

Rt (Method A) 2.89 mins, m/z 428/430 [M+H]+

¹H NMR (400 MHz, DMSO-d6) δ 12.08 (s, 1H), 8.13-7.80 (m, 1H), 7.42 (d, J=8.0 Hz, 1H), 7.21 (t, J=7.8 Hz, 1H), 7.16 (d, J=7.4 Hz, 1H), 6.92 (s, 1H), 5.42-4.85 (m, 3H), 4.40-4.05 (m, 4H), 3.77-3.53 (m, 2H), 3.14-2.84 (m, 3H), 1.30-0.58 (m, 4H).

### Example 14

### 5-(4-ethyl-6-fluoro-1H-indole-2-carbonyl)-N-[1-(hydroxymethyl)cyclopropyl]-N-methyl-4H,5H,6H,7H-pyrazolo[1,5-a]pyrazine-3-carboxamide

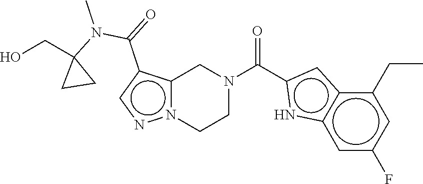

Rt (Method A) 3.01 mins, m/z 440 [M+H]+

¹H NMR (400 MHz, DMSO-d6) δ 11.74 (s, 1H), 8.21-7.74 (m, 1H), 7.02 (s, 1H), 7.01-6.95 (m, 1H), 6.78 (dd, J=10.8, 1.8 Hz, 1H), 5.36-4.72 (m, 3H), 4.41-4.05 (m, 4H), 3.78-3.46 (m, 2H), 3.20-2.80 (m, 5H), 1.28 (t, J=7.5 Hz, 3H), 1.20-0.61 (m, 4H).

### Example 15

### N-[1-(hydroxymethyl)cyclopropyl]-5-(1H-indole-2-carbonyl)-N-methyl-4H,5H,6H,7H-pyrazolo[1,5-a]pyrazine-3-carboxamide

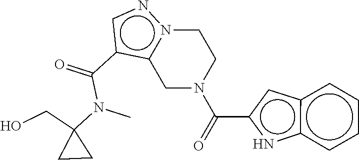

Rt (Method A) 2.68 mins, m/z 394 [M+H]+

¹H NMR (400 MHz, DMSO-d6) δ 11.69 (s, 1H), 8.13-7.80 (m, 1H), 7.65 (d, J=8.0 Hz, 1H), 7.44 (d, J=8.2 Hz, 1H), 7.21 (t, J=7.6 Hz, 1H), 7.07 (t, J=7.5 Hz, 1H), 6.95 (s, 1H), 5.41-4.67 (m, 3H), 4.45-4.00 (m, 4H), 3.79-3.51 (m, 2H), 3.19-2.81 (m, 3H), 1.29-0.60 (m, 4H).

### Example 16

### N-{1-[(difluoromethoxy)methyl]cyclopropyl}-5-(1H-indole-2-carbonyl)-N-methyl-4H,5H,6H,7H-pyrazolo[1,5-a]pyrazine-3-carboxamide

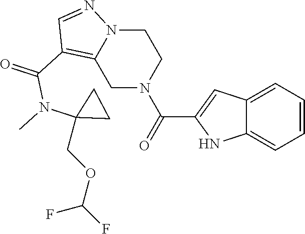 [SMILES: CN(C(=O)c1cnn2c1CN(C(=O)c1cc3ccccc3[nH]1)CC2)C1(COC(F)F)CC1]

Rt (Method B) 3.11 mins, m/z 444 [M+H]+

¹H NMR (400 MHz, DMSO-d6) δ 11.69 (s, 1H), 7.85 (s, 1H), 7.65 (d, J=8.0 Hz, 1H), 7.44 (d, J=8.3 Hz, 1H), 7.22 (ddd, J=8.2, 6.9, 1.1 Hz, 1H), 7.07 (ddd, J=7.9, 6.8, 1.0 Hz, 1H), 6.95 (s, 1H), 6.70 (t, J=75.8 Hz, 1H), 5.41-4.88 (m, 2H), 4.38-4.14 (m, 4H), 4.11-3.94 (m, 2H), 3.21-2.93 (m, 3H), 1.21-0.79 (m, 4H).

### Example 17

### 5-(1H-indole-2-carbonyl)-N-[1-(methoxymethyl)cyclopropyl]-N,6-dimethyl-4H,5H,6H,7H-pyrazolo[1,5-a]pyrazine-3-carboxamide (Enantiomer 2, Absolute Configuration Unknown)

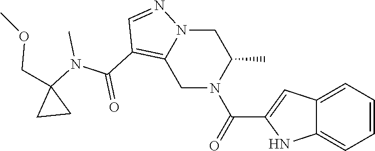 [SMILES: COCC1(N(C)C(=O)c2cnn3c2CN(C(=O)c2cc4ccccc4[nH]2)[C@@H](C)C3)CC1]

Rt (Method A) 3.02 mins, m/z 422 [M+H]+

¹H NMR (400 MHz, DMSO-d6) δ 11.67 (s, 1H), 8.07-7.83 (m, 1H), 7.66 (d, J=7.9 Hz, 1H), 7.45 (d, J=8.2 Hz, 1H), 7.27-7.17 (m, 1H), 7.13-7.03 (m, 1H), 6.93 (s, 1H), 5.80-5.36 (m, 1H), 5.36-5.20 (m, 1H), 4.90-4.49 (m, 1H), 4.43-4.28 (m, 1H), 4.18 (d, J=12.9 Hz, 1H), 3.65-3.47 (m, 2H), 3.28 (s, 3H), 3.20-2.89 (m, 3H), 1.42-0.64 (m, 7H).

Stereochemically pure material was obtained by separation of the racemate (Example 1) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 18

### N-cyclopropyl-5-(1H-indole-2-carbonyl)-N,6-dimethyl-4H,5H,6H,7H-pyrazolo[1,5-a]pyrazine-3-carboxamide (Enantiomer 2, Absolute Configuration Unknown)

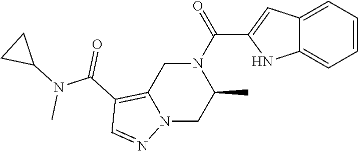 [SMILES: C[C@H]1Cn2ncc(C(=O)N(C)C3CC3)c2CN1C(=O)c1cc2ccccc2[nH]1]

Step 1: 5-(tert-butoxycarbonyl)-6-methyl-4,5,6,7-tetrahydropyrazolo[1,5-a]pyrazine-3-carboxylic acid (100 mg, 0.355 mmol) was dissolved in dry DMSO (3 mL) and HATU (149 mg, 0.391 mmol) was added. The mixture was stirred for 10 min. Triethylamine (0.248 ml, 1.777 mmol) was added followed by a solution of N-methylcyclopropanamine hydrochloride (38.2 mg, 0.355 mmol) in dry DMSO (1 mL) and the reaction mixture was stirred for 1 h. The reaction was quenched with a few drops of water and purified using by reversed phase column chromatography to give tert-butyl 3-[cyclopropyl(methyl)carbamoyl]-6-methyl-4H,5H,6H,7H-pyrazolo[1,5-a]pyrazine-5-carboxylate as a colorless oil (0.109 g, 83% yield.

Step 2: Tert-butyl 3-(cyclopropyl(methyl)carbamoyl)-6-methyl-6,7-dihydropyrazolo[1,5-a]pyrazine5(4H)-carboxylate (109 mg, 0.293 mmol) was dissolved in HCl (4 M in dioxane) (1 mL, 4.00 mmol). The mixture was stirred for overnight, then concentrated and stripped with DCM to give N-cyclopropyl-N,6-dimethyl-4H,5H,6H,7H-pyrazolo[1,5-a]pyrazine-3-carboxamide hydrochloride as a white solid that was used in the next step without further purification (0.076 g, 90% yield).

Step 3: Indole-2-carboxylic acid (13.99 mg, 0.087 mmol) was dissolved in dry DMSO (0.4 mL) and HATU (36.3 mg, 0.095 mmol) was added. In a separate vial, N-cyclopropylN,6-dimethyl-4,5,6,7-tetrahydropyrazolo[1,5-a]pyrazine-3-carboxamide hydrochloride (25 mg, 0.087 mmol) was suspended in dry DMSO (0.4 mL) and triethylamine (0.060 ml, 0.434 mmol) was added. The two mixtures were combined and stirred for 1 h. A few drops of water were added and the reaction mixture was purified directly by reverse phase column chromatography to give the product as a white solid (0.0183 g, 56% yield)

Rt (Method A) 2.97 mins, m/z 378 [M+H]+

¹H NMR (400 MHz, DMSO-d6) δ 11.67 (s, 1H), 8.03 (s, 1H), 7.66 (d, J=7.9 Hz, 1H), 7.45 (d, J=8.2 Hz, 1H), 7.25-7.18 (m, 1H), 7.11-7.04 (m, 1H), 6.93 (s, 1H), 5.56 (d, J=18.6 Hz, 1H), 5.36-5.21 (m, 1H), 4.87-4.64 (m, 1H), 4.47-4.31 (m, 1H), 4.19 (d, J=13.1 Hz, 1H), 3.16-3.02 (m, 1H), 2.94 (s, 3H), 1.24 (d, J=6.9 Hz, 3H), 0.90-0.74 (m, 2H), 0.67-0.51 (m, 2H).

Stereochemically pure material was obtained by separation of the racemate (Example 2) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 19

### 2-(3-{6,6-difluoro-4-azaspiro[2.4]heptane-4-carbonyl}-4H,5H,6H,7H-pyrazolo[1,5-a]pyrazine-5-carbonyl)-1H-indole

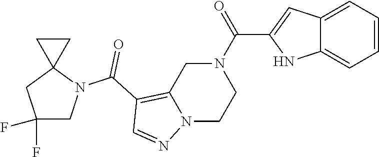 [SMILES: O=C(c1cc2ccccc2[nH]1)N1CCn2ncc(C(=O)N3CC(F)(F)CC34CC4)c2C1]

Rt (Method A) 1.39 mins, m/z 426 [M+H]+

¹H NMR (400 MHz, DMSO-d6) δ 11.69 (s, 1H), 7.88 (s, 1H), 7.65 (d, J=8.0 Hz, 1H), 7.44 (d, J=8.2 Hz, 1H), 7.21 (t, J=7.5 Hz, 1H), 7.07 (t, J=7.4 Hz, 1H), 6.95 (s, 1H), 5.09 (m, 2H), 4.43-4.09 (m, 6H), 2.47-2.37 (m, 2H), 1.94-1.73 (m, 2H), 0.72-0.54 (m, 2H).

### Example 20

### N-{1-[(difluoromethoxy)methyl]cyclopropyl}-5-(1H-indole-2-carbonyl)-N,6-dimethyl-4H,5H,6H,7H-pyrazolo[1,5-a]pyrazine-3-carboxamide

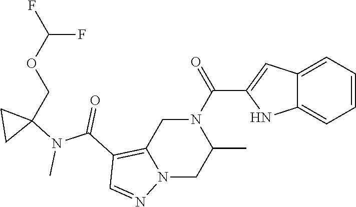 [SMILES: CC1Cn2ncc(C(=O)N(C)C3(COC(F)F)CC3)c2CN1C(=O)c1cc2ccccc2[nH]1]

Rt (Method B) 3.23 mins, m/z 458 [M+H]+

¹H NMR (400 MHz, DMSO-d6) δ 11.68 (s, 1H), 7.87 (s, 1H), 7.66 (d, J=8.0 Hz, 1H), 7.45 (d, J=8.3 Hz, 1H), 7.22 (t, J=7.6 Hz, 1H), 7.07 (t, J=7.4 Hz, 1H), 6.94 (s, 1H), 6.71 (t, J=75.9 Hz, 1H), 5.76-5.41 (m, 1H), 5.37-5.21 (m, 1H), 4.89-4.51 (m, 1H), 4.48-4.28 (m, 1H), 4.18 (d, J=13.0 Hz, 1H), 4.14-3.93 (m, 2H), 3.23-2.87 (m, 3H), 1.24 (d, J=6.7 Hz, 3H), 1.19-0.75 (m, 4H).

### Example 21

### 5-(4,5-difluoro-1H-indole-2-carbonyl)-N-{1-[(difluoromethoxy)methyl]cyclopropyl}-N-methyl-4H,5H,6H,7H-pyrazolo[1,5-a]pyrazine-3-carboxamide

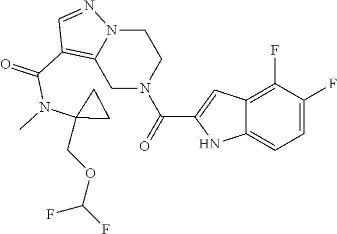 [SMILES: CN(C(=O)c1cnn2c1CN(C(=O)c1cc3c(F)c(F)ccc3[nH]1)CC2)C1(COC(F)F)CC1]

Rt (Method B) 3.27 mins, m/z 480 [M+H]+

¹H NMR (400 MHz, DMSO-d6) δ 12.12 (s, 1H), 7.85 (s, 1H), 7.33-7.19 (m, 2H), 7.06 (s, 1H), 6.69 (t, J=75.8 Hz, 1H), 5.48-4.77 (m, 2H), 4.51-4.14 (m, 4H), 4.14-3.93 (m, 2H), 3.21-2.90 (m, 3H), 1.41-0.71 (m, 4H).

### Example 22

### N-{1-[(difluoromethoxy)methyl]cyclopropyl}-5-(4-ethyl-6-fluoro-1H-indole-2-carbonyl)-N-methyl-4H,5H,6H,7H-pyrazolo[1,5-a]pyrazine-3-carboxamide

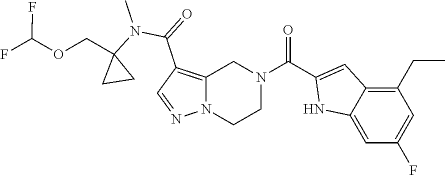 [SMILES: CCc1cc(F)cc2[nH]c(C(=O)N3CCn4ncc(C(=O)N(C)C5(COC(F)F)CC5)c4C3)cc12]

Rt (Method B) 3.43 mins, m/z 490 [M+H]+

¹H NMR (400 MHz, DMSO-d6) δ 11.74 (s, 1H), 7.84 (s, 1H), 7.03 (s, 1H), 7.01-6.95 (m, 1H), 6.92-6.46 (m, 2H), 5.37-4.92 (m, 2H), 4.38-4.15 (m, 4H), 4.13-3.95 (m, 2H), 3.23-2.95 (m, 3H), 2.90 (q, J=7.5 Hz, 2H), 1.28 (t, J=7.6 Hz, 3H), 1.15-0.73 (m, 4H).

### Example 23

### 5-(6-chloro-5-fluoro-1H-indole-2-carbonyl)-N-{1-[(difluoromethoxy)methyl]cyclopropyl}-N-methyl-4H,5H,6H,7H-pyrazolo[1,5-a]pyrazine-3-carboxamide

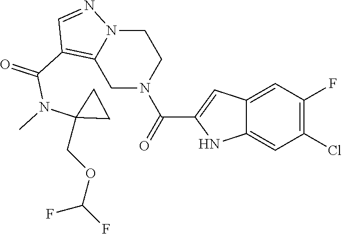 [SMILES: CN(C(=O)c1cnn2c1CN(C(=O)c1cc3cc(F)c(Cl)cc3[nH]1)CC2)C1(COC(F)F)CC1]

Rt (Method B) 3.36 mins, m/z 396/398 [M+H]+

¹H NMR (400 MHz, DMSO-d6) δ 11.93 (s, 1H), 7.85 (s, 1H), 7.66 (d, J=9.9 Hz, 1H), 7.56 (d, J=6.4 Hz, 1H), 6.97 (s, 1H), 6.69 (t, J=76.0 Hz, 1H), 5.38-4.91 (m, 2H), 4.37-4.13 (m, 4H), 4.11-3.92 (m, 2H), 3.21-2.89 (m, 3H), 1.21-0.78 (m, 4H).

### Example 24

### 5-(4,7-difluoro-1H-indole-2-carbonyl)-N-{1-[(difluoromethoxy)methyl]cyclopropyl}-N-methyl-4H,5H,6H,7H-pyrazolo[1,5-a]pyrazine-3-carboxamide

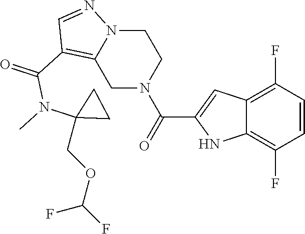 [SMILES: CN(C(=O)c1cnn2c1CN(C(=O)c1cc3c(F)ccc(F)c3[nH]1)CC2)C1(COC(F)F)CC1]

Rt (Method B) 3.25 mins, m/z 480 [M+H]+

¹H NMR (400 MHz, DMSO-d6) δ 12.52 (s, 1H), 7.85 (s, 1H), 7.06-6.93 (m, 2H), 6.93-6.44 (m, 2H), 5.38-4.84 (m, 2H), 4.42-3.88 (m, 6H), 3.21-2.83 (m, 3H), 1.22-0.71 (m, 4H).

### Example 25

### 5-(4-chloro-6-fluoro-1H-indole-2-carbonyl)-N-{1-[(difluoromethoxy)methyl]cyclopropyl}-N-methyl-4H,5H,6H,7H-pyrazolo[1,5-a]pyrazine-3-carboxamide

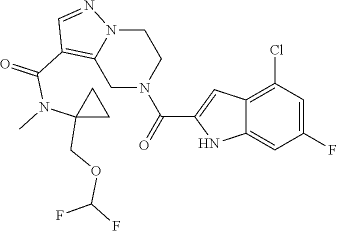 [SMILES: CN(C(=O)c1cnn2c1CN(C(=O)c1cc3c(Cl)cc(F)cc3[nH]1)CC2)C1(COC(F)F)CC1]

Rt (Method B) 3.4 mins, m/z 396/498 [M+H]+

¹H NMR (400 MHz, DMSO-d6) δ 12.17 (s, 1H), 7.85 (s, 1H), 7.22-7.14 (m, 2H), 6.95 (s, 1H), 6.70 (t, J=75.9 Hz, 1H), 5.42-4.91 (m, 2H), 4.46-4.15 (m, 4H), 4.14-3.92 (m, 2H), 3.21-2.88 (m, 3H), 1.31-0.71 (m, 4H).

### Example 26

### 5-(1H-indole-2-carbonyl)-4H,5H,6H,7H-pyrazolo[1,5-a]pyrazine-3-sulfonamide

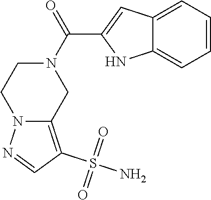 [SMILES: NS(=O)(=O)c1cnn2c1CN(C(=O)c1cc3ccccc3[nH]1)CC2]

Rt (Method H) 1.05 mins, m/z 346 [M+H]+

### Example 27

### N-[(1-hydroxycyclobutyl)methyl]-5-(1H-indole-2-carbonyl)-4H,5H,6H,7H-pyrazolo[1,5-a]pyrazine-3-sulfonamide

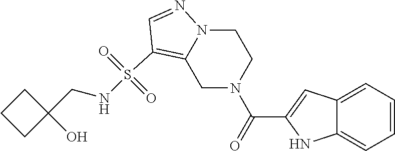 [SMILES: O=C(c1cc2ccccc2[nH]1)N1CCn2ncc(S(=O)(=O)NCC3(O)CCC3)c2C1]

Rt (Method H) 1.18 mins, m/z 430 [M+H]+

### Example 28

### 5-(1H-indole-2-carbonyl)-N-(1,1,1-trifluoropropan-2-yl)-4H,5H,6H,7H-pyrazolo[1,5-a]pyrazine-3-sulfonamide

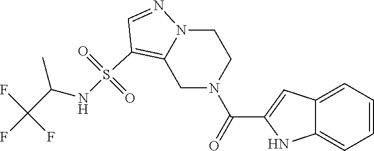 [SMILES: CC(NS(=O)(=O)c1cnn2c1CN(C(=O)c1cc3ccccc3[nH]1)CC2)C(F)(F)F]

Rt (Method H) 1.38 mins, m/z 442 [M+H]+

### Example 29

### N-(2-hydroxyethyl)-5-(1H-indole-2-carbonyl)-4H,5H,6H,7H-pyrazolo[1,5-a]pyrazine-3-sulfonamide

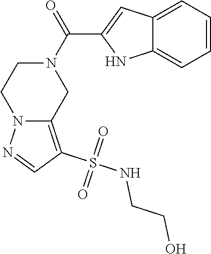 [SMILES: O=C(c1cc2ccccc2[nH]1)N1CCn2ncc(S(=O)(=O)NCCO)c2C1]

Rt (Method H) 1.03 mins, m/z 390 [M+H]+

¹H NMR (400 MHz, DMSO-d6) δ 11.73 (s, 1H), 8.01-7.16 (m, 5H), 7.08 (dd, J=8.0, 6.8 Hz, 1H), 6.98 (s, 1H), 5.41-4.92 (m, 2H), 4.89-4.48 (m, 1H), 4.46-4.01 (m, 4H), 3.44-3.37 (m, 2H), 2.83 (t, J=6.3 Hz, 2H).

### Example 30

### 5-(1H-indole-2-carbonyl)-N-(oxolan-3-yl)-4H,5H,6H,7H-pyrazolo[1,5-a]pyrazine-3-sulfonamide

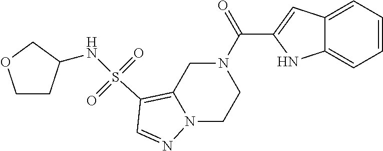 [SMILES: O=C(c1cc2ccccc2[nH]1)N1CCn2ncc(S(=O)(=O)NC3CCOC3)c2C1]

Rt (Method H) 1.15 mins, m/z 416 [M+H]+

### Example 31

### N-(2-hydroxyethyl)-5-(1H-indole-2-carbonyl)-N-methyl-4H,5H,6H,7H-pyrazolo[1,5-a]pyrazine-3-sulfonamide

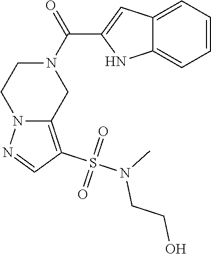 [SMILES: CN(CCO)S(=O)(=O)c1cnn2c1CN(C(=O)c1cc3ccccc3[nH]1)CC2]

Rt (Method H) 1.09 mins, m/z 404 [M+H]+

### Example 32

### 5-(1H-indole-2-carbonyl)-N-[1-(methoxymethyl)cyclopropyl]-N-methyl-4H,5H,6H,7H-pyrazolo[1,5-a]pyrazine-3-sulfonamide

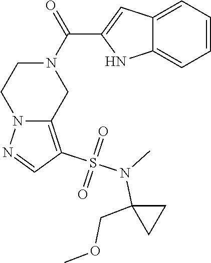 [SMILES: COCC1(N(C)S(=O)(=O)c2cnn3c2CN(C(=O)c2cc4ccccc4[nH]2)CC3)CC1]

Rt (Method H) 1.36 mins, m/z 444 [M+H]+

### Example 33

### 5-(1H-indole-2-carbonyl)-N-[(oxolan-3-yl)methyl]-4H,5H,6H,7H-pyrazolo[1,5-a]pyrazine-3-sulfonamide

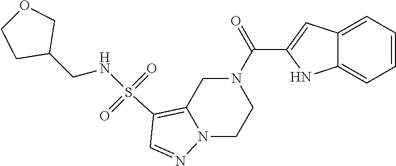 [SMILES: O=C(c1cc2ccccc2[nH]1)N1CCn2ncc(S(=O)(=O)NCC3CCOC3)c2C1]

Rt (Method H) 1.17 mins, m/z 430 [M+H]+

### Example 34

### 2-{3-[(4,4-difluoropiperidin-1-yl)sulfonyl]-4H,5H,6H,7H-pyrazolo[1,5-a]pyrazine-5-carbonyl}-1H-indole

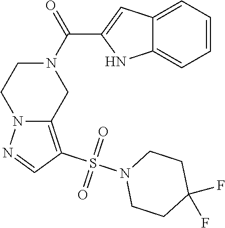 [SMILES: O=C(c1cc2ccccc2[nH]1)N1CCn2ncc(S(=O)(=O)N3CCC(F)(F)CC3)c2C1]

Step 1: To tert-butyl 3-[(4,4-difluoropiperidin-1-yl)sulfonyl]-4H,5H,6H,7H-pyrazolo[1,5-a]pyrazine-5-carboxylate (0.036 g, 0.09 mmol) was added HCl in dioxane (0.5 mL, 2 mmol). The mixture was stirred for 2 h, then concentrated under vacuum to give 4,4-difluoro-1-{4H,5H,6H,7H-pyrazolo[1,5-a]pyrazine-3-sulfonyl}piperidine hydrochloride hydrochloride as a white solid that was used in the next step without further purification.

Step 2: A mixture of indole-2-carboxylic acid (0.232 g, 1.440 mmol) and HATU (0.546 g, 1.436 mmol) in DMF (16 ml) was stirred at r.t. for 5 minutes. One sixteenth of this mixture was then added to 4,4-difluoro-1-{4H,5H,6H,7H-pyrazolo[1,5-a]pyrazine-3-sulfonyl}piperidine hydrochloride (0.09 mmol). DIPEA (0.047 mL, 0.270 mmol) was then added, and the mixture stirred at r.t. for 2 hours. The mixture was concentrated in vacuo and purified directly by chromatography to give the product as a white solid (0.016 g, 40% yield).

Rt (Method H) 1.44 mins, m/z 450 [M+H]+

### Example 35

### 1-{[5-(1H-indole-2-carbonyl)-4H,5H,6H,7H-pyrazolo[1,5-a]pyrazin-3-yl]sulfonyl}piperidin-4-ol

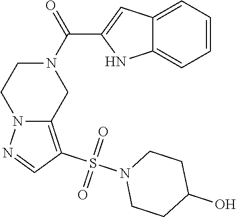 [SMILES: O=C(c1cc2ccccc2[nH]1)N1CCn2ncc(S(=O)(=O)N3CCC(O)CC3)c2C1]

Rt (Method H) 1.14 mins, m/z 430 [M+H]+

### Example 36

### 2-{3-[(4-methylpiperazin-1-yl)sulfonyl]-4H,5H,6H,7H-pyrazolo[1,5-a]pyrazine-5-carbonyl}-1H-indole

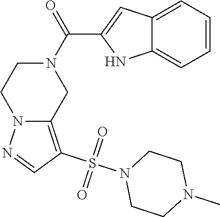 [SMILES: CN1CCN(S(=O)(=O)c2cnn3c2CN(C(=O)c2cc4ccccc4[nH]2)CC3)CC1]

Rt (Method H) 0.77 mins, m/z 429 [M+H]+

### Example 37

### 5-(1H-indole-2-carbonyl)-N-(propan-2-yl)-4H,5H,6H,7H-pyrazolo[1,5-a]pyrazine-3-sulfonamide

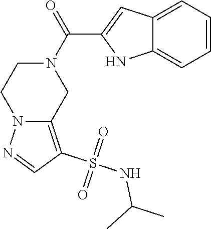 [SMILES: CC(C)NS(=O)(=O)c1cnn2c1CN(C(=O)c1cc3ccccc3[nH]1)CC2]

Rt (Method H) 1.28 mins, m/z 388 [M+H]+

### Example 38

### 2-[3-(morpholine-4-sulfonyl)-4H,5H,6H,7H-pyrazolo[1,5-a]pyrazine-5-carbonyl]-1H-indole

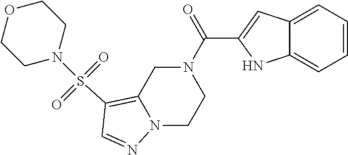 [SMILES: O=C(c1cc2ccccc2[nH]1)N1CCn2ncc(S(=O)(=O)N3CCOCC3)c2C1]

Rt (Method H) 1.25 mins, m/z 416 [M+H]+

### Example 39

### 2-[3-(pyrrolidine-1-sulfonyl)-4H,5H,6H,7H-pyrazolo[1,5-a]pyrazine-5-carbonyl]-1H-indole

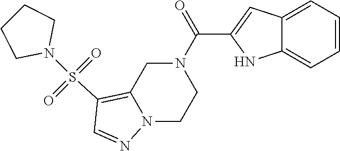 [SMILES: O=C(c1cc2ccccc2[nH]1)N1CCn2ncc(S(=O)(=O)N3CCCC3)c2C1]

Step 1: To tert-butyl 3-(pyrrolidine-1-sulfonyl)-4H,5H,6H,7H-pyrazolo[1,5-a]pyrazine-5-carboxylate (0.0321 g, 0.09 mmol) was added HCl in dioxane (0.5 mL, 2 mmol). The mixture was stirred for 2 h, then concentrated under vacuum to give 1-{4H,5H,6H,7H-pyrazolo[1,5-a]pyrazine-3-sulfonyl}pyrrolidine hydrochloride as a white solid that was used in the next step without further purification.

Step 2: A mixture of indole-2-carboxylic acid (0.232 g, 1.440 mmol) and HATU (0.546 g, 1.436 mmol) in DMF (16 ml) was stirred at r.t. for 5 minutes. One sixteenth of this mixture was then added to 1-{4H,5H,6H,7H-pyrazolo[1,5-a]pyrazine-3-sulfonyl}pyrrolidine hydrochloride (0.09 mmol). DIPEA (0.047 mL, 0.270 mmol) was then added, and the mixture stirred at r.t. for 2 hours. The mixture was concentrated in vacuo and purified directly by chromatography to give the product as a white solid (0.019 g, 53% yield).

Rt (Method H) 1.33 mins, m/z 400 [M+H]+

### Example 40

### 5-(1H-indole-2-carbonyl)-N,N-dimethyl-4H,5H,6H,7H-pyrazolo[1,5-a]pyrazine-3-sulfonamide

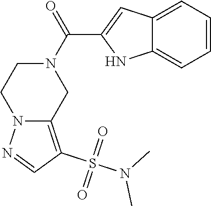 [SMILES: CN(C)S(=O)(=O)c1cnn2c1CN(C(=O)c1cc3ccccc3[nH]1)CC2]

Rt (Method H) 1.26 mins, m/z 374 [M+H]+

¹H NMR (400 MHz, DMSO-d6) δ 11.70 (s, 1H), 7.89 (s, 1H), 7.78-7.61 (m, 1H), 7.51-7.39 (m, 1H), 7.32-7.17 (m, 1H), 7.17-7.03 (m, 1H), 6.97 (s, 1H), 5.40-4.91 (m, 2H), 4.62-4.09 (m, 4H), 2.60 (s, 6H).

### Example 41

### 5-(1H-indole-2-carbonyl)-N-methyl-4H,5H,6H,7H-pyrazolo[1,5-a]pyrazine-3-sulfonamide

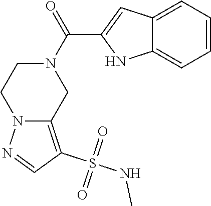 [SMILES: CNS(=O)(=O)c1cnn2c1CN(C(=O)c1cc3ccccc3[nH]1)CC2]

Rt (Method H) 1.14 mins, m/z 360 [M+H]+

### Example 42

### 2-(3-{6,6-difluoro-3-azabicyclo[3.1.0]hexane-3-carbonyl}-4H,5H,6H,7H-pyrazolo[1,5-a]pyrazine-5-carbonyl)-1H-indole

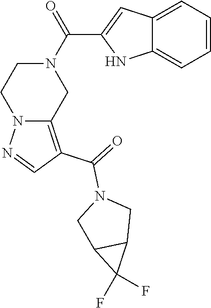 [SMILES: O=C(c1cc2ccccc2[nH]1)N1CCn2ncc(C(=O)N3CC4C(C3)C4(F)F)c2C1]

Rt (Method A) 2.98 mins, m/z 412 [M+H]+

¹H NMR (400 MHz, DMSO-d6) δ 11.73-11.66 (m, 1H), 7.92 (s, 1H), 7.65 (d, J=8.0 Hz, 1H), 7.44 (d, J=8.3 Hz, 1H), 7.22 (ddd, J=8.3, 6.8, 1.2 Hz, 1H), 7.07 (t, J=7.5 Hz, 1H), 6.94 (s, 1H), 4.28 (d, J=25.9 Hz, 4H), 4.09 (s, 1H), 4.04-3.92 (m, 2H), 3.72 (s, 1H), 2.54 (s, 2H).

### Example 43

### 2-(3-{3-azabicyclo[3.1.0]hexane-3-carbonyl}-4H,5H,6H,7H-pyrazolo[1,5-a]pyrazine-5-carbonyl)-1H-indole

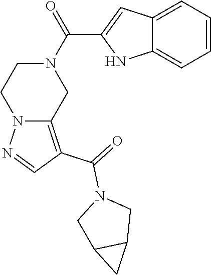 [SMILES: O=C(c1cc2ccccc2[nH]1)N1CCn2ncc(C(=O)N3CC4CC4C3)c2C1]

Rt (Method A) 2.89 mins, m/z 376 [M+H]+

¹H NMR (400 MHz, DMSO-d6) δ 11.70 (s, 1H), 7.85 (s, 1H), 7.65 (d, J=8.0 Hz, 1H), 7.44 (d, J=8.2 Hz, 1H), 7.22 (t, J=7.6 Hz, 1H), 7.07 (t, J=7.5 Hz, 1H), 6.94 (s, 1H), 4.37-4.16 (m, 4H), 3.79 (m, 3H), 1.60 (m, 2H), 0.68 (m, 1H), 0.06 (m, 1H).

### Example 44

### 5-(1H-indole-2-carbonyl)-N-methyl-N-[(pyridin-2-yl)methyl]-4H,5H,6H,7H-pyrazolo[1,5-a]pyrazine-3-carboxamide

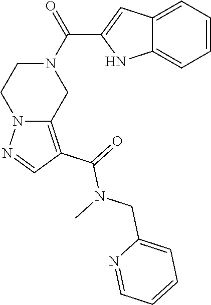 [SMILES: CN(Cc1ccccn1)C(=O)c1cnn2c1CN(C(=O)c1cc3ccccc3[nH]1)CC2]

Rt (Method A) 2.79 mins, m/z 415 [M+H]+

¹H NMR (400 MHz, DMSO-d6) δ 11.71 (s, 1H), 8.59-8.43 (m, 1H), 7.83 (m, 1H), 7.65 (d, J=7.9 Hz, 1H), 7.45 (d, J=8.2 Hz, 1H), 7.25 (dt, J=22.0, 6.7 Hz, 2H), 7.08 (t, J=7.5 Hz, 1H), 6.96 (s, 1H), 5.16 (s, 1H), 4.74 (s, 1H), 4.28 (m, 2H), 2.93 (s, 2H).

### Example 45

### 1-[5-(1H-indole-2-carbonyl)-4H,5H,6H,7H-pyrazolo[1,5-a]pyrazin-3-yl]cyclobutan-1-ol

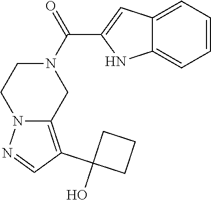 [SMILES: O=C(c1cc2ccccc2[nH]1)N1CCn2ncc(C3(O)CCC3)c2C1]

Rt (Method A) 2.82 mins, m/z 337 [M+H]+

¹H NMR (400 MHz, DMSO-d6) δ 11.67 (s, 1H), 7.63 (d, J=8.0 Hz, 1H), 7.48 (s, 1H), 7.44 (d, J=8.3 Hz, 1H), 7.21 (t, J=7.5 Hz, 1H), 7.07 (t, J=7.4 Hz, 1H), 6.94 (s, 1H), 5.36 (s, 1H), 5.25-4.75 (m, 2H), 4.30-4.16 (m, 4H), 2.35-2.16 (m, 4H), 1.80-1.66 (m, 1H), 1.62-1.47 (m, 1H).

### Example 46

### 5-(1H-indole-2-carbonyl)-N-methyl-N-[1-(pyridin-4-yl)cyclopropyl]-4H,5H,6H,7H-pyrazolo[1,5-a]pyrazine-3-carboxamide

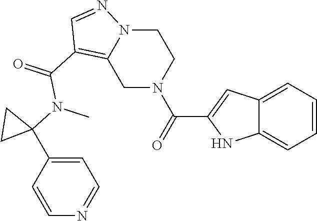 [SMILES: CN(C(=O)c1cnn2c1CN(C(=O)c1cc3ccccc3[nH]1)CC2)C1(c2ccncc2)CC1]

Step 1: Tert-butyl 3-(methyl(1-(pyridin-4-yl)cyclopropyl)carbamoyl)-6,7-dihydropyrazolo[1,5-a]pyrazine-5(4H)-carboxylate (102 mg, 0.257 mmol) was dissolved in 4M HCl in dioxane (1.5 mL, 6.00 mmol) and the resulting solution was stirred at r.t. for 4 h. The reaction mixture was diluted with dioxane (4 mL) and concentrated, then co-evaporated with toluene (2×10 mL) to give N-methyl-N-[1-(pyridin-4-yl)cyclopropyl]-4H,5H,6H,7H-pyrazolo[1,5-a]pyrazine-3-carboxamide hydrochloride as an off-white solid (0.098 g, 100% yield).

Step 2: To a solution of 1H-indole-2-carboxylic acid (20.63 mg, 0.128 mmol) in DMSO (0.6 mL) was added HATU (53.5 mg, 0.141 mmol). The resulting mixture was stirred at r.t. for 30 min. A mixture of of N-methyl-N-(1-(pyridin4-yl)cyclopropyl)-4,5,6,7-tetrahydropyrazolo[1,5-a]pyrazine-3-carboxamide hydrochloride (42.7 mg, 0.128 mmol) and triethylamine (0.089 mL, 0.640 mmol) in DMSO (0.7 mL) was added and the reaction was stirred at r.t. for 1 h. The mixture was filtered and purified directly by chromatography to give the product as a white solid (0.024 g, 42% yield).

Rt (Method A) 2.83 mins, m/z 441 [M+H]+

¹H NMR (400 MHz, DMSO-d6) δ 11.70 (s, 1H), 8.61-8.28 (m, 2H), 7.75-7.57 (m, 1H), 7.51-7.34 (m, 1H), 7.29-7.15 (m, 1H), 7.15-6.83 (m, 5H), 5.43-4.96 (m, 2H), 4.45-3.97 (m, 4H), 3.26-2.93 (m, 3H), 1.80-1.32 (m, 4H).

### Example 47

### 5-(1H-indole-2-carbonyl)-N-methyl-N-[1-(pyrimidin-2-yl)cyclopropyl]-4H,5H,6H,7H-pyrazolo[1,5-a]pyrazine-3-carboxamide

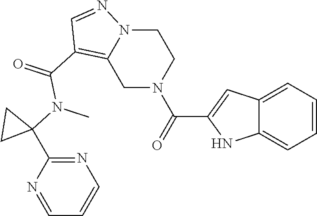 [SMILES: CN(C(=O)c1cnn2c1CN(C(=O)c1cc3ccccc3[nH]1)CC2)C1(c2ncccn2)CC1]

Step 1: To tert-butyl 3-(methyl(1-(pyrimidin-2-yl)cyclopropyl)carbamoyl)-6,7-dihydropyrazolo[1,5-a]pyrazine-5(4H)-carboxylate (100 mg, 0.251 mmol) was added 4M HCl in dioxane (1.4 mL, 5.60 mmol) After a short period the reaction mixture was diluted with dioxane (0.6 mL). Additional 4M HCl in dioxane (3.2 mL, 12.8 mmol) was added and stirring was continued at r.t. for 48 h. The mixture was concentrated to give N-methyl-N-[1-(pyrimidin-2yl)cyclopropyl]-4H,5H,6H,7H-pyrazolo[1,5-a]pyrazine-3-carboxamide hydrochloride as a white solid that was used in the next step without further purification (0.160 g).

Step 2: To a solution of 1H-indole-2-carboxylic acid (38.5 mg, 0.239 mmol) in dry DMSO (0.7 mL) was added HATU (100 mg, 0.263 mmol). The resulting solution was stirred at r.t. for 45 mins, then a mixture of N-methyl-N-(1-(pyrimidin-2-yl)cyclopropyl)-4,5,6,7-tetrahydropyrazolo[1,5-a]pyrazine-3-carboxamide hydrochloride (80 mg, 0.239 mmol) and triethylamine (0.167 mL, 1.195 mmol) in DMSO (0.7 mL) was added and the reaction was stirred at r.t. overnight. The reaction mixture was filtered and the filtrate purified directly by chromatography to give 5-(1H-indole-2-carbonyl)-N-methyl-N-[1-(pyrimidin-2-yl)cyclopropyl]-4H,5H,6H,7H-pyrazolo[1,5-a]pyrazine-3-carboxamide as a fluffy white solid (0.051 g, 48% yield).

Rt (Method A) 2.86 mins, m/z 442 [M+H]+

¹H NMR (400 MHz, DMSO-d6) δ 11.71 (s, 1H), 8.85-8.48 (m, 2H), 7.75-7.58 (m, 1H), 7.44 (d, J=8.2 Hz, 1H), 7.37 (t, J=4.9 Hz, 1H), 7.30-7.13 (m, 1H), 7.13-7.02 (m, 1H), 7.02-6.87 (m, 1H), 6.80 (s, 1H), 5.46-4.90 (m, 2H), 4.45-3.94 (m, 4H), 3.30-2.98 (m, 3H), 1.95-1.30 (m, 4H).

### Example 48

### 2-(3-{2-azabicyclo[3.1.0]hexane-2-carbonyl}-4H,5H,6H,7H-pyrazolo[1,5-a]pyrazine-5-carbonyl)-1H-indole

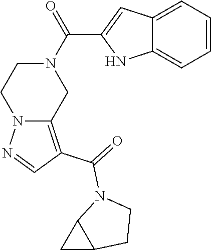 [SMILES: O=C(c1cc2ccccc2[nH]1)N1CCn2ncc(C(=O)N3CCC4CC43)c2C1]

Rt (Method A) 2.89 mins, m/z 376 [M+H]+

¹H NMR (400 MHz, DMSO-d6) δ 11.71 (s, 1H), 8.17-7.71 (m, 1H), 7.66 (d, J=8.1 Hz, 1H), 7.45 (d, J=8.2 Hz, 1H), 7.22 (t, J=7.7 Hz, 1H), 7.15-7.02 (m, 1H), 6.96 (s, 1H), 5.27 (s, 2H), 4.30 (d, J=30.9 Hz, 4H), 3.75 (d, J=117.4 Hz, 2H), 3.27 (m, 1H), 2.19-1.44 (m, 3H), 0.84 (d, J=141.0 Hz, 2H).

### Example 49

### 5-[5-(1H-indole-2-carbonyl)-4H,5H,6H,7H-pyrazolo[1,5-a]pyrazin-3-yl]-5-azaspiro[2.4]heptan-6-one

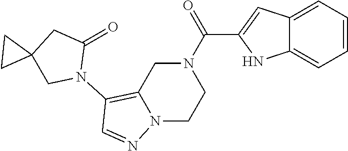 [SMILES: O=C(c1cc2ccccc2[nH]1)N1CCn2ncc(N3CC4(CC4)CC3=O)c2C1]

Step 1: To tert-butyl 3-(6-oxo-5-azaspiro[2.4]heptan-5-yl)-6,7-dihydropyrazolo[1,5-a]pyrazine-5(4H)-carboxylate (21 mg, 0.063 mmol) was added HCl (4M in dioxane) (395 μL, 1.579 mmol). The resulting solution was stirred at r.t. for 2 h. Further HCl was added (4M in dioxane) (95 μL, 0.379 mmol) and the mixture stirred for 45 minutes. The reaction mixture was diluted with dioxane (6 mL) and concentrated, then co-evaporated with toluene (2×6 mL) to give 5-{4H,5H,6H,7H-pyrazolo[1,5-a]pyrazin-3-yl}-5-azaspiro[2.4]heptan-6-one hydrochloride as an off-white solid that was used in the next step without further purification.

Step 2: To a solution of 1H-indole-2-carboxylic acid (5.16 mg, 0.032 mmol) in DMSO (213 μL) was added HATU (13.38 mg, 0.035 mmol). The resulting solution was stirred at r.t. for 40 mins. Then, a mixture of 5-(4,5,6,7-tetrahydropyrazolo[1,5-a]pyrazin3-yl) 5-azaspiro[2.4]heptan-6-one hydrochloride (8.60 mg, 0.032 mmol) and Et₃N (22.30 μL, 0.160 mmol) in DMSO (213 μL) was added and the reaction was stirred at rt. The reaction mixture was then filtered and purified directly by chromatography to give the product as a white solid (0.0029 g, 24% yield).

Rt (Method A) 2.91 mins, m/z 376 [M+H]+

¹H NMR (400 MHz, DMSO-d6) δ 11.68 (s, 1H), 7.64 (d, J=8.0 Hz, 1H), 7.58 (s, 1H), 7.44 (d, J=8.3 Hz, 1H), 7.27-7.15 (m, 1H), 7.11-7.02 (m, 1H), 6.96 (s, 1H), 5.14-4.80 (m, 2H), 4.37-4.10 (m, 4H), 3.65 (s, 2H), 2.47 (s, 2H), 0.72-0.64 (m, 4H).

### Example 50

### 5-(1H-indole-2-carbonyl)-N-[1-(methoxymethyl)cyclopropyl]-4H,5H,6H,7H-pyrazolo[1,5-a]pyrazine-3-carboxamide

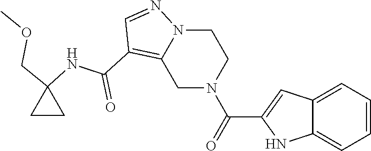 [SMILES: COCC1(NC(=O)c2cnn3c2CN(C(=O)c2cc4ccccc4[nH]2)CC3)CC1]

Rt (Method A) 2.82 mins, m/z 337 [M+H]+

¹H NMR (400 MHz, DMSO-d6) δ 11.71 (s, 1H), 8.37 (s, 1H), 8.03 (s, 1H), 7.67 (d, J=8.0 Hz, 1H), 7.45 (d, J=8.3 Hz, 1H), 7.22 (ddd, J=8.1, 6.8, 1.2 Hz, 1H), 7.08 (ddd, J=8.2, 6.9, 0.9 Hz, 1H), 6.96 (s, 1H), 5.48-4.95 (m, 2H), 4.41-4.09 (m, 4H), 3.41 (s, 2H), 3.24 (s, 3H), 0.82-0.61 (m, 4H).

### Example 51

### 1-[5-(1H-indole-2-carbonyl)-4H,5H,6H,7H-pyrazolo[1,5-a]pyrazin-3-yl]-3-phenylcyclobutan-1-ol

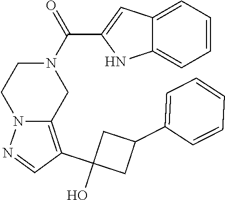 [SMILES: O=C(c1cc2ccccc2[nH]1)N1CCn2ncc(C3(O)CC(c4ccccc4)C3)c2C1]

Rt (Method A) 3.27 mins, m/z 413 [M+H]+

¹H NMR (400 MHz, DMSO-d6) δ 11.70 (s, 1H), 7.68-7.60 (m, 2H), 7.44 (d, J=8.3 Hz, 1H), 7.35-7.23 (m, 4H), 7.25-7.13 (m, 2H), 7.07 (t, J=7.4 Hz, 1H), 6.96 (s, 1H), 5.51 (s, 1H), 5.37-4.72 (m, 2H), 4.46-4.03 (m, 4H), 3.10-2.97 (m, 1H), 2.83-2.71 (m, 2H), 2.41-2.31 (m, 2H).

### Example 52

### 5-(4-chloro-1H-indole-2-carbonyl)-N-(2-hydroxyethyl)-N-methyl-4H,5H,6H,7H-pyrazolo[1,5-a]pyrazine-3-carboxamide

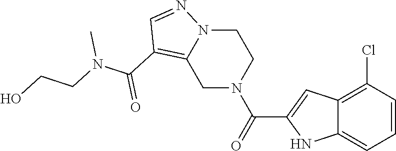 [SMILES: CN(CCO)C(=O)c1cnn2c1CN(C(=O)c1cc3c(Cl)cccc3[nH]1)CC2]

Rt (Method A) 2.8 mins, m/z 402/404 [M+H]+

### Example 53

### N-(1-{[(2,2-difluoroethyl)amino]methyl}cyclopropyl)-5-(1H-indole-2-carbonyl)-N-methyl-4H,5H,6H,7H-pyrazolo[1,5-a]pyrazine-3-carboxamide

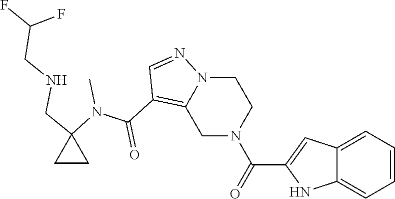 [SMILES: CN(C(=O)c1cnn2c1CN(C(=O)c1cc3ccccc3[nH]1)CC2)C1(CNCC(F)F)CC1]

Rt (Method A) 2.97 mins, m/z 457 [M+H]+

¹H NMR (400 MHz, DMSO-d6) δ 11.70 (s, 1H), 8.09-7.74 (m, 1H), 7.65 (d, J=8.0 Hz, 1H), 7.44 (d, J=8.3 Hz, 1H), 7.21 (ddd, J=8.2, 6.9, 1.2 Hz, 1H), 7.07 (ddd, J=8.0, 6.9, 1.0 Hz, 1H), 6.95 (s, 1H), 5.95 (t, J=56.3 Hz, 1H), 5.36-4.78 (m, 2H), 4.46-3.99 (m, 4H), 3.26-2.70 (m, 7H), 2.24 (s, 1H), 1.19-0.67 (m, 4H).

### Example 54

### N-[2-(difluoromethoxy)ethyl]-5-(1H-indole-2-carbonyl)-4H,5H,6H,7H-pyrazolo[1,5-a]pyrazine-3-carboxamide

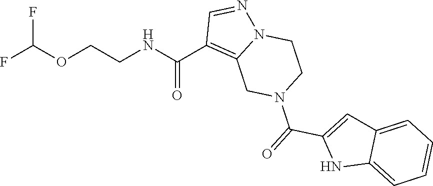 [SMILES: O=C(NCCOC(F)F)c1cnn2c1CN(C(=O)c1cc3ccccc3[nH]1)CC2]

Rt (Method A2) 3.18 mins, m/z 404 [M+H]+

¹H NMR (400 MHz, DMSO-d6) ?? 11.71 (s, 1H), 8.32 (t, J=5.7 Hz, 1H), 8.01 (s, 1H), 7.66 (d, J=7.9 Hz, 1H), 7.44 (d, J=8.1 Hz, 1H), 7.22 (t, J=7.5 Hz, 1H), 7.08 (t, J=7.4 Hz, 1H), 6.96 (s, 1H), 6.67 (t, J=76.0 Hz, 1H), 5.40-4.96 (m, 2H), 4.41-4.12 (m, 4H), 3.89 (t, J=5.8 Hz, 2H), 3.47-3.39 (m, 3H).

### Example 55

### 5-(1H-indole-2-carbonyl)-N-methyl-N-[(1-methyl-1H-pyrazol-5-yl)methyl]-4H,5H,6H,7H-pyrazolo[1,5-a]pyrazine-3-carboxamide

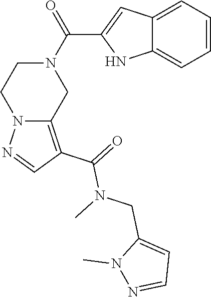 [SMILES: CN(Cc1ccnn1C)C(=O)c1cnn2c1CN(C(=O)c1cc3ccccc3[nH]1)CC2]

Rt (Method A) 2.7 mins, m/z 418 [M+H]+

¹H NMR (400 MHz, DMSO-d6) δ 11.72 (s, 1H), 7.84 (s, 1H), 7.66 (d, J=7.9 Hz, 1H), 7.44 (d, J=8.1 Hz, 1H), 7.32 (s, 1H), 7.27-7.16 (m, 1H), 7.14-7.04 (m, 1H), 6.96 (s, 1H), 6.23-6.11 (m, 1H), 5.33-4.94 (m, 2H), 4.78-4.63 (m, 2H), 4.38-4.15 (m, 4H), 3.73 (s, 3H), 3.08 (s, 3H).

### Example 56

### 2-(3-{7,7-difluoro-4-azaspiro[2.4]heptane-4-carbonyl}-4H,5H,6H,7H-pyrazolo[1,5-a]pyrazine-5-carbonyl)-1H-indole

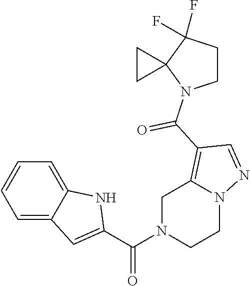 [SMILES: O=C(c1cc2ccccc2[nH]1)N1CCn2ncc(C(=O)N3CCC(F)(F)C34CC4)c2C1]

Rt (Method A) 3.2 mins, m/z 426 [M+H]+

¹H NMR (400 MHz, DMSO-d6) δ 11.71 (s, 1H), 7.94 (s, 1H), 7.65 (d, J=7.9 Hz, 1H), 7.44 (d, J=8.1 Hz, 1H), 7.26-7.17 (m, 1H), 7.13-7.03 (m, 1H), 6.96 (s, 1H), 5.21-4.92 (m, 2H), 4.38-4.15 (m, 4H), 4.01 (t, J=7.3 Hz, 2H), 2.07-1.91 (m, 2H), 0.94-0.78 (m, 2H).

### Example 57

### 5-(1H-indole-2-carbonyl)-N-methyl-N-[1-(1,3-oxazol-4-yl)cyclopropyl]-4H,5H,6H,7H-pyrazolo[1,5-a]pyrazine-3-carboxamide

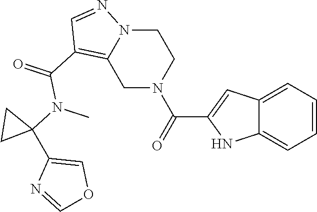 [SMILES: CN(C(=O)c1cnn2c1CN(C(=O)c1cc3ccccc3[nH]1)CC2)C1(c2cocn2)CC1]

To a solution of 5-(1H-indole-2-carbonyl)-4,5,6,7-tetrahydropyrazolo[1,5-a]pyrazine-3-carboxylic acid (0.050 g, 0.161 mmol) in dry DMF (0.5 mL) was added HATU (61.3 mg, 0.161 mmol). The resulting solution was stirred under an N₂ atmosphere for 30 mins, after which time a solution of N-methyl-1-(oxazol-4-yl)cyclopropan-1-amine hydrochloride (28.1 mg, 0.161 mmol) and Et₃N (0.074 mL, 0.532 mmol) in dry DMF (0.5 mL) was added. The mixture was stirred for 1 h, then filtered and purified directly by chromatography to give the product as a white solid (0.032 g, 46% yield).

Rt (Method A) 2.85 mins, m/z 431 [M+H]+

¹H NMR (400 MHz, DMSO-d6) δ 11.72 (s, 1H), 8.38 (s, 1H), 8.09 (s, 1H), 7.66 (d, J=8.1 Hz, 1H), 7.44 (d, J=8.2 Hz, 1H), 7.40-7.14 (m, 2H), 7.07 (t, J=7.5 Hz, 1H), 6.95 (s, 1H), 5.45-4.90 (m, 2H), 4.43-3.97 (m, 4H), 3.03 (s, 3H), 1.65-1.17 (m, 4H).

### Example 58

### 4-[5-(1H-indole-2-carbonyl)-4H,5H,6H,7H-pyrazolo[1,5-a]pyrazine-3-carbonyl]-4-azaspiro[2.5]octan-7-ol

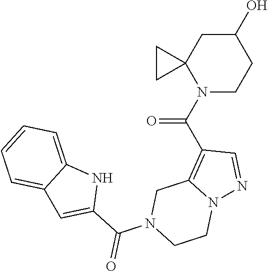 [SMILES: O=C(c1cc2ccccc2[nH]1)N1CCn2ncc(C(=O)N3CCC(O)CC34CC4)c2C1]

Rt (Method A) 2.64 mins, m/z 420 [M+H]+

¹H NMR (400 MHz, DMSO-d6) δ 11.71 (s, 1H), 7.75 (s, 1H), 7.65 (d, J=7.9 Hz, 1H), 7.44 (d, J=8.2 Hz, 1H), 7.22 (t, J=7.8 Hz, 1H), 7.07 (t, J=7.5 Hz, 1H), 6.94 (s, 1H), 5.44-4.88 (m, 2H), 4.84-4.64 (m, 1H), 4.46-4.04 (m, 5H), 3.91-3.73 (m, 1H), 3.18-2.76 (m, 1H), 1.94-1.65 (m, 2H), 1.58-1.07 (m, 2H), 1.00-0.74 (m, 2H), 0.67-0.45 (m, 2H).

### Example 59

### 2-{3-[7-(difluoromethoxy)-4-azaspiro[2.5]octane-4-carbonyl]-4H,5H,6H,7H-pyrazolo[1,5-a]pyrazine-5-carbonyl}-1H-indole

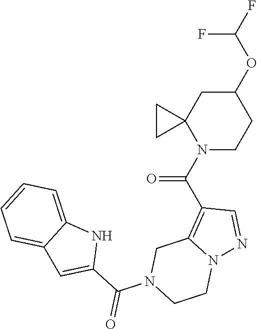 [SMILES: O=C(c1cc2ccccc2[nH]1)N1CCn2ncc(C(=O)N3CCC(OC(F)F)CC34CC4)c2C1]

To a solution of 5-(1H-indole-2-carbonyl)-4,5,6,7-tetrahydropyrazolo[1,5-a]pyrazine-3-carboxylic acid (0.050 g, 0.161 mmol) in dry DMF (0.5 mL) was added HATU (61.3 mg, 0.161 mmol). The resulting solution was stirred under N₂ atmosphere for 30 mins, after which time a solution of 7-(difluoromethoxy)-4-azaspiro[2.5]octane (28.6 mg, 0.161 mmol) and Et₃N (0.074 mL, 0.532 mmol) in dry DMF (0.5 mL) was added. The mixture was stirred for 1 h, then filtered and purified directly by chromatography to give the product as a white solid (0.037 g, 26% yield).

Rt (Method A) 3.16 mins, m/z 470 [M+H]+

¹H NMR (400 MHz, DMSO-d6) δ 11.71 (s, 1H), 7.77 (s, 1H), 7.65 (d, J=8.0 Hz, 1H), 7.44 (d, J=8.2 Hz, 1H), 7.29-7.18 (m, 1H), 7.07 (t, J=7.4 Hz, 1H), 6.99-6.53 (m, 2H), 5.12 (s, 2H), 4.54-4.02 (m, 6H), 2.06-1.33 (m, 4H), 1.03-0.51 (m, 4H).

### Example 60

### N,N-dicyclopropyl-5-(1H-indole-2-carbonyl)-4H,5H,6H,7H-pyrazolo[1,5-a]pyrazine-3-carboxamide

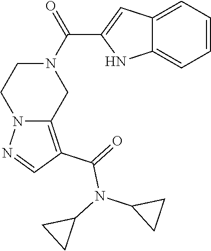 [SMILES: O=C(c1cc2ccccc2[nH]1)N1CCn2ncc(C(=O)N(C3CC3)C3CC3)c2C1]

Rt (Method A) 2.95 mins, m/z 390 [M+H]+

¹H NMR (400 MHz, DMSO-d6) δ 11.69 (s, 1H), 7.95 (s, 1H), 7.66 (d, J=8.0 Hz, 1H), 7.44 (d, J=8.3 Hz, 1H), 7.28-7.15 (m, 1H), 7.12-7.01 (m, 1H), 6.95 (s, 1H), 5.39-4.97 (m, 2H), 4.38-4.10 (m, 4H), 2.86-2.69 (m, 2H), 0.83-0.56 (m, 8H).

### Example 61

### 5-(1H-indole-2-carbonyl)-N-[1-(pyridin-2-yl)cyclopropyl]-4H,5H,6H,7H-pyrazolo[1,5-a]pyrazine-3-carboxamide

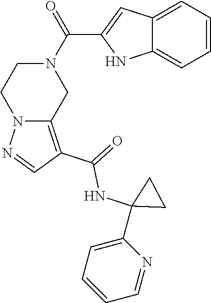 [SMILES: O=C(NC1(c2ccccn2)CC1)c1cnn2c1CN(C(=O)c1cc3ccccc3[nH]1)CC2]

Rt (Method A) 2.82 mins, m/z 427 [M+H]+

¹H NMR (400 MHz, DMSO-d6) δ 11.69 (s, 1H), 8.90 (s, 1H), 8.47-8.36 (m, 1H), 8.14 (s, 1H), 7.72-7.56 (m, 2H), 7.43 (d, 1H), 7.31 (d, J=8.0 Hz, 1H), 7.26-7.01 (m, 3H), 6.94 (s, 1H), 5.39-4.99 (m, 2H), 4.42-4.11 (m, 4H), 1.58-1.41 (m, 2H), 1.28-1.11 (m, 2H).

### Example 62

### N-[2-(difluoromethoxy)ethyl]-5-(1H-indole-2-carbonyl)-N-methyl-4H,5H,6H,7H-pyrazolo[1,5-a]pyrazine-3-carboxamide

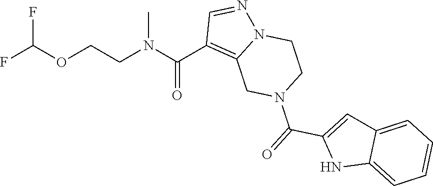 [SMILES: CN(CCOC(F)F)C(=O)c1cnn2c1CN(C(=O)c1cc3ccccc3[nH]1)CC2]

Rt (Method A) 2.91 mins, m/z 418 [M+H]+

¹H NMR (400 MHz, DMSO-d6) δ 11.71 (s, 1H), 7.91-7.74 (m, 1H), 7.65 (d, J=7.9 Hz, 1H), 7.44 (d, J=8.2 Hz, 1H), 7.22 (t, J=7.5 Hz, 1H), 7.07 (t, J=7.5 Hz, 1H), 6.95 (s, 1H), 6.68 (t, J=75.7 Hz, 1H), 5.27-4.94 (m, 2H), 4.38-4.17 (m, 4H), 4.06-3.94 (m, 2H), 3.75-3.60 (m, 2H), 3.29-2.80 (m, 3H).

### Example 63

### N-benzyl-5-(1H-indole-2-carbonyl)-N-methyl-4H,5H,6H,7H-pyrazolo[1,5-a]pyrazine-3-carboxamide

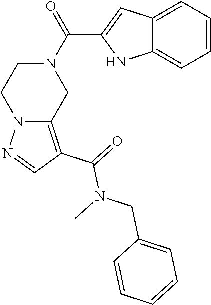 [SMILES: CN(Cc1ccccc1)C(=O)c1cnn2c1CN(C(=O)c1cc3ccccc3[nH]1)CC2]

Rt (Method A) 3.14 mins, m/z 414 [M+H]+

¹H NMR (400 MHz, DMSO-d6) δ 11.72 (s, 1H), 8.03-7.75 (m, 1H), 7.66 (d, J=8.1 Hz, 1H), 7.45 (d, J=8.2 Hz, 1H), 7.39-7.17 (m, 6H), 7.08 (t, J=7.5 Hz, 1H), 6.97 (s, 1H), 5.42-4.90 (m, 2H), 4.80-4.55 (m, 2H), 4.37-4.19 (m, 4H), 3.26-2.73 (m, 3H).

### Example 64

### N-{1-[(2-hydroxyethoxy)methyl]cyclopropyl}-5-(1H-indole-2-carbonyl)-N-methyl-4H,5H,6H,7H-pyrazolo[1,5-a]pyrazine-3-carboxamide

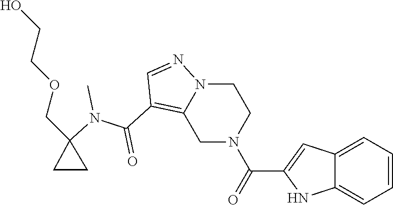 [SMILES: CN(C(=O)c1cnn2c1CN(C(=O)c1cc3ccccc3[nH]1)CC2)C1(COCCO)CC1]

Step 1: To a solution of tert-butyl 3-((1-((2-hydroxyethoxy)methyl)cyclopropyl)(methyl)carbamoyl)-6,7-dihydropyrazolo[1,5-a]pyrazine-5(4H)-carboxylate (100 mg, 0.254 mmol) in DCM (0.5 mL) was added HCl (4M in dioxane) (2 mL, 8.00 mmol). The reaction mixture was stirred for 90 mins, then concentrated and stripped with DCM to yield N-{1-[(2-hydroxyethoxy)methyl]cyclopropyl}-N-methyl-4H,5H,6H,7H-pyrazolo[1,5-a]pyrazine-3-carboxamide as a pink solid that was used in the next step without further purification.

Step 2: Indole-2-carboxylic acid (20.46 mg, 0.127 mmol) was dissolved in dry DMSO (0.4 mL) and HATU (57.9 mg, 0.152 mmol) was added. The mixture was stirred for 10 mins. In a separate vial, N-(1-((2-hydroxyethoxy)methyl)cyclopropyl)-N-methyl-4,5,6,7-tetrahydropyrazolo[1,5-a]pyrazine-3-carboxamide hydrochloride (42 mg, 0.127 mmol) was dissolved in dry DMSO (0.4 mL) and triethylamine (0.088 mL, 0.635 mmol) was added. A few drops of water were added to give an almost clear solution. The mixtures were combined and stirred for 1 h, then filtered, rinsing with methanol (0.1 mL). The filtrate was purified directly by chromatography to give the product as a white powder (0.0387 g, 70% yield).

Rt (Method B) 2.68 mins, m/z 438 [M+H]+

¹H NMR (400 MHz, DMSO-d6) δ 11.71 (s, 1H), 8.10-7.78 (m, 1H), 7.66 (d, J=8.0 Hz, 1H), 7.44 (d, J=8.2 Hz, 1H), 7.25-7.18 (m, 1H), 7.11-7.04 (m, 1H), 6.95 (s, 1H), 5.43-4.84 (m, 2H), 4.59 (s, 1H), 4.38-4.01 (m, 4H), 3.69-3.55 (m, 2H), 3.55-3.40 (m, 4H), 3.25-2.83 (m, 3H), 1.30-0.59 (m, 4H).

### Example 65

### N-{1-[(3-hydroxypropoxy)methyl]cyclopropyl}-5-(1H-indole-2-carbonyl)-N-methyl-4H,5H,6H,7H-pyrazolo[1,5-a]pyrazine-3-carboxamide

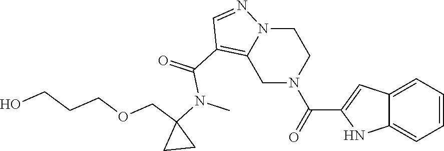 [SMILES: CN(C(=O)c1cnn2c1CN(C(=O)c1cc3ccccc3[nH]1)CC2)C1(COCCCO)CC1]

Rt (Method B) 2.76 mins, m/z 452 [M+H]+

### Example 66

### 2-[3-(1,3-thiazol-4-yl)-4H,5H,6H,7H-pyrazolo[1,5-a]pyrazine-5-carbonyl]-1H-indole

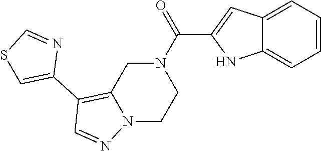 [SMILES: O=C(c1cc2ccccc2[nH]1)N1CCn2ncc(-c3cscn3)c2C1]

To a solution of 4-{4H,5H,6H,7H-pyrazolo[1,5-a]pyrazin-3-yl}-1,3-thiazole hydrochloride (0.0291 g, 0.12 mmol) in DMSO (0.4 mL) was added a drop of water. NEt₃ (0.075 mL, 0.538 mmol) was then added. In a separate vial, indole-2-carboxylic acid (0.212 g) and HATU (0.600 g) were dissolved in DMSO (3.8 mL). After 10 minutes, 0.4 mL of this solution was added to the amine solution, and this mixture stirred for 48 h. The mixture was then filtered, and the filter rinsed with methanol (0.1 mL). The filtrate was purified directly by chromatography to give the product as a white solid (0.0275 g, 66% yield).

Rt (Method B) 3.02 mins, m/z 350 [M+H]+

### Example 67

### 4-[5-(1H-indole-2-carbonyl)-4H,5H,6H,7H-pyrazolo[1,5-a]pyrazine-3-carbonyl]-8-oxa-4-azaspiro[2.6]nonane

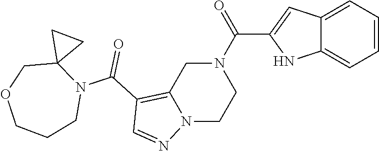 [SMILES: O=C(c1cc2ccccc2[nH]1)N1CCn2ncc(C(=O)N3CCCOCC34CC4)c2C1]

To a solution of indole-2-carboxylic acid (0.019 mg, 0.12 mmol) in DMSO (0.4 mL) was added HATU (0.054 g, 0.144 mmol). A solution of triethylamine (0.075 mL, 0.538 mmol) and 8-oxa-4-azaspiro[2.6]nonane (0.0375 g, 0.12 mmol) in DMSO (0.4 mL) was added, and the mixture was stirred for 48 h. The mixture was then filtered, rinsed with methanol (0.1 mL) and purified directly by chromatography to give the product as a white powder (0.035 g, 65% yield).

Rt (Method B) 2.81 mins, m/z 420 [M+H]+

### Example 68

### 4-[5-(1H-indole-2-carbonyl)-4H,5H,6H,7H-pyrazolo[1,5-a]pyrazine-3-carbonyl]-7-oxa-4-azaspiro[2.6]nonane

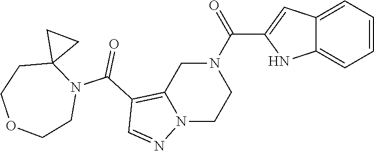 [SMILES: O=C(c1cc2ccccc2[nH]1)N1CCn2ncc(C(=O)N3CCOCCC34CC4)c2C1]

To a solution of indole-2-carboxylic acid (0.019 mg, 0.12 mmol) in DMSO (0.4 mL) was added HATU (0.054 g, 0.144 mmol). A solution of triethylamine (0.075 mL, 0.538 mmol) and 7-oxa-4-azaspiro[2.6]nonane (0.0370 g, 0.12 mmol) in DMSO (0.4 mL) was added, and the mixture was stirred for 48 h. The mixture was then filtered, rinsed with methanol (0.1 mL) and purified directly by chromatography to give the product as a white powder (0.026 g, 53% yield).

Rt (Method B) 2.81 mins, m/z 420 [M+H]+

### Example 69

### 2-[3-(2,2-difluoromorpholine-4-carbonyl)-4H,5H,6H,7H-pyrazolo[1,5-a]pyrazine-5-carbonyl]-1H-indole

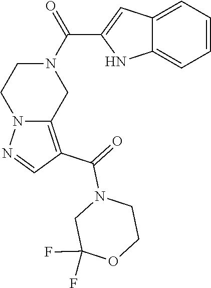 [SMILES: O=C(c1cc2ccccc2[nH]1)N1CCn2ncc(C(=O)N3CCOC(F)(F)C3)c2C1]

Rt (Method B) 3.01 mins, m/z 416 [M+H]+

### Example 70

### 5-(1H-indole-2-carbonyl)-N-[1-(methoxymethyl)cyclopropyl]-N,6-dimethyl-4H,5H,6H,7H-pyrazolo[1,5-a]pyrazine-3-carboxamide (Enantiomer 1, absolute configuration unknown)

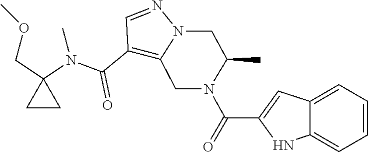 [SMILES: COCC1(N(C)C(=O)c2cnn3c2CN(C(=O)c2cc4ccccc4[nH]2)[C@H](C)C3)CC1]

Rt (Method A) 3.03 mins, m/z 422 [M+H]+

¹H NMR (400 MHz, DMSO-d6) δ 11.67 (s, 1H), 8.04-7.88 (m, 1H), 7.68-7.62 (m, 1H), 7.49-7.41 (m, 1H), 7.26-7.18 (m, 1H), 7.11-7.04 (m, 1H), 6.93 (s, 1H), 5.83-5.38 (m, 1H), 5.38-5.19 (m, 1H), 5.02-4.55 (m, 1H), 4.50-4.26 (m, 1H), 4.17 (d, J=13.5 Hz, 1H), 3.64-3.46 (m, 2H), 3.28 (s, 3H), 3.15-2.90 (m, 3H), 1.34-0.64 (m, 7H).

Stereochemically pure material was obtained by separation of the racemate (Example 1) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 71

### N-cyclopropyl-5-(1H-indole-2-carbonyl)-N,6-dimethyl-4H,5H,6H,7H-pyrazolo[1,5-a]pyrazine-3-carboxamide (Enantiomer 2, Absolute Configuration Unknown)

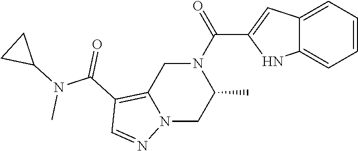 [SMILES: C[C@@H]1Cn2ncc(C(=O)N(C)C3CC3)c2CN1C(=O)c1cc2ccccc2[nH]1]

Step 1: 5-(tert-butoxycarbonyl)-6-methyl-4,5,6,7-tetrahydropyrazolo[1,5-a]pyrazine-3-carboxylic acid (100 mg, 0.355 mmol) was dissolved in dry DMSO (3 mL) and HATU (149 mg, 0.391 mmol) was added. The mixture was stirred for 10 min. Triethylamine (0.248 ml, 1.777 mmol) was added followed by a solution of N-methylcyclopropanamine hydrochloride (38.2 mg, 0.355 mmol) in dry DMSO (1 mL) and the reaction mixture was stirred for 1 h. The reaction was quenched with a few drops of water and purified using by reversed phase column chromatography to give tert-butyl 3-[cyclopropyl(methyl)carbamoyl]-6-methyl-4H,5H,6H,7H-pyrazolo[1,5-a]pyrazine-5-carboxylate as a colorless oil (0.109 g, 83% yield.

Step 2: Tert-butyl 3-(cyclopropyl(methyl)carbamoyl)-6-methyl-6,7-dihydropyrazolo[1,5-a]pyrazine5(4H)-carboxylate (109 mg, 0.293 mmol) was dissolved in HCl (4 M in dioxane) (1 mL, 4.00 mmol). The mixture was stirred for overnight, then concentrated and stripped with DCM to give N-cyclopropyl-N,6-dimethyl-4H,5H,6H,7H-pyrazolo[1,5-a]pyrazine-3-carboxamide hydrochloride as a white solid that was used in the next step without further purification (0.076 g, 90% yield).

Step 3: Indole-2-carboxylic acid (13.99 mg, 0.087 mmol) was dissolved in dry DMSO (0.4 mL) and HATU (36.3 mg, 0.095 mmol) was added. In a separate vial, N-cyclopropylN,6-dimethyl-4,5,6,7-tetrahydropyrazolo[1,5-a]pyrazine-3-carboxamide hydrochloride (25 mg, 0.087 mmol) was suspended in dry DMSO (0.4 mL) and triethylamine (0.060 ml, 0.434 mmol) was added. The two mixtures were combined and stirred for 1 h. A few drops of water were added and the reaction mixture was purified directly by reverse phase column chromatography to give the product as a white solid (0.0183 g, 56% yield).

Rt (Method A) 2.97 mins, m/z 378 [M+H]+

¹H NMR (400 MHz, DMSO-d6) δ 11.67 (s, 1H), 8.03 (s, 1H), 7.66 (d, J=7.9 Hz, 1H), 7.45 (d, J=8.2 Hz, 1H), 7.25-7.18 (m, 1H), 7.11-7.04 (m, 1H), 6.93 (s, 1H), 5.56 (d, J=18.6 Hz, 1H), 5.36-5.21 (m, 1H), 4.87-4.64 (m, 1H), 4.47-4.31 (m, 1H), 4.19 (d, J=13.1 Hz, 1H), 3.16-3.02 (m, 1H), 2.94 (s, 3H), 1.24 (d, J=6.9 Hz, 3H), 0.90-0.74 (m, 2H), 0.67-0.51 (m, 2H).

Stereochemically pure material was obtained by separation of the racemate (Example 2) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 72

### 5-(1H-indole-2-carbonyl)-N-methyl-N-[1-(pyridin-3-yl)cyclopropyl]-4H,5H,6H,7H-pyrazolo[1,5-a]pyrazine-3-carboxamide

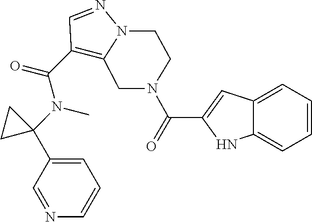 [SMILES: CN(C(=O)c1cnn2c1CN(C(=O)c1cc3ccccc3[nH]1)CC2)C1(c2cccnc2)CC1]

Rt (Method B) 2.36 mins, m/z 441 [M+H]+

### Example 73

### 2-[3-(oxolan-2-yl)-4H,5H,6H,7H-pyrazolo[1,5-a]pyrazine-5-carbonyl]-1H-indole

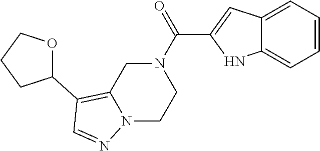 [SMILES: O=C(c1cc2ccccc2[nH]1)N1CCn2ncc(C3CCCO3)c2C1]

Step 1: To a solution of tert-butyl 3-(tetrahydrofuran-2-yl)-6,7-dihydropyrazolo[1,5-a]pyrazine-5(4H)-carboxylate (35 mg, 0.119 mmol) in DCM (0.2 mL) was added HCl (4 M in dioxane) (1 mL, 4.00 mmol). After 1 h, the reaction mixture was concentrated and stripped with DCM to give 3-(oxolan-2-yl)-4H,5H,6H,7H-pyrazolo[1,5-a]pyrazine hydrochloride as an off-white solid that was used in the next step without further purification

Step 2: To a solution of 3-(oxolan-2-yl)-4H,5H,6H,7H-pyrazolo[1,5-a]pyrazine hydrochloride (0.0276 g, 0.12 mmol) in DMSO (0.4 mL) was added a drop of water. NEt₃ (0.075 mL, 0.538 mmol) was then added. In a separate vial, indole-2-carboxylic acid (0.212 g) and HATU (0.600 g) were dissolved in DMSO (3.8 mL). After 10 minutes, 0.4 mL of this solution was added to the amine solution, and this mixture stirred for 48 h. The mixture was then filtered, and the filter rinsed with methanol (0.1 mL). The filtrate was purified directly by chromatography to give the product as a white solid (0.0042 g, 10% yield).

Rt (Method B) 2.93 mins, m/z 337 [M+H]+

### Example 74

### N-cyclopropyl-5-(1H-indole-2-carbonyl)-N-methyl-4H,5H,6H,7H-pyrazolo[1,5-a]pyrazine-3-carboxamide

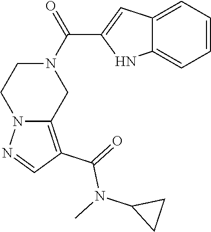 [SMILES: CN(C(=O)c1cnn2c1CN(C(=O)c1cc3ccccc3[nH]1)CC2)C1CC1]

Rt (Method B) 2.86 mins, m/z 346 [M+H]+

¹H NMR (400 MHz, DMSO-d6) δ 11.70 (s, 1H), 8.01 (s, 1H), 7.65 (d, J=8.0 Hz, 1H), 7.44 (d, J=8.3 Hz, 1H), 7.25-7.17 (m, 1H), 7.11-7.03 (m, 1H), 6.94 (s, 1H), 5.44-4.88 (m, 2H), 4.37-4.17 (m, 4H), 3.13-3.03 (m, 1H), 2.93 (s, 3H), 0.84-0.75 (m, 2H), 0.63-0.54 (m, 2H).

### Example 75

### N-(2-hydroxyethyl)-N-[1-(hydroxymethyl)cyclopropyl]-5-(1H-indole-2-carbonyl)-4H,5H,6H,7H-pyrazolo[1,5-a]pyrazine-3-carboxamide

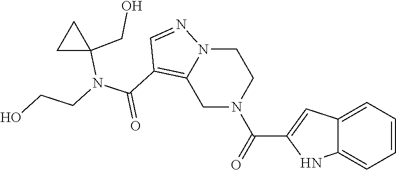 [SMILES: O=C(c1cc2ccccc2[nH]1)N1CCn2ncc(C(=O)N(CCO)C3(CO)CC3)c2C1]

Rt (Method B) 2.59 mins, m/z 424 [M+H]+

¹H NMR (400 MHz, DMSO-d6) δ 11.70 (s, 1H), 7.95 (s, 1H), 7.65 (d, J=8.1 Hz, 1H), 7.44 (d, J=8.2 Hz, 1H), 7.25-7.18 (m, 1H), 7.10-7.04 (m, 1H), 6.94 (s, 1H), 5.36-4.98 (m, 3H), 4.91-4.74 (m, 1H), 4.51-3.99 (m, 4H), 3.82-3.38 (m, 6H), 1.35-0.59 (m, 4H).

### Example 76

### 5-[5-(1H-indole-2-carbonyl)-4H,5H,6H,7H-pyrazolo[1,5-a]pyrazin-3-yl]-5-azaspiro[2.4]heptan-4-one

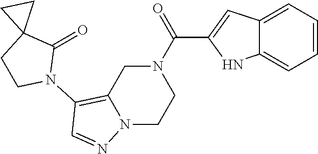 [SMILES: O=C(c1cc2ccccc2[nH]1)N1CCn2ncc(N3CCC4(CC4)C3=O)c2C1]

Step 1: To a solution of tert-butyl 3-(4-oxo-5-azaspiro[2.4]heptan-5-yl)-6,7-dihydropyrazolo[1,5-a]pyrazine-5(4H)-carboxylate (80 mg, 0.241 mmol) in DCM (0.4 mL) was added HCl (4M in dioxane) (2 ml, 8 mmol). The mixture was stirred for 1 hour, then concentrated and stripped with DCM to give 5-{4H,5H,6H,7H-pyrazolo[1,5-a]pyrazin-3-yl}-5-azaspiro[2.4]heptan-4-one hydrochloride as an off-white solid that was used in the next step without further purification.

Step 2: To a solution of indole-2-carboxylic acid (19.19 mg, 0.119 mmol) in DMSO (400 μL) was added HATU (54.3 mg, 0.143 mmol). The mixture was stirred for 10 min. In a separate vial, 5-(4,5,6,7-tetrahydropyrazolo[1,5-a]pyrazin-3-yl)-5-azaspiro[2.4]heptan-4-one hydrochloride (32 mg, 0.119 mmol) was dissolved in DMSO (400 μL). A drop of water and triethylamine (83 μL, 0.595 mmol) were added. The mixtures were combined and stirred for 1 hour, then filtered and purified by chromatography to give the product as a white solid (0.288 g, 65% yield).

Rt (Method B) 2.91 mins, m/z 374 [M+H]+

¹H NMR (400 MHz, DMSO-d6) δ 11.67 (s, 1H), 7.67-7.56 (m, 2H), 7.44 (d, J=8.3 Hz, 1H), 7.25-7.18 (m, 1H), 7.10-7.03 (m, 1H), 6.95 (s, 1H), 5.11-4.81 (m, 2H), 4.36-4.15 (m, 4H), 3.80 (t, J=7.3 Hz, 2H), 2.21 (t, J=7.3 Hz, 2H), 0.96-0.81 (m, 4H).

### Example 77

### N-(2-hydroxyethyl)-5-(1H-indole-2-carbonyl)-N-methyl-4H,5H,6H,7H-pyrazolo[1,5-a]pyrazine-3-carboxamide

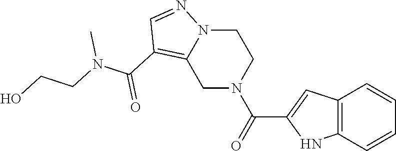 [SMILES: CN(CCO)C(=O)c1cnn2c1CN(C(=O)c1cc3ccccc3[nH]1)CC2]

Rt (Method A) 2.6 mins, m/z 368 [M+H]+

### Example 78

### 5-(1H-indole-2-carbonyl)-N-[(2S)-1,1,1-trifluoropropan-2-yl]-4H,5H,6H,7H-pyrazolo[1,5-a]pyrazine-3-carboxamide

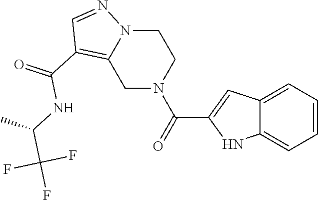 [SMILES: C[C@H](NC(=O)c1cnn2c1CN(C(=O)c1cc3ccccc3[nH]1)CC2)C(F)(F)F]

Rt (Method A) 3.07 mins, m/z 406 [M+H]+

¹H NMR (400 MHz, DMSO-d6) δ 11.71 (s, 1H), 8.47 (d, J=8.8 Hz, 1H), 8.14 (s, 1H), 7.66 (d, J=8.0 Hz, 1H), 7.44 (d, J=8.2 Hz, 1H), 7.22 (t, J=7.5 Hz, 1H), 7.08 (t, J=7.4 Hz, 1H), 6.96 (s, 1H), 5.50-4.91 (m, 2H), 4.89-4.69 (m, 1H), 4.46-4.04 (m, 4H), 1.32 (d, J=7.1 Hz, 3H).

### Example 79

### 5-(1H-indole-2-carbonyl)-N-[(2R)-1,1,1-trifluoropropan-2-yl]-4H,5H,6H,7H-pyrazolo[1,5-a]pyrazine-3-carboxamide

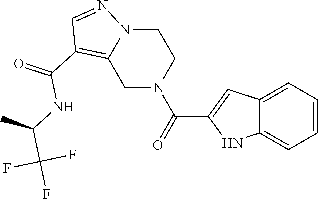 [SMILES: C[C@@H](NC(=O)c1cnn2c1CN(C(=O)c1cc3ccccc3[nH]1)CC2)C(F)(F)F]

To a solution of 5-(1H-indole-2-carbonyl)-4,5,6,7-tetrahydropyrazolo[1,5-a]pyrazine-3-carboxylic acid (50 mg, 0.161 mmol) in dry DMF (0.6 mL) was added HATU (61.3 mg, 0.161 mmol). The mixture was stirred at r.t. for 40 mins, then a solution of (R)-1,1,1-trifluoropropan-2-amine hydrochloride (24.10 mg, 0.161 mmol) in dry DMF (0.6 mL) was added, followed by triethylamine (0.074 mL, 0.532 mmol). The resulting cocktail was stirred at r.t. for 2 h, then filtered and purified directly by chromatography to give the product as a white powder (0.030 g, 46% yield).

Rt (Method A) 3.07 mins, m/z 406 [M+H]+

¹H NMR (400 MHz, DMSO-d6) δ 11.71 (s, 1H), 8.47 (d, J=8.8 Hz, 1H), 8.14 (s, 1H), 7.66 (d, J=8.0 Hz, 1H), 7.44 (d, J=8.2 Hz, 1H), 7.22 (t, J=7.6 Hz, 1H), 7.08 (t, J=7.5 Hz, 1H), 6.96 (s, 1H), 5.44-4.99 (m, 2H), 4.89-4.68 (m, 1H), 4.44-4.04 (m, 4H), 1.32 (d, J=7.1 Hz, 3H).

### Example 80

### 5-(1H-indole-2-carbonyl)-N-methyl-N-(1-{[(2,2,2-trifluoroethyl)amino]methyl}cyclopropyl)-4H,5H,6H,7H-pyrazolo[1,5-a]pyrazine-3-carboxamide

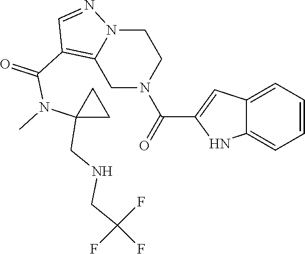 [SMILES: CN(C(=O)c1cnn2c1CN(C(=O)c1cc3ccccc3[nH]1)CC2)C1(CNCC(F)(F)F)CC1]

Rt (Method A) 3.1 mins, m/z 475 [M+H]+

¹H NMR (400 MHz, DMSO-d6) δ 11.71 (s, 1H), 8.02-7.72 (m, 1H), 7.65 (d, J=7.9 Hz, 1H), 7.50-7.36 (m, 1H), 7.32-7.13 (m, 1H), 7.13-7.01 (m, 1H), 6.95 (s, 1H), 5.45-4.80 (m, 2H), 4.45-4.01 (m, 4H), 3.30-2.55 (m, 8H), 1.32-0.60 (m, 4H).

### Example 81

### 5-(4,5-difluoro-1H-indole-2-carbonyl)-N-[1-(hydroxymethyl)cyclopropyl]-N-methyl-4H,5H,6H,7H-pyrazolo[1,5-a]pyrazine-3-carboxamide

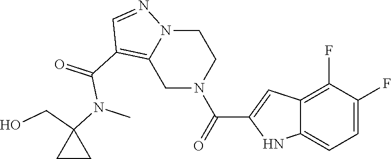 [SMILES: CN(C(=O)c1cnn2c1CN(C(=O)c1cc3c(F)c(F)ccc3[nH]1)CC2)C1(CO)CC1]

Step 1: To tert-butyl 3-((1-(hydroxymethyl)cyclopropyl)(methyl)carbamoyl)-6,7-dihydropyrazolo[1,5-a]pyrazine-5(4H)-carboxylate (0.300 g, 0.856 mmol) was added 4M HCl in dioxane (10 ml, 40.0 mmol). Methanol (2 mL) was then added and the reaction mixture was stirred for 2 h. The mixture was concentrated and co-evaporated with MCCN (50 mL) and DIPE (2×50 mL) at 40° C. under reduced pressure to obtain a off-white semi solid/oily residue that was used in the next step without further purification.

Step 2: To a stirred solution of 4-5-difluoroindole-2-carboxylic acid (0.0306 mg, 0.155 mmol) in DMF (0.4 mL) was added HATU (0.062 g, 0.162 mmol). The mixture was stirred for 30 mins, then a mixture of N-[1-(hydroxymethyl)cyclopropyl]-4H,5H,6H,7H-pyrazolo[1,5-a]pyrazine-3-carboxamide hydrochloride (0.050 g, 0.155 mmol)) and NEt3 (0.108 mL, 0.773 mmol) in DMF (0.4 mL) was added and the mixture stirred overnight. The mixture was filtered, and the filter rinsed with MeCN (0.2 mL). The mixture was purified by chromatography to give 5-(4,5-difluoro-1H-indole-2-carbonyl)-N-[1-(hydroxymethyl)cyclopropyl]-N-methyl-4H,5H,6H,7H-pyrazolo[1,5-a]pyrazine-3-carboxamide as a white solid (0.013 g, 20% yield).

Rt (Method A2) 3.08 mins, m/z 430 [M+H]+

¹H NMR (400 MHz, DMSO-d6) δ 12.13 (s, 1H), 8.23-7.58 (m, 1H), 7.36-7.16 (m, 2H), 7.06 (s, 1H), 5.82-4.60 (m, 3H), 4.54-3.99 (m, 4H), 3.85-3.48 (m, 2H), 3.20-2.76 (m, 3H), 1.20-0.43 (m, 4H).

### Example 82

### 5-(4,7-difluoro-1H-indole-2-carbonyl)-N-[1-(hydroxymethyl)cyclopropyl]-N-methyl-4H,5H,6H,7H-pyrazolo[1,5-a]pyrazine-3-carboxamide

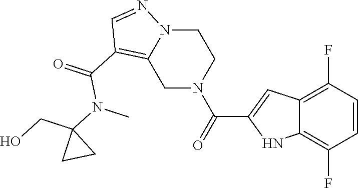 [SMILES: CN(C(=O)c1cnn2c1CN(C(=O)c1cc3c(F)ccc(F)c3[nH]1)CC2)C1(CO)CC1]

Rt (Method A2) 3.03 mins, m/z 430 [M+H]+

¹H NMR (400 MHz, DMSO-d6) δ 12.53 (s, 1H), 8.21-7.64 (m, 1H), 7.15-6.93 (m, 2H), 6.82 (t, J=8.4 Hz, 1H), 5.54-4.73 (m, 3H), 4.51-3.97 (m, 4H), 3.82-3.46 (m, 2H), 3.24-2.77 (m, 3H), 1.21-0.50 (m, 4H).

### Example 83

### 5-(6-fluoro-4-methyl-1H-indole-2-carbonyl)-N-[1-(hydroxymethyl)cyclopropyl]-N-methyl-4H,5H,6H,7H-pyrazolo[1,5-a]pyrazine-3-carboxamide

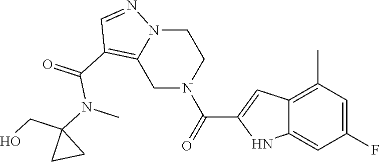 [SMILES: Cc1cc(F)cc2[nH]c(C(=O)N3CCn4ncc(C(=O)N(C)C5(CO)CC5)c4C3)cc12]

Rt (Method A2) 3.13 mins, m/z 426 [M+H]+

¹H NMR (400 MHz, DMSO-d6) δ 11.75 (s, 1H), 8.23-7.62 (m, 1H), 7.07-6.90 (m, 2H), 6.82-6.73 (m, 1H), 5.67-4.57 (m, 3H), 4.56-4.01 (m, 4H), 3.85-3.47 (m, 2H), 3.22-2.76 (m, 3H), 1.26-0.60 (m, 4H)—one signal (3H) coincides with DMSO signal.

### Example 84

### 5-(6-chloro-5-fluoro-1H-indole-2-carbonyl)-N-[1-(hydroxymethyl)cyclopropyl]-N-methyl-4H,5H,6H,7H-pyrazolo[1,5-a]pyrazine-3-carboxamide

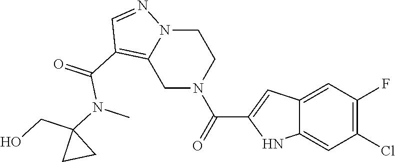 [SMILES: CN(C(=O)c1cnn2c1CN(C(=O)c1cc3cc(F)c(Cl)cc3[nH]1)CC2)C1(CO)CC1]

Step 1: To tert-butyl 3-((1-(hydroxymethyl)cyclopropyl)(methyl)carbamoyl)-6,7-dihydropyrazolo[1,5-a]pyrazine-5(4H)-carboxylate (0.300 g, 0.856 mmol) was added 4M HCl in dioxane (10 ml, 40.0 mmol). Methanol (2 mL) was then added and the reaction mixture was stirred for 2 h. The mixture was concentrated and co-evaporated with MCCN (50 mL) and DIPE (2×50 mL) at 40° C. under reduced pressure to obtain a off-white semi solid/oily residue that was used in the next step without further purification.

Step 2: To a stirred solution of 5-fluoro-6-chloro-indole-2-carboxylic acid (0.0331 mg, 0.155 mmol) in DMF (0.4 mL) was added HATU (0.062 g, 0.162 mmol). The mixture was stirred for 30 mins, then a mixture of N-[1-(hydroxymethyl)cyclopropyl]-4H,5H,6H,7H-pyrazolo[1,5-a]pyrazine-3-carboxamide hydrochloride (0.050 g, 0.155 mmol)) and NEt3 (0.108 mL, 0.773 mmol) in DMF (0.4 mL) was added and the mixture stirred overnight. The mixture was filtered, and the filter rinsed with MCCN (0.2 mL). The mixture was purified by chromatography to give 5-(6-chloro-5-fluoro-1H-indole-2-carbonyl)-N-[1-(hydroxymethyl)cyclopropyl]-N-methyl-4H,5H,6H,7H-pyrazolo[1,5-a]pyrazine-3-carboxamide as a white solid (0.010 g, 14% yield).

Rt (Method A2) 3.21 mins, m/z 446/448 [M+H]+

¹H NMR (400 MHz, DMSO-d6) δ 11.94 (s, 1H), 8.17-7.82 (m, 1H), 7.67 (d, J=10.0 Hz, 1H), 7.56 (d, J=6.4 Hz, 1H), 6.97 (s, 1H), 5.82-4.56 (m, 3H), 4.52-3.91 (m, 4H), 3.78-3.48 (m, 2H), 3.24-2.73 (m, 3H), 1.21-0.54 (m, 4H).

### Example 85

### 5-(4-chloro-6-fluoro-1H-indole-2-carbonyl)-N-[1-(hydroxymethyl)cyclopropyl]-N-methyl-4H,5H,6H,7H-pyrazolo[1,5-a]pyrazine-3-carboxamide

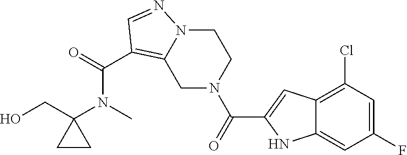 [SMILES: CN(C(=O)c1cnn2c1CN(C(=O)c1cc3c(Cl)cc(F)cc3[nH]1)CC2)C1(CO)CC1]

Rt (Method A2) 3.24 mins, m/z 446/448 [M+H]+

¹H NMR (400 MHz, DMSO-d6) δ 12.23 (s, 1H), 8.21-7.74 (m, 1H), 7.19 (d, J=9.4 Hz, 2H), 6.96 (s, 1H), 5.75-4.61 (m, 3H), 4.54-4.01 (m, 4H), 3.86-3.51 (m, 2H), 3.24-2.78 (m, 3H), 1.34-0.59 (m, 4H).

### Example 86

### 2-{1-[5-(1H-indole-2-carbonyl)-4H,5H,6H,7H-pyrazolo[1,5-a]pyrazin-3-yl]-5-oxopyrrolidin-3-yl}benzoic Acid

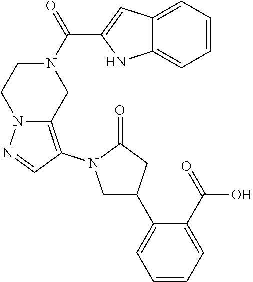 [SMILES: O=C(O)c1ccccc1C1CC(=O)N(c2cnn3c2CN(C(=O)c2cc4ccccc4[nH]2)CC3)C1]

Step 1: To a solution of 2-(1-(5-(tert-butoxycarbonyl)-4,5,6,7-tetrahydropyrazolo[1,5-a]pyrazin-3-yl)-5-oxopyrrolidin-3-yl)benzoic acid (50 mg, 0.117 mmol) in dichloromethane (0.2 mL) was added HCl (4M in dioxane) (1 ml, 4.00 mmol). The mixture was stirred for 90 mins, then concentrated and stripped with DCM to give 2-(5-oxo-1-(4,5,6,7-tetrahydropyrazolo[1,5-a]pyrazin3-yl)pyrrolidin-3-yl)benzoic acid hydrochloride as a yellow solid that was used in the next step without further purification.

Step 2: To a solution of indole-2-carboxylic acid (18.88 mg, 0.117 mmol) in DMSO (400 μL) was added HATU (44.5 mg, 0.117 mmol). The mixture was stirred for 1 hour, then triethylamine (82 μL, 0.586 mmol) was added. This mixture was then added to a solution of 2-(5-oxo-1-(4,5,6,7-tetrahydropyrazolo[1,5-a]pyrazin-3-yl)pyrrolidin-3-yl)benzoic acid hydrochloride (42.5 mg, 0.117 mmol) in DMSO (400 μL). The mixture was stirred overnight, then purified directly by reverse phase column chromatography to give the product as a white solid (0.017 g, 31% yield).

Rt (Method B2) 3.28 mins, m/z 470 [M+H]+

¹H NMR (400 MHz, DMSO-d6) δ 11.68 (s, 1H), 7.72 (d, J=7.7 Hz, 1H), 7.65 (d, J=8.0 Hz, 1H), 7.60 (s, 1H), 7.55-7.34 (m, 3H), 7.29 (t, J=7.6 Hz, 1H), 7.22 (t, J=7.6 Hz, 1H), 7.08 (t, J=7.5 Hz, 1H), 6.96 (s, 1H), 5.22-4.85 (m, 2H), 4.50-4.40 (m, 1H), 4.34-4.15 (m, 4H), 4.10 (t, J=8.7 Hz, 1H), 3.72 (dd, J=9.5, 6.4 Hz, 1H), 2.85 (dd, J=16.8, 8.9 Hz, 1H), 2.58 (dd, J=17.0, 7.6 Hz, 1H). one signal (1H) coincides with water signal.

### Example 87

### N-tert-butyl-5-(1H-indole-2-carbonyl)-4H,5H,6H,7H-pyrazolo[1,5-a]pyrazine-3-carboxamide

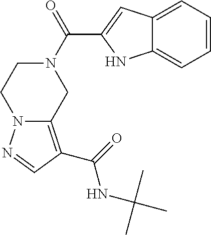 [SMILES: CC(C)(C)NC(=O)c1cnn2c1CN(C(=O)c1cc3ccccc3[nH]1)CC2]

Rt (Method A) 3.03 mins, m/z 366 [M+H]+

1H NMR (400 MHz, DMSO-d6) δ 11.71 (s, 1H), 8.08 (s, 1H), 7.66 (d, J=8.0 Hz, 1H), 7.44 (d, J=8.2 Hz, 1H), 7.35 (s, 1H), 7.26-7.17 (m, 1H), 7.12-7.03 (m, 1H), 6.96 (s, 1H), 5.39-4.95 (m, 2H), 4.33-4.16 (m, 4H), 1.34 (s, 9H).

### Example 88

### 3-fluoro-2-{1-[5-(1H-indole-2-carbonyl)-4H,5H,6H,7H-pyrazolo[1,5-a]pyrazin-3-yl]-5-oxopyrrolidin-3-yl}benzoic Acid

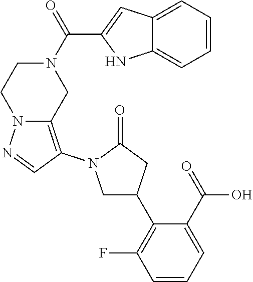 [SMILES: O=C(O)c1cccc(F)c1C1CC(=O)N(c2cnn3c2CN(C(=O)c2cc4ccccc4[nH]2)CC3)C1]

Rt (Method A) 2.33 mins, m/z 488 [M+H]+

¹H NMR (400 MHz, DMSO-d6) δ 11.75-11.64 (m, 1H), 7.70-7.56 (m, 2H), 7.54-7.42 (m, 2H), 7.42-7.16 (m, 3H), 7.08 (t, J=7.5 Hz, 1H), 7.02-6.91 (m, 1H), 5.16-4.84 (m, 2H), 4.62-4.45 (m, 1H), 4.40-4.13 (m, 4H), 4.13-3.99 (m, 1H), 3.87-3.67 (m, 1H), 2.93-2.79 (m, 1H), 2.64-2.52 (m, 2H).

### Example 89

### N-[1-(difluoromethoxy)propan-2-yl]-5-(1H-indole-2-carbonyl)-4H,5H,6H,7H-pyrazolo[1,5-a]pyrazine-3-carboxamide

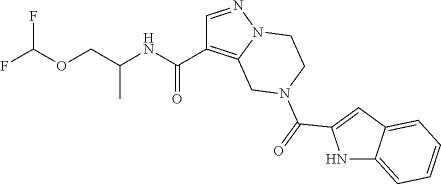 [SMILES: CC(COC(F)F)NC(=O)c1cnn2c1CN(C(=O)c1cc3ccccc3[nH]1)CC2]

Rt (Method A2) 3.34 mins, m/z 418 [M+H]+

¹H NMR (400 MHz, DMSO-d6) ?? 11.71 (s, 1H), 8.09-7.90 (m, 2H), 7.66 (d, J=7.9 Hz, 1H), 7.44 (d, J=8.3 Hz, 1H), 7.22 (t, J=7.5 Hz, 1H), 7.07 (t, J=7.5 Hz, 1H), 6.96 (s, 1H), 6.67 (t, J=75.8 Hz, 1H), 5.44-4.92 (m, 2H), 4.44-4.08 (m, 5H), 3.91-3.67 (m, 2H), 1.15 (d, J=6.7 Hz, 3H).

### Example 90

### N-cyclopropyl-N-[2-(difluoromethoxy)ethyl]-5-(1H-indole-2-carbonyl)-4H,5H,6H,7H-pyrazolo[1,5-a]pyrazine-3-carboxamide

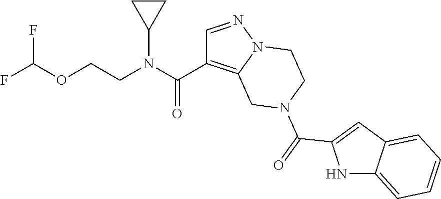 [SMILES: O=C(c1cc2ccccc2[nH]1)N1CCn2ncc(C(=O)N(CCOC(F)F)C3CC3)c2C1]

Rt (Method A2) 3.44 mins, m/z 444 [M+H]+

¹H NMR (400 MHz, DMSO-d6) ?? 11.71 (s, 1H), 8.04 (s, 1H), 7.65 (d, J=8.0 Hz, 1H), 7.44 (d, J=8.3 Hz, 1H), 7.21 (t, J=7.5 Hz, 1H), 7.07 (t, J=7.5 Hz, 1H), 6.95 (s, 1H), 6.68 (t, J=75.8 Hz, 1H), 5.39-4.85 (m, 2H), 4.43-4.11 (m, 4H), 4.08-3.90 (m, 2H), 3.75-3.53 (m, 2H), 3.18-2.97 (m, 1H), 0.96-0.74 (m, 2H), 0.69-0.48 (m, 2H).

### Example 91

### N-{1-[4-(hydroxymethyl)phenyl]cyclopropyl}-5-(1H-indole-2-carbonyl)-N-methyl-4H,5H,6H,7H-pyrazolo[1,5-a]pyrazine-3-carboxamide

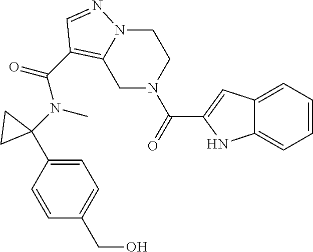 [SMILES: CN(C(=O)c1cnn2c1CN(C(=O)c1cc3ccccc3[nH]1)CC2)C1(c2ccc(CO)cc2)CC1]

Rt (Method J) 1.37 mins, m/z 470 [M+H]+

¹H NMR (400 MHz, DMSO-d6) δ 11.72 (s, 1H), 7.66 (d, J=8.0 Hz, 1H), 7.44 (d, J=8.3 Hz, 1H), 7.37-7.26 (m, 2H), 7.22 (t, J=7.6 Hz, 1H), 7.08 (t, J=7.5 Hz, 1H), 7.05-6.90 (m, 4H), 5.16 (s, 3H), 4.47 (d, J=5.3 Hz, 2H), 4.40-3.92 (m, 4H), 3.27-2.93 (m, 3H), 1.58-1.18 (m, 4H).

### Example 92

### 5-(6-chloro-5-fluoro-1H-indole-2-carbonyl)-N-{1-[(difluoromethoxy)methyl]cyclopropyl}-4H,5H,6H,7H-pyrazolo[1,5-a]pyrazine-3-carboxamide

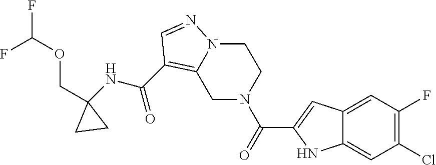 [SMILES: O=C(NC1(COC(F)F)CC1)c1cnn2c1CN(C(=O)c1cc3cc(F)c(Cl)cc3[nH]1)CC2]

To a solution of 5-fluoro-6-chloro-indole-2-carboxylic acid (0.0269 g, 0.126 mmol) in DMF (0.4 mL) was added HATU (0.050 g, 0.132 mmol) and NEt₃ (0.088 mL, 0.629 mmol). The mixture was stirred for 30 mins, then a solution of N-(1-((difluoromethoxy)methyl)cyclopropyl)-4,5,6,7-tetrahydropyrazolo[1,5-a]pyrazine-3-carboxamide (0.036 g, 0.126 mmol) in dry DMF (0.4 mL) was added. The mixture was stirred overnight, then filtered, rinsing with methanol (0.2 mL). The mixture was then purified directly by HPLC to give 5-(6-chloro-5-fluoro-1H-indole-2-carbonyl)-N-{1-[(difluoromethoxy)methyl]cyclopropyl}-4H,5H,6H,7H-pyrazolo[1,5-a]pyrazine-3-carboxamide as a white powder (0.027 g, 44% yield).

Rt (Method A2) 3.60 mins, m/z 482/484 [M+H]+

¹H NMR (400 MHz, DMSO-d6) δ 11.97 (s, 1H), 8.49 (s, 1H), 8.05 (s, 1H), 7.76-7.64 (m, 1H), 7.58 (d, J=6.3 Hz, 1H), 6.99 (s, 1H), 6.67 (t, J=76.3 Hz, 1H), 5.69-4.81 (m, 2H), 4.47-4.06 (m, 4H), 3.94 (s, 2H), 0.94-0.69 (m, 4H).

### Example 93 5-(6-chloro-5-fluoro-1H-indole-2-carbonyl)-N-{1-[(difluoromethoxy)methyl]cyclopropyl}-6-methyl-4H,5H,6H,7H-pyrazolo[1,5-a]pyrazine-3-carboxamide

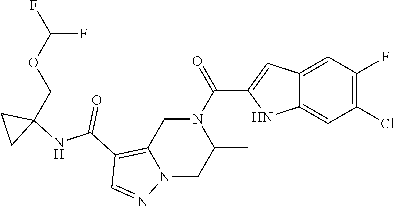 [SMILES: CC1Cn2ncc(C(=O)NC3(COC(F)F)CC3)c2CN1C(=O)c1cc2cc(F)c(Cl)cc2[nH]1]

Rt (Method H) 1.59 mins, m/z 496/498 [M+H]+

¹H NMR (400 MHz, DMSO-d6) Î′ 11.93 (s, 1H), 8.49 (s, 1H), 8.06 (s, 1H), 7.75-7.66 (m, 1H), 7.60-7.54 (m, 1H), 6.98 (s, 1H), 6.67 (t, J=76.2 Hz, 1H), 5.64-5.54 (m, 1H), 5.31-5.18 (m, 1H), 4.91-4.64 (m, 1H), 4.38-4.25 (m, 1H), 4.20-4.12 (m, 1H), 4.00-3.88 (m, 2H), 1.22 (d, J=6.9 Hz, 3H), 0.90-0.73 (m, 4H).

### Example 94

### 5-(5,6-difluoro-1H-indole-2-carbonyl)-N-{1-[(difluoromethoxy)methyl]cyclopropyl}-6-methyl-4H,5H,6H,7H-pyrazolo[1,5-a]pyrazine-3-carboxamide

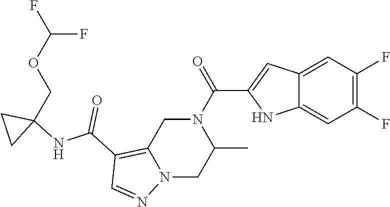 [SMILES: CC1Cn2ncc(C(=O)NC3(COC(F)F)CC3)c2CN1C(=O)c1cc2cc(F)c(F)cc2[nH]1]

Rt (Method H) 1.52 mins, m/z 480 [M+H]+

¹H NMR (400 MHz, DMSO-d6) Î′ 11.89 (s, 1H), 8.49 (s, 1H), 8.06 (s, 1H), 7.77-7.66 (m, 1H), 7.38 (dd, J=11.0, 7.0 Hz, 1H), 6.97 (s, 1H), 6.67 (t, J=76.3 Hz, 1H), 5.64-5.55 (m, 1H), 5.31-5.20 (m, 1H), 4.95-4.62 (m, 1H), 4.39-4.26 (m, 1H), 4.20-4.13 (m, 1H), 4.01-3.87 (m, 2H), 1.22 (d, J=6.8 Hz, 3H), 0.90-0.72 (m, 4H).

### Example 95

### 5-(4-ethyl-1H-indole-2-carbonyl)-N-[1-(hydroxymethyl)cyclopropyl]-N-methyl-4H,5H,6H,7H-pyrazolo[1,5-a]pyrazine-3-carboxamide

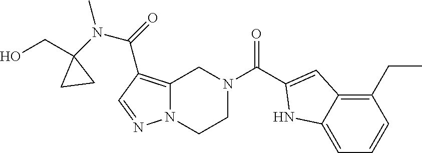 [SMILES: CCc1cccc2[nH]c(C(=O)N3CCn4ncc(C(=O)N(C)C5(CO)CC5)c4C3)cc12]

Rt (Method A2) 3.22 mins, m/z 422 [M+H]+

¹H NMR (400 MHz, DMSO-d6) δ 11.66 (s, 1H), 8.20-7.62 (m, 1H), 7.27 (d, J=8.2 Hz, 1H), 7.13 (dd, J=8.3, 7.1 Hz, 1H), 6.98 (s, 1H), 6.89 (d, J=7.0 Hz, 1H), 5.56-4.56 (m, 3H), 4.53-4.01 (m, 4H), 3.85-3.53 (m, 2H), 3.19-2.93 (m, 3H), 2.89 (q, J=7.6 Hz, 2H), 1.29 (t, J=7.5 Hz, 3H), 1.19-0.53 (m, 4H).

### Example 96

### 5-(4-chloro-5-fluoro-1H-indole-2-carbonyl)-N-[1-(hydroxymethyl)cyclopropyl]-N-methyl-4H,5H,6H,7H-pyrazolo[1,5-a]pyrazine-3-carboxamide

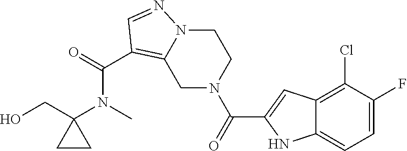 [SMILES: CN(C(=O)c1cnn2c1CN(C(=O)c1cc3c(Cl)c(F)ccc3[nH]1)CC2)C1(CO)CC1]

Rt (Method A2) 3.19 mins, m/z 446/448 [M+H]+

¹H NMR (400 MHz, DMSO-d6) δ 12.18 (s, 1H), 8.26-7.71 (m, 1H), 7.43 (dd, J=9.0, 3.9 Hz, 1H), 7.26 (t, J=9.5 Hz, 1H), 6.96 (s, 1H), 5.53-4.63 (m, 3H), 4.50-4.07 (m, 4H), 3.79-3.58 (m, 2H), 3.20-2.86 (m, 3H), 1.20-0.52 (m, 4H).

### Example 97

### N-{1-[(difluoromethoxy)methyl]cyclopropyl}-5-(1H-indole-2-carbonyl)-4H,5H,6H,7H-pyrazolo[1,5-a]pyrazine-3-carboxamide

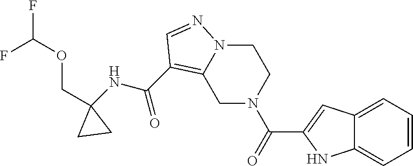 [SMILES: O=C(NC1(COC(F)F)CC1)c1cnn2c1CN(C(=O)c1cc3ccccc3[nH]1)CC2]

Rt (Method A2) 3.32 mins, m/z 430 [M+H]+

¹H NMR (400 MHz, DMSO-d6) δ 11.72 (s, 1H), 8.47 (s, 1H), 8.04 (s, 1H), 7.67 (d, J=8.0 Hz, 1H), 7.44 (d, J=8.2 Hz, 1H), 7.22 (t, J=7.6 Hz, 1H), 7.14-7.03 (m, 1H), 6.96 (s, 1H), 6.66 (t, J=7.6 Hz, 1H), 5.40-4.97 (m, 2H), 4.37-4.07 (m, 4H), 3.93 (s, 2H), 0.90-0.69 (m, 4H).

### Example 98

### 5-(1H-indole-2-carbonyl)-N-[1-(pyridin-4-yl)cyclopropyl]-4H,5H,6H,7H-pyrazolo[1,5-a]pyrazine-3-carboxamide

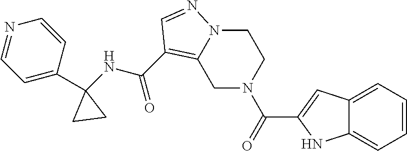 [SMILES: O=C(NC1(c2ccncc2)CC1)c1cnn2c1CN(C(=O)c1cc3ccccc3[nH]1)CC2]

Rt (Method A2) 2.97 mins, m/z 427 [M+H]+

¹H NMR (400 MHz, DMSO-d6) δ 11.70 (s, 1H), 8.90 (s, 1H), 8.43-8.36 (m, 2H), 8.12 (s, 1H), 7.64 (d, J=7.9 Hz, 1H), 7.43 (d, J=8.3 Hz, 1H), 7.21 (t, J=7.5 Hz, 1H), 7.11-7.02 (m, 3H), 6.94 (s, 1H), 5.40-4.93 (m, 2H), 4.40-4.13 (m, 4H), 1.40-1.29 (m, 4H).

### Example 99

### 4-chloro-2-[3-(3,3-difluoropyrrolidine-1-carbonyl)-4H,5H,6H,7H-pyrazolo[1,5-a]pyrazine-5-carbonyl]-1H-indole

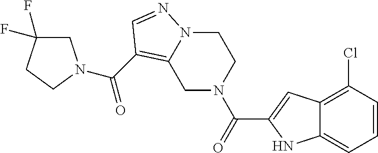 [SMILES: O=C(c1cc2c(Cl)cccc2[nH]1)N1CCn2ncc(C(=O)N3CCC(F)(F)C3)c2C1]

Rt (Method A) 3.25 mins, m/z 434/436 [M+H]+

### Example 100

### 5-(1H-indole-2-carbonyl)-N-methyl-4H,5H,6H,7H-pyrazolo[1,5-a]pyrazine-3-carboxamide

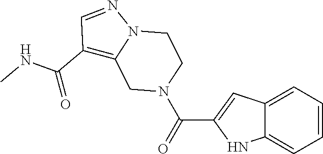 [SMILES: CNC(=O)c1cnn2c1CN(C(=O)c1cc3ccccc3[nH]1)CC2]

Rt (Method A2) 2.80 mins, m/z 324 [M+H]+

¹H NMR (400 MHz, DMSO-d6) δ 11.71 (s, 1H), 8.14-8.00 (m, 1H), 7.94 (s, 1H), 7.66 (d, J=8.1 Hz, 1H), 7.44 (d, J=8.2 Hz, 1H), 7.22 (t, J=7.6 Hz, 1H), 7.08 (t, J=7.5 Hz, 1H), 6.95 (s, 1H), 5.41-4.97 (m, 2H), 4.40-4.08 (m, 4H), 2.75-2.65 (m, 3H).

### Example 101

### 5-(1H-indole-2-carbonyl)-N-[1-(trifluoromethyl)cyclopropyl]-4H,5H,6H,7H-pyrazolo[1,5-a]pyrazine-3-carboxamide

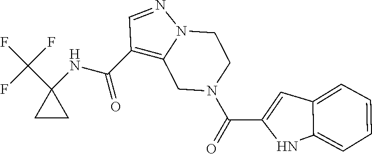 [SMILES: O=C(NC1(C(F)(F)F)CC1)c1cnn2c1CN(C(=O)c1cc3ccccc3[nH]1)CC2]

Step 1: To tert-butyl 3-((1-(trifluoromethyl)cyclopropyl)carbamoyl)-6,7-dihydropyrazolo[1,5-a]pyrazine-5(4H)-carboxylate (100 mg, 0.267 mmol) was added 4M HCl in dioxane (2 mL, 8.00 mmol) and the resulting solution was stirred at r.t. for 1 h. Dioxane (1 mL) was added and stirring was continued overnight. The reaction mixture was diluted with more dioxane and concentrated, The residue was co-evaporated with toluene (2×10 mL) to give N-[1(trifluoromethyl)cyclopropyl]-4H,5H,6H,7H-pyrazolo[1,5-a]pyrazine-3-carboxamide hydrochloride as a white solid that was used in the next step without further purification (0.094 g, 100% yield).

Step 2: A mixture of 1H-indole-2-carboxylic acid (21.60 mg, 0.134 mmol) and HATU (51.0 mg, 0.134 mmol) in DMSO (0.5 mL) was stirred at r.t. for 30 mins. Et₃N (0.093 mL, 0.670 mmol) was added, followed by a solution of N-(1-(trifluoromethyl)cyclopropyl)-4,5,6,7-tetrahydropyrazolo[1,5-a]pyrazine-3-carboxamide hydrochloride (41.6 mg, 0.134 mmol) in DMSO (0.600 mL). The resulting yellow solution was stirred at r.t. for 1 h, filtered, and the filtrate purified by HPLC to give 5-(1H-indole-2-carbonyl)-N-[1-(trifluoromethyl)cyclopropyl]-4H,5H,6H,7H-pyrazolo[1,5-a]pyrazine-3-carboxamide as a white solid (0.030 g, 54% yield).

Rt (Method A2) 3.37 mins, m/z 418 [M+H]+

¹H NMR (400 MHz, DMSO-d6) δ 11.72 (s, 1H), 8.82 (s, 1H), 8.07 (s, 1H), 7.67 (d, J=7.9 Hz, 1H), 7.44 (d, J=8.2 Hz, 1H), 7.22 (t, J=7.5 Hz, 1H), 7.08 (t, J=7.4 Hz, 1H), 6.96 (s, 1H), 5.20 (s, 2H), 4.26 (d, J=26.6 Hz, 4H), 1.36-1.20 (m, 2H), 1.19-1.01 (m, 2H).

### Example 102

### N-{1-[(difluoromethoxy)methyl]cyclopropyl}-5-(6-fluoro-4-methyl-1H-indole-2-carbonyl)-4H,5H,6H,7H-pyrazolo[1,5-a]pyrazine-3-carboxamide

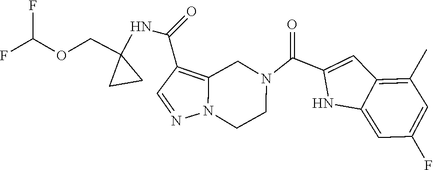 [SMILES: Cc1cc(F)cc2[nH]c(C(=O)N3CCn4ncc(C(=O)NC5(COC(F)F)CC5)c4C3)cc12]

Rt (Method A2) 3.53 mins, m/z 462 [M+H]+

¹H NMR (400 MHz, DMSO-d6) δ 11.77 (s, 1H), 8.48 (s, 1H), 8.06 (s, 1H), 7.02 (s, 1H), 7.01-6.96 (m, 1H), 6.91-6.44 (m, 2H), 5.39-4.99 (m, 2H), 4.44-4.15 (m, 4H), 3.95 (s, 2H), 2.54 (s, 3H), 0.93-0.72 (m, 4H).

### Example 103

### N-{1-[(difluoromethoxy)methyl]cyclopropyl}-5-(5-fluoro-4-methyl-1H-indole-2-carbonyl)-4H,5H,6H,7H-pyrazolo[1,5-a]pyrazine-3-carboxamide

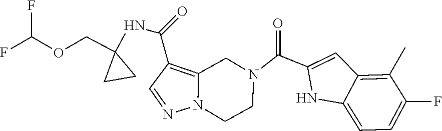 [SMILES: Cc1c(F)ccc2[nH]c(C(=O)N3CCn4ncc(C(=O)NC5(COC(F)F)CC5)c4C3)cc12]

Rt (Method A2) 3.51 mins, m/z 462 [M+H]+

¹H NMR (400 MHz, DMSO-d6) δ 11.78 (s, 1H), 8.48 (s, 1H), 8.06 (s, 1H), 7.27 (dd, J=8.8, 4.2 Hz, 1H), 7.09-7.00 (m, 2H), 6.68 (t, J=76.3 Hz, 1H), 5.19 (s, 2H), 4.48-4.11 (m, 4H), 3.95 (s, 2H), 2.44 (s, 3H), 0.92-0.71 (m, 4H).

### Example 104

### 5-(4,5-difluoro-1H-indole-2-carbonyl)-N-{1-[(difluoromethoxy)methyl]cyclopropyl}-4H,5H,6H,7H-pyrazolo[1,5-a]pyrazine-3-carboxamide

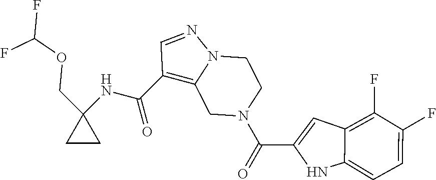 [SMILES: O=C(NC1(COC(F)F)CC1)c1cnn2c1CN(C(=O)c1cc3c(F)c(F)ccc3[nH]1)CC2]

To a solution of 4,5-difluoro-indole-2-carboxylic acid (0.0248 g, 0.126 mmol) in DMF (0.4 mL) was added HATU (0.050 g, 0.132 mmol) and NEt₃ (0.088 mL, 0.629 mmol). The mixture was stirred for 30 mins, then a solution of N-(1-((difluoromethoxy)methyl)cyclopropyl)-4,5,6,7-tetrahydropyrazolo[1,5-a]pyrazine-3-carboxamide (0.036 g, 0.126 mmol) in dry DMF (0.4 mL) was added. The mixture was stirred overnight, then filtered, rinsing with methanol (0.2 mL). The mixture was then purified directly by HPLC to give 5-(4,5-difluoro-1H-indole-2-carbonyl)-N-{1-[(difluoromethoxy)methyl]cyclopropyl}-4H,5H,6H,7H-pyrazolo[1,5-a]pyrazine-3-carboxamide as a white powder (0.037 g, 63% yield).

Rt (Method A2) 3.49 mins, m/z 466 [M+H]+

¹H NMR (400 MHz, DMSO-d6) δ 12.15 (s, 1H), 8.49 (s, 1H), 8.05 (s, 1H), 7.33-7.22 (m, 2H), 7.09 (s, 1H), 6.67 (t, J=76.3 Hz, 1H), 5.49-4.93 (m, 2H), 4.43-4.11 (m, 4H), 3.95 (s, 2H), 0.91-0.73 (m, 4H).

### Example 105

### 5-(4-chloro-1H-indole-2-carbonyl)-N-{1-[(difluoromethoxy)methyl]cyclopropyl}-4H,5H,6H,7H-pyrazolo[1,5-a]pyrazine-3-carboxamide

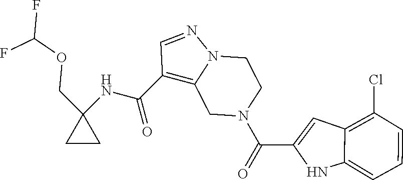 [SMILES: O=C(NC1(COC(F)F)CC1)c1cnn2c1CN(C(=O)c1cc3c(Cl)cccc3[nH]1)CC2]

Rt (Method A2) 3.55 mins, m/z 464/466 [M+H]+

¹H NMR (400 MHz, DMSO-d6) δ 12.11 (s, 1H), 8.48 (s, 1H), 8.06 (s, 1H), 7.44 (d, J=8.1 Hz, 1H), 7.23 (t, J=7.8 Hz, 1H), 7.17 (d, J=7.5 Hz, 1H), 6.95 (s, 1H), 6.67 (t, J=76.3 Hz, 1H), 5.55-4.94 (m, 2H), 4.41-4.15 (m, 4H), 3.95 (s, 2H), 0.91-0.72 (m, 4H).

### Example 106

### 5-(1H-indole-2-carbonyl)-N-[1-(trifluoromethyl)cyclobutyl]-4H,5H,6H,7H-pyrazolo[1,5-a]pyrazine-3-carboxamide

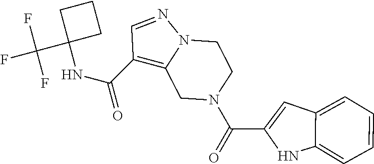 [SMILES: O=C(NC1(C(F)(F)F)CCC1)c1cnn2c1CN(C(=O)c1cc3ccccc3[nH]1)CC2]

Rt (Method A2) 3.56 mins, m/z 432 [M+H]+

¹H NMR (400 MHz, DMSO-d6) δ 11.72 (s, 1H), 8.42 (s, 1H), 8.12 (s, 1H), 7.66 (d, J=8.0 Hz, 1H), 7.44 (d, J=8.3 Hz, 1H), 7.22 (t, J=7.6 Hz, 1H), 7.07 (t, J=7.5 Hz, 1H), 6.96 (s, 1H), 5.43-4.92 (m, 2H), 4.43-4.14 (m, 4H), 2.01-1.82 (m, 2H).

### Example 107

### 4-[5-(6-fluoro-4-methyl-1H-indole-2-carbonyl)-4H,5H,6H,7H-pyrazolo[1,5-a]pyrazine-3-carbonyl]-8-oxa-4-azaspiro[2.6]nonane

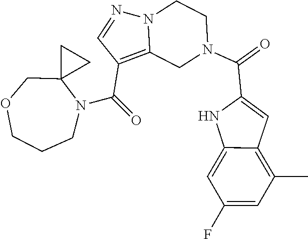 [SMILES: Cc1cc(F)cc2[nH]c(C(=O)N3CCn4ncc(C(=O)N5CCCOCC56CC6)c4C3)cc12]

Rt (Method A2) 3.29 mins, m/z 452 [M+H]+

¹H NMR (400 MHz, DMSO-d6) δ 11.75 (s, 1H), 7.74 (s, 1H), 7.09-6.87 (m, 2H), 6.87-6.63 (m, 1H), 5.39-4.88 (m, 2H), 4.46-3.35 (m, 10H), 2.52 (s, 3H), 2.05-1.84 (m, 2H), 1.17-0.61 (m, 4H).

### Example 108

### 4-[5-(5-fluoro-4-methyl-1H-indole-2-carbonyl)-4H,5H,6H,7H-pyrazolo[1,5-a]pyrazine-3-carbonyl]-8-oxa-4-azaspiro[2.6]nonane

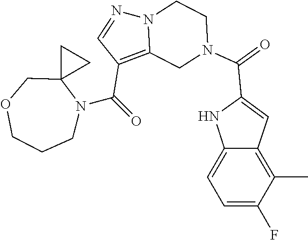 [SMILES: Cc1c(F)ccc2[nH]c(C(=O)N3CCn4ncc(C(=O)N5CCCOCC56CC6)c4C3)cc12]

Rt (Method A2) 3.28 mins, m/z 452 [M+H]+

¹H NMR (400 MHz, DMSO-d6) δ 11.76 (s, 1H), 7.75 (s, 1H), 7.34-7.15 (m, 1H), 7.12-6.87 (m, 2H), 5.35-4.84 (m, 2H), 4.51-3.41 (m, 10H), 2.42 (d, J=1.9 Hz, 3H), 2.03-1.82 (m, 2H), 1.06-0.76 (m, 4H).

### Example 109

### 4-[5-(4,5-difluoro-1H-indole-2-carbonyl)-4H,5H,6H,7H-pyrazolo[1,5-a]pyrazine-3-carbonyl]-8-oxa-4-azaspiro[2.6]nonane

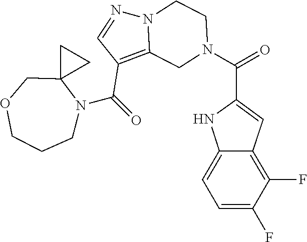 [SMILES: O=C(c1cc2c(F)c(F)ccc2[nH]1)N1CCn2ncc(C(=O)N3CCCOCC34CC4)c2C1]

To 4,5-difluoro-indole-2-carboxylic acid (0.0315 g, 0.16 mmol), in DMSO (0.5 mL) was added HATU (66.9 mg, 0.176 mmol). The resulting mixture was stirred at r.t. for 30 min, after which time Et₃N (0.111 mL, 0.799 mmol) and a solution of (8-oxa-4-azaspiro[2.6]nonan-4-yl)(4,5,6,7-tetrahydropyrazolo[1,5-a]pyrazin-3-yl)methanonehydrochloride (50 mg, 0.16 mmol) in DMSO (0.8 mL) was added. The mixture was stirred overnight, then filtered through a micro filter, diluted with DMSO (0.5 mL), and purified directly by HPLC to give the product as a white solid (0.0151 g, 21% yield).

Rt (Method A2) 3.25 mins, m/z 456 [M+H]+

¹H NMR (400 MHz, DMSO-d6) δ 12.13 (s, 1H), 7.75 (s, 1H), 7.36-7.18 (m, 2H), 7.07 (s, 1H), 5.53-4.74 (m, 2H), 4.52-3.42 (m, 10H), 2.04-1.82 (m, 2H), 1.21-0.60 (m, 4H).

### Example 110

### 4-[5-(4-chloro-1H-indole-2-carbonyl)-4H,5H,6H,7H-pyrazolo[1,5-a]pyrazine-3-carbonyl]-8-oxa-4-azaspiro[2.6]nonane

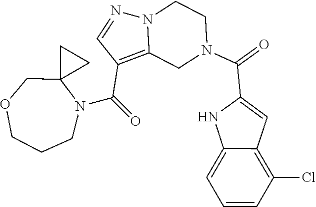 [SMILES: O=C(c1cc2c(Cl)cccc2[nH]1)N1CCn2ncc(C(=O)N3CCCOCC34CC4)c2C1]

Rt (Method A2) 3.31 mins, m/z 454/456 [M+H]+

¹H NMR (400 MHz, DMSO-d6) δ 12.10 (s, 1H), 7.74 (s, 1H), 7.42 (d, J=8.0 Hz, 1H), 7.28-7.09 (m, 2H), 6.93 (s, 1H), 5.44-4.86 (m, 2H), 4.51-3.41 (m, 10H), 2.05-1.83 (m, 2H), 1.18-0.63 (m, 4H).

### Example 111

### 4-[5-(5-chloro-6-fluoro-1H-indole-2-carbonyl)-4H,5H,6H,7H-pyrazolo[1,5-a]pyrazine-3-carbonyl]-8-oxa-4-azaspiro[2.6]nonane

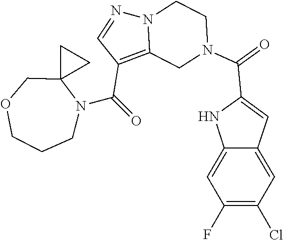 [SMILES: O=C(c1cc2cc(Cl)c(F)cc2[nH]1)N1CCn2ncc(C(=O)N3CCCOCC34CC4)c2C1]

Step 1: To a stirred solution of tert-butyl 3-(8-oxa-4-azaspiro[2.6]nonane-4-carbonyl)-6,7-dihydropyrazolo[1,5-a]pyrazine-5(4H)-carboxylate (393 mg, 1.044 mmol) in 1,4-dioxane (0.6 mL) was added 4M HCl in dioxane (4 mL, 16.00 mmol). The resulting solution was stirred at r.t. overnight. The reaction mixture was diluted with dioxane and concentrated and co-evaporated with toluene (2×10 mL) to give 4-{4H,5H,6H,7H-pyrazolo[1,5-a]pyrazine-3-carbonyl}-8-oxa-4-azaspiro[2.6]nonane hydrochloride as an off-white solid (354 mg, 100% yield).

Step 2: To 5-fluoro-6-chloro-indole-2-carboxylic acid (0.0342 g, 0.16 mmol), in DMSO (0.5 mL) was added HATU (66.9 mg, 0.176 mmol). The resulting mixture was stirred at r.t. for 30 min, after which time Et₃N (0.111 mL, 0.799 mmol) and a solution of (8-oxa-4-azaspiro[2.6]nonan-4-yl)(4,5,6,7-tetrahydropyrazolo[1,5-a]pyrazin-3-yl)methanonehydrochloride (50 mg, 0.16 mmol) in DMSO (0.8 mL) was added. The mixture was stirred overnight, then filtered through a micro filter, diluted with DMSO (0.5 mL), and purified directly by HPLC to give the product as a white solid (0.0267 g, 35% yield).

Rt (Method A2) 3.37 mins, m/z 472/474 [M+H]+

¹H NMR (400 MHz, DMSO-d6) δ 11.95 (s, 1H), 7.70 (d, J=34.4 Hz, 2H), 7.56 (d, J=6.4 Hz, 1H), 6.98 (s, 1H), 5.14 (s, 2H), 4.44-3.36 (m, 10H), 1.94 (d, J=8.3 Hz, 2H), 0.88 (s, 4H).

### Example 112

### 5-(6-fluoro-4-methyl-1H-indole-2-carbonyl)-N-[(2R)-1,1,1-trifluoropropan-2-yl]-4H,5H,6H,7H-pyrazolo[1,5-a]pyrazine-3-carboxamide

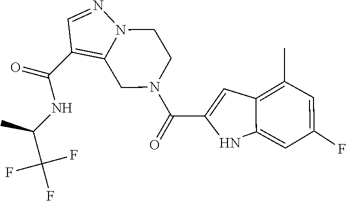 [SMILES: Cc1cc(F)cc2[nH]c(C(=O)N3CCn4ncc(C(=O)N[C@H](C)C(F)(F)F)c4C3)cc12]

Rt (Method A2) 3.63 mins, m/z 438 [M+H]+

¹H NMR (400 MHz, DMSO-d6) δ 11.76 (s, 1H), 8.47 (d, J=8.8 Hz, 1H), 8.14 (s, 1H), 7.09-6.89 (m, 2H), 6.78 (dd, J=10.9, 2.2 Hz, 1H), 5.35-4.99 (m, 2H), 4.89-4.68 (m, 1H), 4.43-4.04 (m, 4H), 2.52 (s, 3H), 1.32 (d, J=7.1 Hz, 3H).

### Example 113

### 5-(5-fluoro-4-methyl-1H-indole-2-carbonyl)-N-[(2R)-1,1,1-trifluoropropan-2-yl]-4H,5H,6H,7H-pyrazolol[1,5-a]pyrazine-3-carboxamide

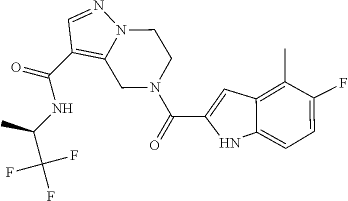 [SMILES: Cc1c(F)ccc2[nH]c(C(=O)N3CCn4ncc(C(=O)N[C@H](C)C(F)(F)F)c4C3)cc12]

To 4-methyl-5-fluoro-indole-2-carboxylic acid (0.0315 g, 0.167 mmol), in DMSO (0.5 mL) was added HATU (70.0 mg, 0.184 mmol). The resulting mixture was stirred at r.t. for 30 min, after which time a mixture of Et₃N (0.117 mL, 0.837 mmol) and (R)—N-(1,1,1-trifluoropropan-2-yl)-4,5,6,7-tetrahydropyrazolo[1,5-a]pyrazine-3-carboxamide hydrochloride (50 mg, 0.167 mmol) in DMSO (0.8 mL) was added. The mixture was stirred overnight, then filtered through a micro filter, diluted with DMSO (0.5 mL), and purified directly by HPLC to give the product as a white solid (0.0338 g, 48% yield).

Rt (Method A2) 3.61 mins, m/z 438 [M+H]+

¹H NMR (400 MHz, DMSO-d6) δ 11.77 (s, 1H), 8.47 (d, J=8.9 Hz, 1H), 8.14 (s, 1H), 7.26 (dd, J=8.8, 4.2 Hz, 1H), 7.16-6.76 (m, 2H), 5.19 (s, 2H), 4.79 (h, J=7.7 Hz, 1H), 4.48-3.94 (m, 4H), 2.42 (m, 3H), 1.32 (d, J=7.3 Hz, 3H).

### Example 114

### 5-(4,5-difluoro-1H-indole-2-carbonyl)-N-[(2R)-1,1,1-trifluoropropan-2-yl]-4H,5H,6H,7H-pyrazolo[1,5-a]pyrazine-3-carboxamide

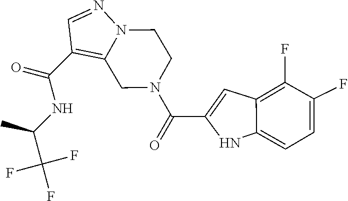 [SMILES: C[C@@H](NC(=O)c1cnn2c1CN(C(=O)c1cc3c(F)c(F)ccc3[nH]1)CC2)C(F)(F)F]

To 4,5-difluoro-indole-2-carboxylic acid (0.0315 g, 0.167 mmol), in DMSO (0.5 mL) was added HATU (70.0 mg, 0.184 mmol). The resulting mixture was stirred at r.t. for 30 min, after which time a mixture of Et₃N (0.117 ml, 0.837 mmol) and (R)—N-(1,1,1-trifluoropropan-2-yl)-4,5,6,7-tetrahydropyrazolo[1,5-a]pyrazine-3-carboxamide hydrochloride (50 mg, 0.167 mmol) in DMSO (0.8 mL) was added. The mixture was stirred overnight, then filtered through a micro filter, diluted with DMSO (0.5 mL), and purified directly by HPLC to give the product as a white solid (0.0437 g, 62% yield).

Rt (Method A2) 3.58 mins, m/z 442 [M+H]+

¹H NMR (400 MHz, DMSO-d6) δ 12.14 (s, 1H), 8.48 (d, J=8.8 Hz, 1H), 8.14 (s, 1H), 7.36-7.17 (m, 2H), 7.07 (s, 1H), 5.60-4.91 (m, 2H), 4.79 (q, J=7.8 Hz, 1H), 4.52-3.90 (m, 4H), 1.32 (d, J=7.1 Hz, 3H).

### Example 115

### 5-(4-chloro-1H-indole-2-carbonyl)-N-[(2R)-1,1,1-trifluoropropan-2-yl]-4H,5H,6H,7H-pyrazolo[1,5-a]pyrazine-3-carboxamide

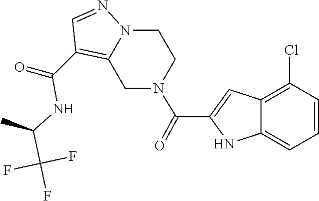 [SMILES: C[C@@H](NC(=O)c1cnn2c1CN(C(=O)c1cc3c(Cl)cccc3[nH]1)CC2)C(F)(F)F]

To 4-chloro-indole-2-carboxylic acid (0.0315 g, 0.167 mmol), in DMSO (0.5 mL) was added HATU (70.0 mg, 0.184 mmol). The resulting mixture was stirred at r.t. for 30 min, after which time a mixture of Et₃N (0.117 mL, 0.837 mmol) and (R)—N-(1,1,1-trifluoropropan-2-yl)-4,5,6,7-tetrahydropyrazolo[1,5-a]pyrazine-3-carboxamide hydrochloride (50 mg, 0.167 mmol) in DMSO (0.8 mL) was added. The mixture was stirred overnight, then filtered through a micro filter, diluted with DMSO (0.5 mL), and purified directly by HPLC to give the product as a white solid (0.0455 g, 65% yield).

Rt (Method A2) 3.65 mins, m/z 440/442 [M+H]+

¹H NMR (400 MHz, DMSO-d6) δ 12.10 (s, 1H), 8.47 (d, J=8.9 Hz, 1H), 8.14 (s, 1H), 7.43 (d, J=8.0 Hz, 1H), 7.28-7.10 (m, 2H), 6.94 (s, 1H), 5.55-4.91 (m, 2H), 4.79 (q, 1H), 4.47-4.04 (m, 4H), 1.32 (d, J=7.1 Hz, 3H).

### Example 116

### 5-(5-chloro-6-fluoro-1H-indole-2-carbonyl)-N-[(2R)-1,1,1-trifluoropropan-2-yl]-4H,5H,6H,7H-pyrazolo[1,5-a]pyrazine-3-carboxamide

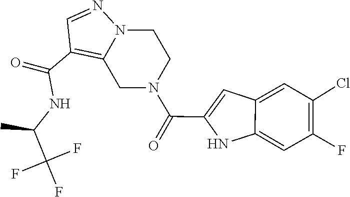 [SMILES: C[C@@H](NC(=O)c1cnn2c1CN(C(=O)c1cc3cc(Cl)c(F)cc3[nH]1)CC2)C(F)(F)F]

Step 1: Tert-butyl (R)-3-((1,1,1-trifluoropropan-2-yl)carbamoyl)-6,7-dihydropyrazolo[1,5-a]pyrazine-5(4H)-carboxylate (353 mg, 0.974 mmol) was dissolved in 4M HCl in dioxane (4 mL, 16.00 mmol) and the resulting sticky suspension was stirred at r.t. for 30 mins. The reaction mixture was diluted with dioxane (4 mL) and stirred for a further 30 mins. The reaction mixture was then diluted with 1,4-dioxane (8 mL) and concentrated, and the residue was co-evaporated with toluene (2×10 mL) to give N-[(2R)-1,1,1-trifluoropropan-2-yl]-4H,5H,6H,7H-pyrazolo[1,5-a]pyrazine-3-carboxamide hydrochloride as an off white solid (0.280 g, 96% yield).

Step 2: To 5-chloro-6-fluoro indole-2-carboxylic acid (0.0342 g, 0.167 mmol), in DMSO (0.5 mL) was added HATU (70.0 mg, 0.184 mmol). The resulting mixture was stirred at r.t. for 30 min, after which time a mixture of Et₃N (0.117 mL, 0.837 mmol) and (R)—N-(1,1,1-trifluoropropan-2-yl)-4,5,6,7-tetrahydropyrazolo[1,5-a]pyrazine-3-carboxamide hydrochloride (50 mg, 0.167 mmol) in DMSO (0.8 mL) was added. The mixture was stirred overnight, then filtered through a micro filter, diluted with DMSO (0.5 mL), and purified directly by HPLC to give the product as a white solid (0.0387 g, 53% yield).

Rt (Method A2) 3.70 mins, m/z 458/460 [M+H]+

¹H NMR (400 MHz, DMSO-d6) δ 11.95 (s, 1H), 8.48 (d, J=8.9 Hz, 1H), 8.14 (s, 1H), 7.67 (d, J=9.9 Hz, 1H), 7.57 (d, J=6.4 Hz, 1H), 6.98 (s, 1H), 5.52-4.91 (m, 2H), 4.78 (q, J=8.0 Hz, 1H), 4.45-3.95 (m, 4H), 1.32 (d, J=7.1 Hz, 3H).

### Example 117

### 4-[5-(6-chloro-5-fluoro-1H-indole-2-carbonyl)-4H,5H,6H,7H-pyrazolo[1,5-a]pyrazine-3-carbonyl]-4-azaspiro[2.5]octan-7-ol

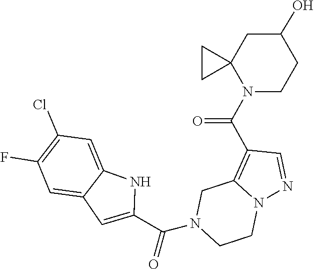 [SMILES: O=C(c1cc2cc(F)c(Cl)cc2[nH]1)N1CCn2ncc(C(=O)N3CCC(O)CC34CC4)c2C1]

Step 1: Tert-butyl 3-(7-hydroxy-4-azaspiro[2.5]octane-4-carbonyl)-6,7-dihydropyrazolo[1,5-a]pyrazine-5(4H)-carboxylate (275 mg, 0.730 mmol) was dissolved in 4M HCl in dioxane (4 mL, 16.00 mmol) and the resulting gel-like suspension was stirred at r.t. for 3 h, then diluted with dioxane (4 mL) and concentrated, after which the residue was co-evaporated with toluene (2×10 mL) to give 4-{4H,5H,6H,7H-pyrazolo[1,5-a]pyrazine-3-carbonyl}-4-azaspiro[2.5]octan-7-ol hydrochloride as a white solid (0.235 g, 100% yield).

Step 2: To 5-chloro-6-fluoro indole-2-carboxylic acid (0.0273 g, 0.128 mmol), in DMSO (0.5 mL) was added HATU (53.5 mg, 0.141 mmol). The resulting mixture was stirred at r.t. for 30 min, after which time a mixture of Et₃N (0.89 mL, 0.837 mmol) and (7-hydroxy-4-azaspiro[2.5]octan-4-yl)(4,5,6,7-tetrahydropyrazolo[1,5-a]pyrazin-3-yl)methanone hydrochloride (40 mg, 0.128 mmol) in DMSO (0.8 mL) was added. The mixture was stirred overnight, then filtered through a micro filter, diluted with DMSO (0.5 mL), and purified directly by HPLC to give the product as a white solid (0.0196 g, 32% yield).

Rt (Method A2) 3.19 mins, m/z 472/474 [M+H]+

¹H NMR (400 MHz, DMSO-d6) δ 11.95 (s, 1H), 7.81-7.61 (m, 2H), 7.56 (d, J=6.4 Hz, 1H), 6.96 (s, 1H), 5.40-4.86 (m, 2H), 4.85-4.68 (m, 1H), 4.46-4.05 (m, 5H), 3.91-3.72 (m, 1H), 3.22-2.80 (m, 1H), 1.92-1.68 (m, 2H), 1.52-1.34 (m, 1H), 1.32-1.08 (m, 1H), 1.02-0.76 (m, 2H), 0.68-0.41 (m, 2H).

### Example 118

### 4-[5-(4-chloro-1H-indole-2-carbonyl)-4H,5H,6H,7H-pyrazolo[1,5-a]pyrazine-3-carbonyl]-4-azaspiro[2.5]octan-7-ol

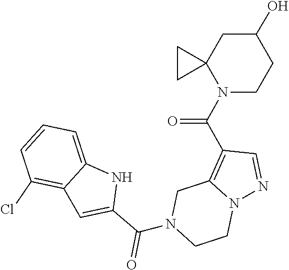 [SMILES: O=C(c1cc2c(Cl)cccc2[nH]1)N1CCn2ncc(C(=O)N3CCC(O)CC34CC4)c2C1]

To 4-chloro-indole-2-carboxylic acid (0.0250 g, 0.128 mmol), in DMSO (0.5 mL) was added HATU (53.5 mg, 0.141 mmol). The resulting mixture was stirred at r.t. for 30 min, after which time a mixture of Et₃N (0.89 mL, 0.837 mmol) and (7-hydroxy-4-azaspiro[2.5]octan-4-yl)(4,5,6,7-tetrahydropyrazolo[1,5-a]pyrazin-3-yl)methanone hydrochloride (40 mg, 0.128 mmol) in DMSO (0.8 mL) was added. The mixture was stirred overnight, then filtered through a micro filter, diluted with DMSO (0.5 mL), and purified directly by HPLC to give the product as a white solid (0.0225 g, 39% yield).

Rt (Method A2) 3.11 mins, m/z 454/456 [M+H]+

¹H NMR (400 MHz, DMSO-d6) δ 12.09 (s, 1H), 7.75 (s, 1H), 7.42 (d, J=8.1 Hz, 1H), 7.27-7.08 (m, 2H), 6.92 (s, 1H), 5.41-4.86 (m, 2H), 4.86-4.67 (m, 1H), 4.47-4.02 (m, 5H), 3.93-3.73 (m, 1H), 3.18-2.78 (m, 1H), 1.98-1.68 (m, 2H), 1.58-1.10 (m, 2H), 1.02-0.73 (m, 2H), 0.67-0.44 (m, 2H).

### Example 119

### 4-[5-(4,5-difluoro-1H-indole-2-carbonyl)-4H,5H,6H,7H-pyrazolo[1,5-a]pyrazine-3-carbonyl]-4-azaspiro[2.5]octan-7-ol

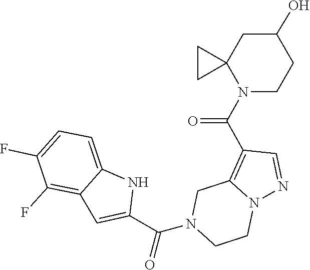 [SMILES: O=C(c1cc2c(F)c(F)ccc2[nH]1)N1CCn2ncc(C(=O)N3CCC(O)CC34CC4)c2C1]

To 4,5-difluoro-indole-2-carboxylic acid (0.0252 g, 0.128 mmol), in DMSO (0.5 mL) was added HATU (53.5 mg, 0.141 mmol). The resulting mixture was stirred at r.t. for 30 min, after which time a mixture of Et₃N (0.89 mL, 0.837 mmol) and (7-hydroxy-4-azaspiro[2.5]octan-4-yl)(4,5,6,7-tetrahydropyrazolo[1,5-a]pyrazin-3-yl)methanone hydrochloride (40 mg, 0.128 mmol) in DMSO (0.8 mL) was added. The mixture was stirred overnight, then filtered through a micro filter, diluted with DMSO (0.5 mL), and purified directly by HPLC to give the product as a white solid (0.0242 g, 42% yield).

Rt (Method A2) 3.06 mins, m/z 456 [M+H]+

¹H NMR (400 MHz, DMSO-d6) δ 12.13 (s, 1H), 7.75 (s, 1H), 7.32-7.16 (m, 2H), 7.06 (s, 1H), 5.49-4.84 (m, 2H), 4.85-4.66 (m, 1H), 4.49-4.01 (m, 5H), 3.92-3.72 (m, 1H), 3.18-2.85 (m, 1H), 1.95-1.63 (m, 2H), 1.57-1.10 (m, 2H), 1.04-0.75 (m, 2H), 0.66-0.45 (m, 2H).

### Example 120

### 4-[5-(5-fluoro-4-methyl-1H-indole-2-carbonyl)-4H,5H,6H,7H-pyrazolo[1,5-a]pyrazine-3-carbonyl]-4-azaspiro[2.5]octan-7-ol

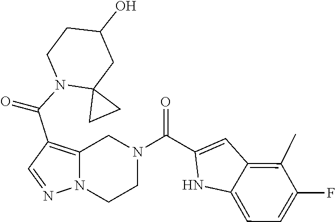 [SMILES: Cc1c(F)ccc2[nH]c(C(=O)N3CCn4ncc(C(=O)N5CCC(O)CC56CC6)c4C3)cc12]

Rt (Method A2) 3.10 mins, m/z 452 [M+H]+

¹H NMR (400 MHz, DMSO-d6) δ 11.77 (s, 1H), 7.75 (s, 1H), 7.34-7.18 (m, 1H), 7.11-6.92 (m, 2H), 5.38-4.88 (m, 2H), 4.88-4.64 (m, 1H), 4.47-4.04 (m, 5H), 3.91-3.73 (m, 1H), 3.20-2.72 (m, 1H), 2.42 (s, 3H), 1.99-1.64 (m, 2H), 1.59-1.08 (m, 2H), 1.01-0.71 (m, 2H), 0.68-0.45 (m, 2H).

### Example 121

### 4-[5-(6-fluoro-4-methyl-1H-indole-2-carbonyl)-4H,5H,6H,7H-pyrazolo[1,5-a]pyrazine-3-carbonyl]-4-azaspiro[2.5]octan-7-ol

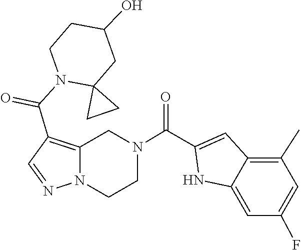 [SMILES: Cc1cc(F)cc2[nH]c(C(=O)N3CCn4ncc(C(=O)N5CCC(O)CC56CC6)c4C3)cc12]

Rt (Method A2) 3.11 mins, m/z 452 [M+H]+

¹H NMR (400 MHz, DMSO-d6) δ 11.76 (s, 1H), 7.75 (s, 1H), 7.10-6.88 (m, 2H), 6.83-6.69 (m, 1H), 5.35-4.90 (m, 2H), 4.87-4.64 (m, 1H), 4.45-4.01 (m, 5H), 3.89-3.76 (m, 1H), 3.22-2.73 (m, 1H), 1.94-1.67 (m, 2H), 1.58-1.07 (m, 2H), 1.02-0.73 (m, 2H), 0.67-0.41 (m, 2H).

### Example 122

### 2-[3-(3,3-difluoropyrrolidine-1-carbonyl)-4H,5H,6H,7H-pyrazolo[1,5-a]pyrazine-5-carbonyl]-1H-indole

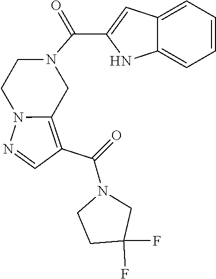 [SMILES: O=C(c1cc2ccccc2[nH]1)N1CCn2ncc(C(=O)N3CCC(F)(F)C3)c2C1]

Rt (Method A) 3.04 mins, m/z 400 [M+H]+

### Example 123

### N-{1-[2-(difluoromethoxy)ethyl]cyclobutyl}-5-(1H-indole-2-carbonyl)-4H,5H,6H,7H-pyrazolo[1,5-a]pyrazine-3-carboxamide

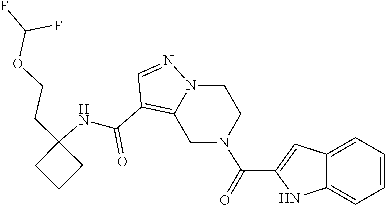 [SMILES: O=C(NC1(CCOC(F)F)CCC1)c1cnn2c1CN(C(=O)c1cc3ccccc3[nH]1)CC2]

Rt (Method A2) 3.62 mins, m/z 458 [M+H]+

¹H NMR (400 MHz, DMSO-d6) δ 11.71 (s, 1H), 8.03 (d, J=22.9 Hz, 2H), 7.66 (d, J=8.0 Hz, 1H), 7.44 (d, J=8.1 Hz, 1H), 7.21 (t, J=7.8 Hz, 1H), 7.07 (t, J=7.5 Hz, 1H), 6.96 (s, 1H), 6.61 (t, J=76.4 Hz, 1H), 5.41-4.92 (m, 2H), 4.35-4.15 (m, 4H), 3.82 (t, J=7.1 Hz, 2H), 2.29-2.04 (m, 6H), 1.89-1.75 (m, 2H).

### Example 124

### N-{1-[2-(difluoromethoxy)ethyl]cyclopentyl}-5-(1H-indole-2-carbonyl)-4H,5H,6H,7H-pyrazolo[1,5-a]pyrazine-3-carboxamide

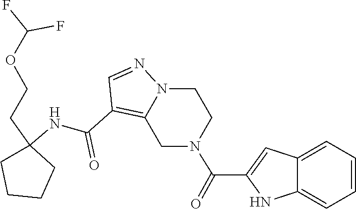 [SMILES: O=C(NC1(CCOC(F)F)CCCC1)c1cnn2c1CN(C(=O)c1cc3ccccc3[nH]1)CC2]

Rt (Method A2) 3.78 mins, m/z 472 [M+H]+

### Example 125

### N-{4-[2-(difluoromethoxy)ethyl]oxan-4-yl}-5-(1H-indole-2-carbonyl)-4H,5H,6H,7H-pyrazolo[1,5-a]pyrazine-3-carboxamide

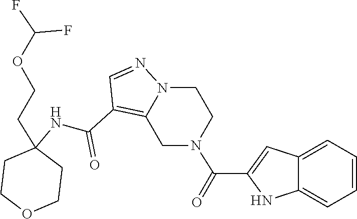 [SMILES: O=C(NC1(CCOC(F)F)CCOCC1)c1cnn2c1CN(C(=O)c1cc3ccccc3[nH]1)CC2]

Rt (Method A2) 3.35 mins, m/z 488 [M+H]+

### Example 126

### N-{1-[2-(difluoromethoxy)ethyl]cyclobutyl}-5-(1H-indole-2-carbonyl)-N-methyl-4H,5H,6H,7H-pyrazolo[1,5-a]pyrazine-3-carboxamide

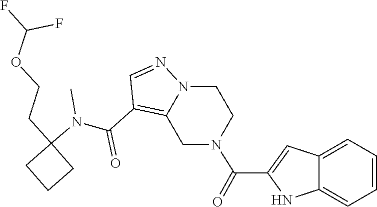 [SMILES: CN(C(=O)c1cnn2c1CN(C(=O)c1cc3ccccc3[nH]1)CC2)C1(CCOC(F)F)CCC1]

Rt (Method A2) 3.68 mins, m/z 472 [M+H]+

### Example 127

### N-{1-[2-(difluoromethoxy)ethyl]cyclopentyl}-5-(1H-indole-2-carbonyl)-N-methyl-4H,5H,6H,7H-pyrazolo[1,5-a]pyrazine-3-carboxamide

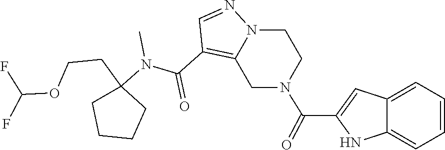 [SMILES: CN(C(=O)c1cnn2c1CN(C(=O)c1cc3ccccc3[nH]1)CC2)C1(CCOC(F)F)CCCC1]

Rt (Method A2) 3.84 mins, m/z 448 [M+H]+

### Example 128

### N-{1-[2-(difluoromethoxy)ethyl]cyclopentyl}-5-(1H-indole-2-carbonyl)-N-methyl-4H,5H,6H,7H-pyrazolo[1,5-a]pyrazine-3-carboxamide

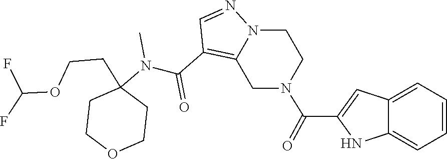 [SMILES: CN(C(=O)c1cnn2c1CN(C(=O)c1cc3ccccc3[nH]1)CC2)C1(CCOC(F)F)CCOCC1]

Rt (Method A2) 3.41 mins, m/z 502 [M+H]+

### Example 129

### 5-(4,6-difluoro-1H-indole-2-carbonyl)-N-(2-hydroxyethyl)-N-methyl-4H,5H,6H,7H-pyrazolo[1,5-a]pyrazine-3-carboxamide

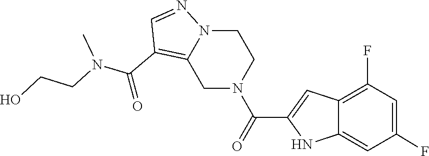 [SMILES: CN(CCO)C(=O)c1cnn2c1CN(C(=O)c1cc3c(F)cc(F)cc3[nH]1)CC2]

Rt (Method A) 2.77 mins, m/z 404 [M+H]+

### Example 130

### N-{1-[(difluoromethoxy)methyl]cyclopropyl}-5-(1H-indole-2-carbonyl)-6-methyl-4H,5H,6H,7H-pyrazolo[1,5-a]pyrazine-3-carboxamide (Racemic Mixture)

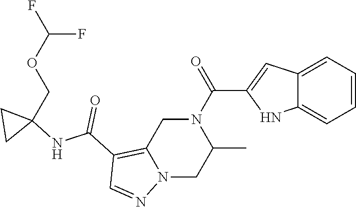 [SMILES: CC1Cn2ncc(C(=O)NC3(COC(F)F)CC3)c2CN1C(=O)c1cc2ccccc2[nH]1]

Rt (Method B2) 3.44 mins, m/z 444 [M+H]+

¹H NMR (400 MHz, DMSO-d6) δ 11.70 (s, 1H), 8.49 (s, 1H), 8.07 (s, 1H), 7.67 (d, J=8.0 Hz, 1H), 7.45 (d, J=8.2 Hz, 1H), 7.22 (t, J=7.6 Hz, 1H), 7.08 (t, J=7.5 Hz, 1H), 6.95 (s, 1H), 6.67 (t, J=7.64 Hz, 1H), 5.62 (d, J=18.6 Hz, 1H), 5.33-5.22 (m, 1H), 5.00-4.51 (m, 1H), 4.41-4.27 (m, 1H), 4.16 (d, J=12.9 Hz, 1H), 3.98-3.89 (m, 2H), 1.23 (d, J=6.8 Hz, 3H), 0.87-0.74 (m, 4H).

### Example 131

### N-{1-[(difluoromethoxy)methyl]cyclopropyl}-5-(1H-indole-2-carbonyl)-6-methyl-4H,5H,6H,7H-pyrazolo[1,5-a]pyrazine-3-carboxamide (Enantiomer 1, Absolute Configuration Unknown)

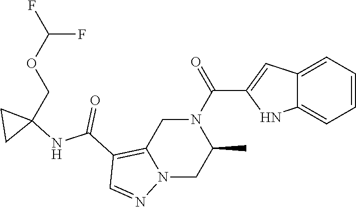 [SMILES: C[C@H]1Cn2ncc(C(=O)NC3(COC(F)F)CC3)c2CN1C(=O)c1cc2ccccc2[nH]1]

Rt (Method B2) 3.44 mins, m/z 444 [M+H]+

¹H NMR (400 MHz, DMSO-d6) δ 11.69 (s, 1H), 8.48 (s, 1H), 8.06 (s, 1H), 7.67 (d, J=8.0 Hz, 1H), 7.45 (d, J=8.2 Hz, 1H), 7.27-7.17 (m, 1H), 7.12-7.04 (m, 1H), 6.99-6.92 (m, 1H), 6.67 (t, J=7.63 Hz, 1H), 5.62 (d, J=18.7 Hz, 1H), 5.35-5.23 (m, 1H), 5.04-4.55 (m, 1H), 4.42-4.25 (m, 1H), 4.16 (d, J=12.8 Hz, 1H), 4.02-3.85 (m, 2H), 1.23 (d, J=6.8 Hz, 3H), 0.92-0.71 (m, 4H).

Stereochemically pure material was obtained by separation of the racemate (Example 130) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 132

### N-{1-[(difluoromethoxy)methyl]cyclopropyl}-5-(1H-indole-2-carbonyl)-6-methyl-4H,5H,6H,7H-pyrazolo[1,5-a]pyrazine-3-carboxamide (Enantiomer 2, Absolute Configuration Unknown)

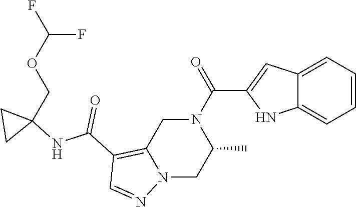 [SMILES: C[C@@H]1Cn2ncc(C(=O)NC3(COC(F)F)CC3)c2CN1C(=O)c1cc2ccccc2[nH]1]

Step 1: To a solution of 5-(tert-butoxycarbonyl)-6-methyl-4,5,6,7-tetrahydropyrazolo[1,5-a]pyrazine-3-carboxylic acid (100 mg, 0.355 mmol) in DMF (3 mL) was added triethylamine (0.248 mL, 1.777 mmol), followed by HATU (149 mg, 0.391 mmol). After 10 min, 1-((difluoromethoxy)methyl)cyclopropan-1-amine hydrochloride (73.2 mg, 0.422 mmol) was added and the mixture was stirred overnight. The reaction mixture was partitioned between EtAOc (20 mL) and brine (20 mL). Some solid NaCl and some brine were added to help phase separation. The aqueous layer was extracted with EtOAc (10 mL). The combined organic layers were washed with brine (4×15 mL), dried over Na₂SO₄ and concentrated. The residue was dissolved in 1 ml DCM and purified by column chromatography to give tert-butyl 3-({1-[(difluoromethoxy)methyl]cyclopropyl}carbamoyl)-6-methyl-4H,5H,6H,7H-pyrazolo[1,5-a]pyrazine-5-carboxylate (0.111 g, 78% yield).

Step 2: To tert-butyl 3-((1-((difluoromethoxy)methyl)cyclopropyl)carbamoyl)-6-methyl-6,7-dihydropyrazolo[1,5-a]pyrazine-5(4H)-carboxylate (27.8 mg, 0.069 mmol) was added HCl (4 M in dioxane) (0.5 mL, 2 mmol). The mixture was stirred for 2 h then concentrated and stripped with DCM to give N-{1-[(difluoromethoxy)methyl]cyclopropyl}-6-methyl-4H,5H,6H,7H-pyrazolo[1,5-a]pyrazine-3-carboxamide hydrochloride as a white solid that was used in the next step without further purification.

Step 3: To a solution of indole-2-carboxylic acid (11.20 mg, 0.069 mmol) in DMSO was added HATU (29.1 mg, 0.076 mmol). The mixture was stirred for 10 min. In a separate vial, N-(1-((difluoromethoxy)methyl)cyclopropyl)-6-methyl-4,5,6,7-tetrahydropyrazolo[1,5-a]pyrazine-3-carboxamide hydrochloride (23.4 mg, 0.069 mmol) was dissolved in DMSO and triethylamine (48.4 μL, 0.347 mmol) was added. The mixtures were combined and stirred overnight. The mixture was purified directly by reverse phase chromatography to give the product as a white solid (0.0199 g, 64% yield).

Rt (Method B2) 3.44 mins, m/z 444 [M+H]+

¹H NMR (400 MHz, DMSO-d6) δ 11.69 (s, 1H), 8.48 (s, 1H), 8.06 (s, 1H), 7.67 (d, J=7.9 Hz, 1H), 7.49-7.41 (m, 1H), 7.27-7.17 (m, 1H), 7.12-7.04 (m, 1H), 6.99-6.92 (m, 1H), 6.67 (t, J=76.3 Hz, 1H), 5.62 (d, J=18.6 Hz, 1H), 5.35-5.20 (m, 1H), 4.97-4.58 (m, 1H), 4.40-4.26 (m, 1H), 4.16 (d, J=13.0 Hz, 1H), 4.01-3.86 (m, 2H), 1.23 (d, J=6.8 Hz, 3H), 0.91-0.66 (m, 4H).

Stereochemically pure material was obtained by separation of the racemate (Example 130) by chiral SFC, using a Phenomenex Cellulose-1 column (250×21.2 mm, 5 μm), flow rate 70 mL/min, column temperature 35° C., 170 bar. Eluent A—CO₂, Eluent B—methanol/20 mM ammonia, linear elution gradient t=0 mins 10% B, t=6.5 mins 40% B, t=8 mins, 40% B.

### Example 133

### N-cyclobutyl-5-(1H-indole-2-carbonyl)-N-methyl-4H,5H,6H,7H-pyrazolo[1,5-a]pyrazine-3-carboxamide

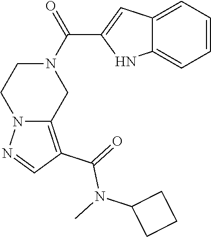 [SMILES: CN(C(=O)c1cnn2c1CN(C(=O)c1cc3ccccc3[nH]1)CC2)C1CCC1]

Rt (Method A2) 3.31 mins, m/z 378 [M+H]+

¹H NMR (400 MHz, DMSO-d6) δ 11.71 (s, 1H), 7.73 (s, 1H), 7.65 (d, J=7.9 Hz, 1H), 7.44 (d, J=8.2 Hz, 1H), 7.25-7.17 (m, 1H), 7.11-7.03 (m, 1H), 6.95 (s, 1H), 5.30-4.88 (m, 2H), 4.77-4.64 (m, 1H), 4.43-4.13 (m, 4H), 2.98 (s, 3H), 2.30-2.15 (m, 2H), 2.13-2.01 (m, 2H), 1.69-1.49 (m, 2H).

### Example 134

### 5-(1H-indole-2-carbonyl)-N-(oxetan-3-yl)-4H,5H,6H,7H-pyrazolo[1,5-a]pyrazine-3-carboxamide

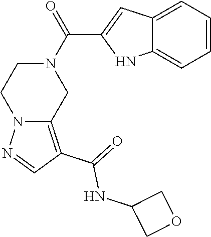 [SMILES: O=C(NC1COC1)c1cnn2c1CN(C(=O)c1cc3ccccc3[nH]1)CC2]

Rt (Method A2) 2.80 mins, m/z 366 [M+H]+

¹H NMR (400 MHz, DMSO-d6) δ 11.71 (s, 1H), 8.76 (d, J=6.7 Hz, 1H), 8.07 (s, 1H), 7.66 (d, J=7.9 Hz, 1H), 7.44 (d, J=8.2 Hz, 1H), 7.22 (t, J=7.6 Hz, 1H), 7.07 (t, J=7.5 Hz, 1H), 6.95 (s, 1H), 5.41-5.03 (m, 2H), 5.00-4.88 (m, 1H), 4.74 (t, J=6.9 Hz, 2H), 4.53 (t, J=6.4 Hz, 2H), 4.37-4.14 (m, 4H).

### Example 135

### 5-(1H-indole-2-carbonyl)-N-methyl-N-(oxetan-3-yl)-4H,5H,6H,7H-pyrazolo[1,5-a]pyrazine-3-carboxamide

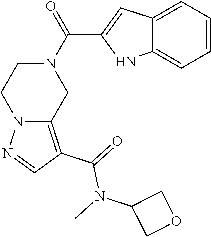 [SMILES: CN(C(=O)c1cnn2c1CN(C(=O)c1cc3ccccc3[nH]1)CC2)C1COC1]

Rt (Method A2) 2.82 mins, m/z 380 [M+H]+

¹H NMR (400 MHz, DMSO-d6) δ 11.71 (s, 1H), 7.79 (s, 1H), 7.65 (d, J=8.0 Hz, 1H), 7.44 (d, J=8.2 Hz, 1H), 7.22 (t, J=7.5 Hz, 1H), 7.07 (t, J=7.5 Hz, 1H), 6.95 (s, 1H), 5.30-4.93 (m, 3H), 4.76-4.60 (m, 4H), 4.36-4.18 (m, 4H), 3.16 (s, 3H).

### Example 136

### 5-(1H-indole-2-carbonyl)-N-methyl-N-(3-methyloxetan-3-yl)-4H,5H,6H,7H-pyrazolo[1,5-a]pyrazine-3-carboxamide

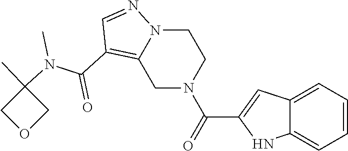 [SMILES: CN(C(=O)c1cnn2c1CN(C(=O)c1cc3ccccc3[nH]1)CC2)C1(C)COC1]

Rt (Method A2) 2.95 mins, m/z 394 [M+H]+

¹H NMR (400 MHz, DMSO-d6) δ 11.71 (s, 1H), 7.89 (s, 1H), 7.65 (d, J=7.9 Hz, 1H), 7.44 (d, J=8.3 Hz, 1H), 7.28-7.13 (m, 1H), 7.12-6.80 (m, 2H), 5.36-4.85 (m, 2H), 4.72-4.49 (m, 2H), 4.48-3.94 (m, 6H), 2.97 (s, 3H), 1.57 (s, 3H).

### Example 137

### N-[(1-hydroxycyclobutyl)methyl]-5-(1H-indole-2-carbonyl)-N-methyl-4H,5H,6H,7H-pyrazolo[1,5-a]pyrazine-3-carboxamide

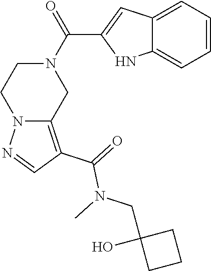 [SMILES: CN(CC1(O)CCC1)C(=O)c1cnn2c1CN(C(=O)c1cc3ccccc3[nH]1)CC2]

Rt (Method A2) 3.00 mins, m/z 408 [M+H]+

¹H NMR (400 MHz, DMSO-d6) δ 11.70 (s, 1H), 7.84 (s, 1H), 7.65 (d, J=7.9 Hz, 1H), 7.44 (d, J=8.2 Hz, 1H), 7.25-7.18 (m, 1H), 7.11-7.03 (m, 1H), 6.95 (s, 1H), 5.34-4.93 (m, 3H), 4.38-4.16 (m, 4H), 3.60 (s, 2H), 3.30-2.87 (m, 3H), 2.01-1.81 (m, 4H), 1.67-1.21 (m, 2H).

### Example 138

### N-[1-(1,3-dioxolan-2-yl)cyclopropyl]-5-(1H-indole-2-carbonyl)-N-methyl-4H,5H,6H,7H-pyrazolo[1,5-a]pyrazine-3-carboxamide

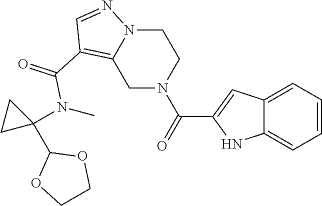 [SMILES: CN(C(=O)c1cnn2c1CN(C(=O)c1cc3ccccc3[nH]1)CC2)C1(C2OCCO2)CC1]

Rt (Method A2) 3.22 mins, m/z 436 [M+H]+

¹H NMR (400 MHz, DMSO-d6) δ 11.70 (s, 1H), 7.94 (s, 1H), 7.66 (d, J=8.0 Hz, 1H), 7.44 (d, J=8.2 Hz, 1H), 7.21 (t, J=7.6 Hz, 1H), 7.07 (t, J=7.4 Hz, 1H), 6.95 (s, 1H), 5.42-4.73 (m, 3H), 4.44-4.03 (m, 4H), 4.02-3.70 (m, 4H), 3.21-2.84 (m, 3H), 1.30-0.72 (m, 4H).

### Example 139

### 5-(5,6-difluoro-1H-indole-2-carbonyl)-N-{1-[(difluoromethoxy)methyl]cyclopropyl}-4H,5H,6H,7H-pyrazolo[1,5-a]pyrazine-3-carboxamide

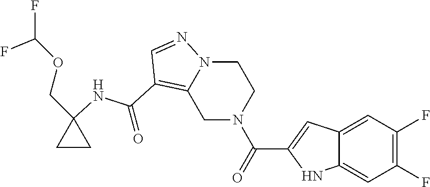 [SMILES: O=C(NC1(COC(F)F)CC1)c1cnn2c1CN(C(=O)c1cc3cc(F)c(F)cc3[nH]1)CC2]

To a solution of 5,6-difluoroindole-2-carboxylic acid (0.0248 g, 0.126 mmol) in DMF (0.4 mL) was added HATU (0.050 g, 0.132 mmol) and NEt₃ (0.088 mL, 0.629 mmol). The mixture was stirred for 30 mins, then a solution of N-(1-((difluoromethoxy)methyl)cyclopropyl)-4,5,6,7-tetrahydropyrazolo[1,5-a]pyrazine-3-carboxamide (0.036 g, 0.126 mmol) in dry DMF (0.4 mL) was added. The mixture was stirred overnight, then filtered, rinsing with methanol (0.2 mL). The mixture was then purified directly by HPLC to give 5-(5,6-difluoro-1H-indole-2-carbonyl)-N-{1-[(difluoromethoxy)methyl]cyclopropyl}-4H,5H,6H,7H-pyrazolo[1,5-a]pyrazine-3-carboxamide as a white powder (0.027 g, 46% yield).

Rt (Method A2) 3.46 mins, m/z 466 [M+H]+

¹H NMR (400 MHz, DMSO-d6) δ 11.93 (s, 1H), 8.49 (s, 1H), 8.05 (s, 1H), 7.79-7.62 (m, 1H), 7.39 (dd, J=11.0, 7.0 Hz, 1H), 6.99 (s, 1H), 6.68 (t, J=76.3 Hz, 1H), 5.59-4.95 (m, 2H), 4.39-4.11 (m, 4H), 3.94 (s, 2H), 0.97-0.67 (m, 4H).

### Example 140

### 4-chloro-2-[3-(3,3-difluoropyrrolidine-1-carbonyl)-4H,5H,6H,7H-pyrazolo[1,5-a]pyrazine-5-carbonyl]-6-fluoro-1H-indole

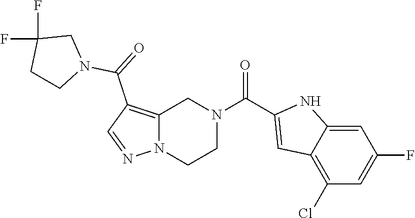 [SMILES: O=C(c1cc2c(Cl)cc(F)cc2[nH]1)N1CCn2ncc(C(=O)N3CCC(F)(F)C3)c2C1]

Step 1: 5-(tert-butoxycarbonyl)-4,5,6,7-tetrahydropyrazolo[1,5-a]pyrazine-3-carboxylic acid (160 mg, 0.599 mmol) was suspended in dry DMF (3 mL) and HATU (273 mg, 0.718 mmol) was added. 3,3-difluoropyrrolidine hydrochloride (86 mg, 0.599 mmol) was added, followed by triethylamine (0.417 ml, 2.99 mmol). The mixture was stirred for 30 mins, then the suspension was filtered and purified directly by reversed phase column chromatography. The residue was stripped with toluene and DCM to yield tert-butyl 3-(3,3-difluoropyrrolidine-1-carbonyl)-4H,5H,6H,7H-pyrazolo[1,5-a]pyrazine-5-carboxylate as a white solid (0.195 g, 91% yield).

Step 2: To tert-butyl 3-(3,3-difluoropyrrolidine-1-carbonyl)-6,7-dihydropyrazolo[1,5-a]pyrazine-5(4H)-carboxylate (195 mg, 0.547 mmol) was added HCl (4 M in dioxane) (5 mL, 20.00 mmol). The suspension was stirred for 40 min. The reaction mixture was concentrated and stripped with toluene and DCM to yield 3,3-difluoro-1-{4H,5H,6H,7H-pyrazolo[1,5-a]pyrazine-3-carbonyl}pyrrolidine as a white solid (162 mg, 100% yield) that was used in the next step without further purification.

Step 3: To a solution of 4-chloro-6-fluoro-1H-indole-2-carboxylic acid (21.89 mg, 0.102 mmol) in dry DMF (0.4 mL) was added HATU (46.8 mg, 0.123 mmol). The solution was stirred for 10 minutes. A mixture of (3,3-difluoropyrrolidin-1-yl)(4,5,6,7-tetrahydropyrazolo[1,5-a]pyrazin-3-yl)methanone hydrochloride (10 mg, 0.102 mmol), triethylamine (0.071 mL, 0.512 mmol) and a drop of water in dry DMF (1 mL) was then added to the activated acid and the resulting solution was stirred overnight. The suspension was filtered and the filter was rinsed with DMSO. The filtrate was purified by reverse phase chromatography to give the product as a white solid (0.0256 g, 55% yield).

Rt (Method A) 3.33 mins, m/z 452/454 [M+H]+

¹H NMR (400 MHz, DMSO-d6) δ 12.17 (s, 1H), 7.95 (s, 1H), 7.22-7.16 (m, 2H), 6.96 (s, 1H), 5.43-4.98 (m, 2H), 4.46-4.04 (m, 5H), 4.04-3.55 (m, 3H), 2.48-2.35 (m, 2H).

### Example 141

### 5-(1H-indole-2-carbonyl)-N-methyl-N-{1-[(2r,5r)-5-amino-1,3-dioxan-2-yl]cyclopropyl}-4H,5H,6H,7H-pyrazolo[1,5-a]pyrazine-3-carboxamide

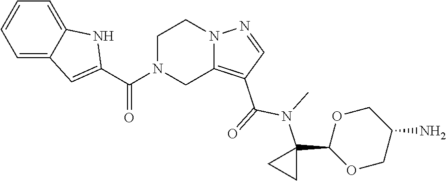 [SMILES: CN(C(=O)c1cnn2c1CN(C(=O)c1cc3ccccc3[nH]1)CC2)C1([C@H]2OC[C@H](N)CO2)CC1]

Step 1: To a solution of 5-(1H-indole-2-carbonyl)-4,5,6,7-tetrahydropyrazolo[1,5-a]pyrazine-3-carboxylic acid (49.6 mg, 0.160 mmol) in dry DMF (0.5 mL) was added HATU (60.7 mg, 0.160 mmol). The resulting suspension was stirred at r.t. for 30 min. Then, N Et₃ (0.051 mL, 0.367 mmol) was added, followed by a suspension of 2-((2r,5r)-2-(1-(methylamino)cyclopropyl)-1,3-dioxan-5-yl)isoindoline-1,3-dione (48.3 mg, 0.160 mmol) in dry DMF (0.6 mL). The mixture was stirred overnight, then filtered through a microfilter and purified directly by HPLC to give 5-(1H-indole-2-carbonyl)-N-methyl-N-{1-[(2r,5r)-5-(1,3-dioxo-2,3-dihydro-1H-isoindol-2-yl)-1,3-dioxan-2-yl]cyclopropyl}-4H,5H,6H,7H-pyrazolo[1,5-a]pyrazine-3-carboxamide (0.046 g. 48% yield).

Step 2: To a solution of N-(1-((2r,5r)-5-(1,3-dioxoisoindolin-2-yl)-1,3-dioxan-2-yl)cyclopropyl)-5-(1H-indole-2-carbonyl)-N-methyl-4,5,6,7-tetrahydropyrazolo[1,5-a]pyrazine-3-carboxamide (46 mg, 0.077 mmol) in absolute ethanol (2 mL) was added hydrazine monohydrate (10 μL, 0.205 mmol). The suspension was stirred at 40° C. for 2 h, 50° C. for 6 h, then 60° C. overnight. Further hydrazine monohydrate (9.98 μL, 0.205 mmol) was added and stirring was continued at 60° C. The reaction mixture was concentrated and the residue was co-evaporated with EtOH (2×10 mL). The resulting off-white solids were suspended in DCM (15 mL) and stirred for 15 mins, after which the precipitate was filtered off and washed with DCM (15 mL). The filtrate was concentrated, dissolved in DMSO (1.5 mL), filtered through a micro filter and purified by HPLC to give the product as a white powder (0.005 g, 14% yield).

Rt (Method A2) 2.87 mins, m/z 465 [M+H]+

¹H NMR (400 MHz, DMSO-d6) δ 11.70 (s, 1H), 7.86 (s, 1H), 7.66 (d, J=8.1 Hz, 1H), 7.44 (d, J=8.2 Hz, 1H), 7.22 (t, J=7.6 Hz, 1H), 7.07 (t, J=7.5 Hz, 1H), 6.95 (s, 1H), 5.41-4.93 (m, 2H), 4.94-4.62 (m, 1H), 4.51-4.07 (m, 4H), 4.07-3.82 (m, 2H), 3.11-2.87 (m, 3H), 2.85-2.68 (m, 1H), 1.73-0.52 (m, 6H).

### Example 142

### 5-(1H-indole-2-carbonyl)-6-methyl-N-[(2R)-1,1,1-trifluoropropan-2-yl]-4H,5H,6H,7H-pyrazolo[1,5-a]pyrazine-3-carboxamide

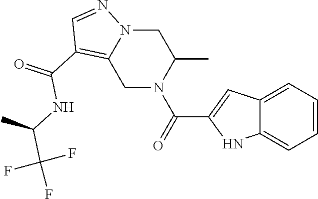 [SMILES: CC1Cn2ncc(C(=O)N[C@H](C)C(F)(F)F)c2CN1C(=O)c1cc2ccccc2[nH]1]

Rt (Method B2) 3.56 mins, m/z 420 [M+H]+

¹H NMR (400 MHz, DMSO-d6) δ 11.70 (s, 1H), 8.49 (t, J=8.1 Hz, 1H), 8.17 (s, 1H), 7.66 (d, J=8.1 Hz, 1H), 7.45 (d, J=8.2 Hz, 1H), 7.22 (t, J=7.5 Hz, 1H), 7.08 (t, J=7.4 Hz, 1H), 6.95 (s, 1H), 5.63 (d, J=18.7 Hz, 1H), 5.35-5.25 (m, 1H), 4.91-4.69 (m, 2H), 4.41-4.30 (m, 1H), 4.23-4.15 (m, 1H), 1.37-1.28 (m, 3H), 1.28-1.18 (m, 3H).

### Example 143

### 5-(4,5-difluoro-1H-indole-2-carbonyl)-6-methyl-N-[(2R)-1,1,1-trifluoropropan-2-yl]-4H,5H,6H,7H-pyrazolo[1,5-a]pyrazine-3-carboxamide

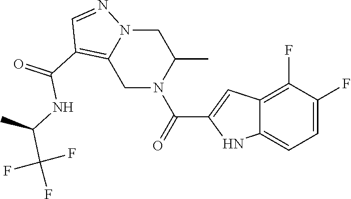 [SMILES: CC1Cn2ncc(C(=O)N[C@H](C)C(F)(F)F)c2CN1C(=O)c1cc2c(F)c(F)ccc2[nH]1]

Rt (Method B2) 3.73 mins, m/z 456 [M+H]+

¹H NMR (400 MHz, DMSO-d6) δ 12.12 (s, 1H), 8.52-8.46 (m, 1H), 8.17 (s, 1H), 7.31-7.21 (m, 2H), 7.08-7.04 (m, 1H), 5.59 (dd, J=18.6, 5.2 Hz, 1H), 5.32-5.22 (m, 1H), 4.97-4.65 (m, 2H), 4.43-4.31 (m, 1H), 4.22-4.14 (m, 1H), 1.36-1.28 (m, 3H), 1.27-1.19 (m, 3H).

### Example 144

### 5-(5-fluoro-4-methyl-1H-indole-2-carbonyl)-6-methyl-N-[(2R)-1,1,1-trifluoropropan-2-yl]-4H,5H,6H,7H-pyrazolo[1,5-a]pyrazine-3-carboxamide

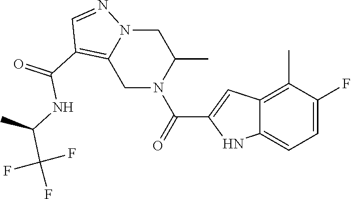 [SMILES: Cc1c(F)ccc2[nH]c(C(=O)N3Cc4c(C(=O)N[C@H](C)C(F)(F)F)cnn4CC3C)cc12]

Rt (Method B2) 3.74 mins, m/z 452 [M+H]+

¹H NMR (400 MHz, DMSO-d6) δ 11.75 (s, 1H), 8.52-8.43 (m, 1H), 8.17 (s, 1H), 7.26 (dd, J=8.7, 4.2 Hz, 1H), 7.07-6.98 (m, 2H), 5.60 (dd, J=18.8, 7.6 Hz, 1H), 5.28 (p, J=8.2, 7.3 Hz, 1H), 4.87-4.66 (m, 2H), 4.45-4.33 (m, 1H), 4.23-4.14 (m, 1H), 2.45-2.39 (m, 3H), 1.36-1.29 (m, 3H), 1.28-1.21 (m, 3H).

### Example 145

### 5-(4-chloro-1H-indole-2-carbonyl)-6-methyl-N-[(2R)-1,1,1-trifluoropropan-2-yl]-4H,5H,6H,7H-pyrazolo[1,5-a]pyrazine-3-carboxamide

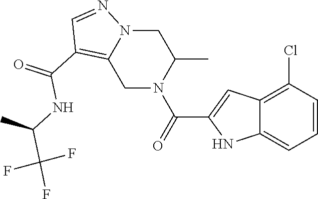 [SMILES: CC1Cn2ncc(C(=O)N[C@H](C)C(F)(F)F)c2CN1C(=O)c1cc2c(Cl)cccc2[nH]1]

Rt (Method B2) 3.79 mins, m/z 454/456 [M+H]+

¹H NMR (400 MHz, DMSO-d6) δ 12.08 (s, 1H), 8.54-8.43 (m, 1H), 8.17 (s, 1H), 7.43 (d, J=7.9 Hz, 1H), 7.25-7.14 (m, 2H), 6.96-6.93 (m, 1H), 5.60 (dd, J=18.6, 7.1 Hz, 1H), 5.33-5.21 (m, 1H), 4.93-4.71 (m, 2H), 4.44-4.30 (m, 1H), 4.24-4.15 (m, 1H), 1.36-1.29 (m, 3H), 1.28-1.20 (m, 3H).

### Example 146

### 5-(6-fluoro-4-methyl-1H-indole-2-carbonyl)-6-methyl-N-[(2R)-1,1,1-trifluoropropan-2-yl]-4H,5H,6H,7H-pyrazolo[1,5-a]pyrazine-3-carboxamide

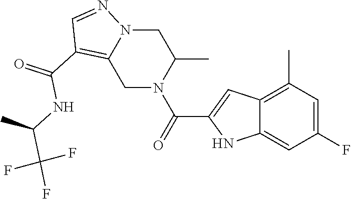 [SMILES: Cc1cc(F)cc2[nH]c(C(=O)N3Cc4c(C(=O)N[C@H](C)C(F)(F)F)cnn4CC3C)cc12]

Rt (Method B2) 3.78 mins, m/z 452 [M+H]+

¹H NMR (400 MHz, DMSO-d6) δ 11.74 (s, 1H), 8.52-8.44 (m, 1H), 8.17 (s, 1H), 7.06-6.92 (m, 2H), 6.78 (d, J=10.6 Hz, 1H), 5.61 (dd, J=18.8, 7.2 Hz, 1H), 5.29 (p. J=6.9, 6.1 Hz, 1H), 4.90-4.68 (m, 2H), 4.45-4.32 (m, 1H), 4.23-4.15 (m, 1H), 2.52 (s, 3H), 1.37-1.29 (m, 3H), 1.28-1.20 (m, 3H).

### Example 147

### 5-(6-chloro-5-fluoro-1H-indole-2-carbonyl)-6-methyl-N-[(2R)-1,1,1-trifluoropropan-2-yl]-4H,5H,6H,7H-pyrazolo[1,5-a]pyrazine-3-carboxamide

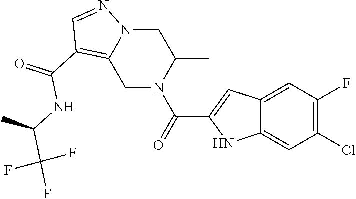 [SMILES: CC1Cn2ncc(C(=O)N[C@H](C)C(F)(F)F)c2CN1C(=O)c1cc2cc(F)c(Cl)cc2[nH]1]

Rt (Method B2) 3.85 mins, m/z 472/474 [M+H]+

¹H NMR (400 MHz, DMSO-d6) δ 11.93 (s, 1H), 8.53-8.45 (m, 1H), 8.17 (s, 1H), 7.68 (d, J=9.9 Hz, 1H), 7.57 (d, J=6.4 Hz, 1H), 6.97 (s, 1H), 5.64-5.53 (m, 1H), 5.31-5.20 (m, 1H), 4.93-4.67 (m, 2H), 4.41-4.27 (m, 1H), 4.23-4.14 (m, 1H), 1.37-1.28 (m, 3H), 1.27-1.18 (m, 3H).

### Example 148

### N-{3-[2-(difluoromethoxy)ethyl]oxetan-3-yl}-5-(1H-indole-2-carbonyl)-4H,5H,6H,7H-pyrazolo[1,5-a]pyrazine-3-carboxamide

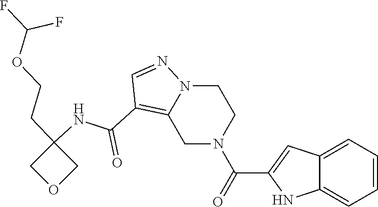 [SMILES: O=C(NC1(CCOC(F)F)COC1)c1cnn2c1CN(C(=O)c1cc3ccccc3[nH]1)CC2]

Rt (Method A2) 3.20 mins, m/z 460 [M+H]+

### Biochemical Capsid Assembly Assay

The screening for assembly effector activity was done based on a fluorescence quenching assay published by Zotnick et al. (2007). The C-terminal truncated core protein containing 149 amino acids of the N-terminal assembly domain fused to a unique cysteine residue at position 150 and was expressed in E. coli using the pET expression system (Merck Chemicals, Darmstadt). Purification of core dimer protein was performed using a sequence of size exclusion chromatography steps. In brief, the cell pellet from 1 L BL21 (DE3) Rosetta2 culture expressing the coding sequence of core protein cloned NdeI/XhoI into expression plasmid pET21b was treated for 1 h on ice with a native lysis buffer (Qproteome Bacterial Protein Prep Kit; Qiagen, Hilden). After a centrifugation step the supernatant was precipitated during 2 h stirring on ice with 0.23 g/ml of solid ammonium sulfate. Following further centrifugation the resulting pellet was resolved in buffer A (100 mM Tris, pH 7.5; 100 mM NaCl; 2 mM DTT) and was subsequently loaded onto a buffer A equilibrated CaptoCore 700 column (GE HealthCare, Frankfurt). The column flow through containing the assembled HBV capsid was dialyzed against buffer N (50 mM NaHCO3 pH 9.6; 5 mM DTT) before urea was added to a final concentration of 3M to dissociate the capsid into core dimers for 1.5 h on ice. The protein solution was then loaded onto a IL Sephacryl S300 column. After elution with buffer N core dimer containing fractions were identified by SDS-PAGE and subsequently pooled and dialyzed against 50 mM HEPES pH 7.5; 5 mM DTT. To improve the assembly capacity of the purified core dimers a second round of assembly and disassembly starting with the addition of 5 M NaCl and including the size exclusion chromatography steps described above was performed. From the last chromatography step core dimer containing fractions were pooled and stored in aliquots at concentrations between 1.5 to 2.0 mg/ml at −80° C.

Immediately before labelling the core protein was reduced by adding freshly prepared DTT in a final concentration of 20 mM. After 40 min incubation on ice storage buffer and DTT was removed using a Sephadex G-25 column (GE HealthCare, Frankfurt) and 50 mM HEPES, pH 7.5. For labelling 1.6 mg/ml core protein was incubated at 4° C. and darkness overnight with BODIPY-FL maleimide (Invitrogen, Karlsruhe) in a final concentration of 1 mM. After labelling the free dye was removed by an additional desalting step using a Sephadex G-25 column. Labelled core dimers were stored in aliquots at 4° C. In the dimeric state the fluorescence signal of the labelled core protein is high and is quenched during the assembly of the core dimers to high molecular capsid structures. The screening assay was performed in black 384 well microtiter plates in a total assay volume of 10 μl using 50 mM HEPES pH 7.5 and 1.0 to 2.0 μM labelled core protein. Each screening compound was added in 8 different concentrations using a 0.5 log-unit serial dilution starting at a final concentration of 100 μM, 31.6 μM or 10 μM, In any case the DMSO concentration over the entire microtiter plate was 0.5%. The assembly reaction was started by the injection of NaCl to a final concentration of 300 μM which induces the assembly process to approximately 25% of the maximal quenched signal. 6 min after starting the reaction the fluorescence signal was measured using a Clariostar plate reader (BMG Labtech, Ortenberg) with an excitation of 477 nm and an emission of 525 nm. As 100% and 0% assembly control HEPES buffer containing 2.5 M and 0 M NaCl was used. Experiments were performed thrice in triplicates. EC₅₀ values were calculated by non-linear regression analysis using the Graph Pad Prism 6 software (GraphPad Software, La Jolla, USA).

Determination of HBV DNA from the Supernatants of HepAD38 Cells

The anti-HBV activity was analysed in the stable transfected cell line HepAD38, which has been described to secrete high levels of HBV virion particles (Ladner et al., 1997). In brief, HepAD38 cells were cultured at 37° C. at 5% CO₂ and 95% humidity in 200 μl maintenance medium, which was Dulbecco's modified Eagle's medium/Nutrient Mixture F-12 (Gibco, Karlsruhe), 10% fetal bovine serum (PAN Biotech Aidenbach) supplemented with 50 μg/ml penicillin/streptomycin (Gibco, Karlsruhe), 2 mM L-glutamine (PAN Biotech, Aidenbach), 400 μg/ml G418 (AppliChem, Darmstadt) and 0.3 μg/ml tetracycline. Cells were subcultured once a week in a 1:5 ratio, but were usually not passaged more than ten times. For the assay 60,000 cells were seeded in maintenance medium without any tetracycline into each well of a 96-well plate and treated with serial half-log dilutions of test compound. To minimize edge effects the outer 36 wells of the plate were not used but were filled with assay medium. On each assay plate six wells for the virus control (untreated HepAD38 cells) and six wells for the cell control (HepAD38 cells treated with 0.3 μg/ml tetracycline) were allocated, respectively. In addition, one plate set with reference inhibitors like BAY 41-4109, entecavir, and lamivudine instead of screening compounds were prepared in each experiment. In general, experiments were performed thrice in triplicates. At day 6 HBV DNA from 100 μl filtrated cell culture supernatant (AcroPrep Advance 96 Filter Plate, 0.45 μM Supor membran, PALL GmbH, Dreieich) was automatically purified on the MagNa Pure LC instrument using the MagNA Pure 96 DNA and Viral NA Small Volume Kit (Roche Diagnostics, Mannheim) according to the instructions of the manufacturer. EC50 values were calculated from relative copy numbers of HBV DNA In brief, 5 μl of the 100 μl eluate containing HBV DNA were subjected to PCR LC480 Probes Master Kit (Roche) together with 1 μM antisense primer tgcagaggtgaagcgaagtgcaca, 0.5 μM sense primer gacgtcctttgtttacgtcccgtc, 0.3 μM hybprobes acggggcgcacctctctttacgcgg-FL and LC640-ctccccgtctgtgccttctcatctgc-PH (TIBMolBiol, Berlin) to a final volume of 12.5 μl. The PCR was performed on the Light Cycler 480 real time system (Roche Diagnostics, Mannheim) using the following protocol: Pre-incubation for 1 min at 95° C., amplification: 40 cycles×(10 sec at 95° C., 50 sec at 60° C., 1 sec at 70° C.), cooling for 10 sec at 40° C. Viral load was quantitated against known standards using HBV plasmid DNA of pCH-9/3091 (Nassal et al., 1990, Cell 63: 1357-1363) and the LightCycler 480 SW 1.5 software (Roche Diagnostics, Mannheim) and ECso values were calculated using non-linear regression with GraphPad Prism 6 (GraphPad Software Inc., La Jolla, USA).

### Cell Viability Assay

Using the AlamarBlue viability assay cytotoxicity was evaluated in HepAD38 cells in the presence of 0.3 μg/ml tetracycline, which blocks the expression of the HBV genome. Assay condition and plate layout were in analogy to the anti-HBV assay, however other controls were used. On each assay plate six wells containing untreated HepAD38 cells were used as the 100% viability control, and six wells filled with assay medium only were used as 0% viability control. In addition, a geometric concentration series of cycloheximide starting at 60 μM final assay concentration was used as positive control in each experiment. After six days incubation period Alamar Blue Presto cell viability reagent (ThermoFisher, Dreieich) was added in 1/11 dilution to each well of the assay plate. After an incubation for 30 to 45 min at 37° C. the fluorescence signal, which is proportional to the number of living cells, was read using a Tecan Spectrafluor Plus plate reader with an excitation filter 550 nm and emission filter 595 nm, respectively. Data were normalized into percentages of the untreated control (100% viability) and assay medium (0% viability) before CC50 values were calculated using non-linear regression and the GraphPad Prism 6.0 (GraphPad Software, La Jolla, USA). Mean EC₅₀ and CC₅₀ values were used to calculate the selectivity index (SI=CC₅₀/EC₅₀) for each test compound.

### In Vivo Efficacy Models

HBV research and preclinical testing of antiviral agents are limited by the narrow species- and tissue-tropism of the virus, the paucity of infection models available and the restrictions imposed by the use of chimpanzees, the only animals fully susceptible to HBV infection. Alternative animal models are based on the use of HBV-related hepadnaviruses and various antiviral compounds have been tested in woodchuck hepatitis virus (WHV) infected woodchucks or in duck hepatitis B virus (DHBV) infected ducks or in woolly monkey HBV (WM-HBV) infected tupaia (overview in Dandri et al., 2017, Best Pract Res Clin Gastroenterol 31, 273-279). However, the use of surrogate viruses has several limitations. For example is the sequence homology between the most distantly related DHBV and HBV is only about 40% and that is why core protein assembly modifiers of the HAP family appeared inactive on DHBV and WHV but efficiently suppressed HBV (Campagna et al., 2013, J. Virol. 87, 6931-6942). Mice are not HBV permissive but major efforts have focused on the development of mouse models of HBV replication and infection, such as the generation of mice transgenic for the human HBV (HBV tg mice), the hydrodynamic injection (HDI) of HBV genomes in mice or the generation of mice having humanized livers and/or humanized immune systems and the intravenous injection of viral vectors based on adenoviruses containing HBV genomes (Ad-HBV) or the adenoassociated virus (AAV-HBV) into immune competent mice (overview in Dandri et al., 2017, Best Pract Res Clin Gastroenterol 31, 273-279). Using mice transgenic for the full HBV genome the ability of murine hepatocytes to produce infectious HBV virions could be demonstrated (Guidotti et al., 1995, J. Virol., 69: 6158-6169). Since transgenic mice are immunological tolerant to viral proteins and no liver injury was observed in HBV-producing mice, these studies demonstrated that HBV itself is not cytopathic. HBV transgenic mice have been employed to test the efficacy of several anti-HBV agents like the polymerase inhibitors and core protein assembly modifiers (Weber et al., 2002, Antiviral Research 54 69-78; Julander et al., 2003, Antivir. Res., 59: 155-161), thus proving that HBV transgenic mice are well suitable for many type of preclinical antiviral testing in vivo.

As described in Paulsen et al., 2015, PLOSone, 10: e0144383 HBV-transgenic mice (Tg [HBV1.3 fsX⁻3′5′]) carrying a frameshift mutation (GC) at position 2916/2917 could be used to demonstrate antiviral activity of core protein assembly modifiers in vivo. In brief, The HBV-transgenic mice were checked for HBV-specific DNA in the serum by qPCR prior to the experiments (see section “Determination of HBV DNA from the supernatants of HepAD38 cells”). Each treatment group consisted of five male and five female animals approximately 10 weeks age with a titer of 10⁷-10⁸ virions per ml serum. Compounds were formulated as a suspension in a suitable vehicle such as 2% DMSO/98% tylose (0.5% Methylcellulose/99.5% PBS) or 50% PEG400 and administered per os to the animals one to three times/day for a 10 day period. The vehicle served as negative control, whereas 1 μg/kg entecavir in a suitable vehicle was the positive control. Blood was obtained by retro bulbar blood sampling using an Isoflurane Vaporizer. For collection of terminal heart puncture six hours after the last treatment blood or organs, mice were anaesthetized with isoflurane and subsequently sacrificed by CO₂ exposure. Retro bulbar (100-150 μl) and heart puncture (400-500 μl) blood samples were collected into a Microvette 300 LH or Microvette 500 LH, respectively, followed by separation of plasma via centrifugation (10 min, 2000 g, 4° C.). Liver tissue was taken and snap frozen in liquid N2. All samples were stored at −80° C. until further use. Viral DNA was extracted from 50 μl plasma or 25 mg liver tissue and eluted in 50 μl AE buffer (plasma) using the DNeasy 96 Blood & Tissue Kit (Qiagen, Hilden) or 320 μl AE buffer (liver tissue) using the DNeasy Tissue Kit (Qiagen, Hilden) according to the manufacturer's instructions. Eluted viral DNA was subjected to qPCR using the LightCycler 480 Probes Master PCR kit (Roche, Mannheim) according to the manufacturer's instructions to determine the HBV copy number. HBV specific primers used included the forward primer 5′-CTG TAC CAA ACC TTC GGA CGG-3′, the reverse primer 5′-AGG AGA AAC GGG CTG AGG C-3′ and the FAM labelled probe FAM-CCA TCA TCC TGG GCT TTC GGA AAA TT-BBQ. One PCR reaction sample with a total volume of 20 μl contained 5 μl DNA eluate and 15 μl master mix (comprising 0.3 μM of the forward primer, 0.3 μM of the reverse primer, 0.15 μM of the FAM labelled probe). qPCR was carried out on the Roche LightCycler1480 using the following protocol: Pre-incubation for 1 min at 95° C., amplification: (10 sec at 95° C., 50 sec at 60° C., 1 sec at 70° C.)×45 cycles, cooling for 10 sec at 40° C. Standard curves were generated as described above. All samples were tested in duplicate. The detection limit of the assay is ˜50 HBV DNA copies (using standards ranging from 250-2.5×107 copy numbers). Results are expressed as HBV DNA copies/10 μl plasma or HBV DNA copies/100 ng total liver DNA (normalized to negative control).

It has been shown in multiple studies that not only transgenic mice are a suitable model to proof the antiviral activity of new chemical entities in vivo the use of hydrodynamic injection of HBV genomes in mice as well as the use of immune deficient human liver chimeric mice infected with HBV positive patient serum have also frequently used to profile drugs targeting HBV (Li et al., 2016, Hepat. Mon. 16: e34420; Qiu et al., 2016, J. Med. Chem. 59: 7651-7666; Lutgehetmann et al., 2011, Gastroenterology, 140: 2074-2083). In addition chronic HBV infection has also been successfully established in immunecompetent mice by inoculating low doses of adenovirus-(Huang et al., 2012, Gastroenterology 142: 1447-1450) or adeno-associated virus (AAV) vectors containing the HBV genome (Dion et al., 2013, J Virol. 87: 5554-5563). This models could also be used to demonstrate the in vivo antiviral activity of novel anti-HBV agents.

TABLE 1
Biochemical and antiviral activities

| Example | CC₅₀ (μM) | Cell Activity | Assembly Activity |
|---|---|---|---|
| Example 1 | >10 | +++ | A |
| Example 2 | >10 | +++ | A |
| Example 3 | >10 | +++ | A |
| Example 4 | >10 | +++ | A |
| Example 5 | >10 | +++ | A |
| Example 6 | >10 | +++ | A |
| Example 7 | >10 | +++ | A |
| Example 8 | >10 | +++ | A |
| Example 9 | >10 | +++ | A |
| Example 10 | >10 | ++ | B |
| Example 11 | >10 | +++ | A |
| Example 12 | >10 | +++ | A |
| Example 13 | >10 | +++ | A |
| Example 14 | >10 | +++ | A |
| Example 15 | >10 | +++ | A |
| Example 16 | >10 | +++ | A |
| Example 17 | >10 | +++ | B |
| Example 18 | >10 | ++ | C |
| Example 19 | >10 | +++ | A |
| Example 20 | >10 | +++ | A |
| Example 21 | >10 | +++ | A |
| Example 22 | >10 | +++ | A |
| Example 23 | >10 | +++ | A |
| Example 24 | >10 | +++ | A |
| Example 25 | >10 | +++ | A |
| Example 26 | >10 | +++ | A |
| Example 27 | >10 | ++ | B |
| Example 28 | >10 | ++ | A |
| Example 29 | >10 | +++ | A |
| Example 30 | >10 | +++ | A |
| Example 31 | >10 | +++ | A |
| Example 32 | >10 | +++ | A |
| Example 33 | >10 | ++ | B |
| Example 34 | >10 | +++ | solubility |
| Example 35 | >10 | +++ | A |
| Example 36 | >10 | +++ | B |
| Example 37 | >10 | +++ | A |
| Example 38 | >10 | +++ | A |
| Example 39 | >10 | +++ | A |
| Example 40 | >10 | +++ | A |
| Example 41 | >10 | +++ | A |
| Example 42 | >10 | +++ | A |
| Example 43 | >10 | + | A |
| Example 44 | >10 | +++ | A |
| Example 45 | >10 | ++ | B |
| Example 46 | >10 | +++ | A |
| Example 47 | >10 | +++ | A |
| Example 48 | >10 | ++ | A |
| Example 49 | >10 | +++ | A |
| Example 50 | >10 | +++ | A |
| Example 51 | >10 | ++ | B |
| Example 52 | >10 | +++ | A |
| Example 53 | >10 | +++ | A |
| Example 54 | >10 | ++ | A |
| Example 55 | >10 | +++ | A |
| Example 56 | >10 | +++ | A |
| Example 57 | >10 | +++ | A |
| Example 58 | >10 | +++ | A |
| Example 59 | >10 | +++ | A |
| Example 60 | >10 | +++ | A |
| Example 61 | >10 | +++ | A |
| Example 62 | >10 | >10 | A |
| Example 63 | >10 | +++ | A |
| Example 64 | >10 | +++ | A |
| Example 65 | >10 | +++ | A |
| Example 66 | >10 | +++ | A |
| Example 67 | >10 | +++ | A |
| Example 68 | >10 | +++ | A |
| Example 69 | >10 | +++ | A |
| Example 70 | >10 | +++ | A |
| Example 71 | >10 | +++ | A |
| Example 72 | >10 | +++ | A |
| Example 73 | >10 | +++ | A |
| Example 74 | >10 | +++ | A |
| Example 75 | >10 | +++ | A |
| Example 76 | >10 | ++ | B |
| Example 77 | >10 | +++ | A |
| Example 78 | >10 | +++ | A |
| Example 79 | >10 | +++ | A |
| Example 80 | >10 | +++ | A |
| Example 81 | >10 | +++ | A |
| Example 82 | >10 | +++ | A |
| Example 83 | >10 | +++ | A |
| Example 84 | >10 | +++ | A |
| Example 85 | >10 | +++ | A |
| Example 86 | >10 | ++ | A |
| Example 87 | >10 | +++ | A |
| Example 88 | >10 | >10 | A |
| Example 89 | >10 | +++ | A |
| Example 90 | >10 | +++ | A |
| Example 91 | >10 | +++ | A |
| Example 92 | >10 | +++ | A |
| Example 93 | >10 | +++ | A |
| Example 94 | >10 | +++ | A |
| Example 95 | >10 | +++ | A |
| Example 96 | >10 | +++ | A |
| Example 97 | >10 | +++ | A |
| Example 98 | >10 | +++ | A |
| Example 99 | >10 | +++ | solubility |
| Example 100 | >10 | + | B |
| Example 101 | >10 | +++ | A |
| Example 102 | >10 | +++ | A |
| Example 103 | >10 | +++ | A |
| Example 104 | >10 | +++ | A |
| Example 105 | >10 | +++ | A |
| Example 106 | >10 | +++ | A |
| Example 107 | >10 | +++ | A |
| Example 108 | >10 | +++ | A |
| Example 109 | >10 | +++ | A |
| Example 110 | >10 | +++ | A |
| Example 111 | >10 | +++ | A |
| Example 112 | >10 | +++ | A |
| Example 113 | >10 | +++ | A |
| Example 114 | >10 | +++ | A |
| Example 115 | >10 | +++ | A |
| Example 116 | >10 | +++ | A |
| Example 117 | >10 | +++ | A |
| Example 118 | >10 | +++ | A |
| Example 119 | >10 | +++ | A |
| Example 120 | >10 | +++ | A |
| Example 121 | >10 | +++ | A |
| Example 122 | >10 | +++ | A |
| Example 123 | >10 | +++ | A |
| Example 124 | >10 | +++ | A |
| Example 125 | >10 | +++ | A |
| Example 126 | >10 | ++ | A |
| Example 127 | >10 | ++ | A |
| Example 128 | >10 | ++ | A |
| Example 129 | >10 | +++ | A |
| Example 130 | >10 | +++ | A |
| Example 131 | >10 | +++ | A |
| Example 132 | >10 | +++ | A |
| Example 133 | >10 | ++ | A |
| Example 134 | >10 | ++ | A |
| Example 135 | >10 | +++ | A |
| Example 136 | >10 | ++ | A |
| Example 137 | >10 | +++ | A |
| Example 138 | >10 | +++ | A |
| Example 139 | >10 | +++ | A |
| Example 140 | >10 | +++ | solubility |
| Example 141 | >10 | +++ | A |
| Example 142 | >10 | +++ | A |
| Example 143 | >10 | +++ | A |
| Example 144 | >10 | +++ | A |
| Example 145 | >10 | +++ | A |
| Example 146 | >10 | +++ | A |
| Example 147 | >10 | +++ | A |
| Example 148 | >10 | +++ | A |

In Table 1, “+++” represents an EC₅₀<1 μM; “++” represents 1 μM<EC₅₀<10 μM; “+” represents EC₅₀<100 μM (Cell activity assay)

In Table 1, “A” represents an IC₅₀<5 μM; “B” represents 5 μM<IC₅₀<10 μM; “C” represents IC₅₀<100 μM (Assembly assay activity)

In Table 1, “solubility” indicates that the compound was insufficiently soluble in the assay buffer to determine an IC₅₀.

1. A compound of Formula I 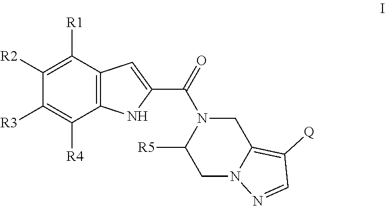
in which
R1, R2, R3 and R4 are for each position independently selected from the group comprising H, CF₂H, CF₃, CF₂CH₃, F, Cl, Br, CH₃, ethyl, iso-propyl, cyclopropyl, CH₂OH, CH(CH₃)OH, CH₂F, CH(F)CH₃, I, C═C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C6-aryl, heteroaryl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-NH—C1-C6-haloalkyl, C1-C6-alkyl-S—C1-C6-alkyl, C1-C6-alkyl-SO₂—C1-C6-alkyl, and C1-C6-alkyl-C≡N, wherein C3-C7-heterocycloalkyl is optionally substituted with 1 or 2 amino groups
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen, O-C1-C6-haloalkyl and C≡N
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof.

2. The compound of Formula I according to claim 1 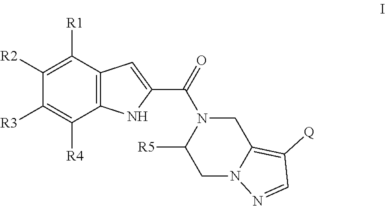
in which
R1, R2, R3 and R4 are for each position independently selected from the group comprising H, CF₂H, CF₃, CF₂CH₃, F, Cl, Br, CH₃, ethyl, iso-propyl, cyclopropyl, D, CH₂OH, CH(CH₃)OH, CH₂F, CH(F)CH₃, I, C═C, C≡C, C≡N, C(CH₃)₂OH, SCH₃, OH, and OCH₃
R5 is H or methyl
Q is selected from the group comprising C1-C6-alkyl, C3-C6-cycloalkyl, C3-C7-heterocycloalkyl, SO₂—C1-C6-alkyl, SO₂—C3-C7-cycloalkyl, SO₂—C3-C7-heterocycloalkyl, aryl, heteroaryl, N(R^{a})(R^{b}), C(═O)N(R^{a})(R^{b}), O(R^{a}) and SO₂N(R^{a})(R^{b}) optionally substituted with 1, 2, 3 or 4 groups each independently selected from OH, halo, C≡N, C3-C7-cycloalkyl, C1-C6-alkoxy, C3-C7-heterocycloalkyl, C1-C6-alkyl, C1-C6-haloalkyl, C1-C6-carboxyalkyl, heteroaryl, C6-aryl, NH-C6-aryl, C1-C6-hydroxyalkyl, C1-C6-alkyl-O—C1-C6-alkyl, C1-C6-alkyl-S—C1-C6-alkyl, C1-C6-alkyl-SO₂—C1-C6-alkyl, C1-C6-alkyl-C≡N, and N(C1-C6-carboxyalkyl)(C1-C6-alkyl), wherein C3-C7-heterocycloalkyl, C1-C6-carboxyalkyl, heteroaryl, C6-aryl and NH-C6-aryl are optionally substituted with 1 or 2 groups each independently selected from carboxy and halo
R^{a} and R^{b} are independently selected from the group comprising H, C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring or hetero-spirocyclic system consisting of 2 or 3 C3-C7 rings, optionally substituted with 1, 2, or 3 groups selected from OH, halogen and C≡N
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof.

3. The compound of Formula I according to claim 1, wherein aryl is C6-aryl, and/or heteroaryl is C1-C9-hereroaryl and wherein heteroaryl and heterocycloalkyl each has 1 to 4 heteroatoms each independently selected from N, O and S,
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof.

4. The compound of Formula I according to claim 1,
or a pharmaceutically acceptable salt thereof or a solvate or a hydrate of a compound of Formula I or the pharmaceutically acceptable salt thereof or a prodrug of a compound of Formula I or a pharmaceutically acceptable salt or a solvate or a hydrate thereof,
wherein the prodrug is selected from the group comprising esters, carbonates, acetyloxy derivatives, amino acid derivatives and phosphoramidate derivatives.

5. The compound of Formula I according to claim 1, wherein said compound is a compound of Formula II 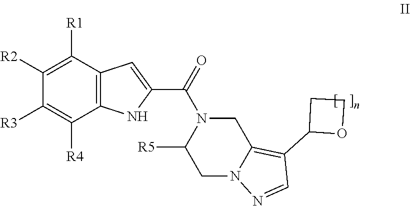
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
n is 1, 2 or 3
or a pharmaceutically acceptable salt thereof or a solvate or a hydrate of a compound of Formula II or the pharmaceutically acceptable salt thereof or a prodrug of a compound of Formula II or a pharmaceutically acceptable salt or a solvate or a hydrate thereof.

6. The compound of Formula I according claim 1, wherein said compound is a compound of Formula III 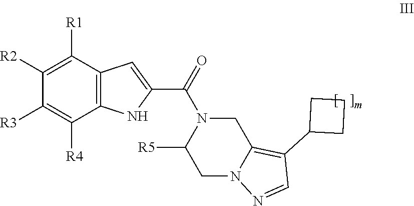
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
m is 0, 1, 2 or 3
or a pharmaceutically acceptable salt thereof or a solvate or a hydrate of a compound of Formula III or the pharmaceutically acceptable salt thereof or a prodrug of a compound of Formula III or a pharmaceutically acceptable salt or a solvate or a hydrate thereof.

7. The compound of Formula I according to claim 1, wherein said compound is a compound of Formula IV 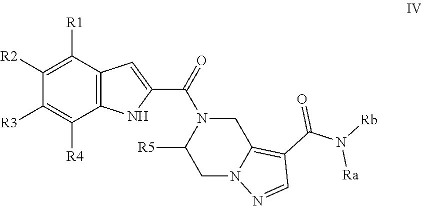
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring, optionally substituted with 1, 2, or 3 groups selected from OH, halogen and C≡N
or a pharmaceutically acceptable salt thereof or a solvate or a hydrate of a compound of Formula IV or the pharmaceutically acceptable salt thereof or a prodrug of a compound of Formula IV or a pharmaceutically acceptable salt or a solvate or a hydrate thereof.

8. The compound of Formula I according to claim 1, wherein said compound is a compound of Formula V 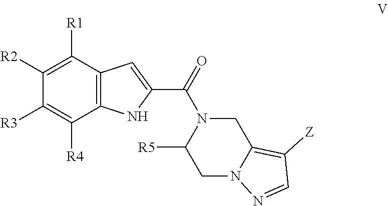
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
Z is selected from C6-C12-aryl and C1-C9-heteroaryl, optionally substituted with 1, 2, 3, or 4 groups each independently selected from —OH, halo, C1-C6-alkyl, C3-C7-cycloalkyl, C1-C6-haloalkyl, C1-C6-alkoxy, C1-C6-hydroxyalkyl, and C≡N
or a pharmaceutically acceptable salt thereof or a solvate or a hydrate of a compound of Formula V or the pharmaceutically acceptable salt thereof or a prodrug of a compound of Formula V or a pharmaceutically acceptable salt or a solvate or a hydrate thereof.

9. The compound of Formula I according to claim 1, wherein said compound is a compound of Formula VI 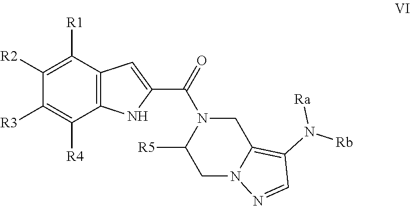
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring, optionally substituted with 1, 2, or 3 groups selected from OH, halogen and C≡N
or a pharmaceutically acceptable salt thereof or a solvate or a hydrate of a compound of Formula VI or the pharmaceutically acceptable salt thereof or a prodrug of a compound of Formula VI or a pharmaceutically acceptable salt or a solvate or a hydrate thereof.

10. The compound of Formula I according claim 1, wherein said compound is a compound of Formula VII 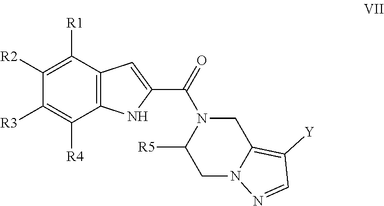
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
Y is oxooxadiazabicyclo[3.3.1]nonanyl substituted by C1-C6-carboxyalkyl; or oxopyrrolidinyl, said oxopyrrolidinyl optionally being once substituted by N(C1-C6-carboxyalkyl)(C1-C6-alkyl), carboxyphenyl, carboxypyridinyl, carboxyphenylamino, halocarboxyphenyl or carboxypyrrolidinyl; or twice substituted by carboxypyrrolidinyl and C1-C6-alkyl
or a pharmaceutically acceptable salt thereof or a solvate or a hydrate of a compound of Formula VII or the pharmaceutically acceptable salt thereof or a prodrug of a compound of Formula VII or a pharmaceutically acceptable salt or a solvate or a hydrate thereof.

11. The compound of Formula I according to claim 1, wherein said compound is a compound of Formula VIII 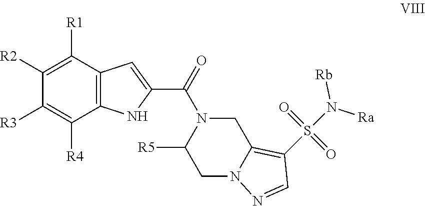
in which
R1, R2, R3 and R4 are for each position independently selected from the group comprising H, CF₂H, CF₃, CF₂CH₃, F, Cl, Br, CH₃, ethyl, iso-propyl, cyclopropyl, D, and CH₂OH
R5 is selected from H and methyl
R^{a} and R^{b} are independently selected from the group comprising C1-C6-alkyl, C1-C6-haloalkyl, C3-C6-cycloalkyl, C3-C7-heterocycloalkyl, C2-C6-hydroxyalkyl, and C2-C6-alkyl-O—C1-C6-alkyl, optionally substituted with 1, 2, or 3 groups each independently selected from OH, halo, C3-C7-heterocycloalkyl, C1-C6-alkyl, C1-C6-haloalkyl, C1-C6-hydroxyalkyl, C1-C6-alkyl-O—C1-C6-alkyl, C1-C6-alkyl-O—C1-C6-haloalkyl, C1-C6-alkyl-S—C1-C6-alkyl, C1-C6-alkyl-SO₂—C1-C6-alkyl, and C1-C6-alkyl-C≡N
R^{a} and R^{b} are optionally connected to form a C3-C7-heterocycloalkyl ring, optionally substituted with 1, 2, or 3 groups selected from OH, halogen and C≡N
or a pharmaceutically acceptable salt thereof or a solvate or a hydrate of a compound of Formula VIII or the pharmaceutically acceptable salt thereof or a prodrug of a compound of Formula VIII or a pharmaceutically acceptable salt or a solvate or a hydrate thereof.

12. The compound according claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof for use in the prevention or treatment of an HBV infection in subject.

13. A pharmaceutical composition comprising a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof, together with a pharmaceutically acceptable carrier.

14. A method of treating an HBV infection in an individual in need thereof, comprising administering to the individual a therapeutically effective amount of a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.

15. A method for preparation of a compound according to claim 1, comprising reacting a compound of Formula IX 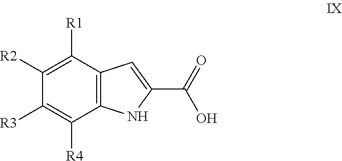
in which R1, R2, R3 and R4 are as defined in claim 1, with a compound of Formula X 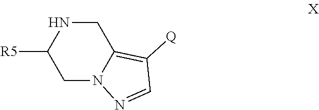
in which R5 and Q are as defined in claim 1.

16. The compound according to claim 5, wherein R1, R2, R3 and R4 are for each position independently selected from H, CF₂H, CF₃, CF₂CH₃, F, Cl, Br, CH₃, ethyl, iso-propyl, cyclopropyl, D, and CH₂OH.

17. The compound according to claim 6, wherein R1, R2, R3 and R4 are for each position independently selected from H, CF₂H, CF₃, CF₂CH₃, F, Cl, Br, CH₃, ethyl, iso-propyl, cyclopropyl, D, and CH₂OH.

18. The compound according to claim 7, wherein R1, R2, R3 and R4 are for each position independently selected from H, CF₂H, CF₃, CF₂CH₃, F, Cl, Br, CH₃, ethyl, iso-propyl, cyclopropyl, D, and CH₂OH.

19. The compound according to claim 8, wherein R1, R2, R3 and R4 are for each position independently selected from H, CF₂H, CF₃, CF₂CH₃, F, Cl, Br, CH₃, ethyl, iso-propyl, cyclopropyl, D, and CH₂OH.

20. The compound according to claim 9, wherein R1, R2, R3 and R4 are for each position independently selected from H, CF₂H, CF₃, CF₂CH₃, F, Cl, Br, CH₃, ethyl, iso-propyl, cyclopropyl, D, and CH₂OH.
